(19) United States
(12) Patent Application Publication
(10) Pub. No.: US 2025/0004367 A1
(43) Pub. Date: Jan. 2, 2025

(54) RESIST UNDERLAYER FILM-FORMING COMPOSITION

(71) Applicant: NISSAN CHEMICAL CORPORATION, Tokyo (JP)

(72) Inventors: Hikaru TOKUNAGA, Toyama-shi (JP); Makoto NAKAJIMA, Toyama-shi (JP); Hirokazu NISHIMAKI, Toyama-shi (JP)

(73) Assignee: NISSAN CHEMICAL CORPORATION, Tokyo (JP)

(21) Appl. No.: 18/695,265

(22) PCT Filed: Sep. 13, 2022
(86) PCT No.: PCT/JP2022/034228

§ 371 Date: Mar. 25, 2024

(30) Foreign Application Priority Data
Sep. 24, 2021 (JP) 2021-155967

(51) Int. Cl. G03F 7/004; G03F 7/025; G03F 7/027; G03F 7/038; G03F 7/16; G03F 7/20

(52) U.S. Cl. CPC G03F 7/0045; G03F 7/0048; G03F 7/025; G03F 7/0275; G03F 7/0385; G03F 7/168; G03F 7/2004

(57) ABSTRACT

A resist underlayer film-forming composition has excellent embedding and flattening properties for stepped substrates, excellent storage stability, low film-curing start temperature and small amount of sublimate generation, and can form a film that does not dissolve in photoresist solvents. The composition contains: a thermal acid generator; a Novolac resin polymer in which (i) a unit structure having an aromatic ring optionally having a substituent and (ii) a unit structure containing an aromatic cyclic organic group optionally having a substituent, a non-aromatic monocyclic organic group optionally having a substituent, or a 4—to 25-membered bicyclic, tricyclic, or tetracyclic organic group containing at least one non-aromatic monocyclic ring and optionally having a substituent are bonded via a covalent bond between carbon atoms on the aromatic ring of (i) and the non-aromatic monocyclic ring of (ii); and a solvent.

### TECHNICAL FIELD

The present invention relates to a resist underlayer film-forming composition suited for lithographic processing of semiconductor substrates, a resist underlayer film-forming composition resistant to degeneration, a resist underlayer film obtained from the resist underlayer film-forming composition, and a method for manufacturing a semiconductor device using the composition.

### BACKGROUND ART

In the recent lithographic process of semiconductor device manufacturing, semiconductor processing materials including resist underlayer films are required to have excellent material properties and also to be further enhanced in stability of resist underlayer film-forming compositions.

When, for example, a workpiece substrate as a base has steps or when a wafer has a densely patterned region and a pattern-free region, such irregularities need to be covered with a flat surface of an underlayer film. Resins suited for this purpose have been proposed (Patent Literature 1).

To form thermoset films, resist underlayer film-forming compositions contain a polymer resin as a main component as well asn a crosslinking compound (a crosslinking agent) and a catalyst (a crosslinking catalyst) for promoting the crosslinking reaction. These components have not been fully studied from the point of view of flattening of the surface with an underlayer film.

A new problem that has been encountered recently is that crosslinking agents and polymer resins that are the main components of resist underlayer films are degenerated by crosslinking catalysts and solvents present in the resist underlayer film-forming compositions. The resistance to such degeneration is also desired.

Patent Literature 2 discloses an ionic thermal acid generator of the formula (A⁻)(BH)⁺ in which A⁻ is an anion of an organic or inorganic acid having a pKa of not more than 3; and (BH)'⁰ is a nitrogen-containing base B in a monoprotonated form, which has a pKa between 0 and 5.0 and a boiling point of less than 170° C. Specifically, combinations of perfluorobutane sulfonate with ammonium, pyridinium, 3-fluoropyridinium, or pyridazinium are described.

Patent Literature 3 discloses a thermal acid generator of the formula X-YH+where X is an anionic component and Y is a substituted pyridine. Specifically, combinations of methylbenzene sulfonate with fluoropyridinium or trifluoromethylpyridinium are described.

Patent Literature 4 discloses a thermal acid generator including a hydroxy-free sulfonic acid component and a pyridinium component having a ring substituent. Specifically, combinations of methylbenzene sulfonate with methylpyridinium, methoxypyridinium, or trimethylpyridinium are described.

Patent Literature 5 discloses a thermal acid generator including para-toluenesulfonic acid triethylamine salt, para-toluenesulfonic acid ammonia salt, mesitylenesulfonic acid ammonia salt, dodecylbenzenesulfonic acid ammonia salt, or para-toluenesulfonic acid dimethylamine salt.

Patent Literature 6 discloses a thermal acid generator containing a sulfonic acid and NH₄ or a primary, secondary, tertiary, or quaternary ammonium ion.

### CITATION LIST

### Patent Literature

Patent Literature 1: WO 2014/024836
Patent Literature 2: JP 6334900 B
Patent Literature 3: JP 2019-56903 A
Patent Literature 4: JP 6453378 B
Patent Literature 5: JP 4945091 B
Patent Literature 6: JP 6256719 B

### SUMMARY OF INVENTION

### Technical Problem

However, the thermal acid generators disclosed in the related art documents are aimed at improving the shape of resists, and the documents are completely silent with respect to the performance in gap-filling and flattening on non-planar substrates. Some inventions disclose a relationship between storage stability and sublimates, but do not specifically evaluate or mention the degeneration of polymers associated with the thermal acid generators, and do not study the performance in gap-filling and flattening on non-planar substrates. In recent years, it has become clear that the thermal acid generators described above degenerate polymers unless an appropriate amine component is selected. Thus, such thermal acid generators are desired that do not degenerate polymers and satisfy both gap-filling properties and flattening properties on non-planar substrates.

Objects of the present invention are therefore to provide a resist underlayer film-forming composition that has excellent performance in gap-filling and flattening on a non-planar substrate, has high storage stability of a polymer that is the main component of a resist underlayer film, and can form a film that is not dissolved by a photoresist solvent; and to provide a method for manufacturing a semiconductor device using the composition.

### Solution to Problem

Aspects of the present invention include the following.

[1]

A resist underlayer film-forming composition comprising:
a thermal acid generator represented by formula (I) below;
a polymer (G) which is a novolac resin comprising:
   (i) a unit structure having an optionally substituted aromatic ring, and
   (ii) a unit structure containing:
an optionally substituted aromatic cyclic organic group; an optionally substituted non-aromatic monocyclic organic group; or an optionally substituted, 4—to 25-membered bicyclic, tricyclic, or tetracyclic organic group containing at least one non-aromatic monocyclic ring;
the unit structure (i) and the unit structure (ii) being bonded to each other via a covalent bond between a carbon atom on the aromatic ring of the unit structure (i) and a carbon atom on a non-aromatic monocyclic ring of the unit structure (ii); and
   a solvent, [Chem. 1]

(A—SO₃)⁻(BH)⁺ (I)

[in formula (I),

A is an optionally substituted, linear, branched, or cyclic, saturated or unsaturated, aliphatic hydrocarbon group; an optionally substituted aryl group; or an optionally substituted heteroaryl group; and

B is a base having a pKa of 6.5 or more].

[2]

The resist underlayer film-forming composition according to [1], wherein

the polymer (G) includes a structure represented by formula (X) below:

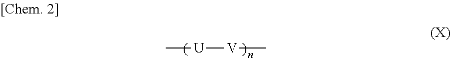

[in formula (X), n indicates a number of composite unit structures U-V,

the unit structure U comprises one, or two or more types of unit structures having an optionally substituted aromatic ring, and is optionally such that:
the optionally substituted aromatic ring is substituted with a substituent that contains a heteroatom;
the unit structure contains aromatic rings connected to one another by a linking group that contains a heteroatom; or
the aromatic ring is an aromatic heterocyclic ring or is an aromatic ring forming a condensed ring with one or more heterocyclic rings; and

the unit structure V comprises one, or two or more types of unit structures containing at least one structure selected from:

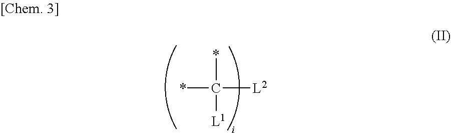

(in formula (II),

* indicates a bonding site to the unit structure U,

### L¹ is:

an optionally substituted, saturated or unsaturated, linear, branched, or cyclic, aliphatic hydrocarbon group optionally containing a heteroatom;
an optionally substituted aromatic hydrocarbon group optionally containing a heteroatom;
a group comprising a combination or a condensation product of the above groups; or
a hydrogen atom,

### L² is:

an optionally substituted, saturated or unsaturated, linear, branched, or cyclic, aliphatic hydrocarbon group optionally containing a heteroatom;
an optionally substituted aromatic hydrocarbon group optionally containing a heteroatom;
a group comprising a combination or a condensation product of the above groups;
a direct bond; or
a hydrogen atom,

L¹ and L² are optionally condensed to each other or are optionally bonded to each other via or without a heteroatom to form a ring,

i is an integer of 1 or more and 8 or less,

when i is 2 or more, L² is not a hydrogen atom, and

when i is 2 or more, the two to i quantity of carbon atoms are optionally connected to one another by the aliphatic hydrocarbon group or the aromatic hydrocarbon group L¹);

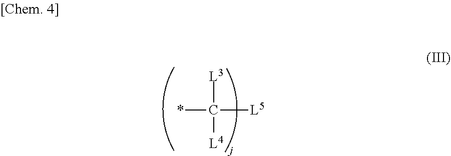

(in formula (III),

* indicates a bonding site to the unit structure U,

### L³ is:

an optionally substituted, saturated or unsaturated, linear, branched, or cyclic, aliphatic hydrocarbon group optionally containing a heteroatom;
an optionally substituted aromatic hydrocarbon group optionally containing a heteroatom;
a group comprising a combination or a condensation product of the above groups;
a hydroxy group; or
a hydrogen atom;

### L⁴ is:

an optionally substituted, saturated or unsaturated, linear, branched, or cyclic, aliphatic hydrocarbon group optionally containing a heteroatom;
an optionally substituted aromatic hydrocarbon group optionally containing a heteroatom;
a group comprising a combination or a condensation product of the above groups;
a hydroxy group; or
a hydrogen atom,

### L⁵ is:

an optionally substituted, saturated or unsaturated, linear, branched, or cyclic, aliphatic hydrocarbon group optionally containing a heteroatom;
an optionally substituted aromatic hydrocarbon group optionally containing a heteroatom;
a group comprising a combination or a condensation product of the above groups; or
a direct bond;

j is an integer of 2 or more and 4 or less; and

L³, L⁴, and L⁵ are optionally condensed to one another or are optionally bonded to one another via or without a heteroatom to form a ring); and

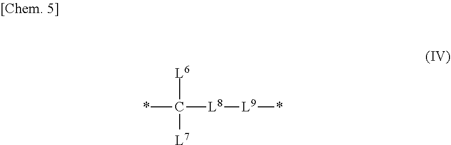

(in formula (IV),

* indicates a bonding site to the unit structure U;

### L⁶ is:

an optionally substituted, saturated or unsaturated, linear, branched, or cyclic, aliphatic hydrocarbon group optionally containing a heteroatom;
an optionally substituted aromatic hydrocarbon group optionally containing a heteroatom;
a group comprising a combination or a condensation product of the above groups; or
a hydrogen atom;

### L⁷ is:

an optionally substituted, saturated or unsaturated, linear, branched, or cyclic, aliphatic hydrocarbon group optionally containing a heteroatom;
an optionally substituted aromatic hydrocarbon group optionally containing a heteroatom;
a group comprising a combination or a condensation product of the above groups; or
a hydrogen atom;

L⁶, L⁷, and L⁹ are optionally condensed to one another or are optionally bonded to one another via or without a heteroatom to form a ring;

### L⁸ is:

a direct bond;
an optionally substituted, saturated or unsaturated, linear or branched hydrocarbon group; or
an aromatic ring optionally containing a heteroatom; and

### L⁹ is:

an aromatic ring optionally containing a heteroatom)].
   [3]

The resist underlayer film-forming composition according to [1], wherein the polymer (G) comprises:
a structural unit derived from (D) an aromatic compound having at least one hydroxy group or amino group, or a compound in which two or more optionally substituted aromatic rings are connected to one another via at least one direct bond, —O—, —S—, —C(═O)—, —SO₂—, —NR—, in which R denotes a hydrogen atom or a hydrocarbon group, or —(CR₁₁₁R₁₁₂)ₙ—, in which R₁₁₁ and R₁₁₂ each denote a hydrogen atom, an optionally substituted, C1-C10 linear or cyclic alkyl group, or an aromatic ring, n is 1 to 10, and R₁₁₁ and R₁₁₂ are optionally bonded to each other to form a ring, and (E) an optionally substituted aldehyde compound or aldehyde equivalent.
   [4]

The resist underlayer film-forming composition according to any one of [1] to [3], wherein
B in formula (I) is R R2R3N,
R¹ and R² each independently denote a hydrogen atom or an optionally substituted, linear or branched, saturated or unsaturated, aliphatic hydrocarbon group,
R¹ and R² optionally form a ring together via or without a heteroatom, or optionally form a ring together via an aromatic ring,
R³ denotes a hydrogen atom, an optionally substituted aromatic group, or an optionally substituted, linear or branched, saturated or unsaturated, aliphatic hydrocarbon group, and
when R¹ and R² do not form a ring, R³ is a hydrogen atom or an optionally substituted aromatic group.
   [5]

The resist underlayer film-forming composition according to any one of [1] to [3], wherein

B in formula (I) is a base represented by:

R¹R²R³N [Chem. 6]

[wherein
R¹ and R² each independently denote an optionally substituted, linear or branched, saturated or unsaturated, aliphatic hydrocarbon group, and
R³ denotes a hydrogen atom or an optionally substituted aromatic group] or a base represented by formula (II) below:

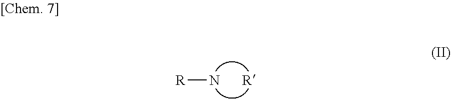

[in formula (II),
R is a hydrogen atom, a nitro group, a cyano group, an amino group, a carboxyl group, a hydroxy group, an amide group, an aldehyde group, a (meth)acryloyl group, a halogen atom, a C1-C10 alkoxy group, a C1-C10 alkyl group, a C2-C10 alkenyl group, a C2-C10 alkynyl group, a C1-C10 hydroxyalkyl group, a C6-C40 aryl group, an ether bond-containing organic group, a ketone bond-containing organic group, an ester bond-containing organic group, or a combination thereof, and
R′ is a ring through an aromatic ring or is:

—(R^{a})ₙ—X—(R^{b})ₘ— [Chem. 8]

wherein R^{a} and R^{b} each independently denote an optionally substituted alkyl,
X is O, S, SO₂, CO, CONH, COO, or NH, and
n and m are each independently 2, 3, 4, 5, or 6].
   [6]

The resist underlayer film-forming composition according to [5], wherein the base is such that:
R³ in the formula denotes an optionally substituted phenyl, naphthyl, anthracenyl, pyrenyl, or phenanthrenyl group, or
R in formula (II) is a hydrogen atom, a methyl group, an ethyl group, an isobutyl group, an allyl group, or a cyanomethyl group, and
R′ in formula (II) is represented by:

—(OH₂)ₙ—O—(CH₂)ₘ— [Chem. 9]

[7]

The resist underlayer film-forming composition according to any one of [1] to [3], wherein B in formula (I) is N-methylmorpholine, N-isobutylmorpholine, N-allylmorpholine, or N,N-diethylaniline.

[8]

The resist underlayer film-forming composition according to any one of [1] to [3], wherein A in formula (I) is a methyl group, a fluoromethyl group, a naphthyl group, a norbornanylmethyl group, a dimethylphenyl group, or a tolyl group.

[9]

The resist underlayer film-forming composition according to [3], wherein the compound (D) is selected from the following group:

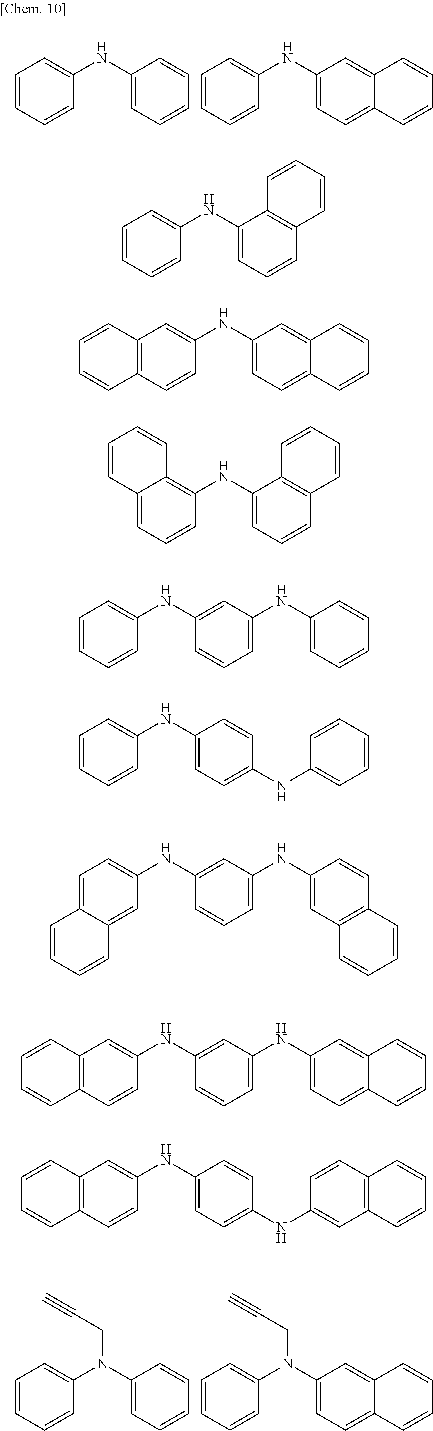

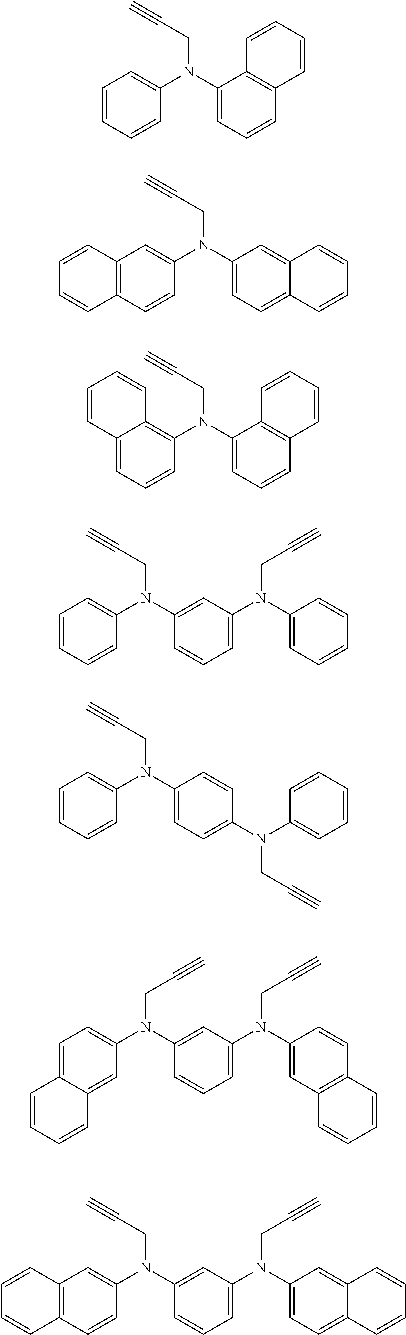

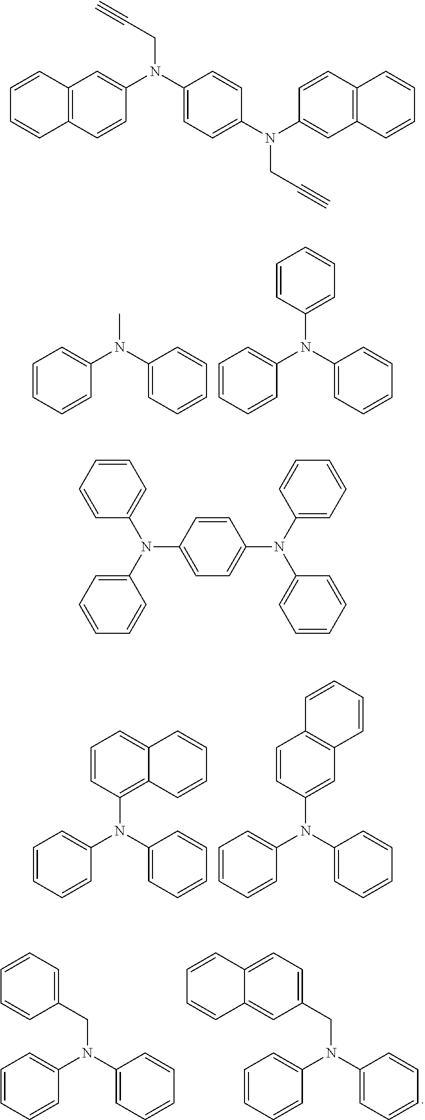

The resist underlayer film-forming composition according to [3], wherein the compound (D) is selected from the following group.

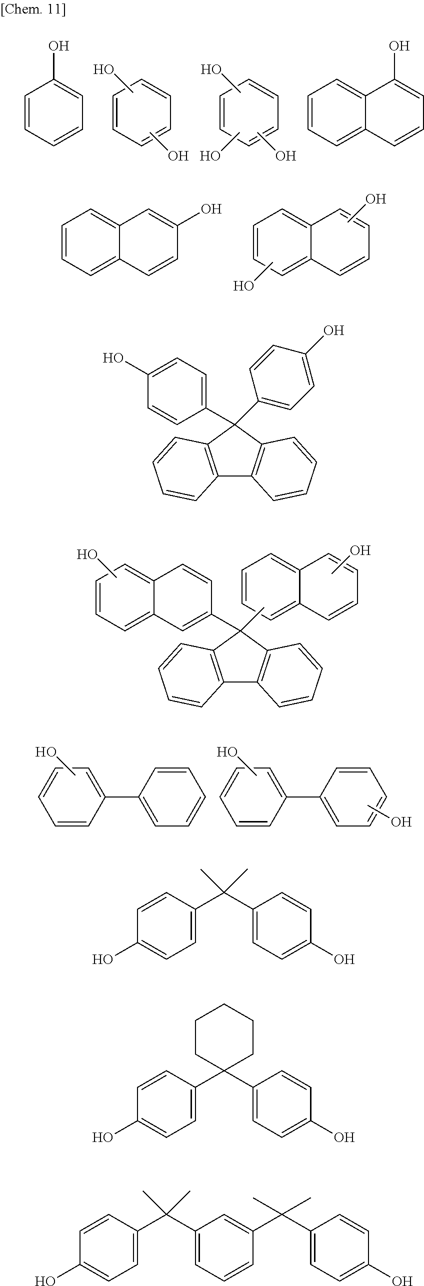

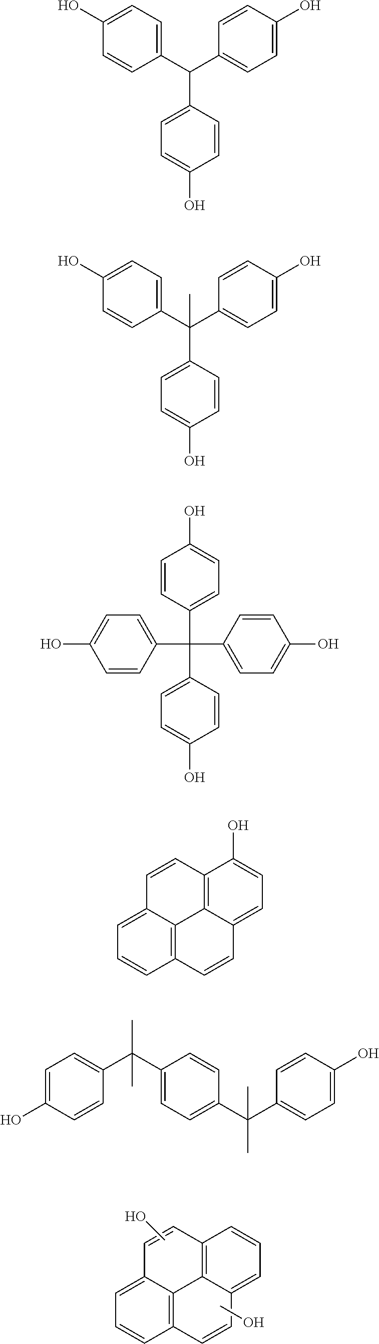

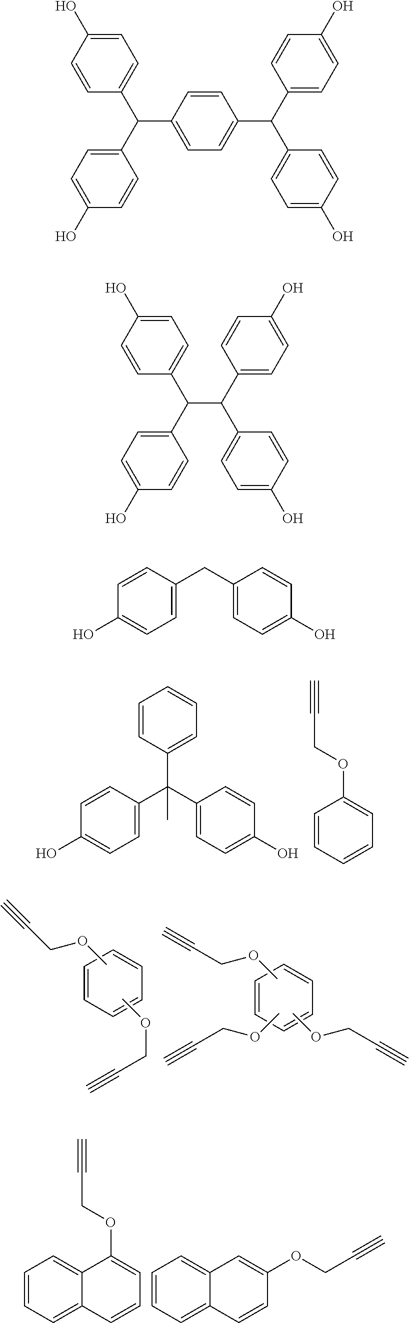

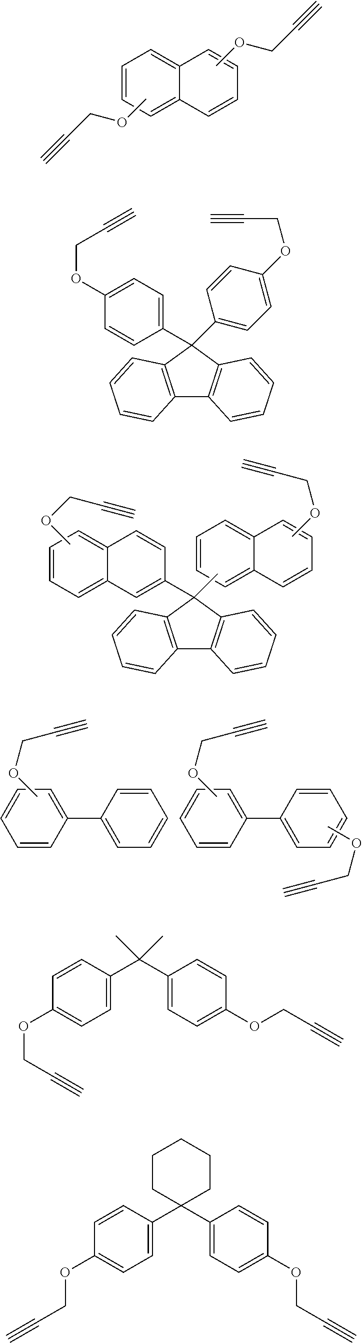

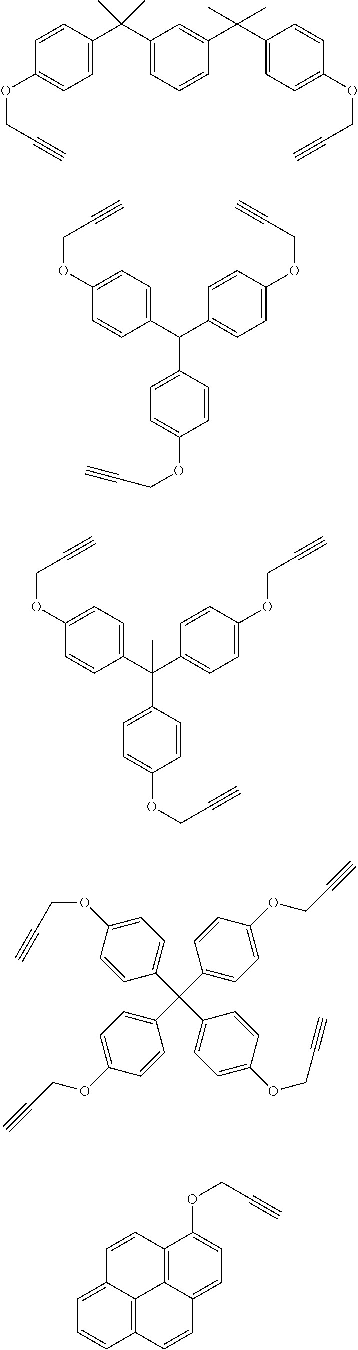

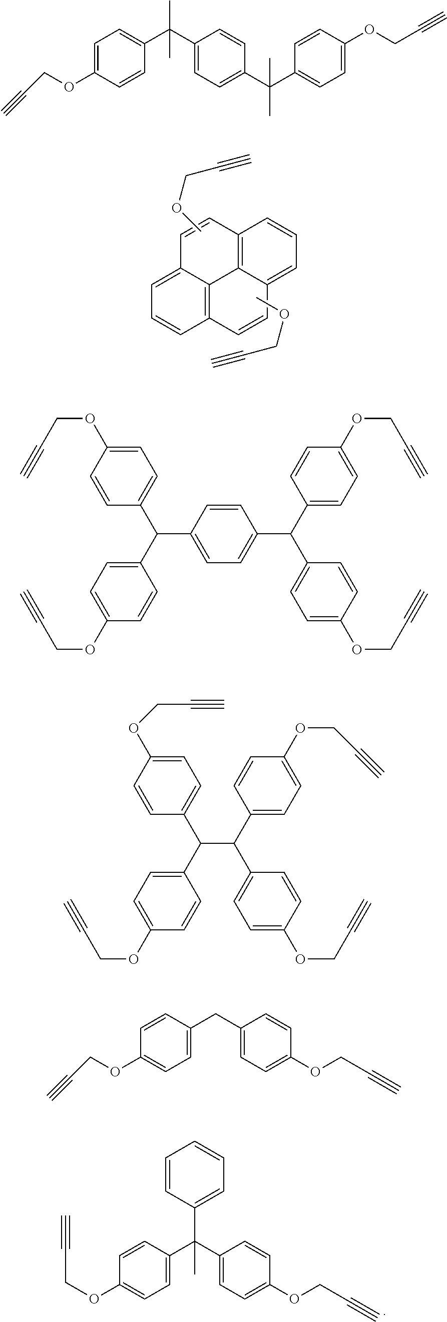

The resist underlayer film-forming composition according to [3], wherein the aldehyde compound or the aldehyde equivalent (E) is selected from the following group:

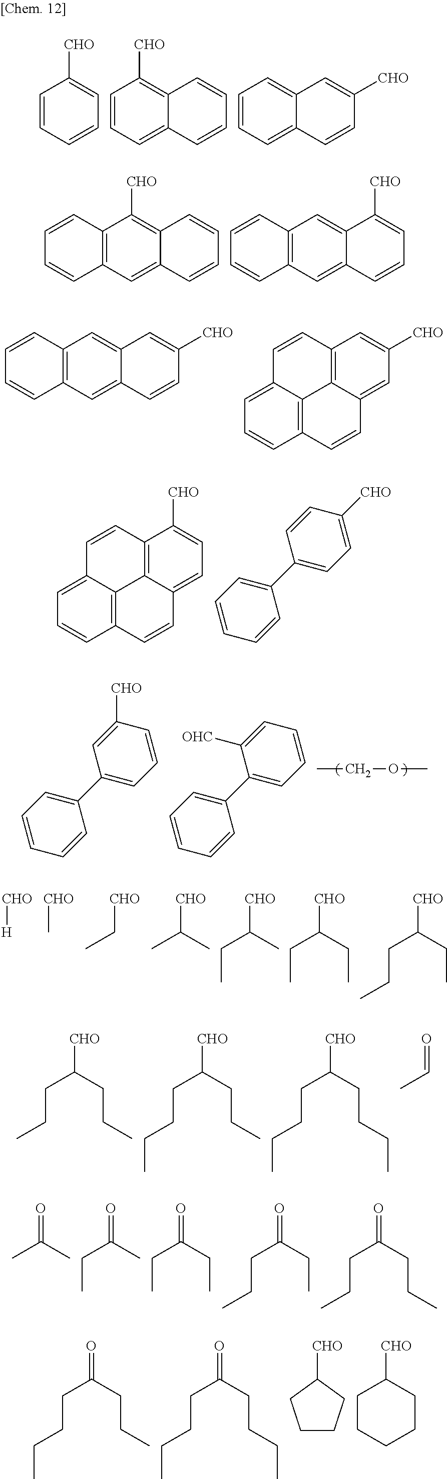

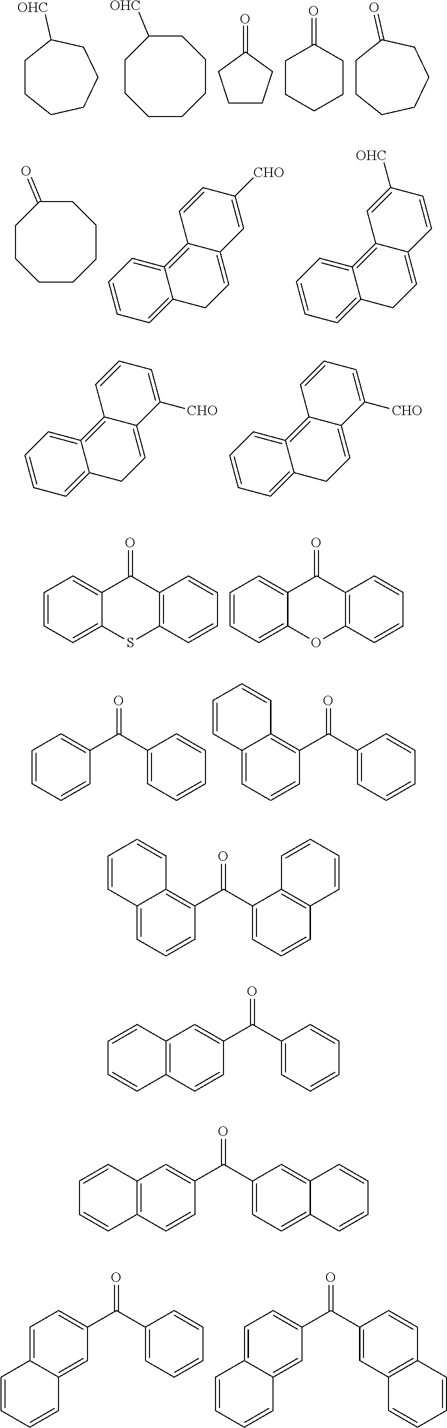

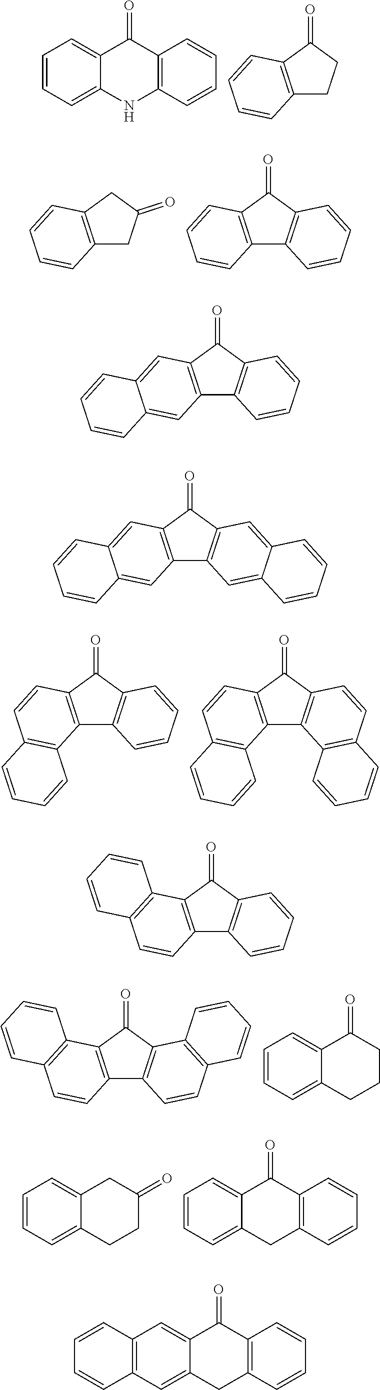

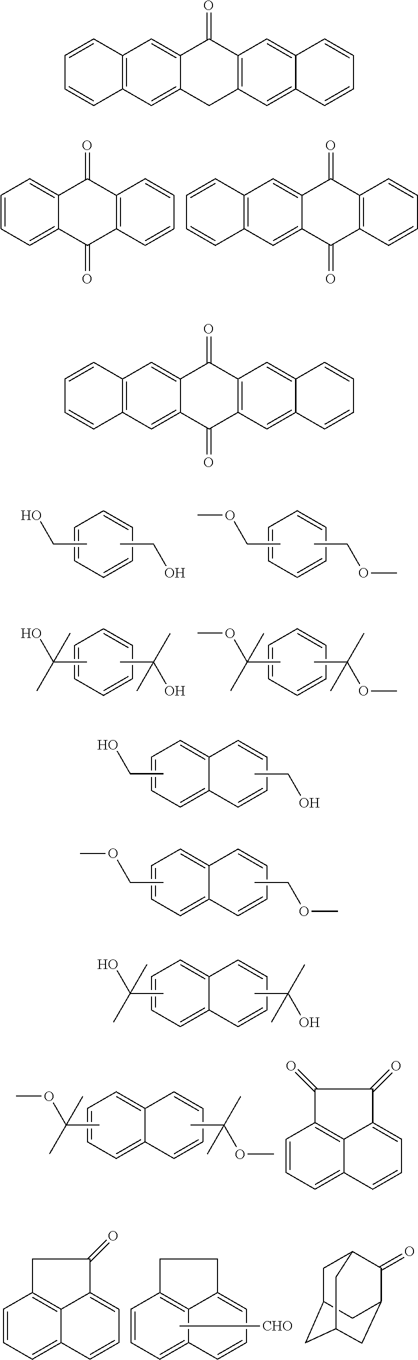

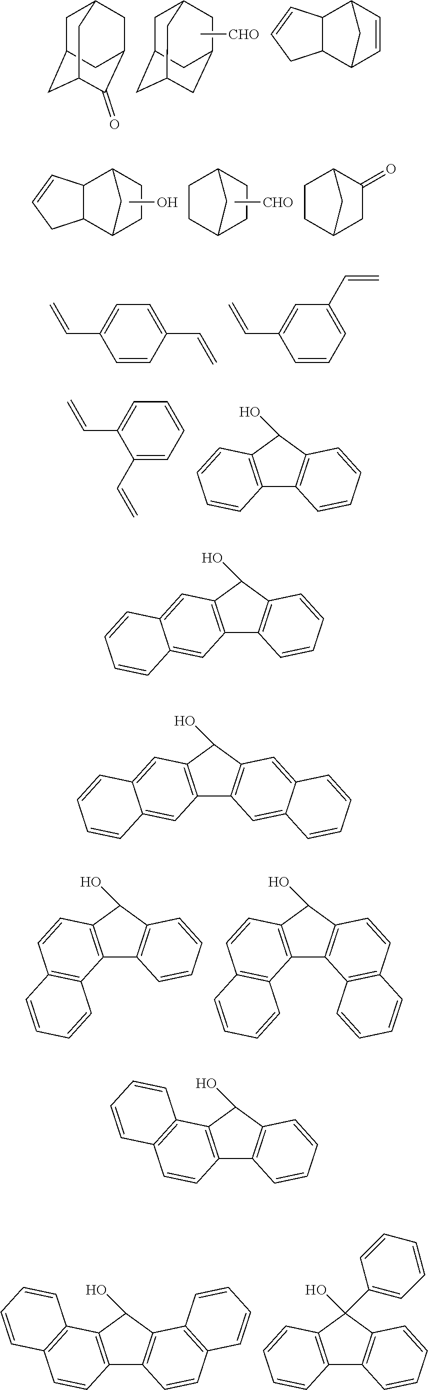

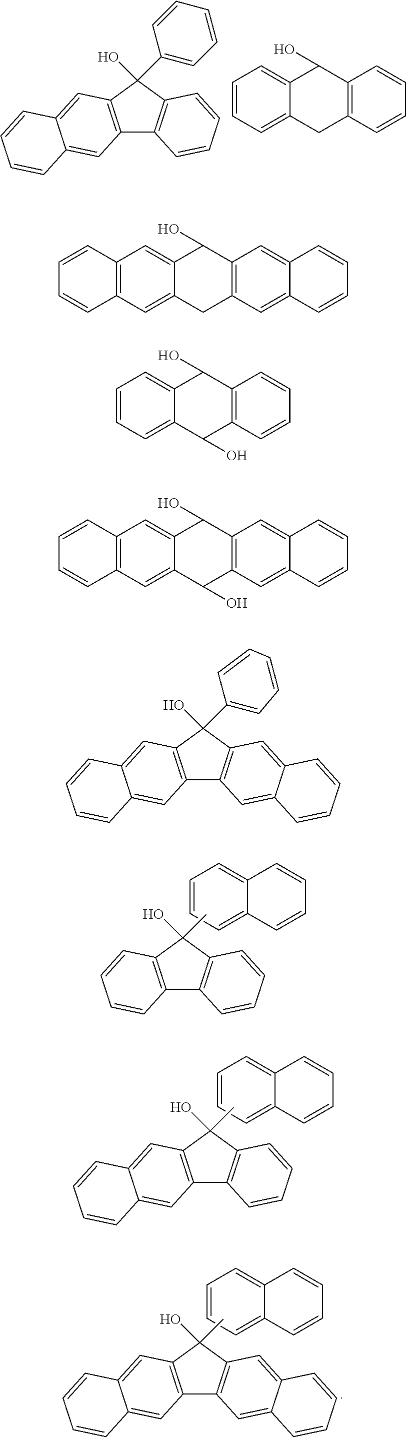

The resist underlayer film-forming composition according to any one of [1] to [3], further comprising a crosslinking agent.

The resist underlayer film-forming composition according to [12], wherein the crosslinking agent is an aminoplast crosslinking agent or a phenoplasts crosslinking agent.

the resist underlayer film-forming composition according to [13], wherein the aminoplast crosslinking agent is a highly alkylated, alkoxylated, or alkoxyalkylated melamine, benzoguanamine, glycoluryl, or urea, or a polymer thereof.

The resist underlayer film-forming composition according to [12], wherein the aminoplast crosslinking agent is a highly alkylated, alkoxylated, or alkoxyalkylated aromatic, or a polymer thereof.

[16]

The resist underlayer film-forming composition according to any one of [1] to [3], further comprising a compound having an alcoholic hydroxy group or a compound having a group capable of forming an alcoholic hydroxy group.

[17]

The resist underlayer film-forming composition according to [16], wherein the compound having an alcoholic hydroxy group or the compound having a group capable of forming an alcoholic hydroxy group is a propylene glycol solvent, a cycloaliphatic ketone solvent, an oxyisobutyric acid ester solvent, or a butylene glycol solvent.

[18]

The resist underlayer film-forming composition according to [16], wherein the compound having an alcoholic hydroxy group or the compound having a group capable of forming an alcoholic hydroxy group is propylene glycol monomethyl ether, propylene glycol monomethyl acetate, cyclohexanone, or methyl 2-hydroxy-2-methylpropionate.

[19]

The resist underlayer film-forming composition according to any one of [1] to [3], further comprising a surfactant.

[20]

A resist underlayer film, which is a baked product of a coating film of the resist underlayer film-forming composition according to any one of [1] to [3] on a semiconductor substrate.

[21]

A method for forming a resist pattern used in semiconductor manufacturing, the method comprising the step of applying the resist underlayer film-forming composition according to any one of [1] to [3] onto a semiconductor substrate, and baking the resist underlayer film-forming composition to form a resist underlayer film.

[22]

A method for manufacturing a semiconductor device, comprising the steps of:
forming on a semiconductor substrate a resist underlayer film from the resist underlayer film-forming composition according to any one of [1] to [3]; underlayer film-forming composition according to any one of claims 1 to 3;
forming a resist film on the resist underlayer film;
forming a resist pattern by irradiation with a light or electron beam followed by development;
etching the resist underlayer film through the resist pattern; and
processing the semiconductor substrate through the patterned resist underlayer film.
   [23]

A method for manufacturing a semiconductor device, comprising the steps of
forming on a semiconductor substrate a resist underlayer film from the resist underlayer film-forming composition according to any one of [1] to [3];
forming a hard mask on the resist underlayer film;
forming a resist film on the hard mask;
forming a resist pattern by irradiation with a light or electron beam followed by development;
etching the hard mask through the resist pattern;
etching the resist underlayer film through the patterned hard mask; and
processing the semiconductor substrate through the patterned resist underlayer film.
   [24]

A method for manufacturing a semiconductor device, comprising the steps of
forming on a semiconductor substrate a resist underlayer film from the resist underlayer film-forming composition according to any one of [1] to [3];
forming a hard mask on the resist underlayer film;
forming a resist film on the hard mask;
forming a resist pattern by irradiation with a light or electron beam followed by development;
etching the hard mask through the resist pattern;
etching the resist underlayer film through the patterned hard mask;
removing the hard mask; and
processing the semiconductor substrate through the patterned resist underlayer film.
   [25]

A method for manufacturing a semiconductor device, comprising the steps of
forming on a semiconductor substrate a resist underlayer film from the resist underlayer film-forming composition according to any one of [1] to [3];
forming a hard mask on the resist underlayer film;
forming a resist film on the hard mask;
forming a resist pattern by irradiation with a light or electron beam followed by development;
etching the hard mask through the resist pattern formed;
etching the resist underlayer film through the patterned hard mask;
removing the hard mask;
forming a deposited film or spacer on the hard mask-removed, resist underlayer film;
processing the deposited film or spacer by etching;
removing the patterned resist underlayer film to leave the patterned deposited film or spacer; and
processing the semiconductor substrate through the patterned deposited film or spacer.
   [26]

The manufacturing method according to [23], wherein the hard mask is formed by applying a composition containing an inorganic substance or by depositing an inorganic substance.

[27]

The manufacturing method according to [24], wherein the hard mask is formed by applying a composition containing an inorganic substance or by depositing an inorganic substance.

[28]

The manufacturing method according to [25], wherein the hard mask is formed by applying a composition containing an inorganic substance or by depositing an inorganic substance.

[29]

The manufacturing method according to [23], wherein the resist film is patterned by a nanoimprinting method or by using a self-assembled film.

[30]

The manufacturing method according to [24], wherein the resist film is patterned by a nanoimprinting method or by using a self-assembled film.

[31]

The manufacturing method according to [25], wherein the resist film is patterned by a nanoimprinting method or by using a self-assembled film.

[32]

The manufacturing method according to [23], wherein the hard mask is removed by etching or with an alkaline chemical solution.

[33]

The manufacturing method according to [24], wherein the hard mask is removed by etching or with an alkaline chemical solution.

[34]

The manufacturing method according to [25], wherein the hard mask is removed by etching or with an alkaline chemical solution.

### Advantageous Effects of Invention

According to the underlayer film-forming composition according to the present invention, a highly basic amine is adopted as an acid generator and thus the temperature for the generation of an acid is high to keep the fluidity of the polymer for a long period of time. Thus, the composition can provide cured films on various types of films, such as SiO₂, TiN, and SiN, with high flattening properties and high gap-filling properties. Furthermore, the acid generator does not adversely affect the polymer and the storage stability of the resist underlayer film main component can be ensured. Thus, the composition is free from coloration and can form films that are not dissolved in photoresist solvents. The present invention further provides a resist underlayer film obtained from the resist underlayer film-forming composition, and a method for manufacturing a semiconductor device using the composition.

### [1. Thermal Acid Generator]

(1-1)

A thermal acid generator in the present invention is represented by formula (I) below:

[Chem. 13]

(A—SO₃)⁻(BH)⁺ (I)

[in formula (I),

A is an optionally substituted, linear, branched, or cyclic, saturated or unsaturated, aliphatic hydrocarbon group, an optionally substituted aryl group, or an optionally substituted heteroaryl group, and

B is a base having a pKa of 6.5 or more].

Here, pKa (acid dissociation constant) is an indicator that quantitatively represents the acid strength of a compound having a protic functional group. The acid strength is expressed by the negative common logarithm of the equilibrium constant Ka of a dissociation reaction, in which the acid releases a proton. pKa can be calculated by a known method, for example, a titration method.

The letter B is preferably R¹R2R³N in which:
R¹ and R² each independently denote a hydrogen atom or an optionally substituted, linear or branched, saturated or unsaturated, aliphatic hydrocarbon group,
R¹ and R² optionally form a ring together via a heteroatom or without a heteroatom, or optionally form a ring together via an aromatic ring,
R³ denotes a hydrogen atom, an optionally substituted aromatic group, or an optionally substituted, linear or branched, saturated or unsaturated, aliphatic hydrocarbon group, and
when R¹ and R² do not form a ring, R³ is a hydrogen atom or an optionally substituted aromatic group.

Preferably, R¹ and R² each independently denote an optionally substituted, linear or branched, saturated or unsaturated, aliphatic hydrocarbon group, and R³ denotes a hydrogen atom or an optionally substituted aromatic group.

Preferably, R¹ and R² each independently denote an optionally substituted, linear or branched, saturated or unsaturated, aliphatic hydrocarbon group, and R³ denotes an optionally substituted phenyl, naphthyl, anthracenyl, pyrenyl, or phenanthrenyl group.

(1-2)

Preferably, B is represented by formula (II) below:

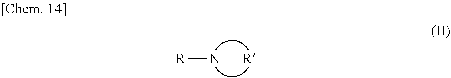

[in formula (II),

R is a hydrogen atom, a nitro group, a cyano group, an amino group, a carboxyl group, a halogen atom, a C1-C10 alkoxy group, a C1-C10 alkyl group, a C2-C10 alkenyl group, a C6-C40 aryl group, an ether bond-containing organic group, a ketone bond-containing organic group, an ester bond-containing organic group, or a combination thereof, and

R′ is:

—(R^{a})ₙ—X—(R^{b})ₘ— [Chem. 15]

wherein R^{a} and R^{b} each independently denote an optionally substituted alkyl,
X is O, S, SO₂, CO, CONH, COO, or NH, and
n and m are each independently 2, 3, 4, 5, or 6].

Preferably, R is a hydrogen atom, a methyl group, an ethyl group, an isobutyl group, an allyl group, or a cyanomethyl group,

R′ is:

—(CH₂)ₙ—O—(CH₂)ₘ— [Chem. 16]

, and
n and m are each independently 2, 3, 4, 5, or 6.
(1-3)
(1-3-1)

Examples of the “linear or branched, saturated aliphatic hydrocarbon group” in the definition of A in formula (I) or in the definition of R¹ and R² in R¹R2R³N include methyl group, ethyl group, n-propyl group, i-propyl group, n-butyl group, i-butyl group, s-butyl group, t-butyl group, n-pentyl group, 1-methyl-n-butyl group, 2-methyl-n-butyl group, 3-methyl-n-butyl group, 1,1-dimethyl-n-propyl group, 1,2-dimethyl-n-propyl group, 2,2-dimethyl-n-propyl group, 1-ethyl-n-propyl group, n-hexyl group, 1-methyl-n-pentyl group, 2-methyl-n-pentyl group, 3-methyl-n-pentyl group, 4-methyl-n-pentyl group, 1,1-dimethyl-n-butyl group, 1,2-dimethyl-n-butyl group, 1,3-dimethyl-n-butyl group, 2,2-dimethyl-n-butyl group, 2,3-dimethyl-n-butyl group, 3,3-dimethyl-n-butyl group, 1-ethyl-n-butyl group, 2-ethyl-n-butyl group, 1,1,2-trimethyl-n-propyl group, 1,2,2-trimethyl-n-propyl group, 1-ethyl-1-methyl-n-propyl group, and 1-ethyl-2-methyl-n-propyl group.

(1-3-2)

Examples of the “cyclic saturated aliphatic hydrocarbon group” in the definition of A in formula (I) include cyclopropyl group, cyclobutyl group, 1-methyl-cyclopropyl group, 2-methyl-cyclopropyl group, cyclopentyl group, 1-methyl-cyclobutyl group, 2-methyl-cyclobutyl group, 3-methyl-cyclobutyl group, 1,2-dimethyl-cyclopropyl group, 2,3-dimethyl-cyclopropyl group, 1-ethyl-cyclopropyl group, 2-ethyl-cyclopropyl group, cyclohexyl group, 1-methyl-cyclopentyl group, 2-methyl-cyclopentyl group, 3-methyl-cyclopentyl group, 1-ethyl-cyclobutyl group, 2-ethyl-cyclobutyl group, 3-ethyl-cyclobutyl group, 1,2-dimethyl-cyclobutyl group, 1,3-dimethyl-cyclobutyl group, 2,2-dimethyl-cyclobutyl group, 2,3-dimethyl-cyclobutyl group, 2,4-dimethyl-cyclobutyl group, 3,3-dimethyl-cyclobutyl group, 1-n-propyl-cyclopropyl group, 2-n-propyl-cyclopropyl group, 1-i-propyl-cyclopropyl group, 2-i-propyl-cyclopropyl group, 1,2,2-trimethyl-cyclopropyl group, 1,2,3-trimethyl-cyclopropyl group, 2,2,3-trimethyl-cyclopropyl group, 1-ethyl-2-methyl-cyclopropyl group, 2-ethyl-1-methyl-cyclopropyl group, 2-ethyl-2-methyl-cyclopropyl group, and 2-ethyl-3-methyl-cyclopropyl group.

(1-3-3)

Examples of the “linear or branched, unsaturated aliphatic hydrocarbon group” in the definition of A in formula (I) or in the definition of R¹ and R² in R¹R²R³N include ethenyl group, 1-propenyl group, 2-propenyl group, 1-methyl-1-ethenyl group, 1-butenyl group, 2-butenyl group, 3-butenyl group, 2-methyl-1-propenyl group, 2-methyl-2-propenyl group, 1-ethylethenyl group, 1-methyl-1-propenyl group, 1-methyl-2-propenyl group, 1-pentenyl group, 2-pentenyl group, 3-pentenyl group, 4-pentenyl group, 1-n-propylethenyl group, 1-methyl-1-butenyl group, 1-methyl-2-butenyl group, 1-methyl-3-butenyl group, 2-ethyl-2-propenyl group, 2-methyl-1-butenyl group, 2-methyl-2-butenyl group, 2-methyl-3-butenyl group, 3-methyl-1-butenyl group, 3-methyl-2-butenyl group, 3-methyl-3-butenyl group, 1,1-dimethyl-2-propenyl group, 1-i-propylethenyl group, 1,2-dimethyl-1-propenyl group, 1,2-dimethyl-2-propenyl group, 1-hexenyl group, 2-hexenyl group, 3-hexenyl group, 4-hexenyl group, 5-hexenyl group, 1-methyl-1-pentenyl group, 1-methyl-2-pentenyl group, 1-methyl-3-pentenyl group, 1-methyl-4-pentenyl group, 1-n-butylethenyl group, 2-methyl-1-pentenyl group, 2-methyl-2-pentenyl group, 2-methyl-3-pentenyl group, 2-methyl-4-pentenyl group, 2-n-propyl-2-propenyl group, 3-methyl-1-pentenyl group, 3-methyl-2-pentenyl group, 3-methyl-3-pentenyl group, 3-methyl-4-pentenyl group, 3-ethyl-3-butenyl group, 4-methyl-1-pentenyl group, 4-methyl-2-pentenyl group, 4-methyl-3-pentenyl group, 4-methyl-4-pentenyl group, 1,1-dimethyl-2-butenyl group, 1,1-dimethyl-3-butenyl group, 1,2-dimethyl-1-butenyl group, 1,2-dimethyl-2-butenyl group, 1,2-dimethyl-3-butenyl group, 1-methyl-2-ethyl-2-propenyl group, 1-s-butylethenyl group, 1,3-dimethyl-1-butenyl group, 1,3-dimethyl-2-butenyl group, 1,3-dimethyl-3-butenyl group, 1-i-butylethenyl group, 2,2-dimethyl-3-butenyl group, 2,3-dimethyl-1-butenyl group, 2,3-dimethyl-2-butenyl group, 2,3-dimethyl-3-butenyl group, 2-i-propyl-2-propenyl group, 3,3-dimethyl-1-butenyl group, 1-ethyl-I-butenyl group, 1-ethyl-2-butenyl group, 1-ethyl-3-butenyl group, 1-n-propyl-1-propenyl group, 1-n-propyl-2-propenyl group, 2-ethyl-1-butenyl group, 2-ethyl-2-butenyl group, 2-ethyl-3-butenyl group, 1,1,2-trimethyl-2-propenyl group, 1-t-butylethenyl group, 1-methyl-1-ethyl-2-propenyl group, 1-ethyl-2-methyl-1-propenyl group, 1-ethyl-2-methyl-2-propenyl group, 1-i-propyl-1-propenyl group, and 1-i-propyl-2-propenyl group.

(1-3-4)

Examples of the “cyclic unsaturated aliphatic hydrocarbon group” in the definition of A in formula (I) include 1-cyclopentenyl group, 2-cyclopentenyl group, 3-cyclopentenyl group, 1-methyl-2-cyclopentenyl group, 1-methyl-3-cyclopentenyl group, 2-methyl-1-cyclopentenyl group, 2-methyl-2-cyclopentenyl group, 2-methyl-3-cyclopentenyl group, 2-methyl-4-cyclopentenyl group, 2-methyl-5-cyclopentenyl group, 2-methylene-cyclopentyl group, 3-methyl-1-cyclopentenyl group, 3-methyl-2-cyclopentenyl group, 3-methyl-3-cyclopentenyl group, 3-methyl-4-cyclopentenyl group, 3-methyl-5-cyclopentenyl group, 3-methylene-cyclopentyl group, 1-cyclohexenyl group, 2-cyclohexenyl group, and 3-cyclohexenyl group.

(1-3-5)

The “aromatic ring residue” in the definition of A in formula (I) or in the definition of R in formula (II) may be an aromatic hydrocarbon group, with examples including phenyl group, o-methylphenyl group, m-methylphenyl group, p-methylphenyl group, 2,3-dimethylphenyl group, 2,4-dimethylphenyl group, 2,5-dimethylphenyl group, 2,6-dimethylphenyl group, 3,4-dimethylphenyl group, 3,5-dimethylphenyl group, o-chlorophenyl group, m-chlorophenyl group, p-chlorophenyl group, o-fluorophenyl group, p-fluorophenyl group, o-methoxyphenyl group, p-methoxyphenyl group, p-nitrophenyl group, p-cyanophenyl group, a-naphthyl group, [3-naphthyl group, o-biphenylyl group, m-biphenylyl group, p-biphenylyl group, 1-anthryl group, 2-anthryl group, 9-anthryl group, 1-phenanthryl group, 2-phenanthryl group, 3-phenanthryl group, 4-phenanthryl group, 9-phenanthryl group, 1-pyrenyl group, 2-pyrenyl group, and 3-pyrenyl group.

Furthermore, the “aromatic ring residue” in the definition of A in formula (I) may be an aromatic heterocyclic residue, with examples including furanyl group, thiophenyl group, pyrrolyl group, imidazolyl group, pyranyl group, pyridinyl group, pyrimidinyl group, pyrazinyl group, pyrrolidinyl group, piperidinyl group, piperazinyl group, morpholinyl group, quinuclidinyl group, indolyl group, purinyl group, quinolinyl group, isoquinolinyl group, chromenyl group, thianthrenyl group, phenothiazinyl group, phenoxazinyl group, xanthenyl group, acridinyl group, phenazinyl group, and carbazolyl group.

Examples of the “aromatic ring” or the “aromatic ring” in the definition of R³ in R¹R²R³N are the same as mentioned above.

(1-3-6)

Examples of the substituent expressed by the phrase “optionally substituted” in the definition of A in formula (I), in the definition of R^{I}, R^{II}, and R^{III} in R^{I}R^{II}R^{III}N, in the definition of R¹, R², and R³ in R¹R²R³N, and in the definition of R^{a} and R^{b} include nitro group, amino group, cyano group, sulfo group, hydroxy group, carboxyl group, aldehyde group, propargylamino group, propargyloxy group, halogen atoms, C1-C10 alkoxy groups, C1-C10 alkyl groups, C2-C10 alkenyl groups, C2-C10 alkynyl groups, C6-C40 aryl groups, ether bond-containing organic groups, ketone bond-containing organic groups, ester bond-containing organic groups, and combinations thereof.

The ether bond-containing organic groups, the ketone bond-containing organic groups, and the ester bond-containing organic groups may be exemplified by the groups mentioned later in (1-3-9).

(1-3-7)

Examples of the “alkoxy group” in the definition of R in formula (II) include methoxy group, ethoxy group, n-propoxy group, i-propoxy group, n-butoxy group, i-butoxy group, s-butoxy group, t-butoxy group, n-pentyloxy group, 1-methyl-n-butoxy group, 2-methyl-n-butoxy group, 3-methyl-n-butoxy group, 1,1-dimethyl-n-propoxy group, 1,2-dimethyl-n-propoxy group, 2,2-dimethyl-n-propoxy group, 1-ethyl-n-propoxy group, n-hexyloxy group, 1-methyl-n-pentyloxy group, 2-methyl-n-pentyloxy group, 3-methyl-n-pentyloxy group, 4-methyl-n-pentyloxy group, 1,1-dimethyl-n-butoxy group, 1,2-dimethyl-n-butoxy group, 1,3-dimethyl-n-butoxy group, 2,2-dimethyl-n-butoxy group, 2,3-dimethyl-n-butoxy group, 3,3-dimethyl-n-butoxy group, 1-ethyl-n-butoxy group, 2-ethyl-n-butoxy group, 1,1,2-trimethyl-n-propoxy group, 1,2,2-trimethyl-n-propoxy group, 1-ethyl-I-methyl-n-propoxy group, and 1-ethyl-2-methyl-n-propoxy group.

(1-3-8)

Examples of the “alkylene group” in the definition of R in formula (II) or in the definition of R^{a} and R^{b} include alkylene groups derived from the alkyl groups mentioned in (1-3-1) and (1-3-2) by replacing a hydrogen atom with an additional valence bond.

Examples of the “alkenyl group” in the definition of R in formula (II) include those mentioned in (1-3-3) and (1-3-4).

Examples of the “hydroxyalkyl group” in the definition of R in formula (II) include the following organic groups. In the formulas, * indicates a valence bond on the carbon atom.

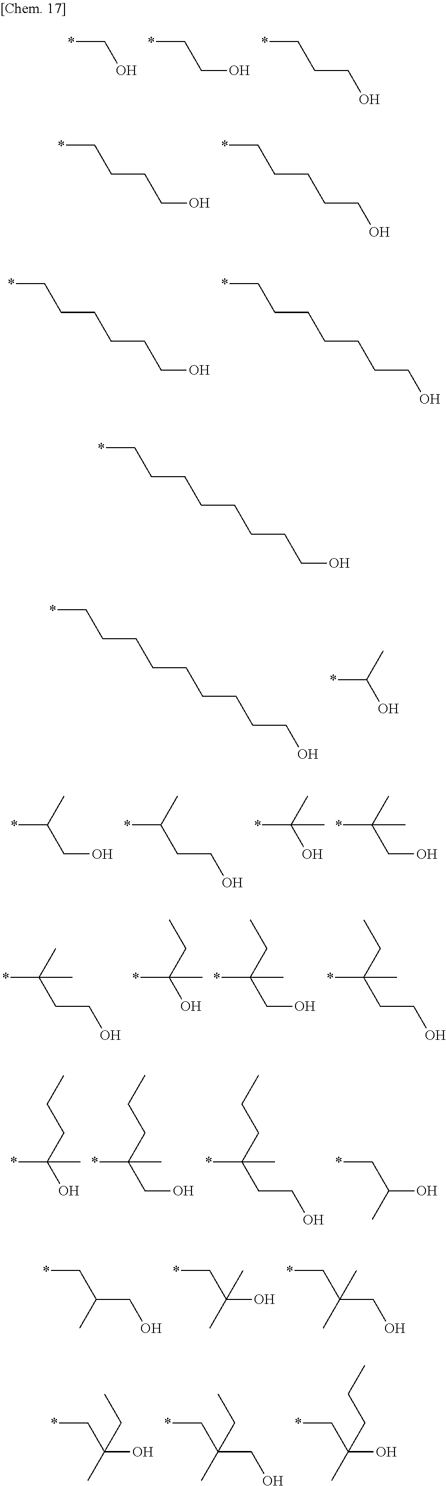

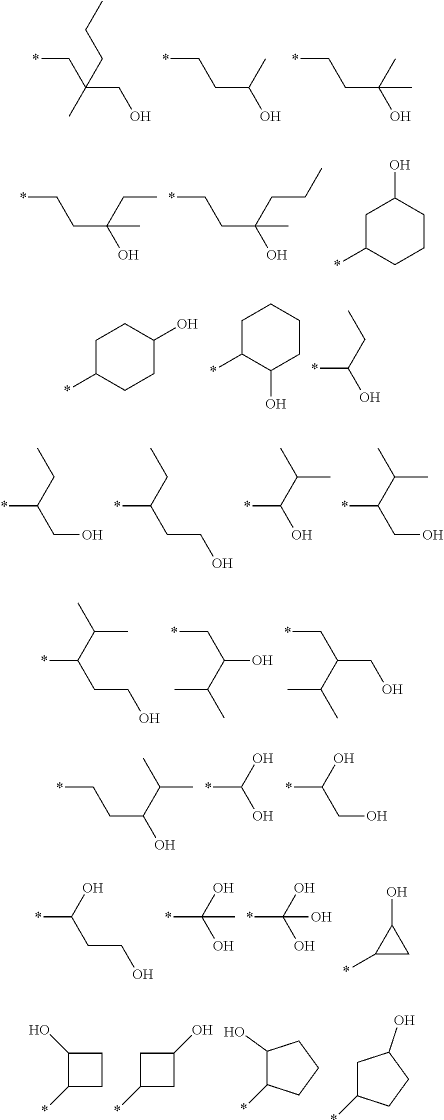

The “alkynyl group” in the definition of R in formula (II) may be such that an aliphatic hydrocarbon chain contains a carbon-carbon triple bond (at an end of the chain or in the middle of the chain) and optionally further contains a heteroatom (such as an oxygen atom or a nitrogen atom) or may be such that alkynyl groups are connected to one another. Examples include the following organic groups. In the formulas, indicates a valence bond on the carbon atom.

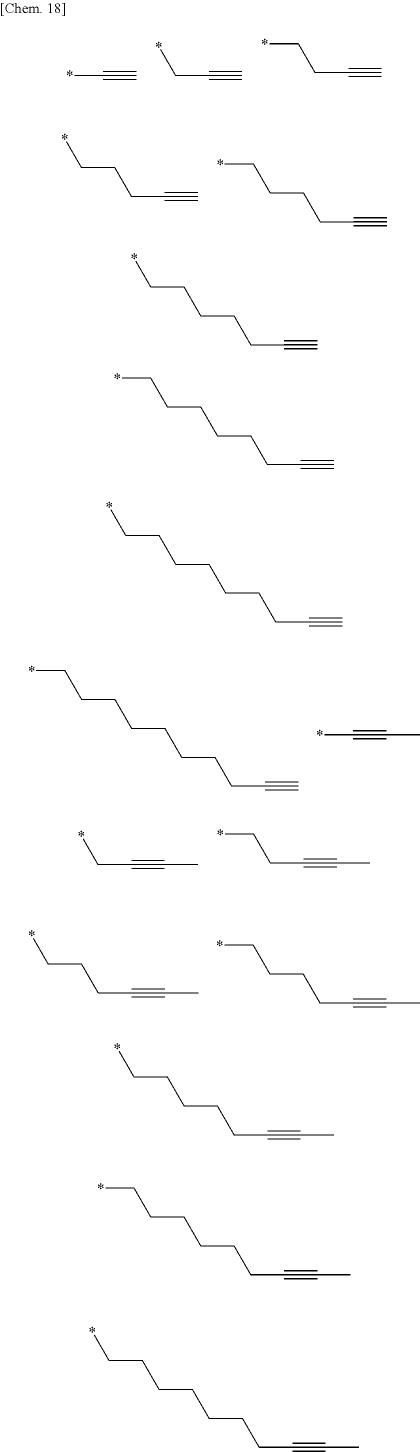

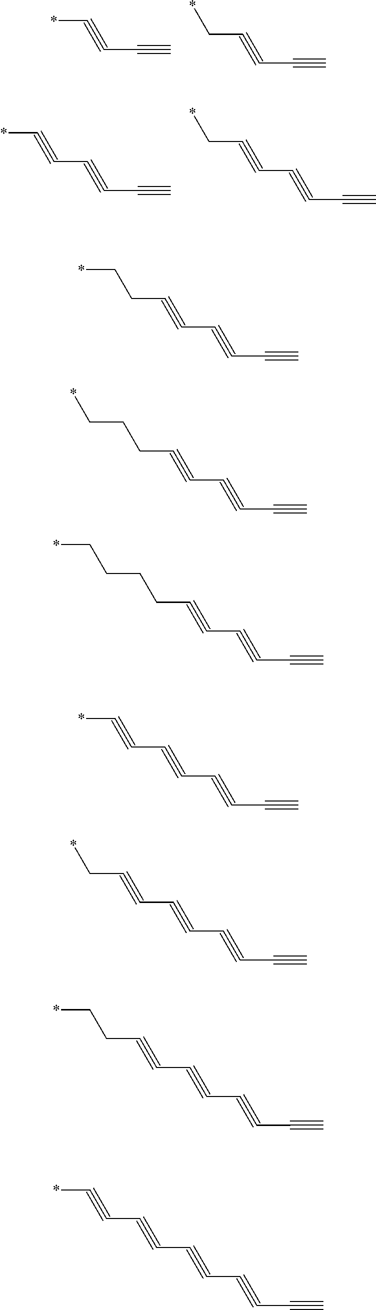

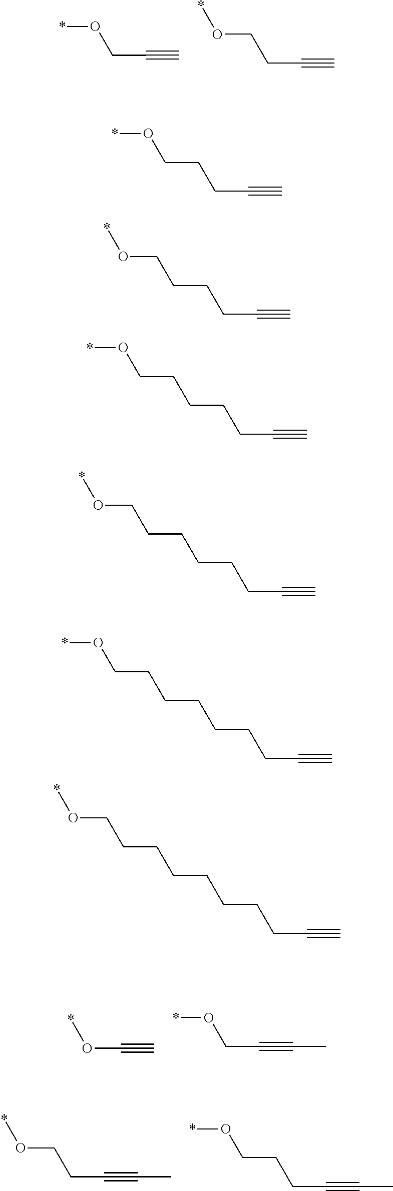

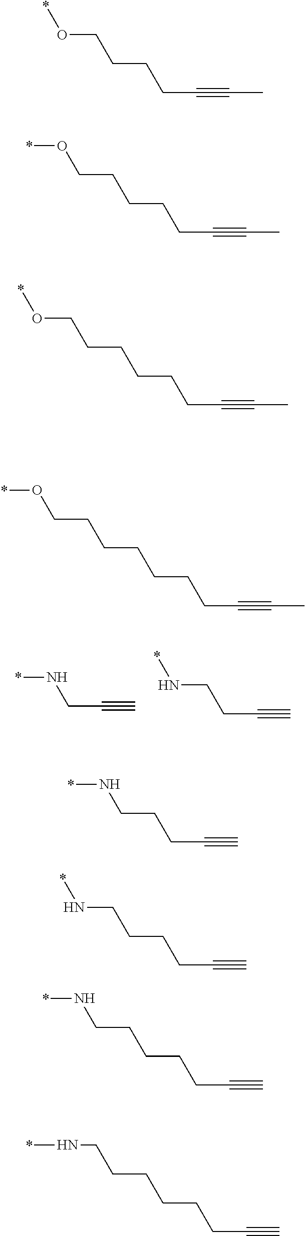

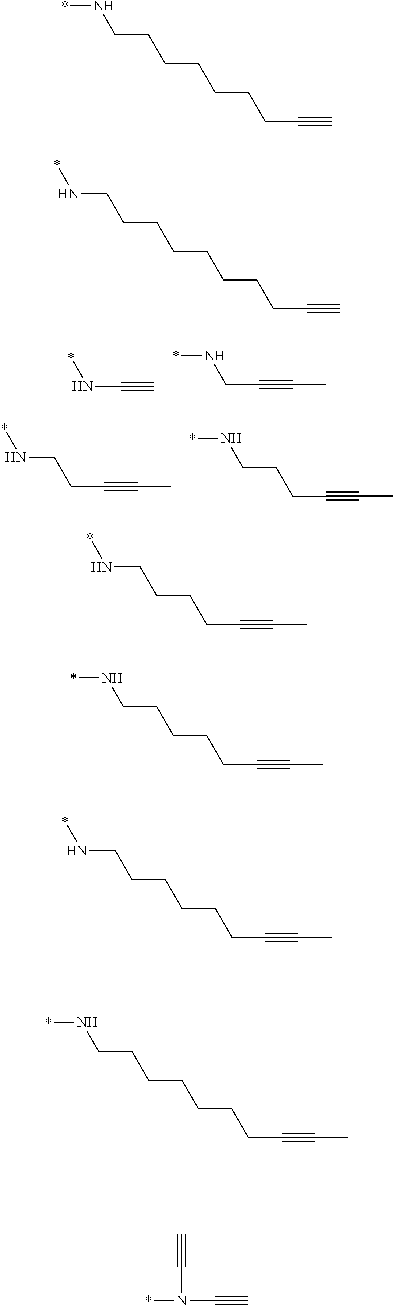

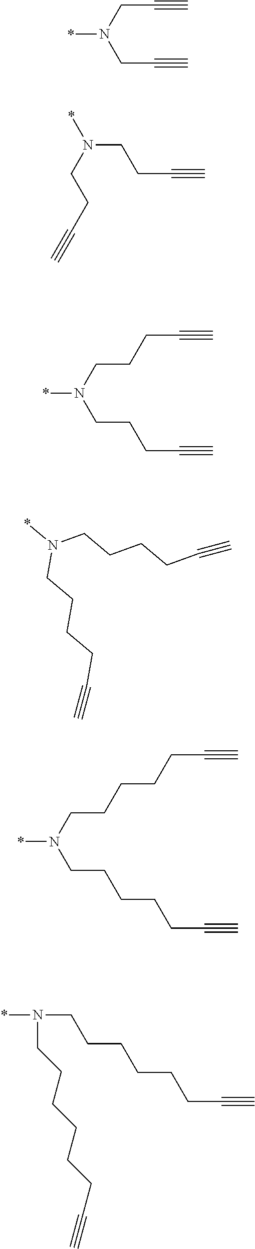

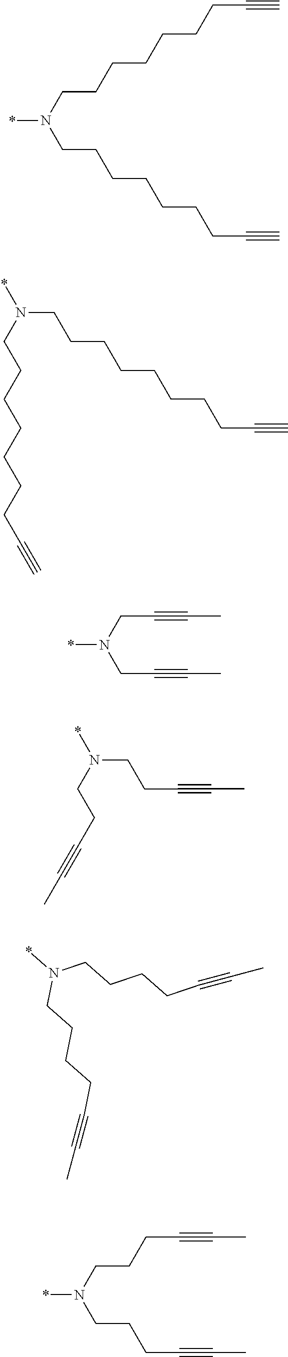

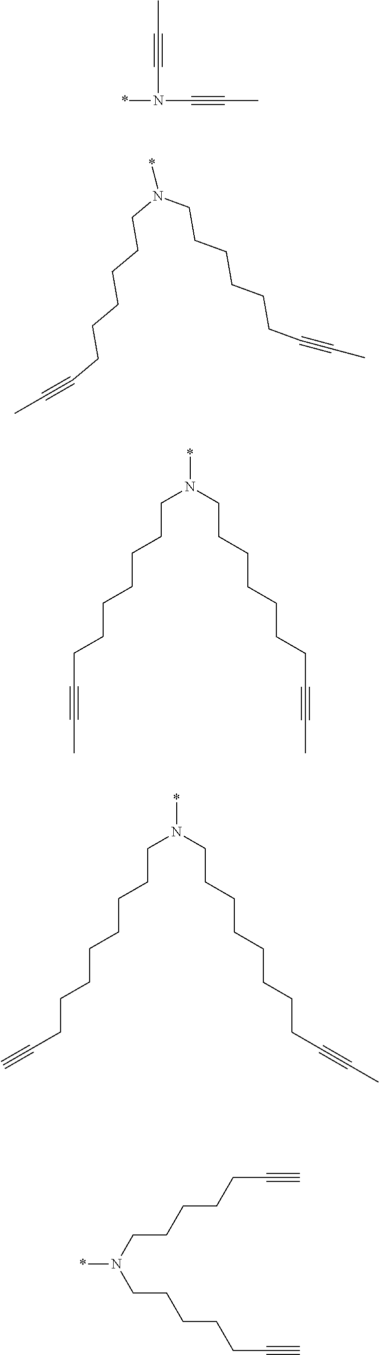

(1-3-9)

The “ether bond-containing organic group” in the definition of R in formula (II) may be a residue of an ether compound represented by R¹¹—O—R¹¹ (R¹¹ independently at each occurrence denotes a C1-C6 alkyl group, such as a methyl group or an ethyl group, an alkylene group, a phenyl group, a phenylene group, a naphthyl group, a naphthylene group, an anthranyl group, or a pyrenyl group). Examples of the ether bond-containing organic groups include those containing a methoxy group, an ethoxy group, or a phenoxy group.

The “ketone bond-containing organic group” in the definition of R in formula (II) may be a residue of a ketone compound represented by R²¹—C(═O)—R² (R² independently at each occurrence denotes a C1-C6 alkyl group, such as a methyl group or an ethyl group, an alkylene group, a phenyl group, a phenylene group, a naphthyl group, a naphthylene group, an anthranyl group, or a pyrenyl group). Examples of the ketone bond-containing organic groups include those containing an acetoxy group or a benzoyl group.

The “ester bond-containing organic group” in the definition of R in formula (II) may be a residue of an ester compound represented by R³¹—C(═O)O—R³¹ (R³¹ independently at each occurrence denotes a C1-C6 alkyl group, such as a methyl group or an ethyl group, an alkylene group, a phenyl group, a phenylene group, a naphthyl group, a naphthylene group, an anthranyl group, or a pyrenyl group). Examples of the ester bond-containing organic groups include those containing a methyl ester, an ethyl ester, or a phenyl ester.

(1-4)

Examples of the thermal acid generators represented by formula (I) include, but are not limited to, those that appropriately combine at least one of the exemplary counter base cations illustrated below and at least one of the exemplary sulfonate anions illustrated below so that the charges are neutralized.

### 1-4-1. Examples of Counter Base Cations

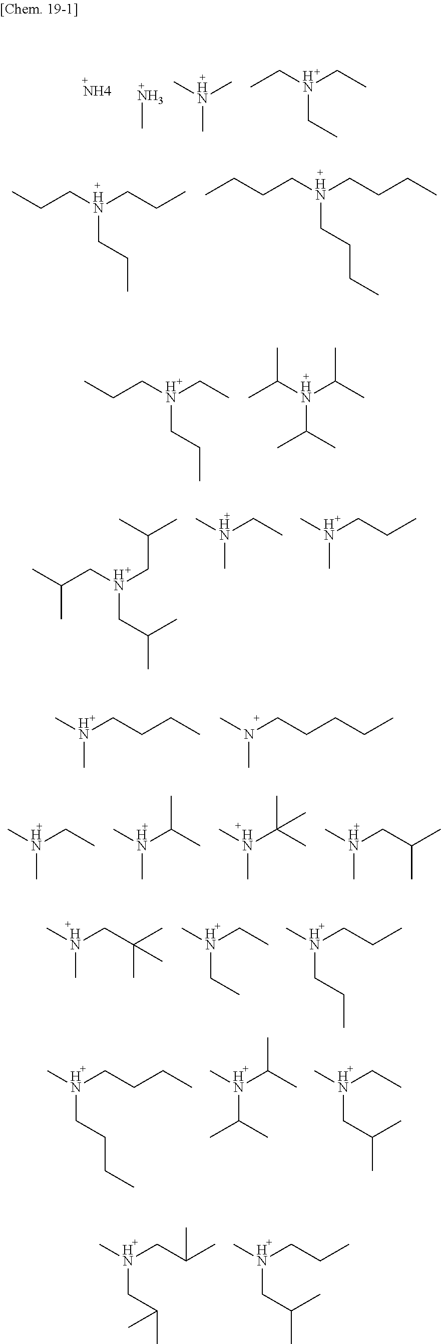

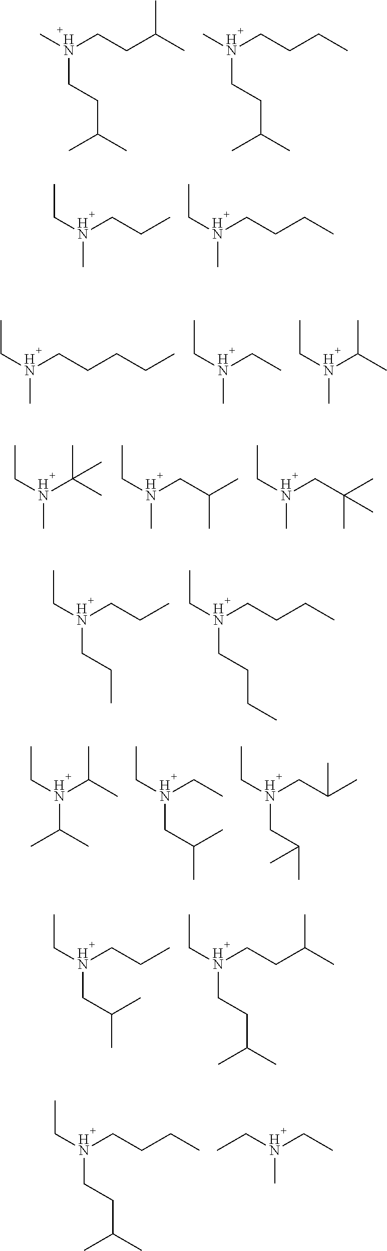

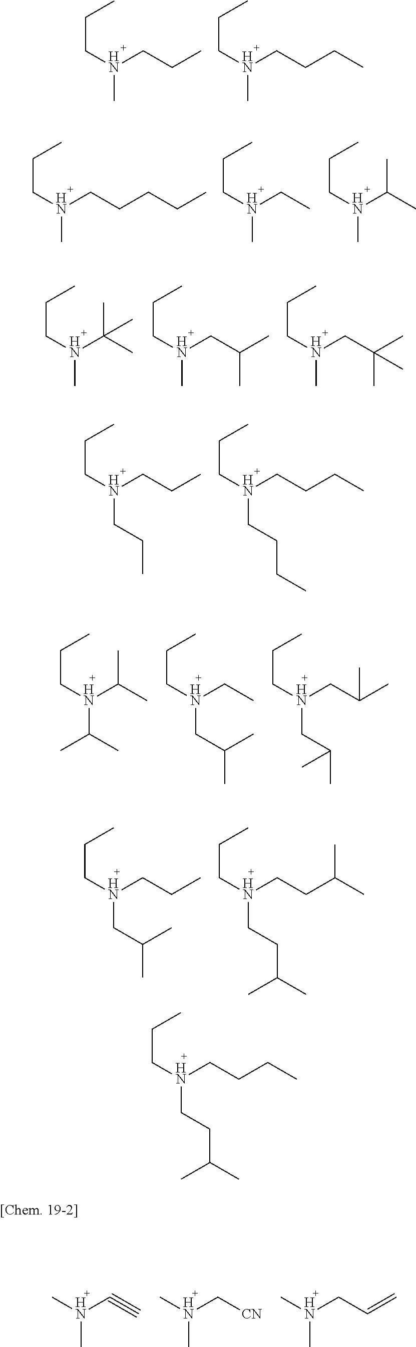

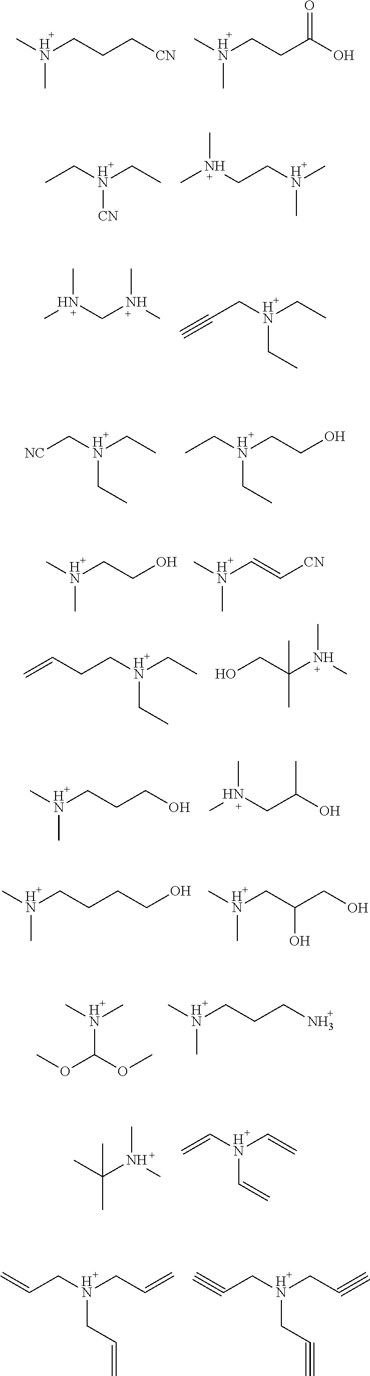

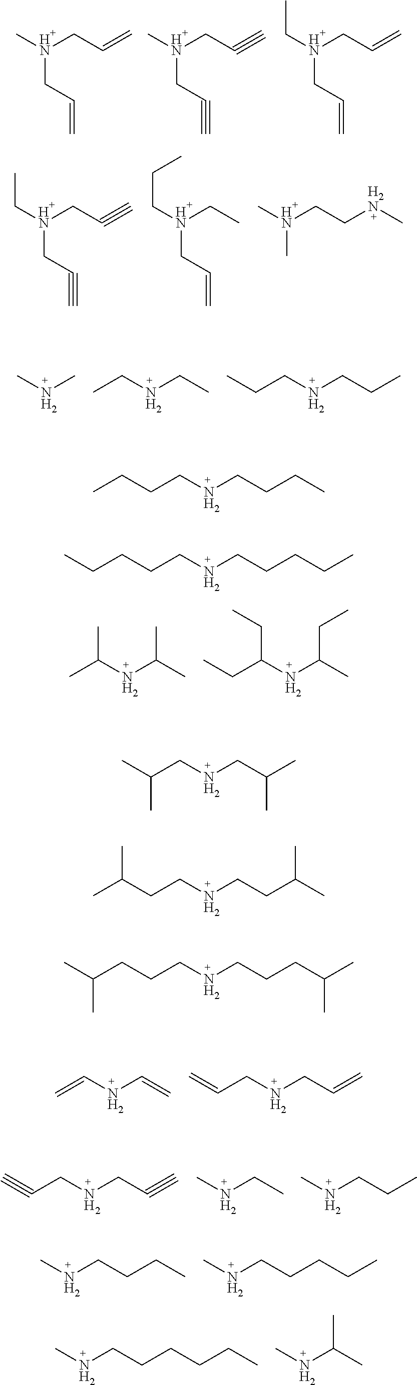

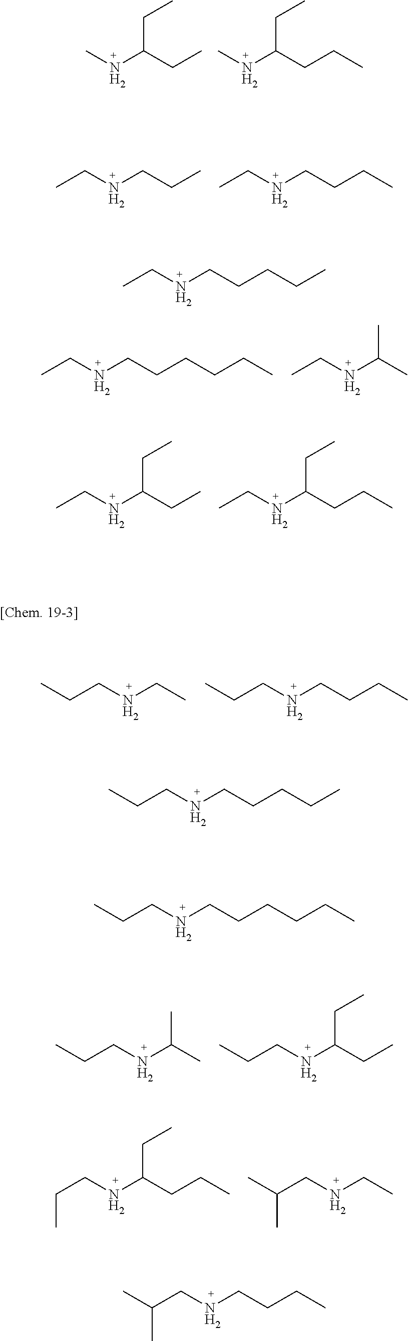

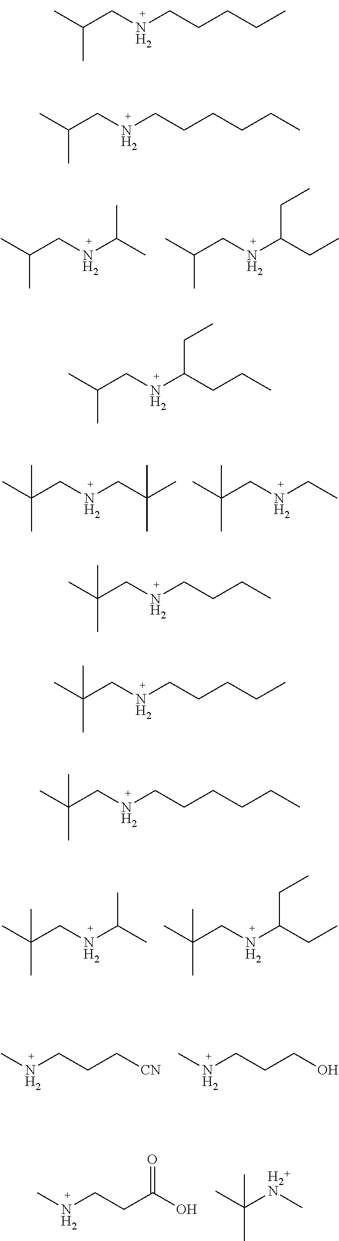

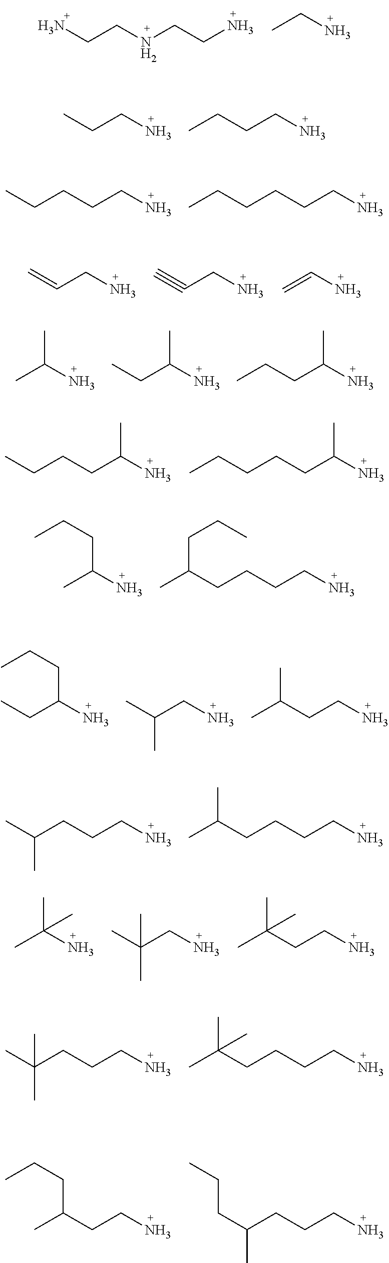

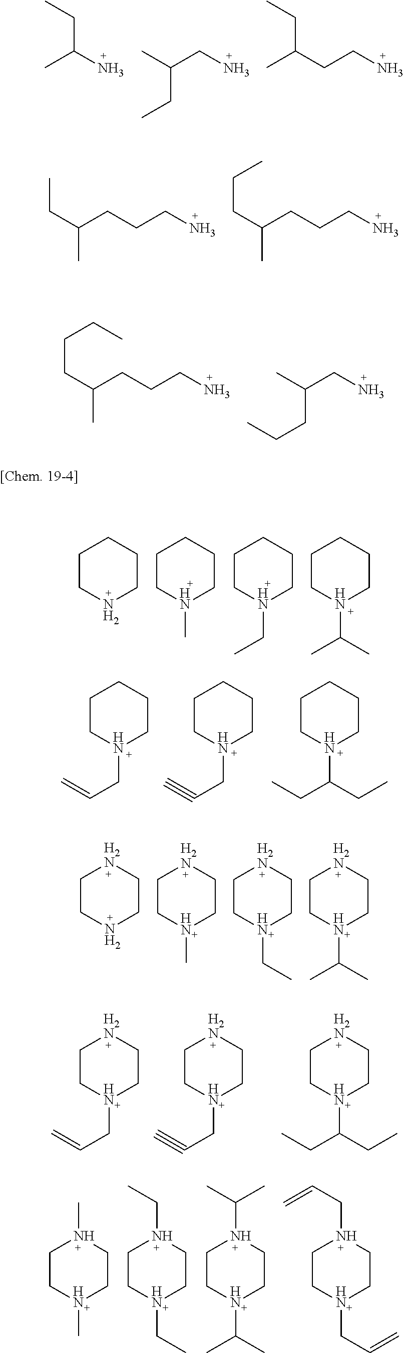

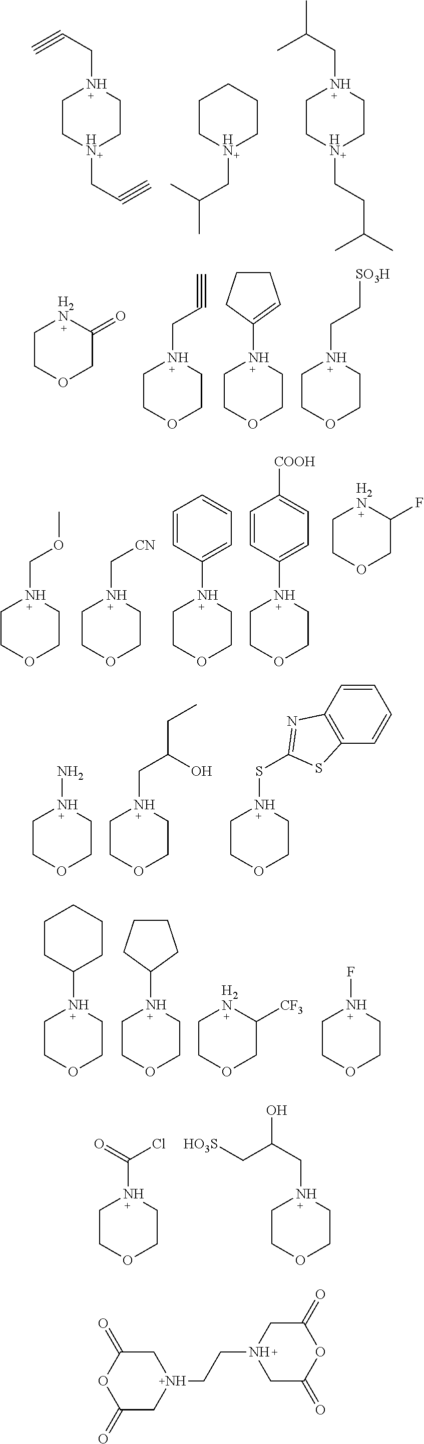

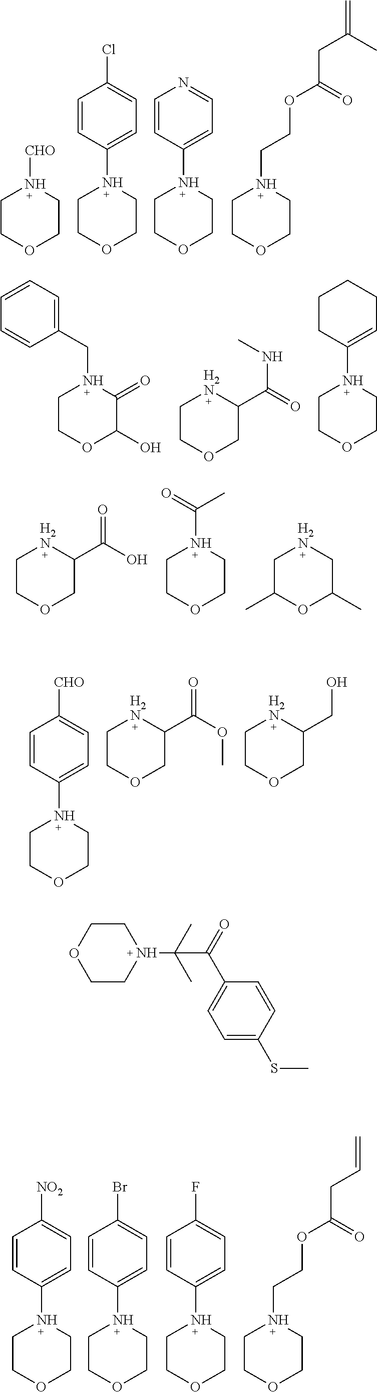

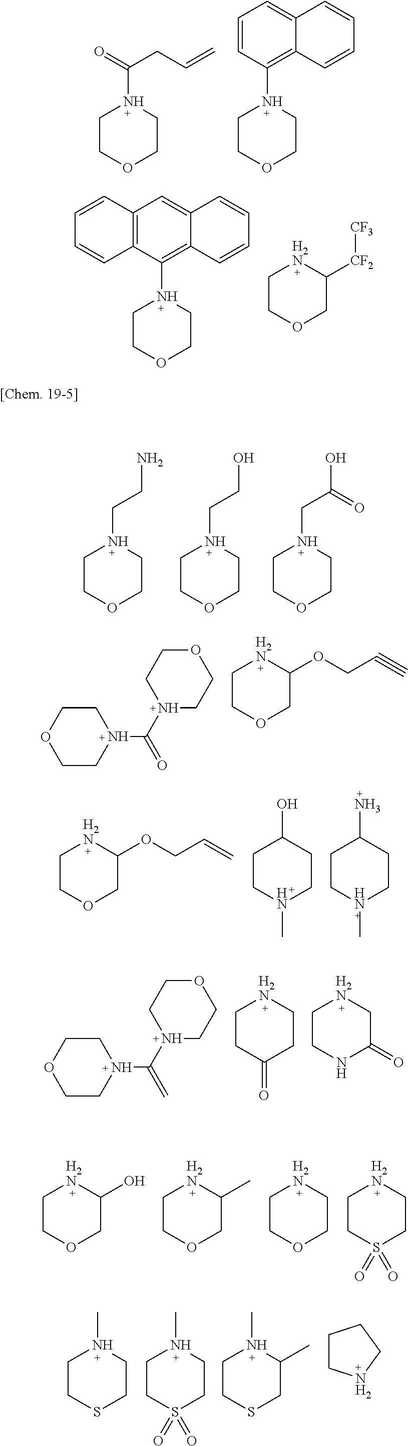

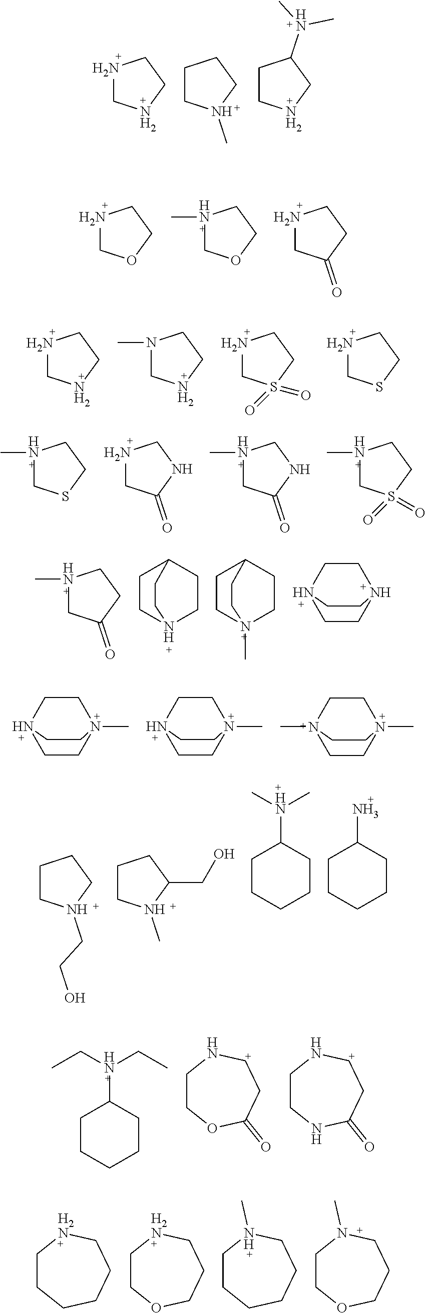

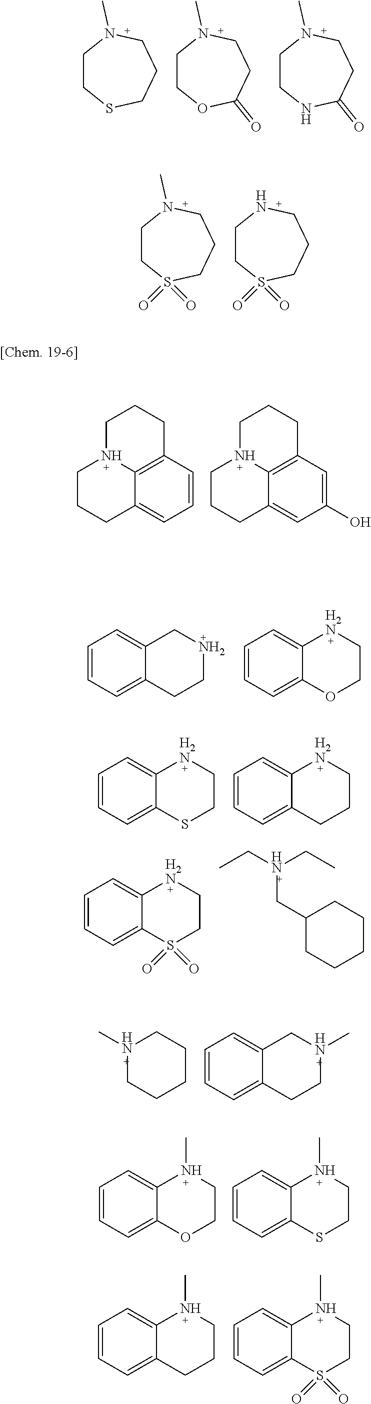

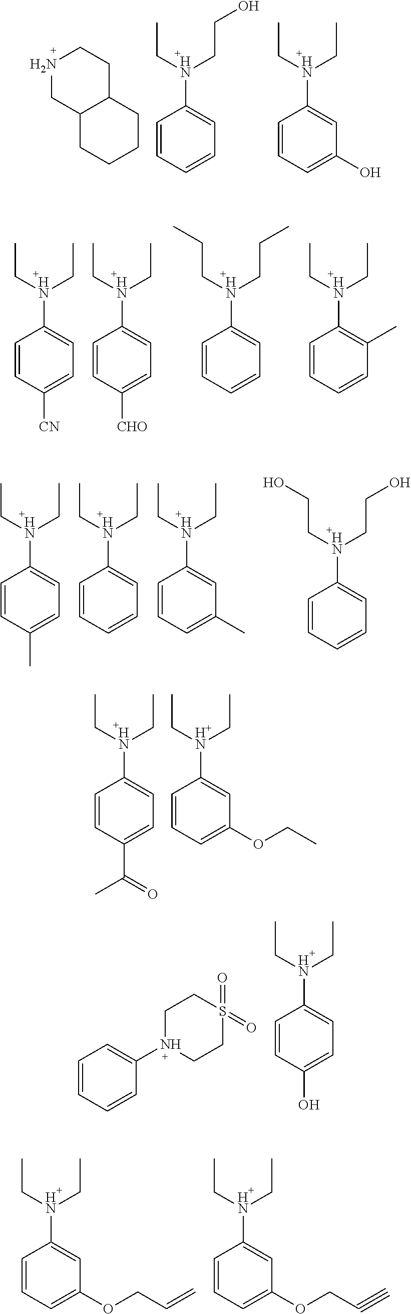

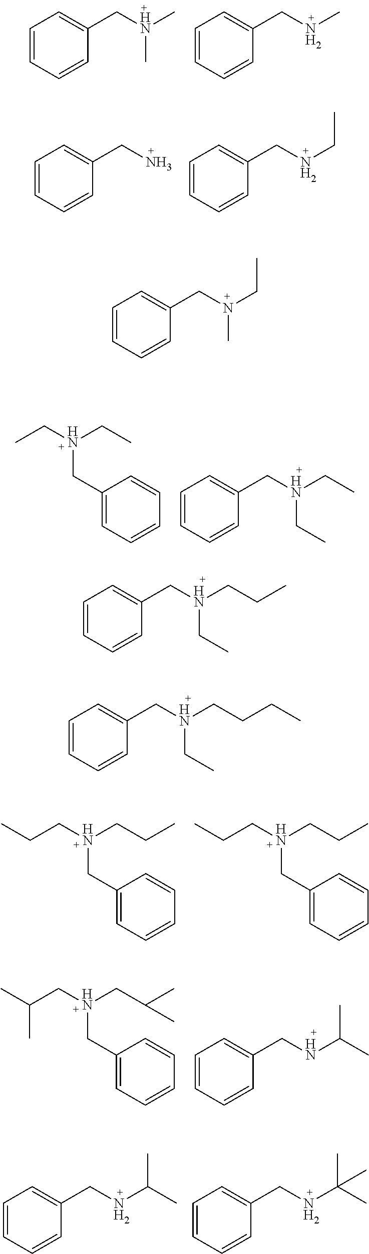

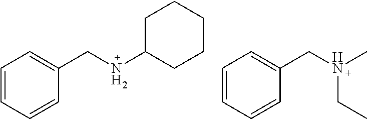

### 1-4-2: Examples of Sulfonate Anions

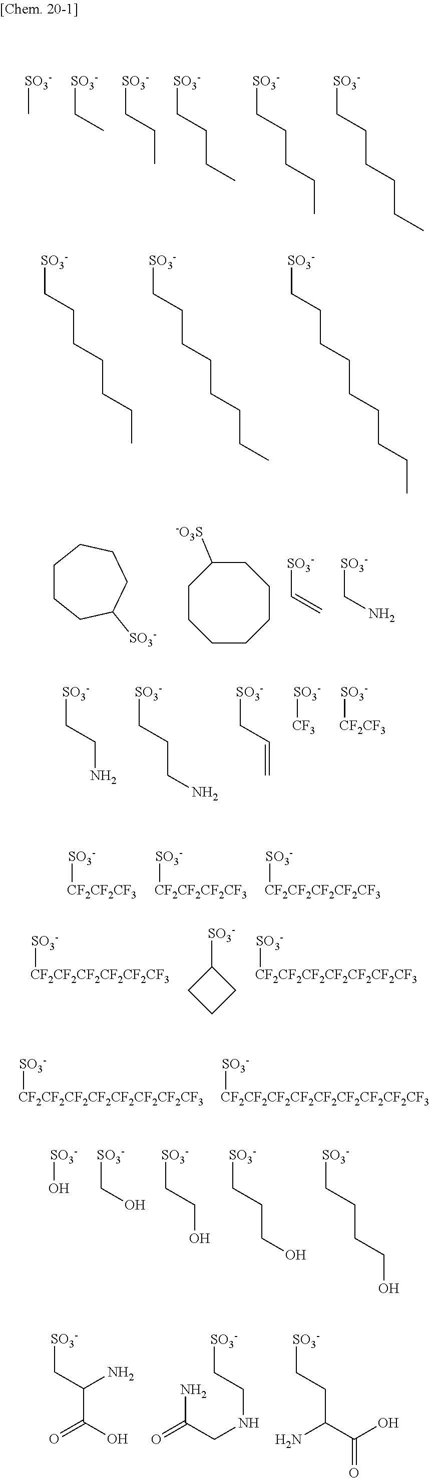

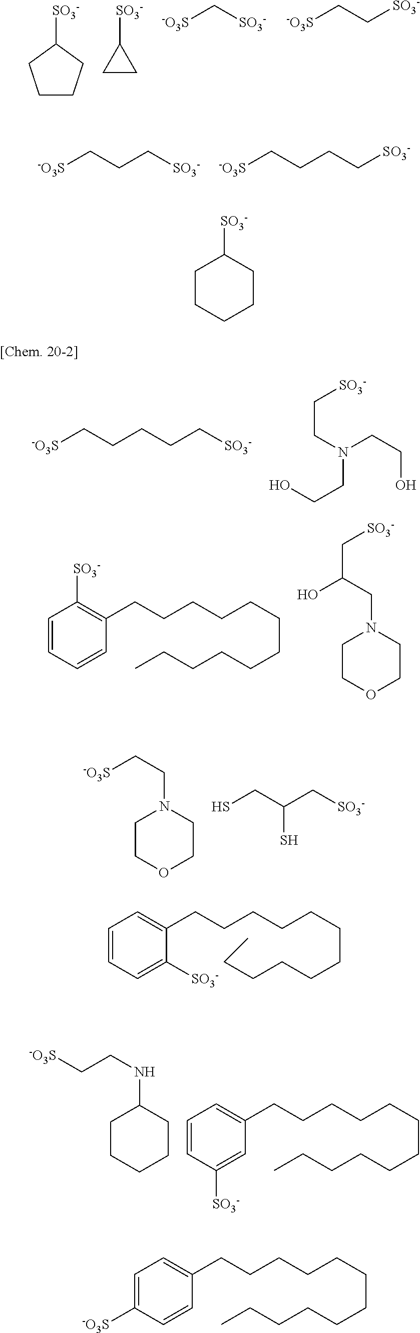

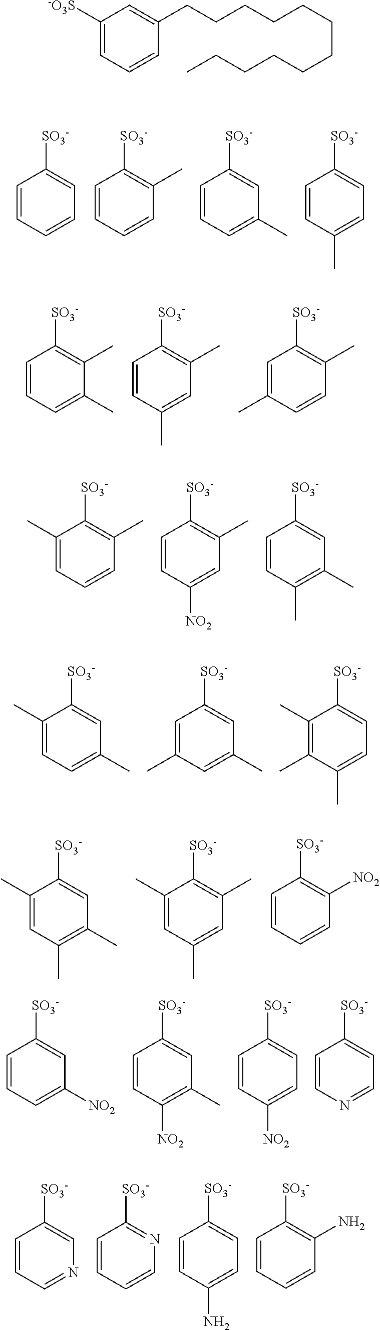

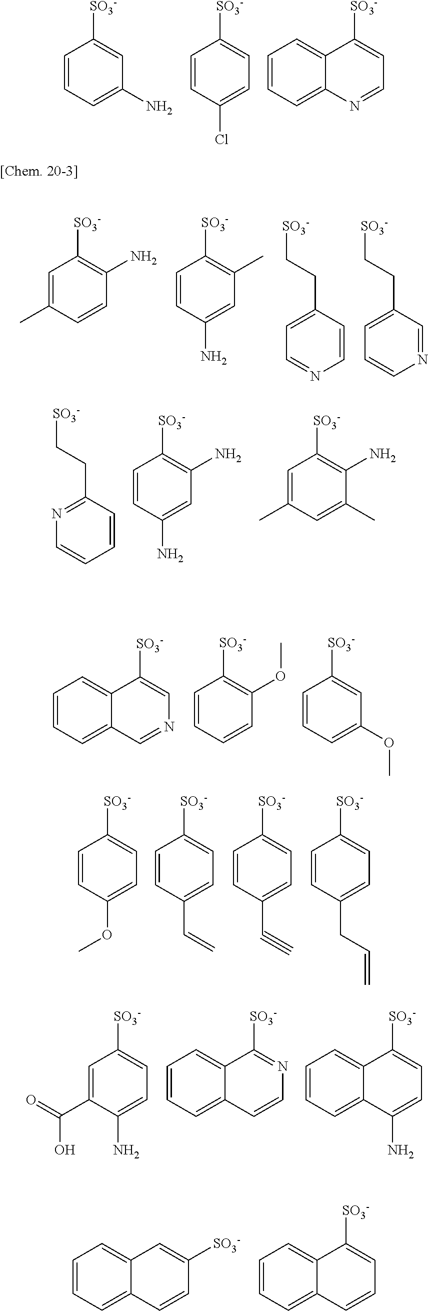

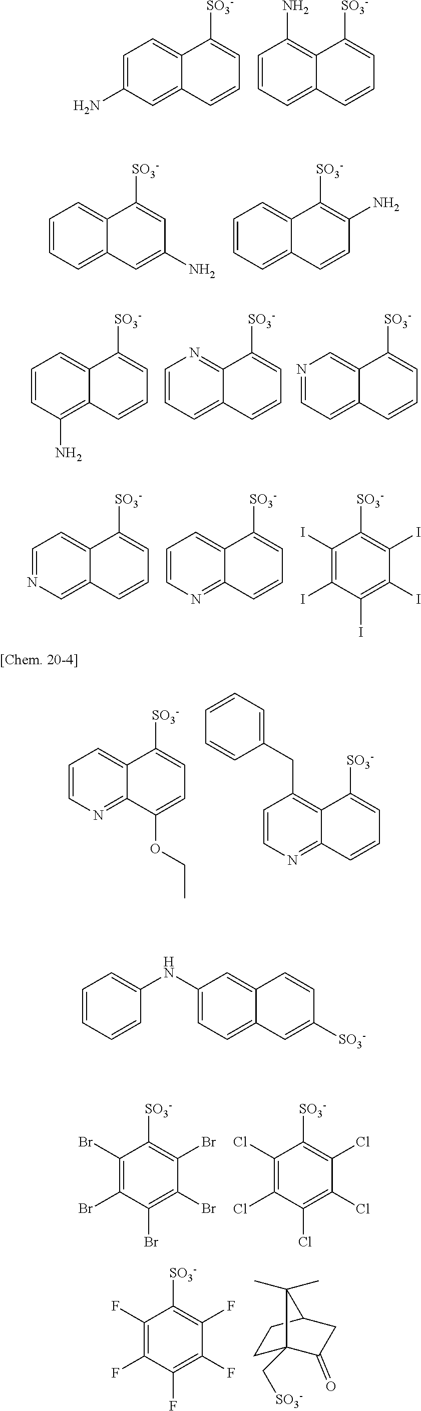

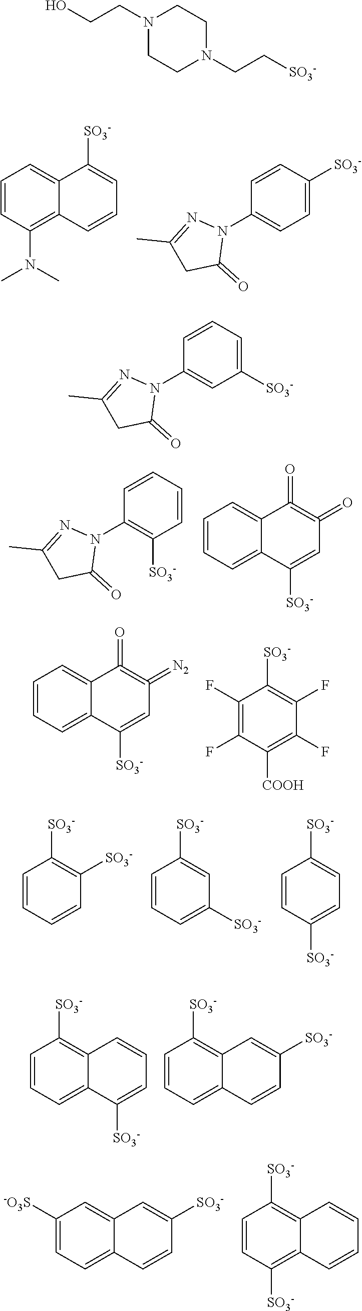

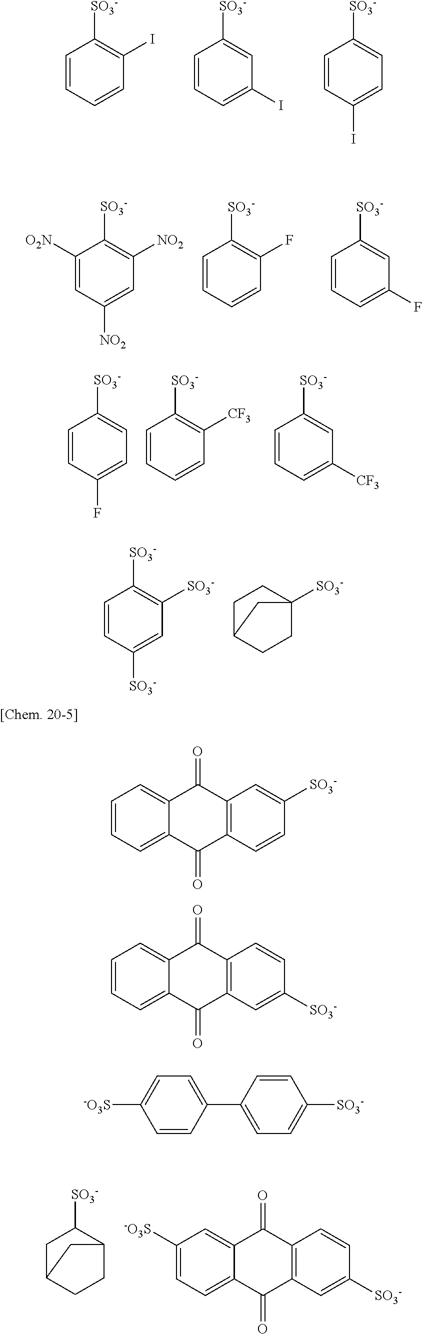

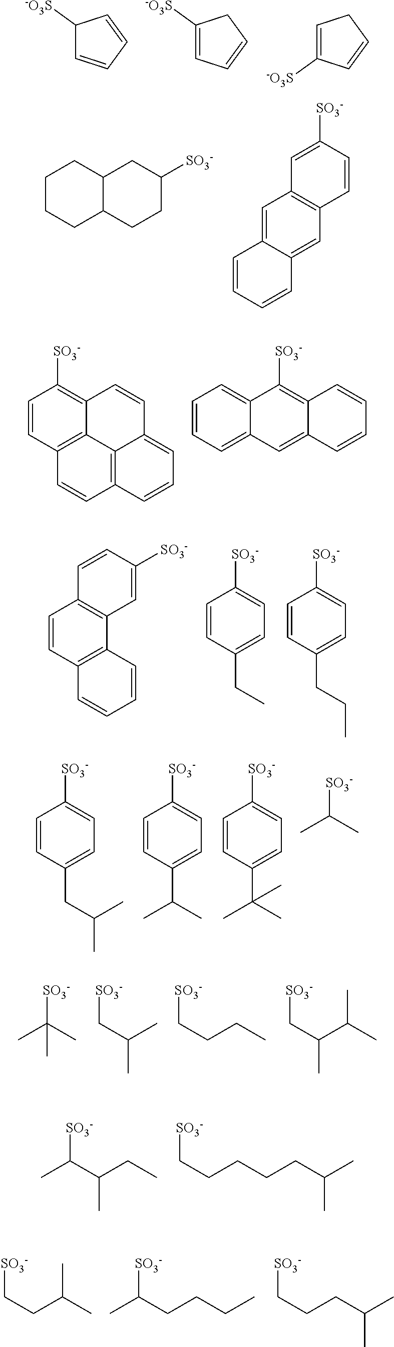

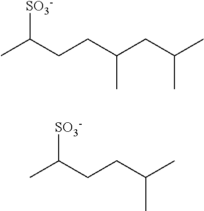

(1-4-3)

More specific examples of the thermal acid generators include, but are not limited to, the following combinations of a counter base cation and a sulfonate anion.

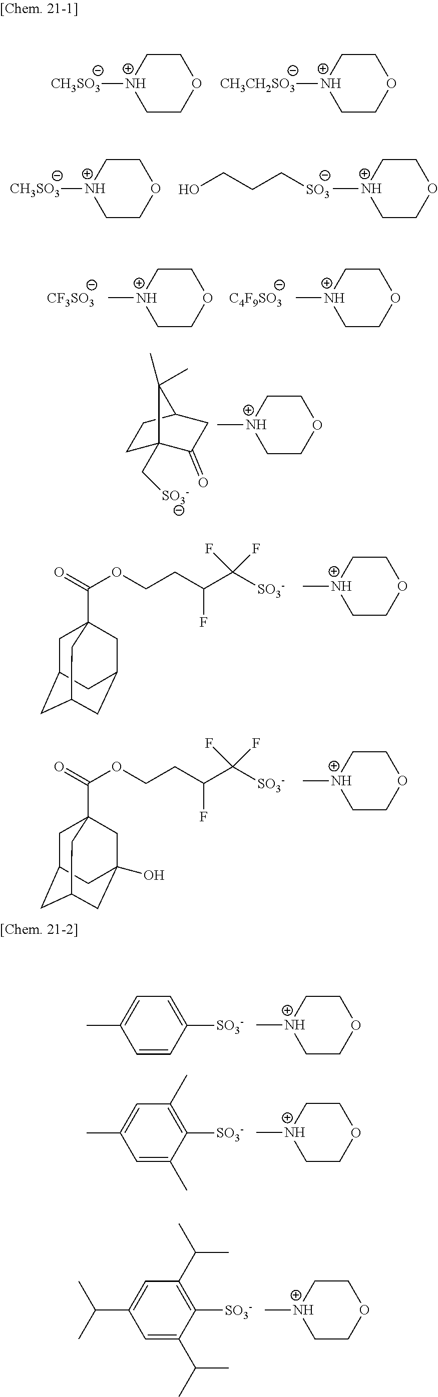

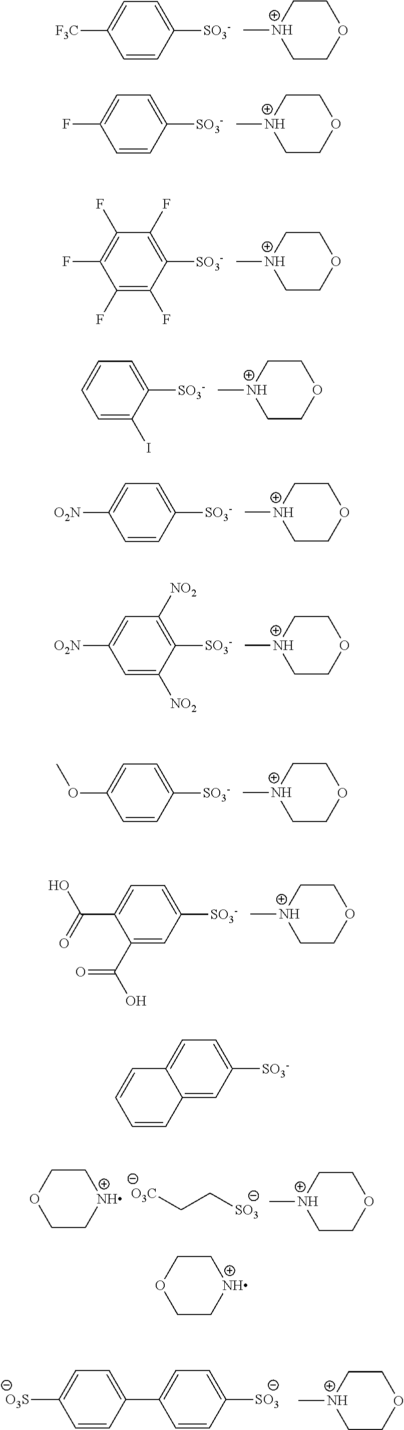

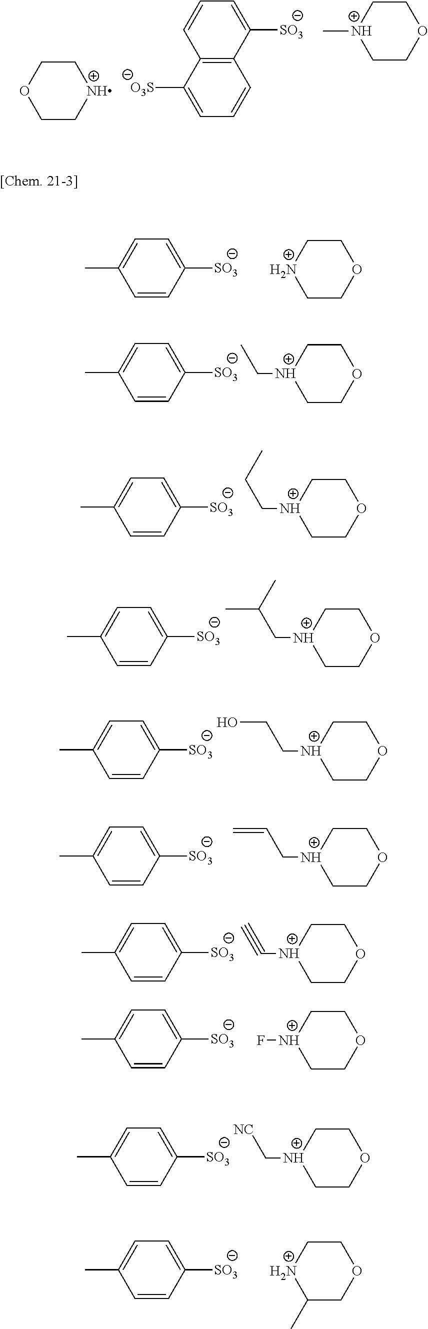

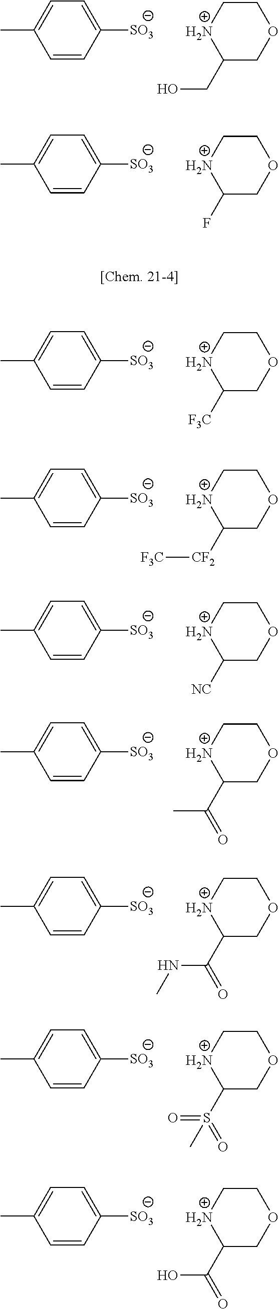

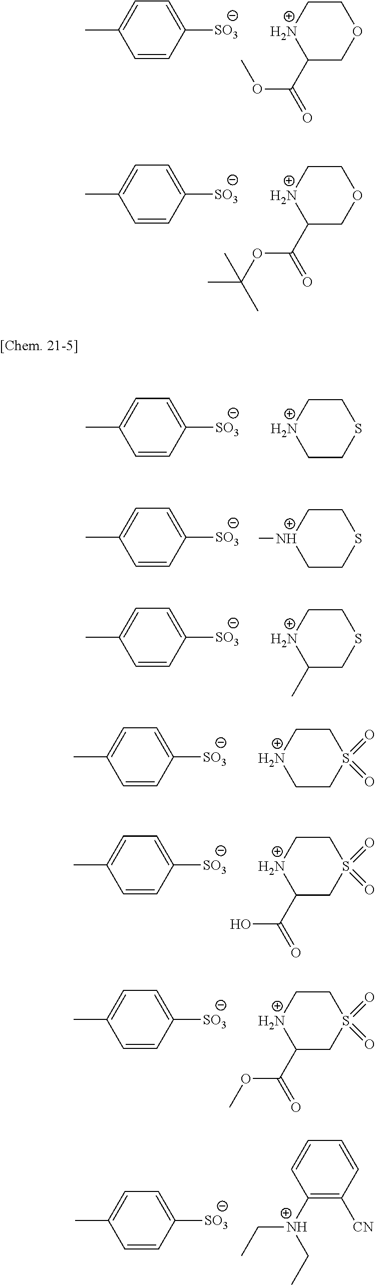

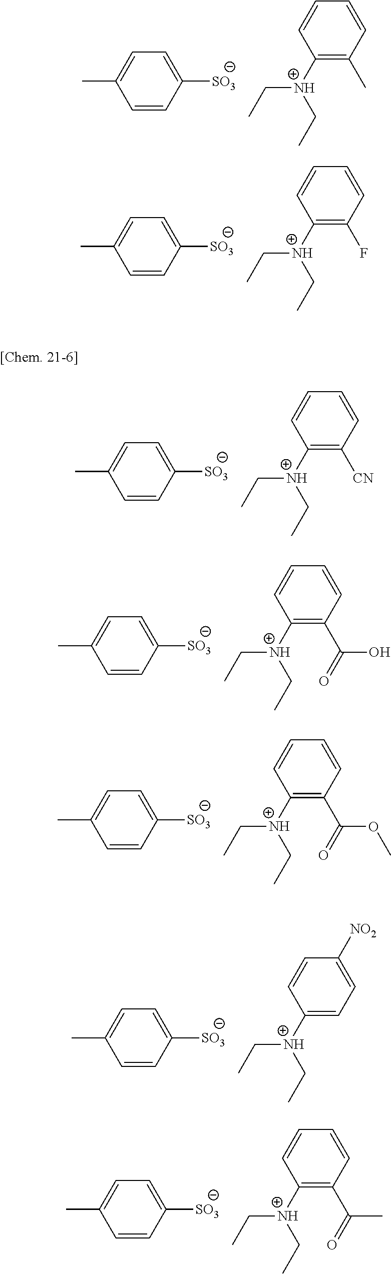

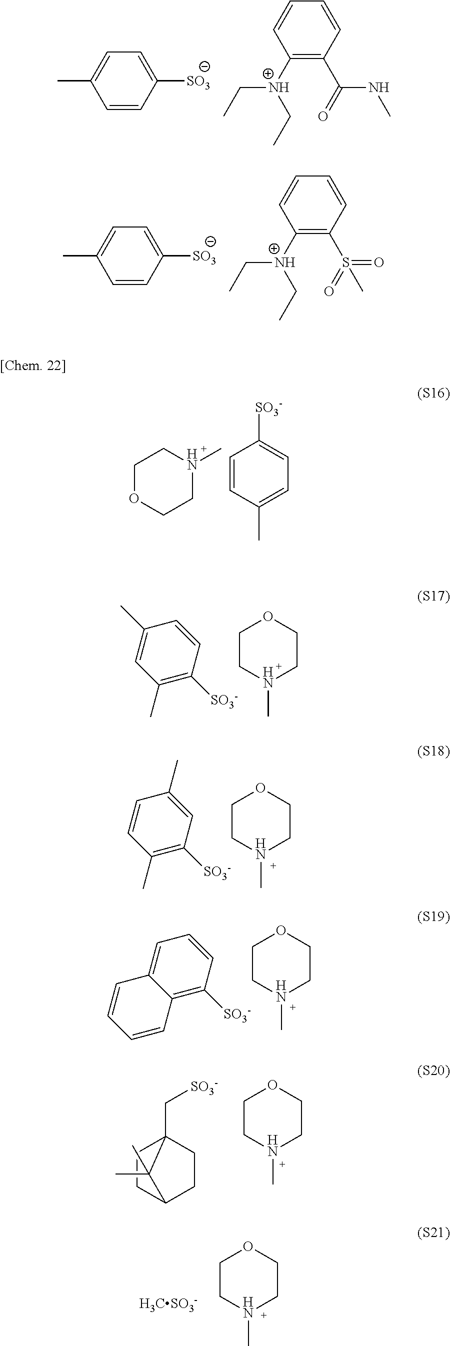

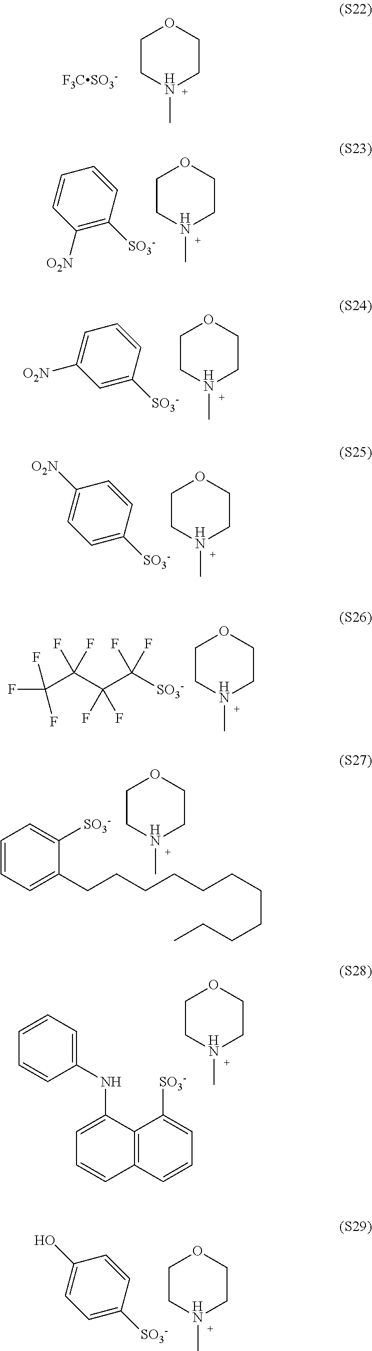

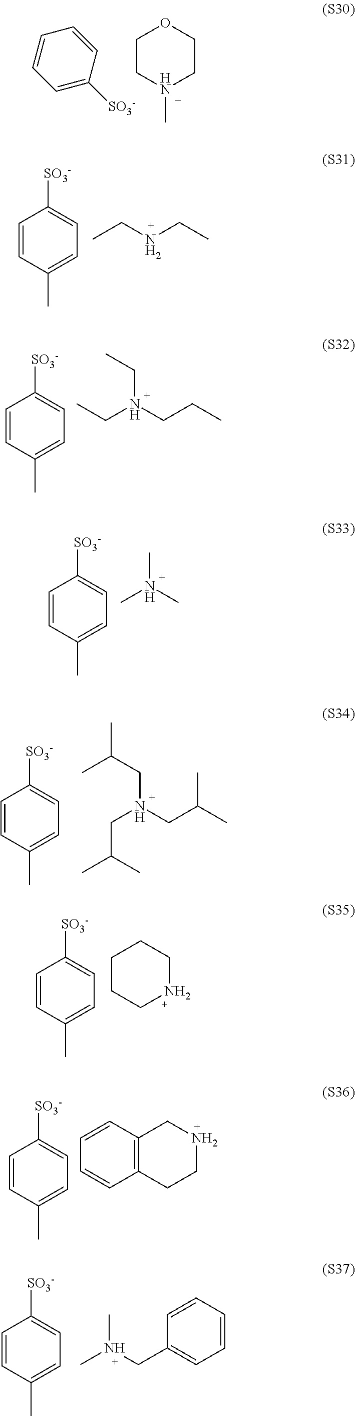

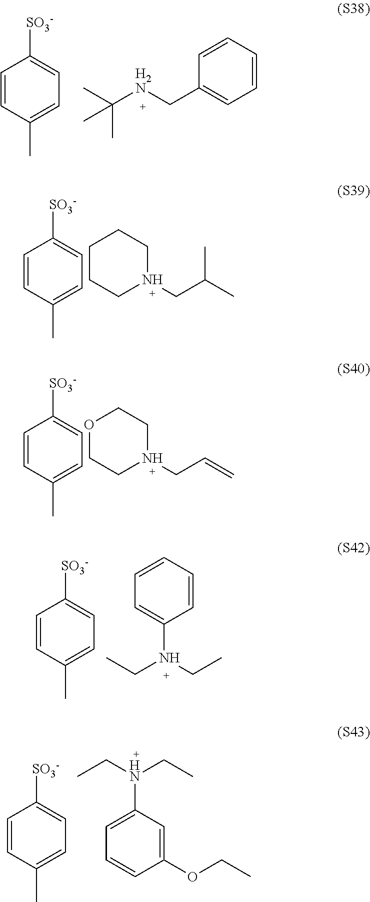

(1-5)

The amount of the thermal acid generator is within the range of 0.0001 to 20% by mass, preferably 0.0005 to 10% by mass, and more preferably 0.01 to 3% by mass based on the total solid content in the resist underlayer film-forming composition.

In an embodiment of the present invention, the thermal decomposition onset temperature of the thermal acid generator, that is, the thermal acid generation temperature is preferably 50° C. or above, more preferably 100° C. or above, and still more preferably 150° C. or above, and is preferably 400° C. or below.

### [2. Polymer (G)]

(2-1)

A polymer (G) in the present invention is not particularly limited. For example, the polymer may be at least one member selected from the group consisting of polyvinyl alcohols, polyacrylamides, (meth)acrylic resins, polyamide acids, polyhydroxystyrenes, polyhydroxystyrene derivatives, polymethacrylate-maleic anhydride copolymers, epoxy resins, phenolic resins, novolac resins, resole resins, maleimide resins, polyether ether ketone resins, polyether ketone resins, polyether sulfone resins, polyketone resins, polyester resins, polyether resins, urea resins, polyamides, polyimides, celluloses, cellulose derivatives, starches, chitins, chitosans, gelatins, zeins, sugar-skeleton polymer compounds, polyethylene terephthalates, polycarbonates, polyurethanes, and polysiloxanes, each of these having an aromatic ring. These resins are used each alone or in combination of two or more thereof.

The polymer (G) in the present invention includes a structural unit derived from (D) an aromatic compound having at least one hydroxy group or amino group, or a compound in which two or more optionally substituted aromatic rings are connected to one another via at least one direct bond, —O—, —S—, —C(═O)—, —SO₂—, —NR— (R denotes a hydrogen atom or a hydrocarbon group), or —(CR₁₁₁R₁₁₂)ₙ—(R₁₁₁ and R₁₁₂ each denote a hydrogen atom, an optionally substituted, C1-C10 linear or cyclic alkyl group, or an aromatic ring, n is 1 to 10, and R₁₁₁ and R₁₁₂ are optionally bonded to each other to form a ring), and (E) an optionally substituted aldehyde compound or aldehyde equivalent. The linear alkyl group may contain an ether bond, a ketone bond, or an ester bond.

Preferably, the polymer (G) is at least one member selected from the group consisting of novolac resins, polyester resins, polyimide resins, and acrylic resins.

The term aromatic ring generally refers to a cyclic organic compound having a 4n+π electron system. Examples of such cyclic organic compounds include substituted or unsubstituted benzene, naphthalene, biphenyl, furan, thiophene, pyrrole, pyridine, indole, quinoline, and carbazole.

The term hydrocarbon group refers to a linear, branched, or cyclic, saturated or unsaturated, aliphatic group or an aromatic group. Preferred hydrocarbon groups are C1-C10 linear or cyclic, aliphatic groups or alkyl groups, and C6-C20 aromatic rings. The alkyl groups may contain an ether bond, a ketone bond, a thioether bond, an amide bond, an NH bond, or an ester bond.

The term aldehyde compound refers to a compound having a —CHO group. The term aldehyde equivalent refers to a compound that can form a novolac resin similarly to aldehyde groups.

### EXAMPLES OF THE SUBSTITUENTS INCLUDE

halogen groups, nitro groups, amino groups, carboxyl groups, carboxylic acid ester groups, nitrile groups, hydroxy groups, epoxy groups, methylol groups, and methoxymethyl groups; C1-C10 alkyl groups, C2-C10 alkenyl groups, C2-C10 alkynyl groups, and C6-C40 aryl groups that are each optionally substituted with any of the above groups; and combinations thereof that optionally contain an ether bond, a ketone bond, a thioether bond, an amide bond, an NH bond, or an ester bond.

The phrase “structural unit derived from” means that the structural unit contains basic skeletons of the compound (D) and of the aldehyde compound or aldehyde equivalent (E). Examples thereof include structural units obtained by chemical reaction of the two compounds.

(2-2)
(2-2-1)

The polymer (G) is preferably a novolac resin.

The term “novolac resins” as used herein refers not only to narrowly defined phenol-formaldehyde resins (the so-called novolac-type phenol resins) and aniline-formaldehyde resins (the so-called novolac-type aniline resins) but also to a broad range of general polymers formed by the formation of covalent bonds (such as a substitution reaction, an addition reaction, or an addition condensation reaction) between an organic compound that has a functional group capable of forming a covalent bond with an aromatic ring in the presence of an acid catalyst or under similar reaction conditions [such as, for example, an aldehyde group, a ketone group, an acetal group, a ketal group; a hydroxy or alkoxy group bonded to a secondary or tertiary carbon; a hydroxy or alkoxy group bonded to an a-carbon atom in an alkylaryl group (such as a benzyl carbon atom); or a carbon-carbon unsaturated bond, such as divinylbenzene or dicyclopentadiene], and an aromatic ring in an aromatic ring-containing compound (preferably having a heteroatom, such as an oxygen atom, a nitrogen atom, or a sulfur atom, on the aromatic ring).

Thus, the novolac resins referred to in the present specification are polymers, in which the organic compound that contains the carbon atom(s) derived from the above functional group (the “linking carbon atom(s)”) connects molecules of the aromatic ring-containing compound by forming covalent bonds with the aromatic rings in the molecules of the aromatic ring-containing compound.

(2-2-2)

More preferably, the polymer (G) is a novolac resin that contains a unit structure having an optionally substituted aromatic ring and:

(i)

the aromatic ring contains a heteroatom in a substituent on the aromatic ring;

(ii)

the unit structure contains two or more aromatic rings and at least two of the aromatic rings are connected to one another by a linking group, and the linking group contains a heteroatom; or

(iii)

the aromatic ring is an aromatic heterocyclic ring or an aromatic ring forming a condensed ring with one or more heterocyclic rings.

The concept of aromatic rings includes not only aromatic hydrocarbon rings but also aromatic heterocyclic rings, and includes not only monocyclic rings but also polycyclic rings. In the case of polycyclic rings, at least one monocyclic ring is an aromatic monocyclic ring, and the rest of the monocyclic rings may be heteromonocyclic rings or alicyclic monocyclic rings.

Furthermore, the concept of heterocyclic rings includes both aliphatic heterocyclic rings and aromatic heterocyclic rings, and includes not only monocyclic rings but also polycyclic rings. In the case of polycyclic rings, at least one monocyclic ring is a heteromonocyclic ring, and the rest of the monocyclic rings may be aromatic hydrocarbon monocyclic rings or alicyclic monocyclic rings.

More preferably, the unit structure of (i) or (ii) is a unit structure having at least one, more preferably two aromatic rings each having an oxygen-containing substituent, or two or more aromatic rings connected to one another by at least one —NH—, respectively.

Examples of the oxygen-containing substituents include hydroxy group; hydroxy group substituted by a saturated or unsaturated, linear, branched, or cyclic hydrocarbon group in place of a hydrogen atom (namely, alkoxy groups); and saturated or unsaturated, linear, branched, or cyclic hydrocarbon groups and aromatic ring residues each interrupted with an oxygen atom one or more times. In addition to the above oxygen-containing substituents, the aromatic rings may have substituents, such as halogen atoms, saturated or unsaturated, linear, branched, or cyclic hydrocarbon groups, hydroxy group, amino group, carboxyl group, cyano group, nitro group, alkoxy groups, ester groups, amide groups, sulfonyl group, sulfide group, ether groups, and aryl groups.

Examples of the aromatic rings include, but are not limited to, aromatic hydrocarbon rings, such as benzene, indene, naphthalene, azulene, styrene, toluene, xylene, mesitylene, cumene, anthracene, phenanthrene, triphenylene, benzanthracene, pyrene, chrysene, fluorene, biphenyl, corannulene, perylene, fluoranthene, benzo[k]fluoranthene, benzo[b]fluoranthene, benzo[ghi]perylene, coronene, dibenzo[g,p]chrysene, acenaphthylene, acenaphthene, naphthacene, and pentacene; and aromatic heterocyclic rings, such as furan, thiophene, pyrrole, imidazole, pyridine, pyrimidine, pyrazine, triazine, thiazole, indole, purine, quinoline, isoquinoline, chromene, thianthrene, phenothiazine, phenoxazine, xanthene, acridine, phenazine, and carbazole.

The aromatic rings may have substituents. Examples of the substituents include halogen atoms, saturated or unsaturated, linear, branched, or cyclic hydrocarbon groups, hydroxy group, amino group, carboxyl group, cyano group, nitro group, alkoxy groups, ester groups, amide groups, sulfonyl group, sulfide group, ether groups, and aryl groups.

The aromatic compounds exemplified in the present specification may have the above substituents unless otherwise specified.

(2-2-3)

More preferably, the polymer (G) has:
(i) one, or two or more types of unit structures having an optionally substituted aromatic ring, and
(ii) a unit structure that includes an optionally substituted monocyclic organic group, in which the monocyclic ring is an aromatic monocyclic ring, or an optionally substituted 4—to 25-membered monocyclic, bicyclic, tricyclic, or tetracyclic organic group, in which the monocyclic ring is a non-aromatic monocyclic ring and in which:

at least one of the monocyclic rings constituting the bicyclic, tricyclic, or tetracyclic ring is a non-aromatic monocyclic ring, and the rest of the monocyclic rings are aromatic monocyclic rings or non-aromatic monocyclic rings. For example, the unit structures may be in the form of dimers or trimers, in which two or three same or different organic groups are connected to one another via a divalent or trivalent linking group.

The monocyclic, bicyclic, tricyclic, or tetracyclic organic group may be condensed with one or more aromatic rings to form a pentacyclic or higher cyclic ring.

Here, the term non-aromatic monocyclic ring refers to a monocyclic ring that is not aromatic, and typically refers to an aliphatic monocyclic ring (that may be an aliphatic heteromonocyclic ring). Examples of the non-aromatic monocyclic rings include cyclopropane, cyclobutane, cyclopentane, cyclohexane, and cyclohexene. Examples of the non-aromatic bicyclic rings include bicyclopentane, bicyclooctane, and bicycloheptene. Examples of the non-aromatic tricyclic rings include tricyclooctane, tricyclononane, and tricyclodecane. Examples of the non-aromatic tetracyclic rings include hexadecahydropyrene.

Preferred examples of the aromatic monocyclic rings or the aromatic rings include those mentioned in (2-2-2) hereinabove, specifically, optionally substituted benzene ring, naphthalene ring, anthracene ring, and pyrene ring. Examples of the substituents include halogen atoms, saturated or unsaturated, linear, branched, or cyclic hydrocarbon groups optionally containing a heteroatom, hydroxy group, amino group, carboxyl group, cyano group, nitro group, alkoxy groups, ester groups, amide groups, sulfonyl group, sulfide group, ether groups, and aryl groups. These examples are not limiting as long as the advantageous effects of the present invention are not impaired.

In the novolac resin, (i) and (ii) are bonded to one another at least by covalently bonding between a carbon atom (a linking carbon atom) on the non-aromatic monocyclic ring in (ii) and a carbon atom on the aromatic ring in (i).

Here, typical examples of (ii) include unit structures, in which the keto group in a cyclic ketone is substituted by two valence bonds; and unit structures, in which an organic group is added to the keto group in a cyclic ketone to convert the ketone into a tertiary alcohol, and the tertiary hydroxy group is substituted by one valence bond.

When the unit structure (ii) contains an aromatic ring, the aromatic ring may be bonded to each of the linking carbon atoms in other two unit structures (ii). The unit structure resulting from this manner of bonding may be used as a type of the unit structure (i).

When the unit structure (ii) contains an aromatic ring, the linking carbon atom in the unit structure (ii) may be bonded to the aromatic ring in the unit structure (i), and further the aromatic ring X in the unit structure (ii) may be bonded to the linking carbon atom in another of the unit structure (ii). The unit structure resulting from this manner of bonding is equivalent to the composite unit structure consisting of one unit structure (i) and one unit structure (ii) and may be used in place of at least part of the composite unit structures. For example, reference may be made to (2-3-10) below.

(2-3)
(2-3-1)

Preferably, the polymer (G) is a novolac resin that includes a structure represented by formula (X) below:

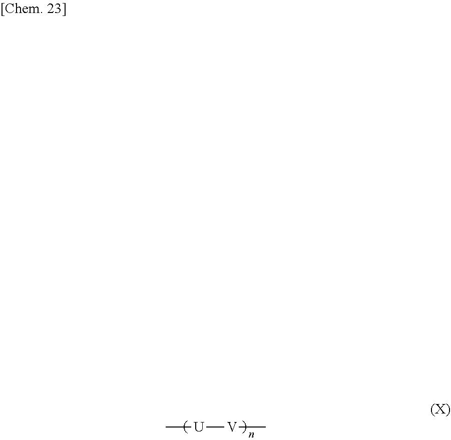

[in formula (X), n indicates a number of composite unit structures U-V,

the unit structure U comprises one, or two or more types of unit structures having an optionally substituted aromatic ring, and is optionally such that:

the optionally substituted aromatic ring is substituted with a substituent that contains a heteroatom; the unit structure contains aromatic rings connected to one another by a linking group that contains a heteroatom; or

the aromatic ring is an aromatic heterocyclic ring or is an aromatic ring forming a condensed ring with one or more heterocyclic rings; and

the unit structure V comprises one, or two or more types of unit structures containing at least one structure selected from formula (II), (III), or (IV) described later].

The unit structure U comprises one, or two or more types of unit structures having an optionally substituted aromatic ring.

Examples of the substituents include halogen atoms, saturated or unsaturated, linear, branched, or cyclic hydrocarbon groups optionally containing a heteroatom, hydroxy group, amino group, carboxyl group, cyano group, nitro group, alkoxy groups, ester groups, amide groups, sulfonyl group, sulfide group, ether groups, and aryl groups, but are not limited thereto as long as the advantageous effects of the present invention are not impaired.

The substituent may contain a heteroatom; the unit structure may contains two or more aromatic rings, the aromatic rings may be connected to one another by a linking group, and the linking group may contain a heteroatom; or the aromatic ring may be an aromatic heterocyclic ring or may be an aromatic ring forming a condensed ring with one or more heterocyclic rings.

(2-3-2)

As mentioned in (2-2-2), the concept of the “aromatic rings” in the unit structure U includes not only aromatic hydrocarbon rings but also aromatic heterocyclic rings and includes not only monocyclic rings but also polycyclic rings. In the case of polycyclic rings, at least one monocyclic ring is an aromatic monocyclic ring, and the rest of the monocyclic rings may be heteromonocyclic rings or alicyclic monocyclic rings.

Examples of the aromatic rings include groups derived from benzene, cyclooctatetraene, and aromatic compounds having an appropriate substituent, such as indene, naphthalene, azulene, styrene, toluene, xylene, mesitylene, cumene, anthracene, phenanthrene, naphthacene, triphenylene, benzanthracene, pyrene, chrysene, fluorene, biphenyl, corannulene, perylene, fluoranthene, benzo[k]fluoranthene, benzo[b]fluoranthene, benzo[ghi]perylene, coronene, dibenzo[g,p]chrysene, acenaphthylene, acenaphthene, naphthacene, pentacene, N-alkylpyrroles, and N-arylpyrroles.

Examples further include organic groups that have a condensed ring formed between one or more aromatic hydrocarbon rings (such as benzene, naphthalene, anthracene, and pyrene) and one or more aliphatic rings or heterocyclic rings. Examples of the aliphatic rings include cyclobutane, cyclobutene, cyclopentane, cyclopentene, cyclohexane, cyclohexene, methylcyclohexane, methylcyclohexene, cycloheptane, and cycloheptene. Examples of the heterocyclic rings include furan, thiophene, pyrrole, imidazole, pyran, pyridine, pyrimidine, pyrazine, pyrrolidine, piperidine, piperazine, and morpholine.

As mentioned in (2-2-2), the concept of the “heterocyclic rings” includes both aliphatic heterocyclic rings and aromatic heterocyclic rings and includes not only monocyclic rings but also polycyclic rings. In the case of polycyclic rings, at least one monocyclic ring is a heteromonocyclic ring, and the rest of the monocyclic rings may be aromatic hydrocarbon monocyclic rings or alicyclic monocyclic rings.

The aromatic rings may be organic groups that have a structure, in which two or more aromatic rings are connected to one another by a linking group, such as an alkylene group.

Preferably, the “aromatic ring” in the unit structure U has 6 to 30 carbon atoms or 6 to 24 carbon atoms.

Preferably, the “aromatic ring” in the unit structure U is one or more benzene, naphthalene, anthracene, or pyrene rings; or is a condensed ring formed between a benzene ring, a naphthalene ring, an anthracene ring, or a pyrene ring and a heterocyclic ring or an aliphatic ring.

The aromatic ring in the unit structure U may be optionally substituted, and the substituent preferably contains a heteroatom.

The aromatic ring in the unit structure U may include two or more aromatic rings connected to one another by a linking group, and the linking group preferably contains a heteroatom.

Examples of the heteroatoms include oxygen atom, nitrogen atom, and sulfur atom.

Preferably, the “aromatic ring” in the unit structure U is a C6-C30 or C6-C24 organic group that contains at least one heteroatom selected from N, S, and O on the ring, within the ring, or between the rings.

Examples of the heteroatoms contained on the ring include nitrogen atom in amino groups (for example, propargylamino group) and in cyano group; oxygen atom in oxygen-containing substituents, such as formyl group, hydroxy group, carboxyl group, and alkoxy groups (for example, propargyloxy group); and nitrogen atom and oxygen atom in nitro group that is an oxygen-containing and nitrogen-containing substituent.

Examples of the heteroatoms contained within the ring include oxygen atom in xanthene and nitrogen atom in carbazole.

Examples of the heteroatoms contained in the linking group between two or more aromatic rings include nitrogen atom, oxygen atom, and sulfur atom in —NH— bond, —NHCO— bond, —O— bond, —COO— bond, —CO— bond, —S— bond, —SS— bond, and —SO₂— bond.

Preferably, the unit structure U is a unit structure that has an aromatic ring having an oxygen-containing substituent mentioned above, a unit structure that has two or more aromatic rings connected to one another by —NH—, or a unit structure that has a condensed ring formed between one or more aromatic hydrocarbon rings and one or more heterocyclic rings.

(2-3-3)

Preferably, the unit structure U is at least one member selected from the following.

### Examples of Amine Skeletons

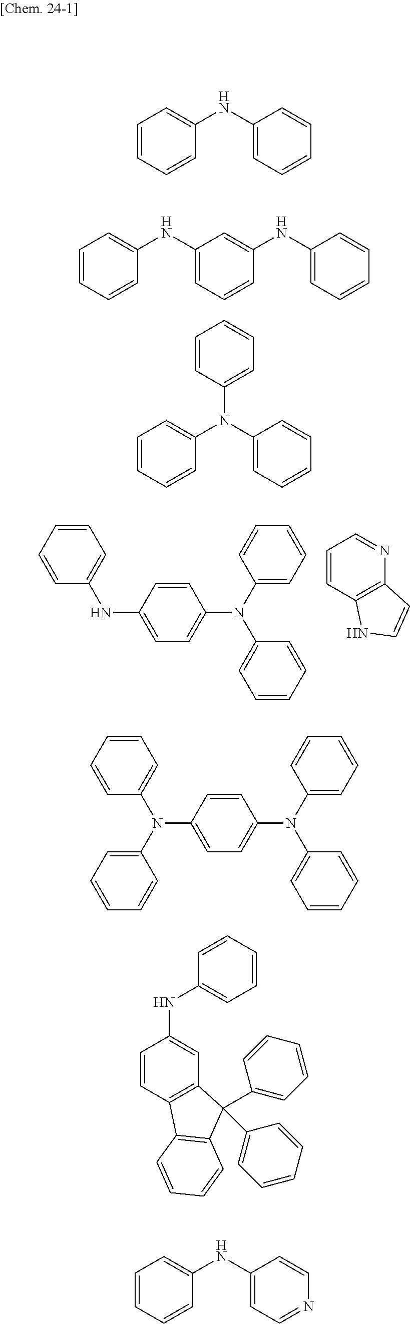

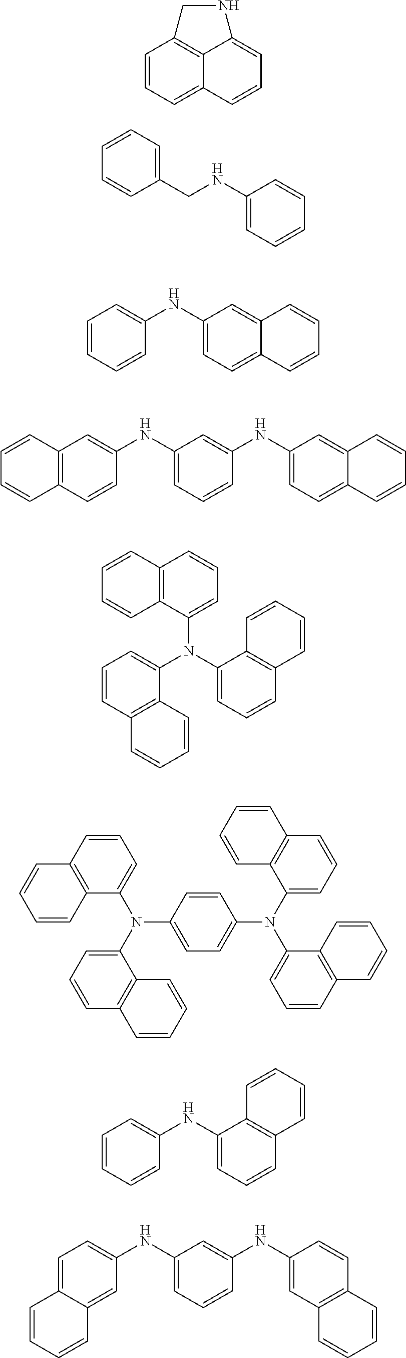

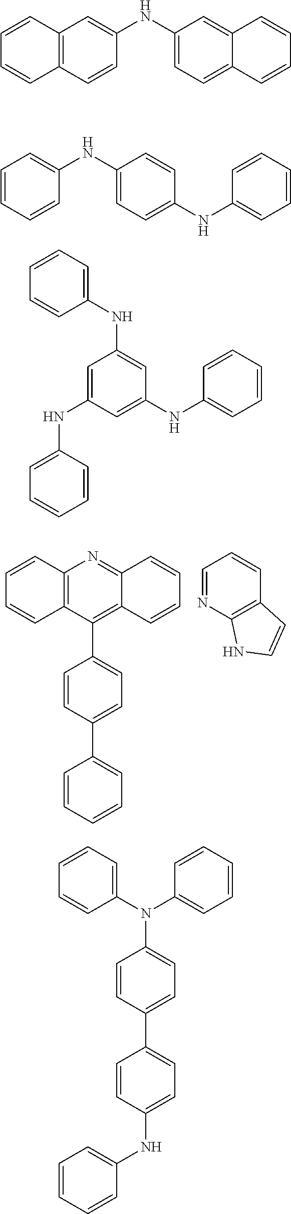

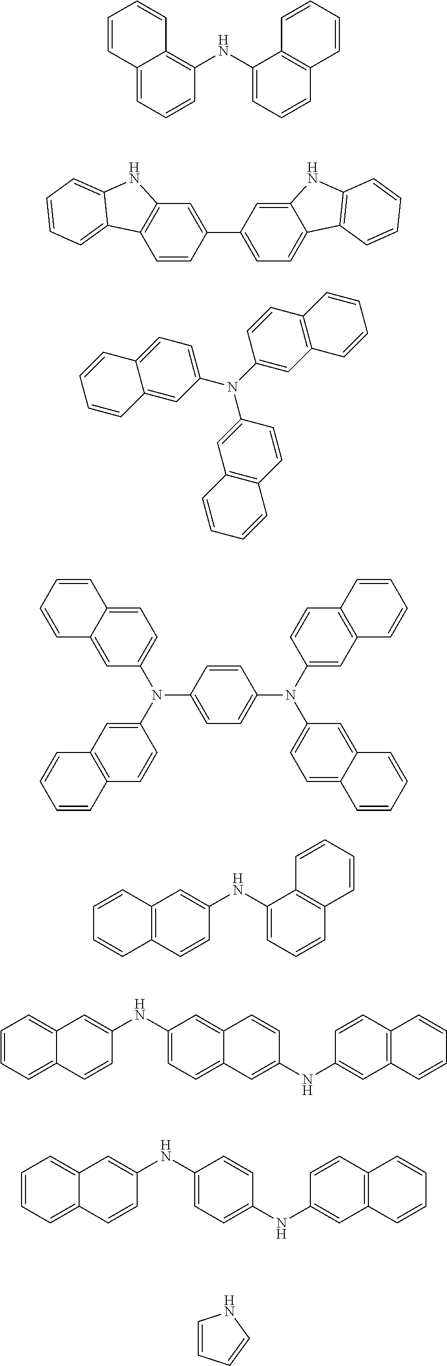

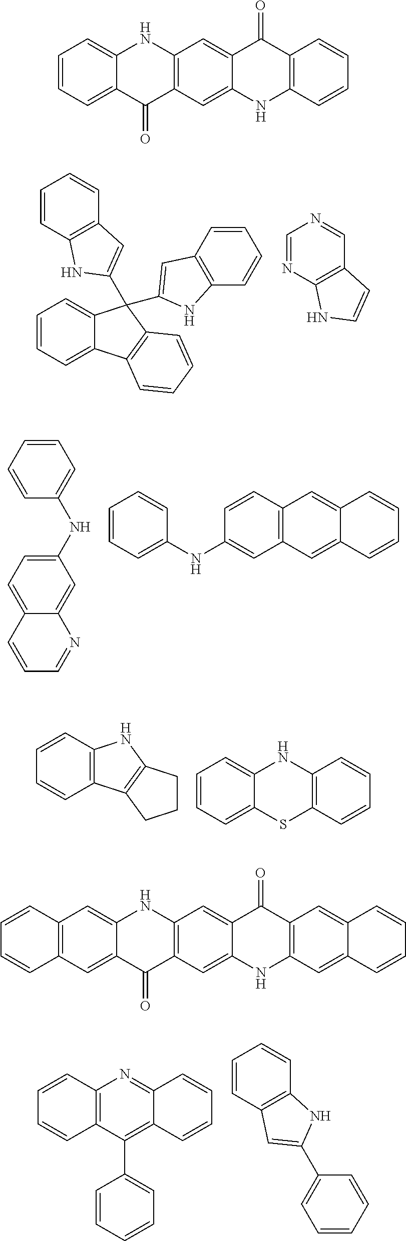

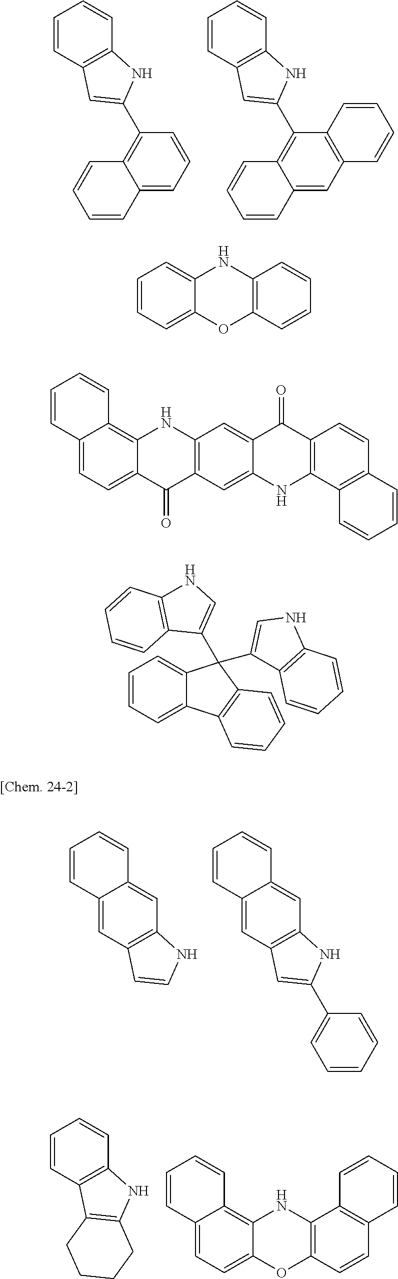

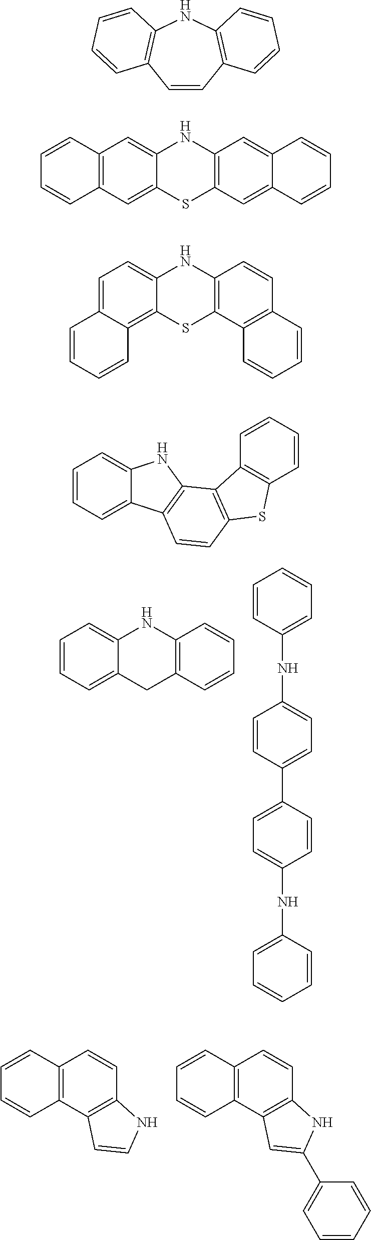

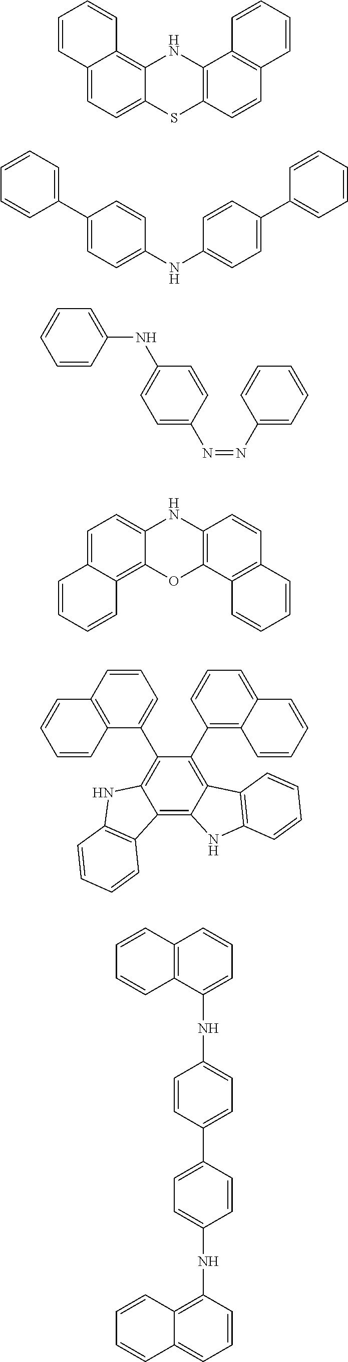

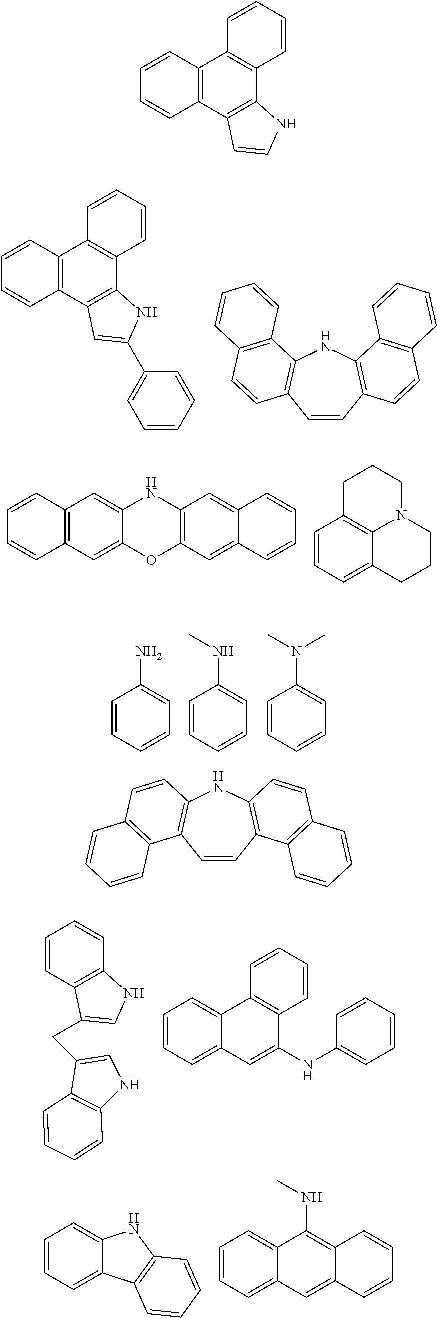

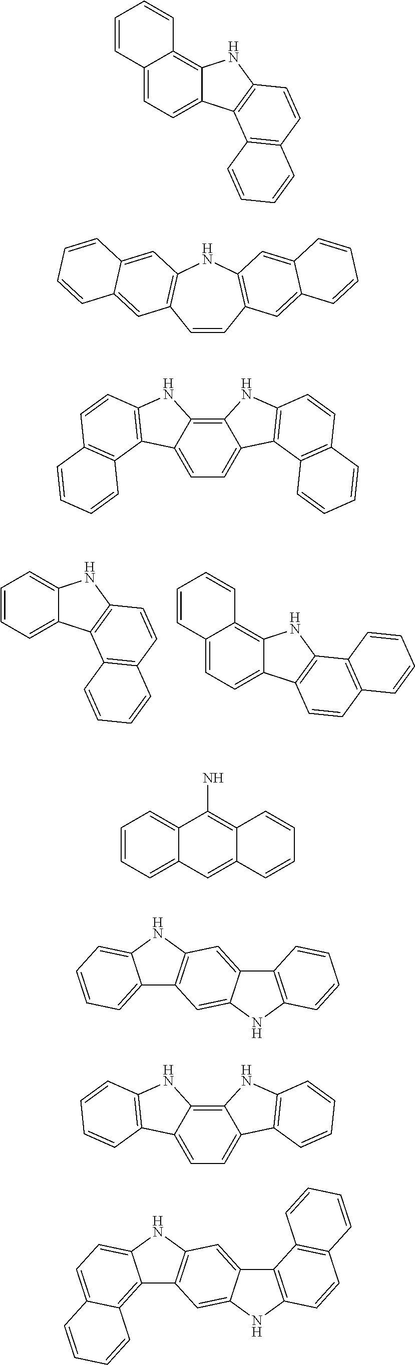

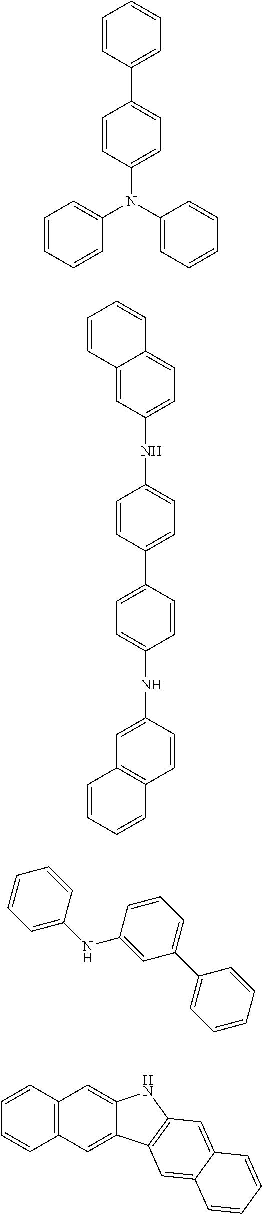 [SMILES: c1ccc(-c2ccc(N(c3ccccc3)c3ccccc3)cc2)cc1 ; c1ccc2cc(Nc3ccc(-c4ccc(Nc5ccc6ccccc6c5)cc4)cc3)ccc2c1 ; c1ccc(Nc2cccc(-c3ccccc3)c2)cc1 ; c1ccc2cc3c(cc2c1)[nH]c1cc2ccccc2cc13]

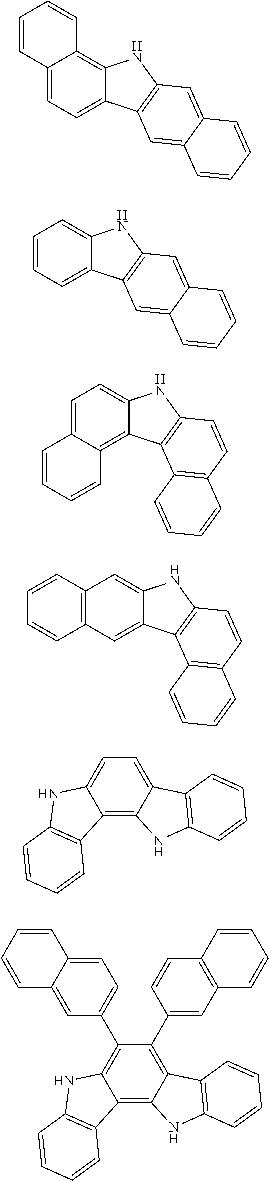

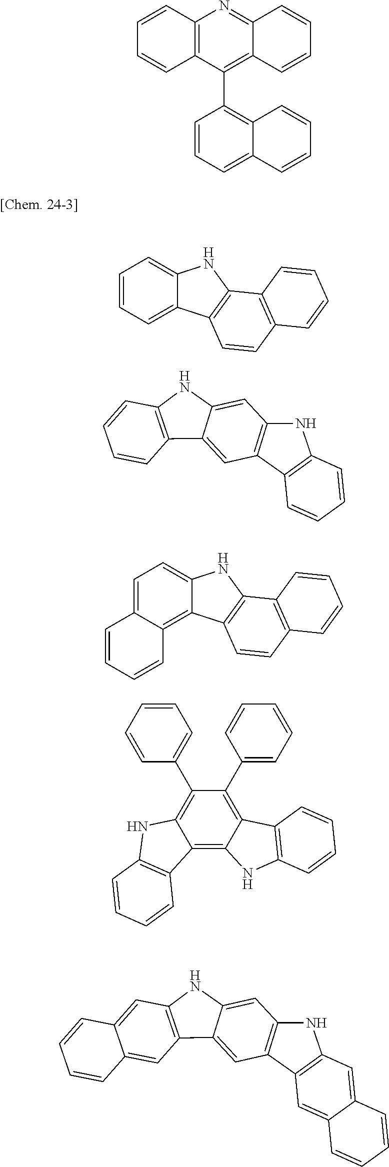

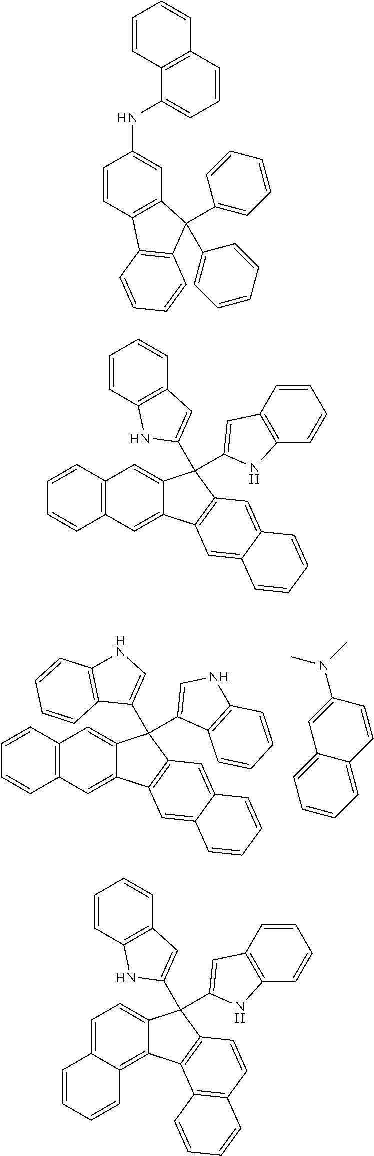

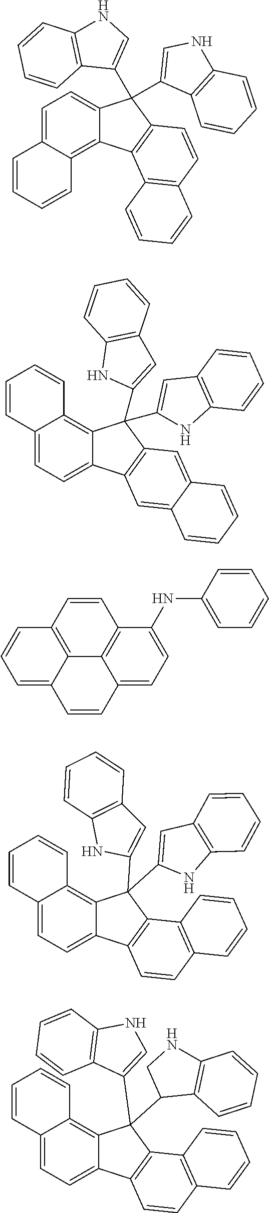

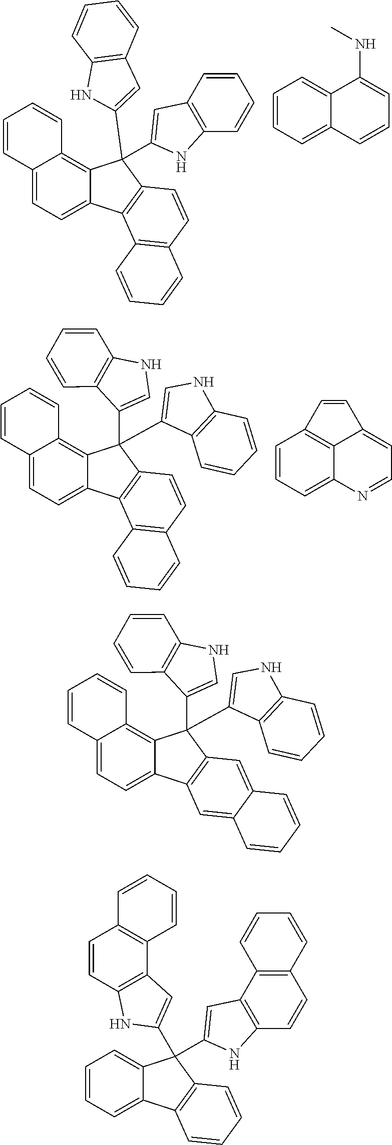

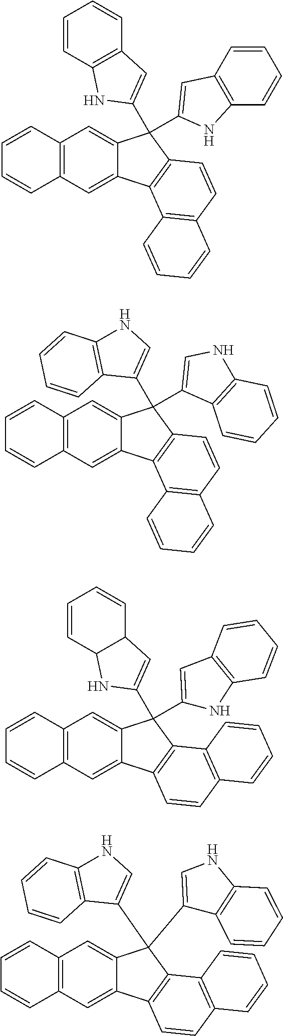 [SMILES: c1ccc2cc3c(cc2c1)-c1c(ccc2ccccc12)C3(c1cc2ccccc2[nH]1)c1cc2ccccc2[nH]1 ; c1ccc2cc3c(cc2c1)-c1c(ccc2ccccc12)C3(c1c[nH]c2ccccc12)c1c[nH]c2ccccc12 ; C1=CC2C=C(C3(c4cc5ccccc5[nH]4)c4cc5ccccc5cc4-c4ccc5ccccc5c43)NC2C=C1 ; c1ccc2cc3c(cc2c1)-c1ccc2ccccc2c1C3(c1c[nH]c2ccccc12)c1c[nH]c2ccccc12]

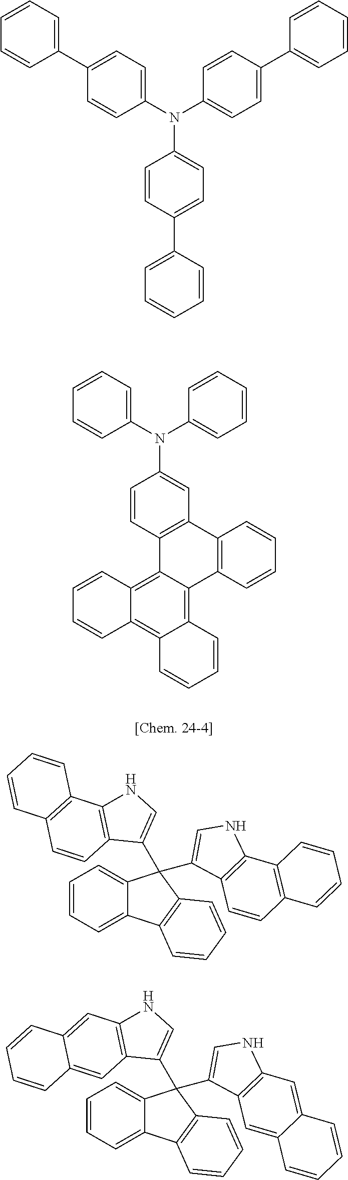 [SMILES: c1ccc(-c2ccc(N(c3ccc(-c4ccccc4)cc3)c3ccc(-c4ccccc4)cc3)cc2)cc1 ; c1ccc(N(c2ccccc2)c2ccc3c(c2)c2ccccc2c2c4ccccc4c4ccccc4c32)cc1 ; c1ccc2c(c1)-c1ccccc1C2(c1c[nH]c2c1ccc1ccccc12)c1c[nH]c2c1ccc1ccccc12 ; c1ccc2c(c1)-c1ccccc1C2(c1c[nH]c2cc3ccccc3cc12)c1c[nH]c2cc3ccccc3cc12]

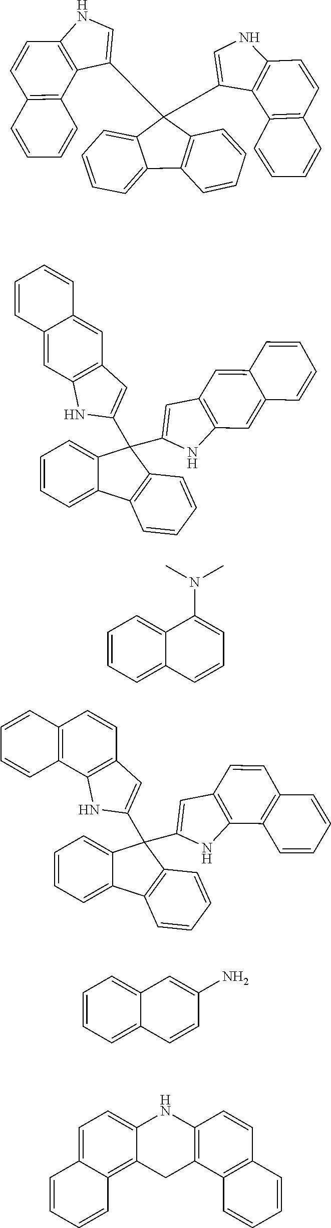

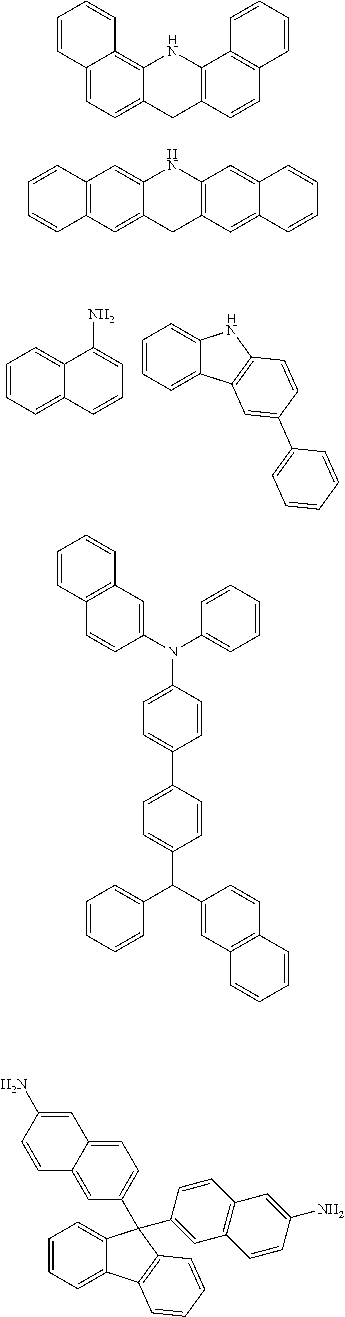

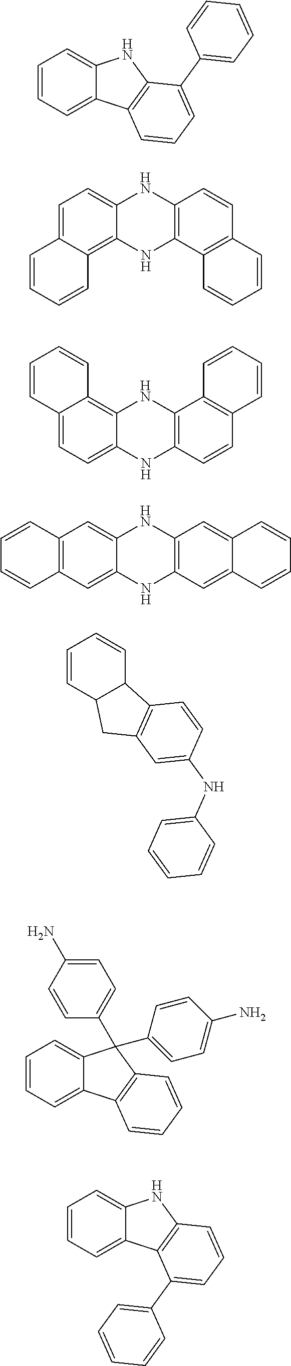

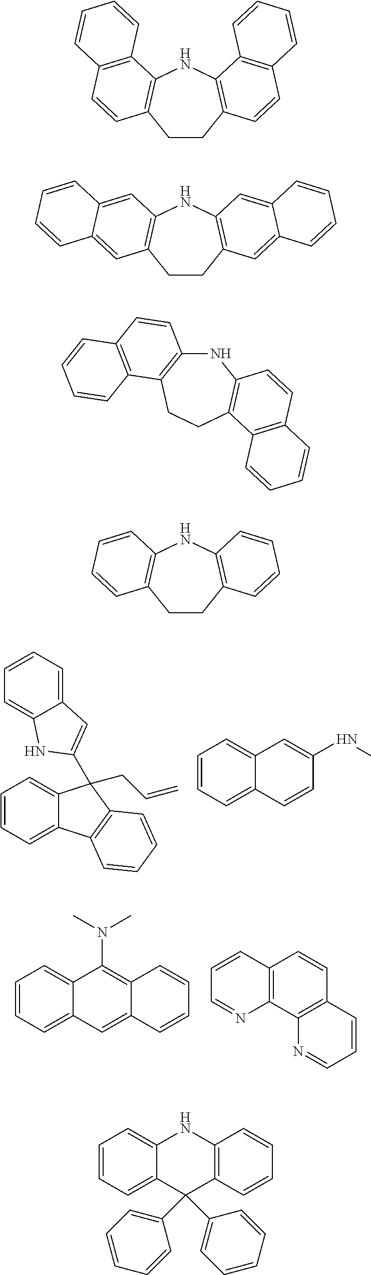

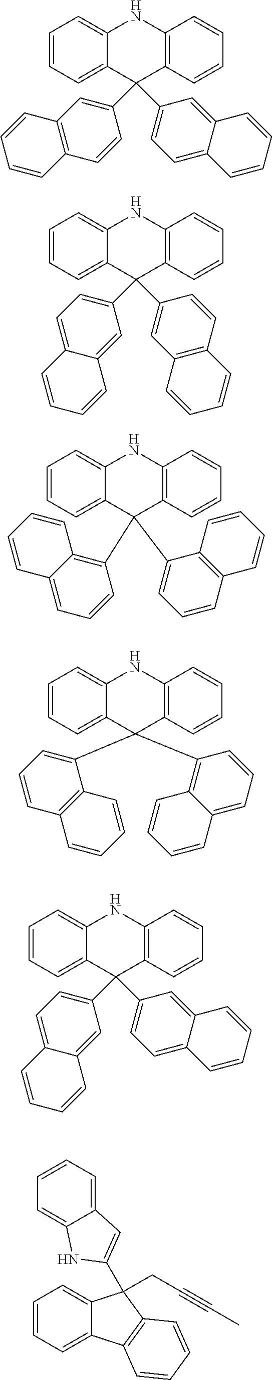

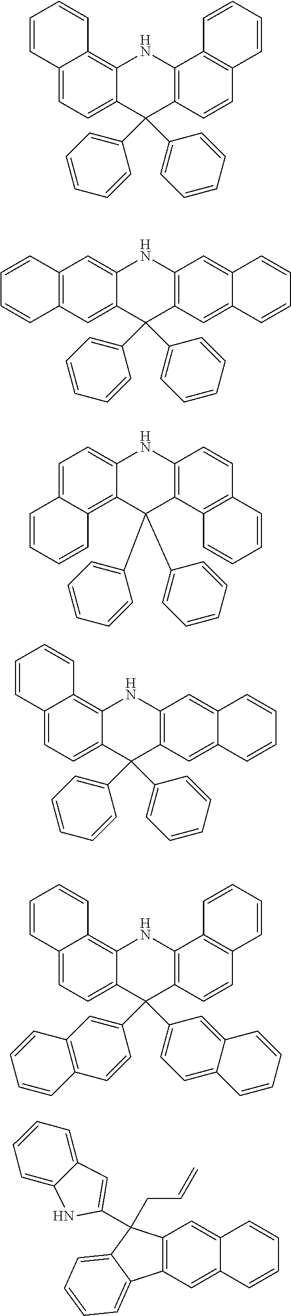

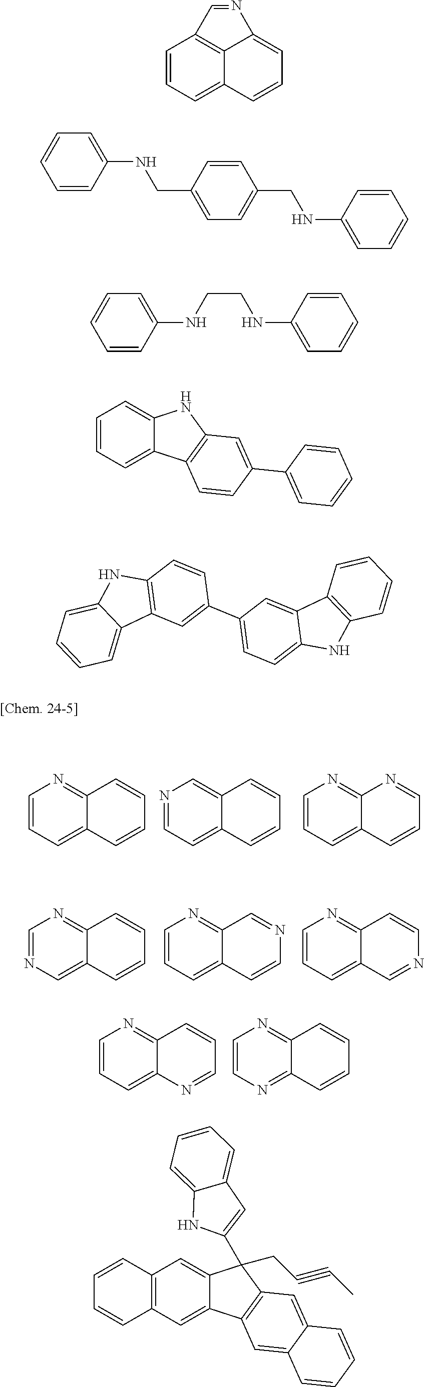

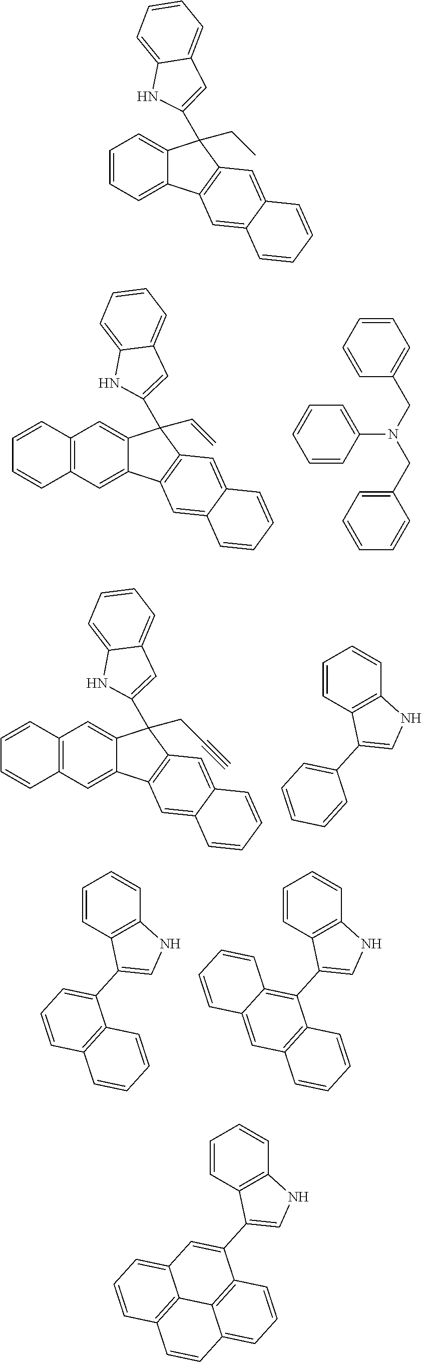

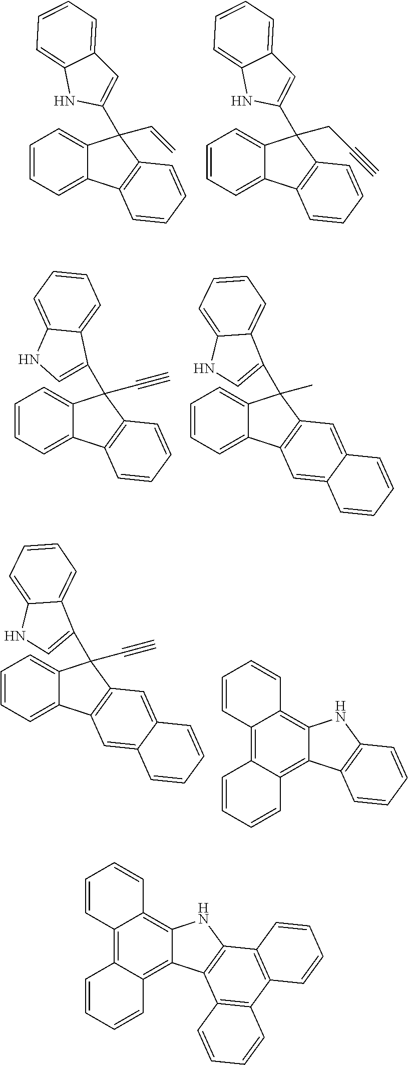

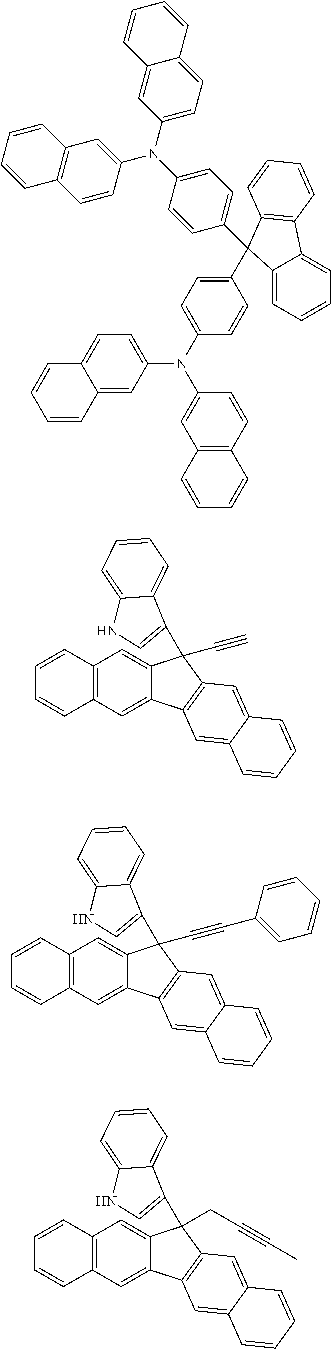 [SMILES: c1ccc2c(c1)-c1ccccc1C2(c1ccc(N(c2ccc3ccccc3c2)c2ccc3ccccc3c2)cc1)c1ccc(N(c2ccc3ccccc3c2)c2ccc3ccccc3c2)cc1 ; C#CC1(c2c[nH]c3ccccc23)c2cc3ccccc3cc2-c2cc3ccccc3cc21 ; C(#CC1(c2c[nH]c3ccccc23)c2cc3ccccc3cc2-c2cc3ccccc3cc21)c1ccccc1 ; CC#CCC1(c2c[nH]c3ccccc23)c2cc3ccccc3cc2-c2cc3ccccc3cc21]

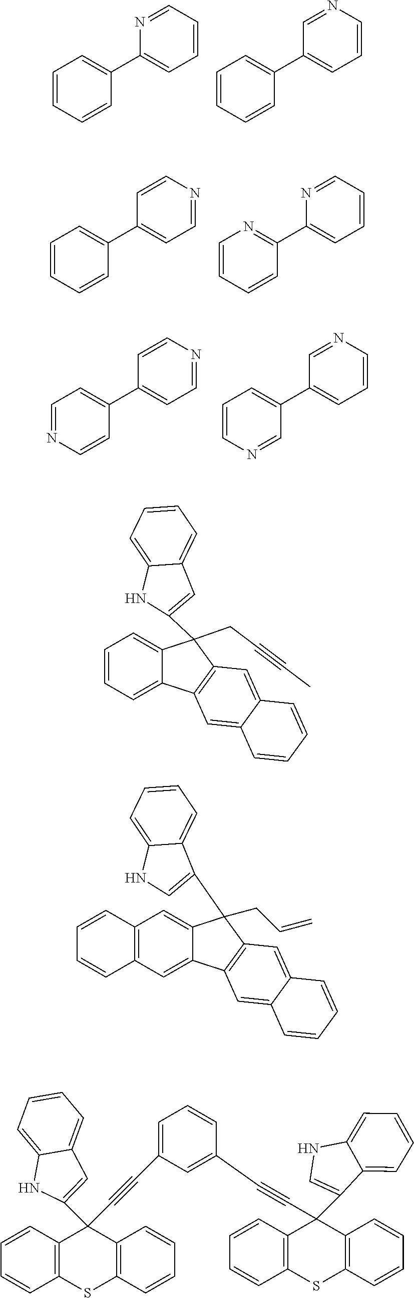

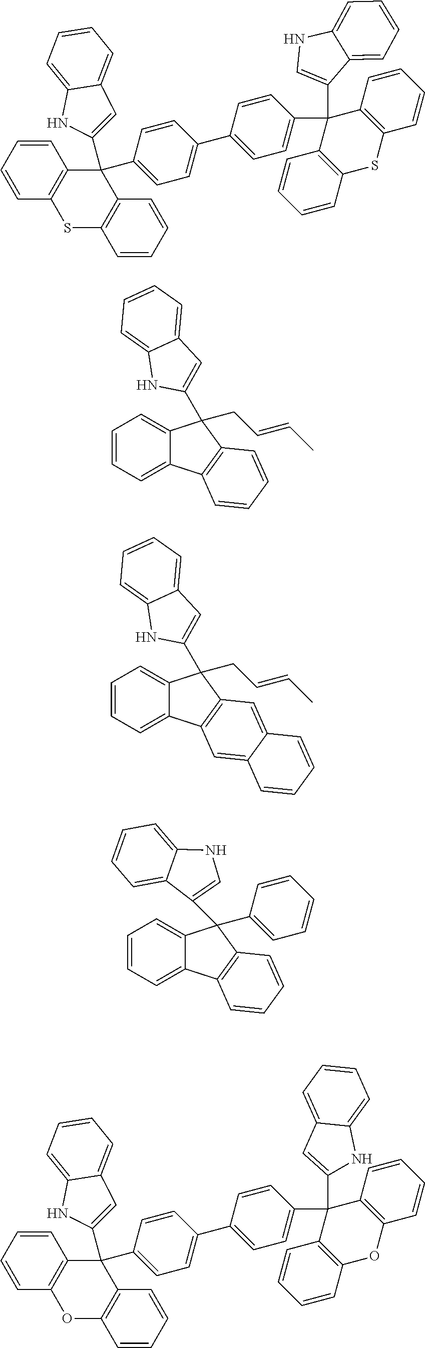

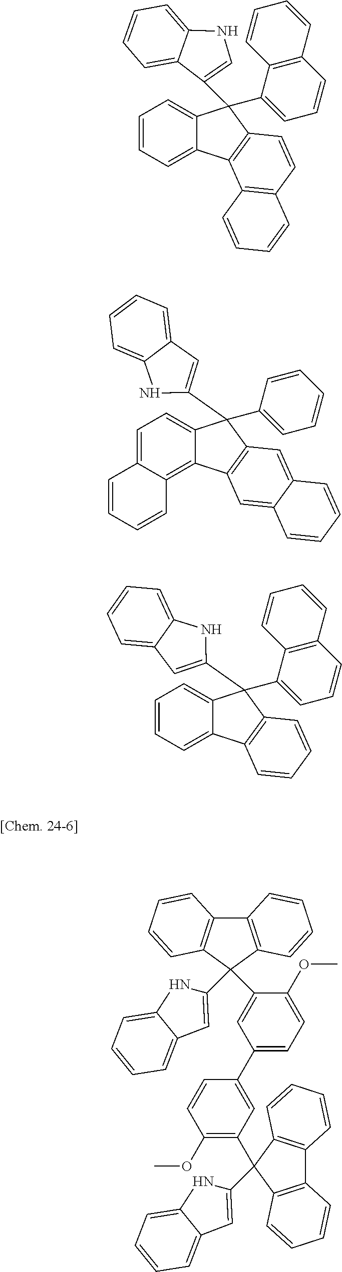 [SMILES: c1ccc2c(c1)-c1c(ccc3ccccc13)C2(c1cccc2ccccc12)c1c[nH]c2ccccc12 ; c1ccc(C2(c3cc4ccccc4[nH]3)c3cc4ccccc4cc3-c3c2ccc2ccccc32)cc1 ; c1ccc2c(c1)-c1ccccc1C2(c1cc2ccccc2[nH]1)c1cccc2ccccc12 ; COc1ccc(-c2ccc(OC)c(C3(c4cc5ccccc5[nH]4)c4ccccc4-c4ccccc43)c2)cc1C1(c2cc3ccccc3[nH]2)c2ccccc2-c2ccccc21]

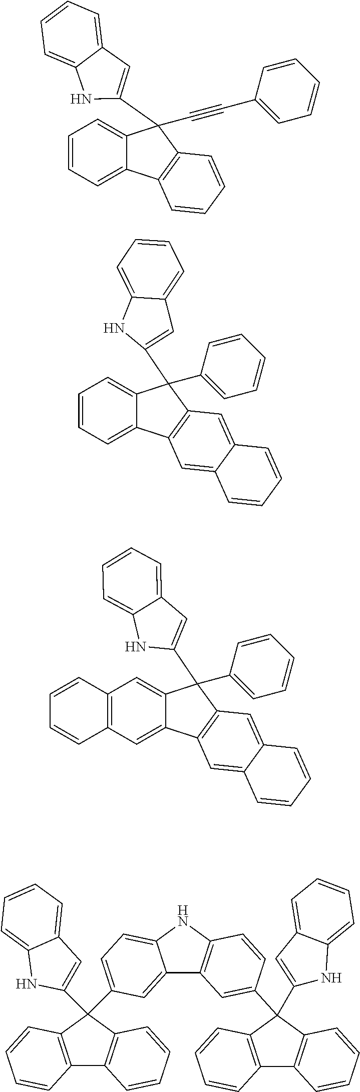

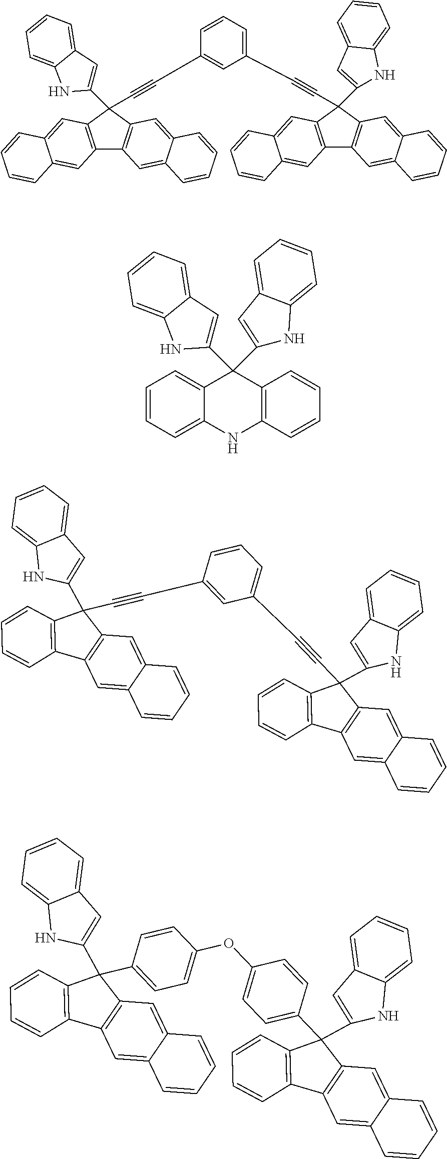 [SMILES: C(#CC1(c2cc3ccccc3[nH]2)c2cc3ccccc3cc2-c2cc3ccccc3cc21)c1cccc(C#CC2(c3cc4ccccc4[nH]3)c3cc4ccccc4cc3-c3cc4ccccc4cc32)c1 ; c1ccc2c(c1)Nc1ccccc1C2(c1cc2ccccc2[nH]1)c1cc2ccccc2[nH]1 ; C(#CC1(c2cc3ccccc3[nH]2)c2ccccc2-c2cc3ccccc3cc21)c1cccc(C#CC2(c3cc4ccccc4[nH]3)c3ccccc3-c3cc4ccccc4cc32)c1 ; c1ccc2c(c1)-c1cc3ccccc3cc1C2(c1ccc(Oc2ccc(C3(c4cc5ccccc5[nH]4)c4ccccc4-c4cc5ccccc5cc43)cc2)cc1)c1cc2ccccc2[nH]1]

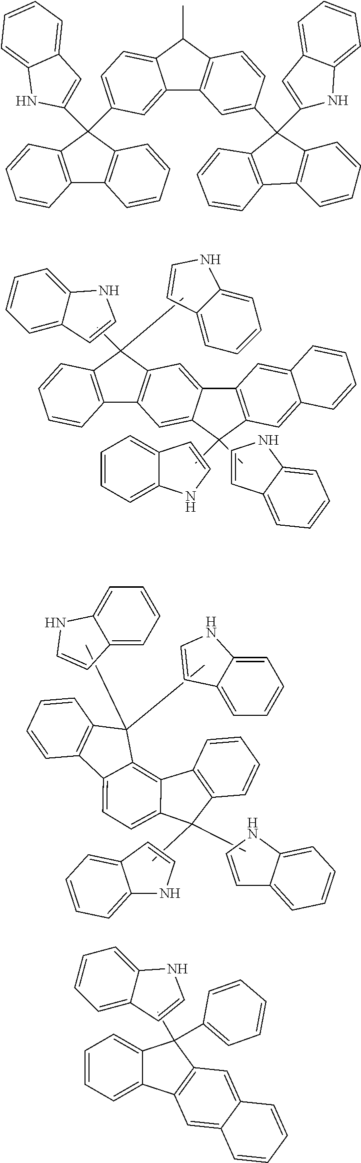

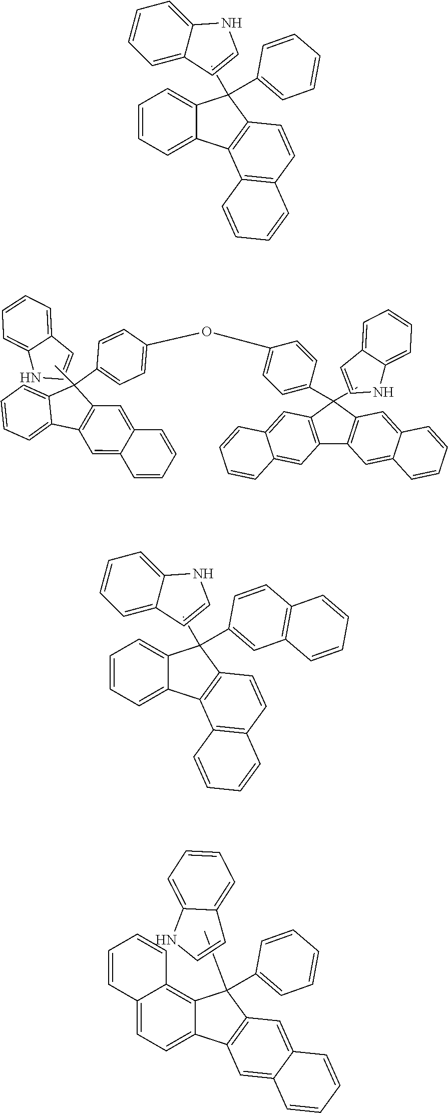

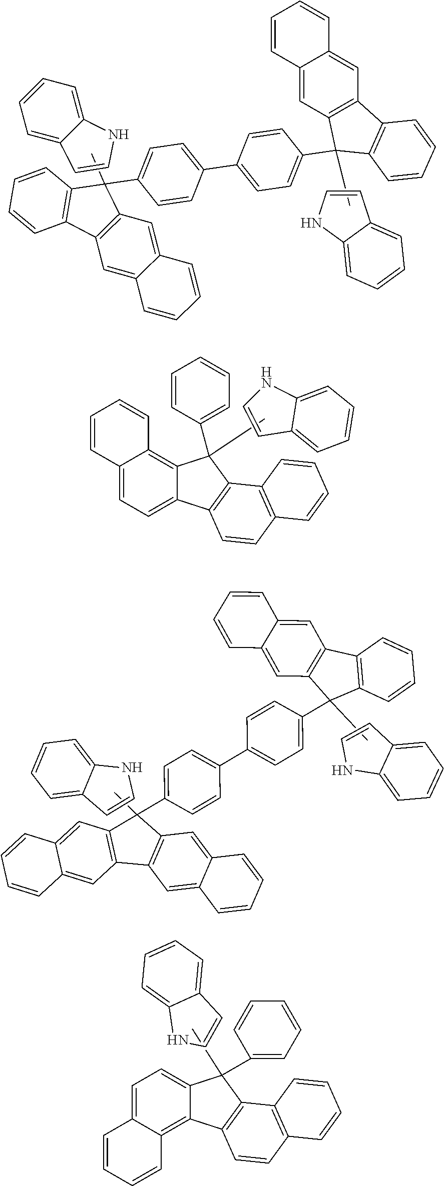

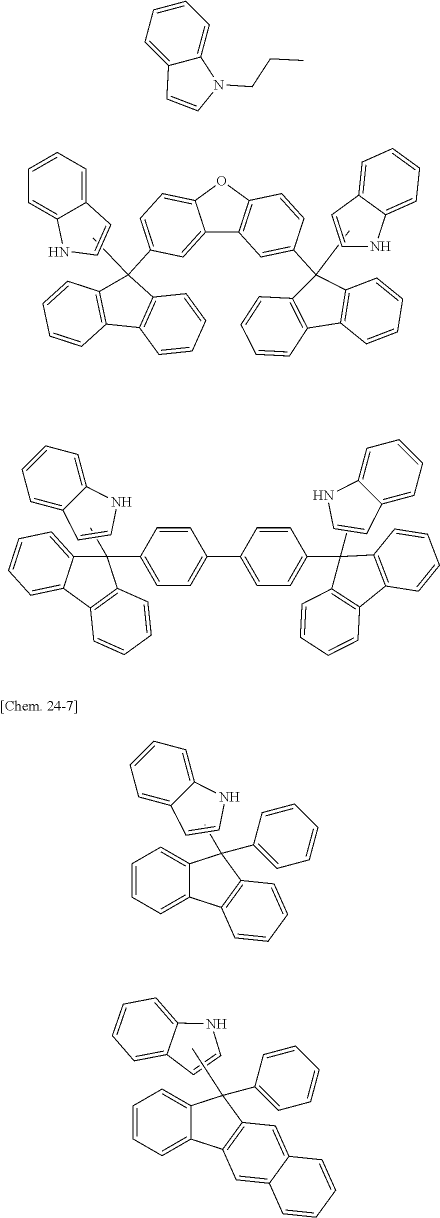

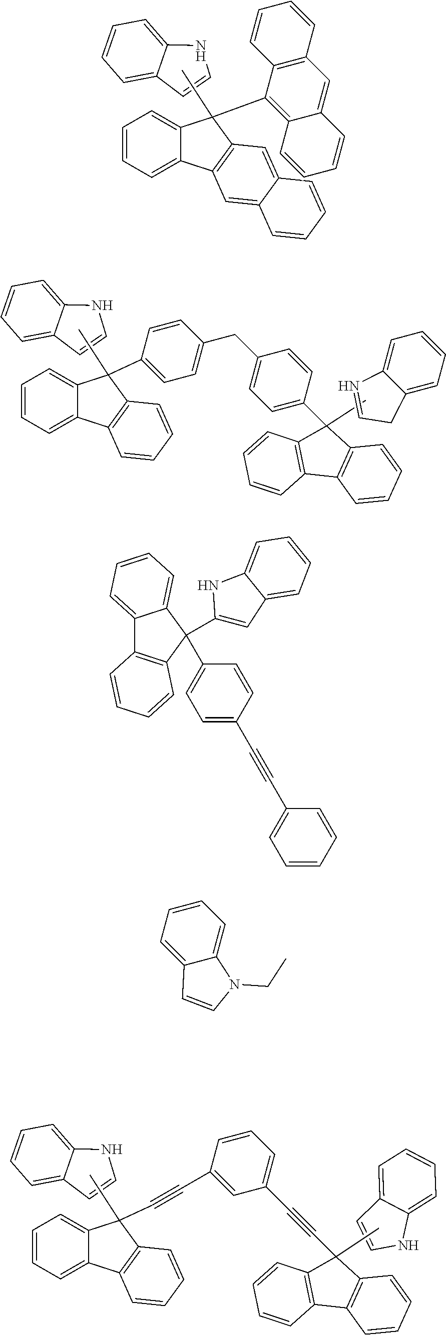

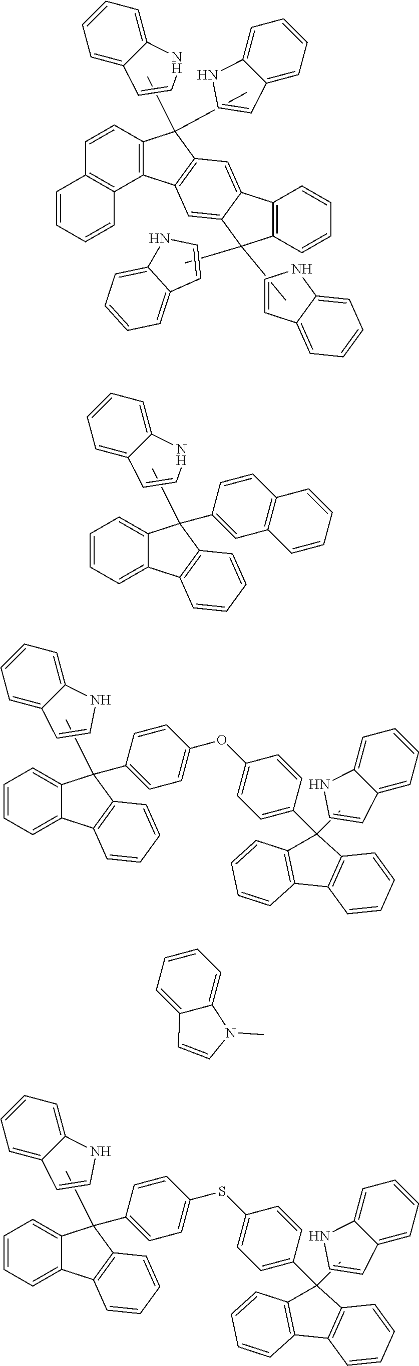

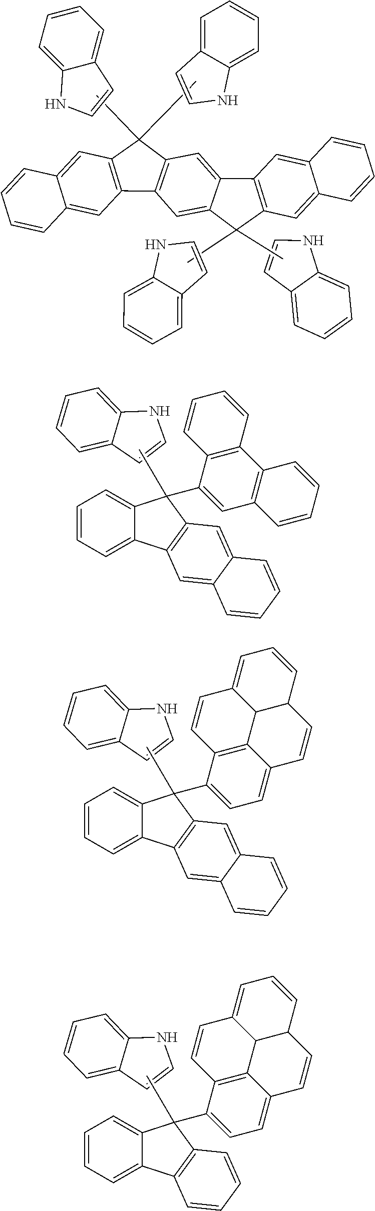

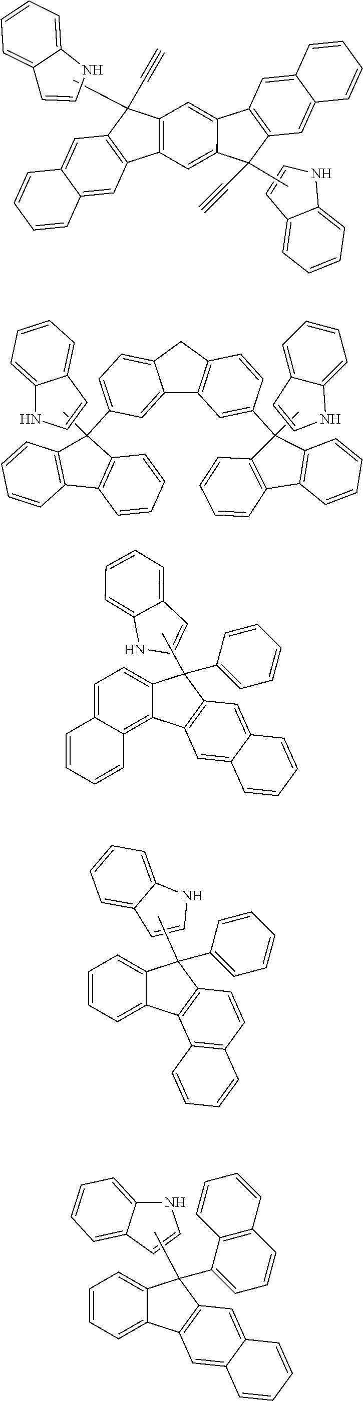

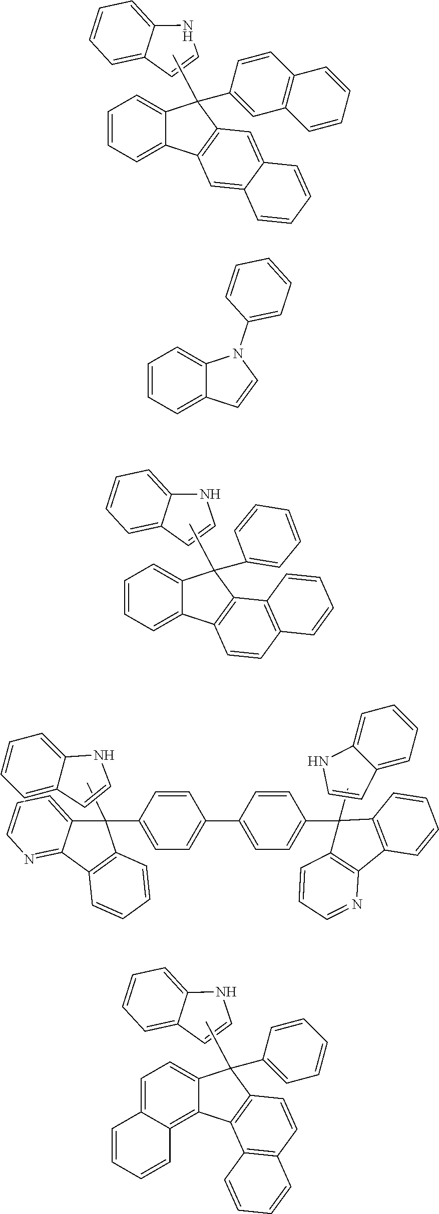

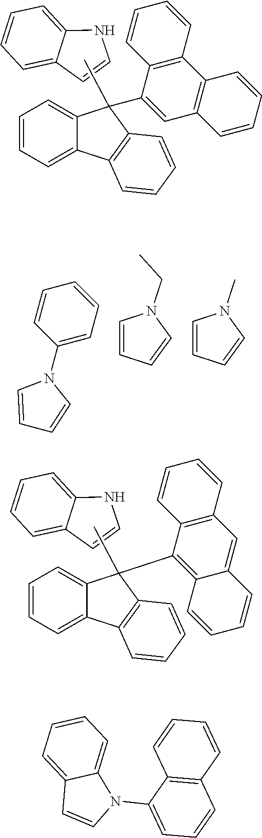

### Examples of Phenol Skeletons

The compounds below are only illustrative, and the number and the substitution positions of hydroxy groups are not limited to the illustrated structures.

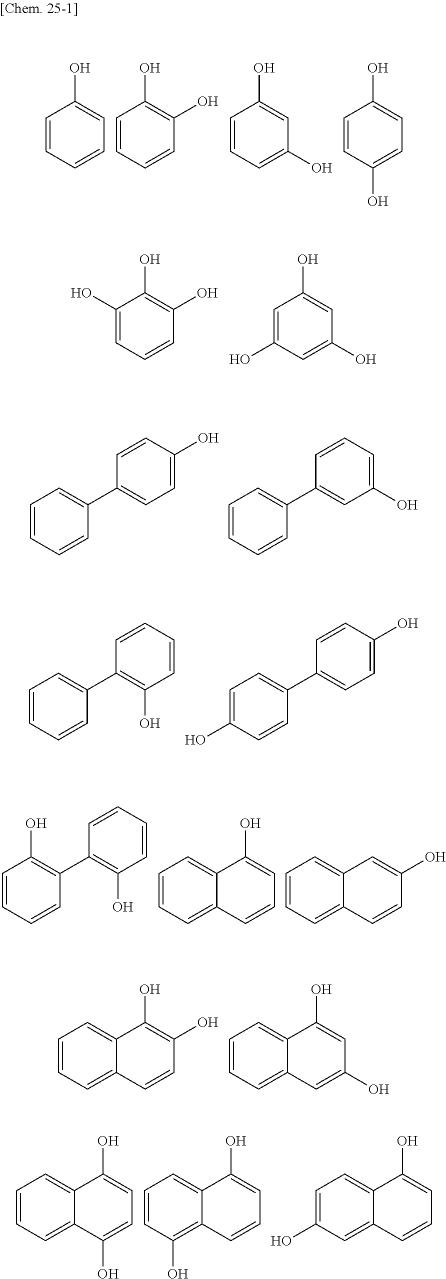

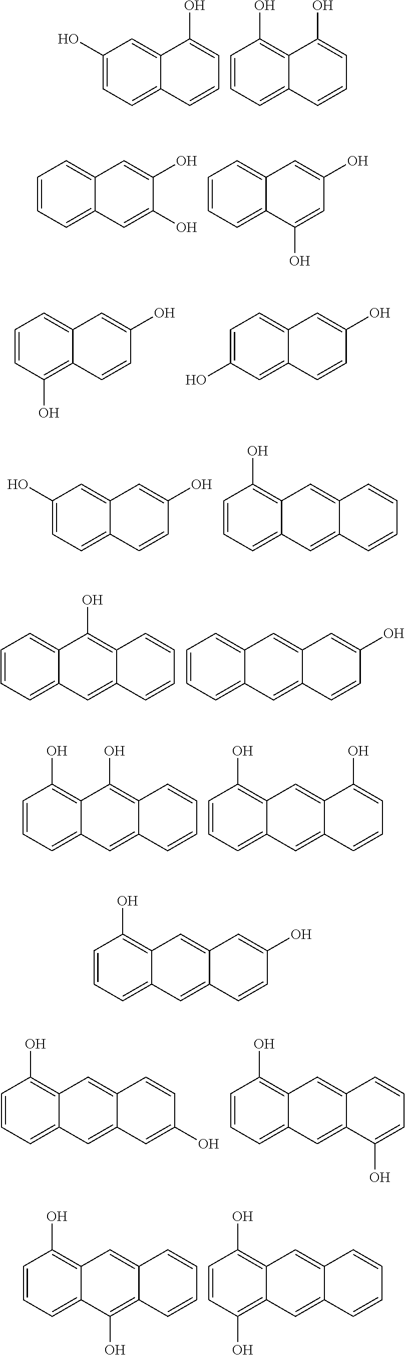

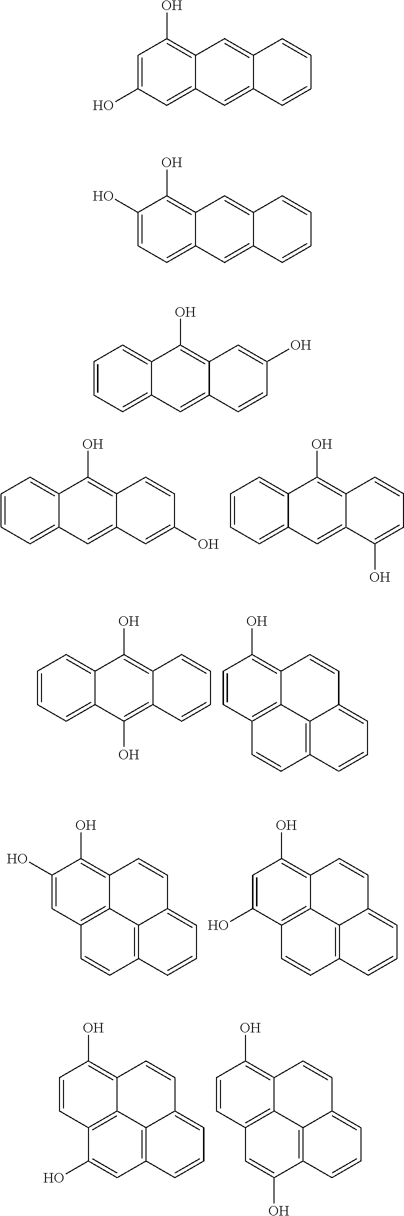

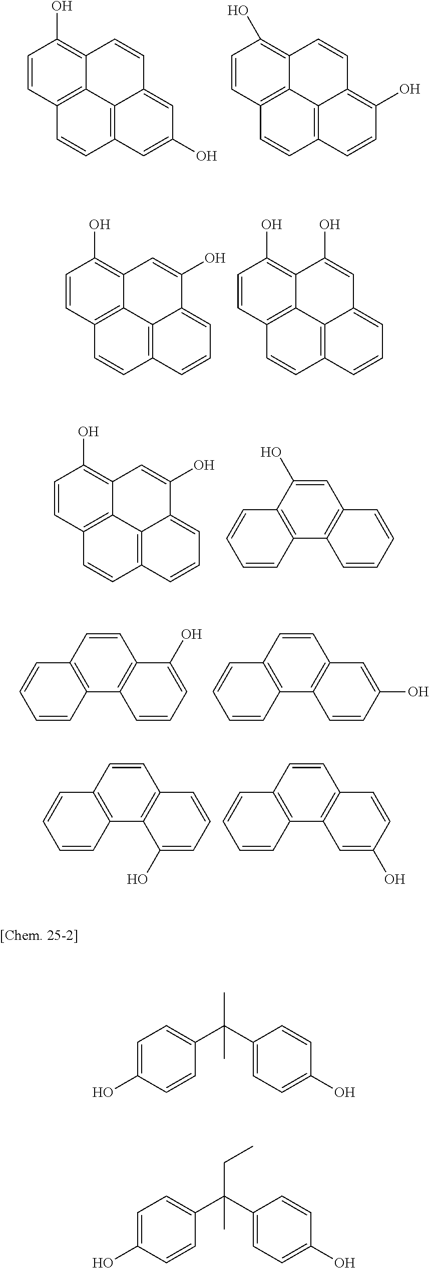

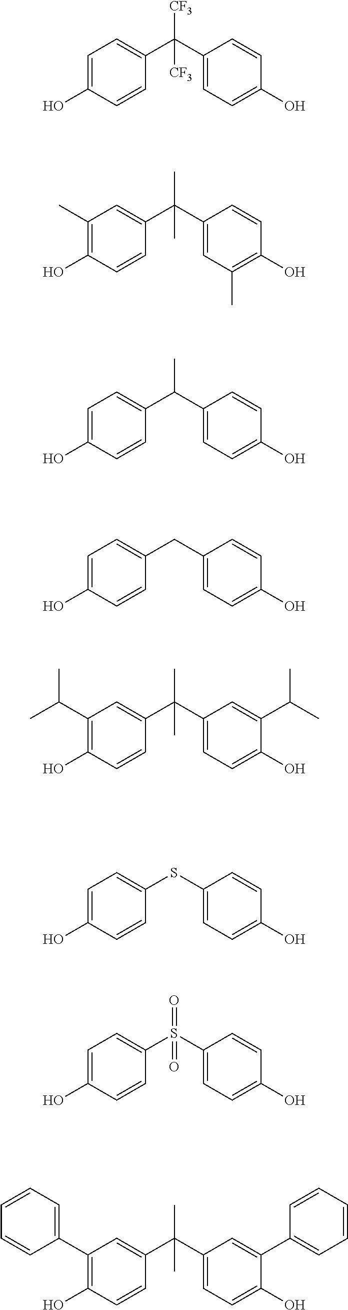

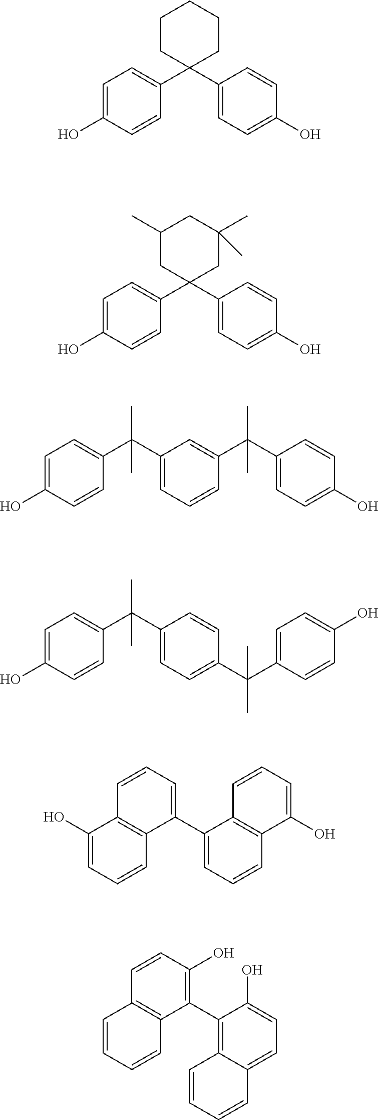 [SMILES: Oc1ccc(C2(c3ccc(O)cc3)CCCCC2)cc1 ; CC1CC(C)(C)CC(c2ccc(O)cc2)(c2ccc(O)cc2)C1 ; CC(C)(c1ccc(O)cc1)c1cccc(C(C)(C)c2ccc(O)cc2)c1 ; CC(C)(c1ccc(O)cc1)c1ccc(C(C)(C)c2ccc(O)cc2)cc1 ; Oc1cccc2c(-c3cccc4c(O)cccc34)cccc12 ; Oc1ccc2ccccc2c1-c1c(O)ccc2ccccc12]

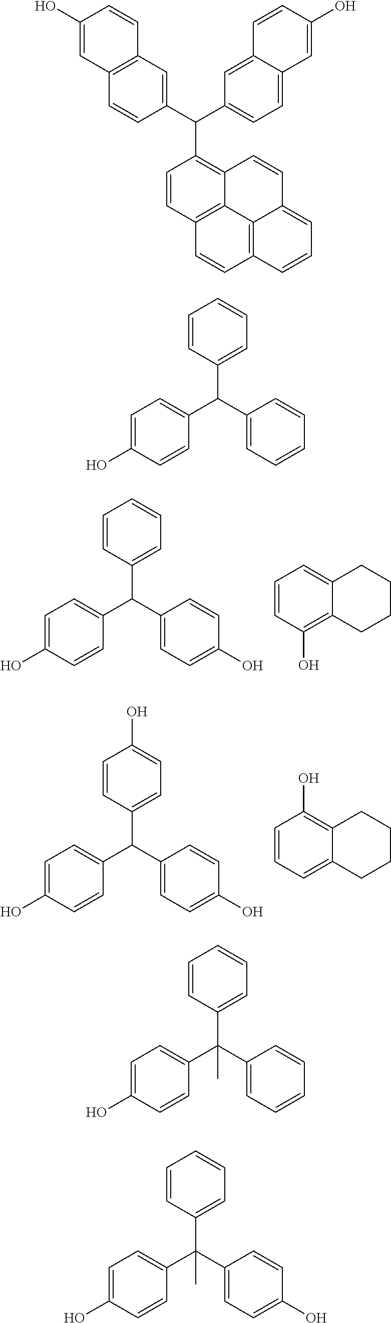

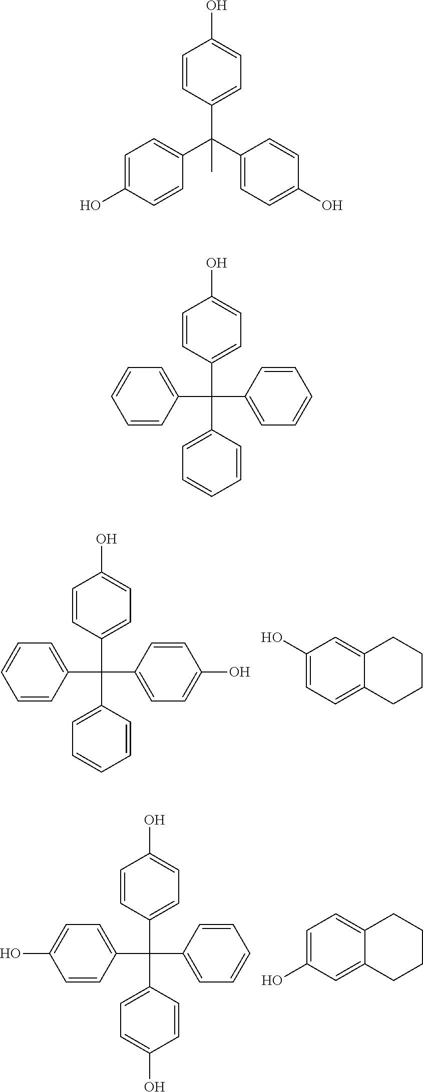

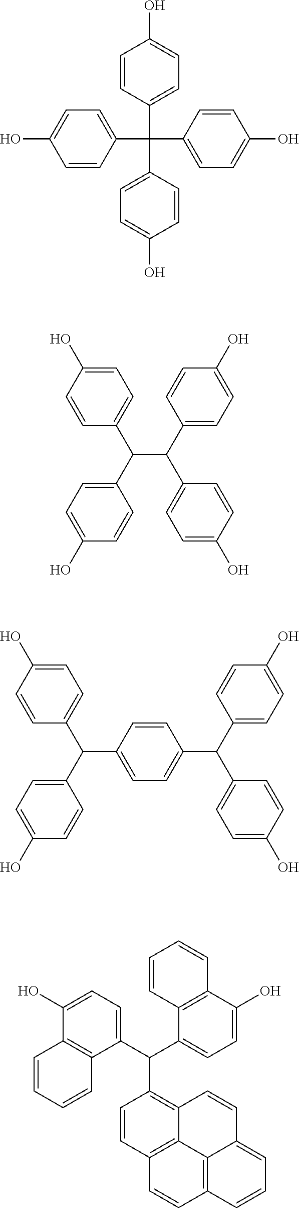 [SMILES: Oc1ccc(C(c2ccc(O)cc2)(c2ccc(O)cc2)c2ccc(O)cc2)cc1 ; Oc1ccc(C(c2ccc(O)cc2)C(c2ccc(O)cc2)c2ccc(O)cc2)cc1 ; Oc1ccc(C(c2ccc(O)cc2)c2ccc(C(c3ccc(O)cc3)c3ccc(O)cc3)cc2)cc1 ; Oc1ccc(C(c2ccc(O)c3ccccc23)c2ccc3ccc4cccc5ccc2c3c45)c2ccccc12]

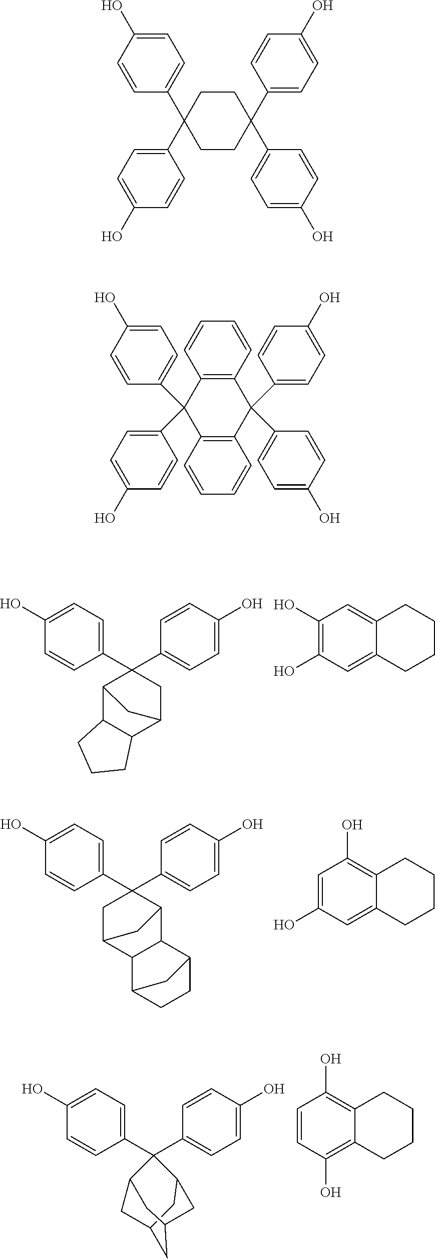

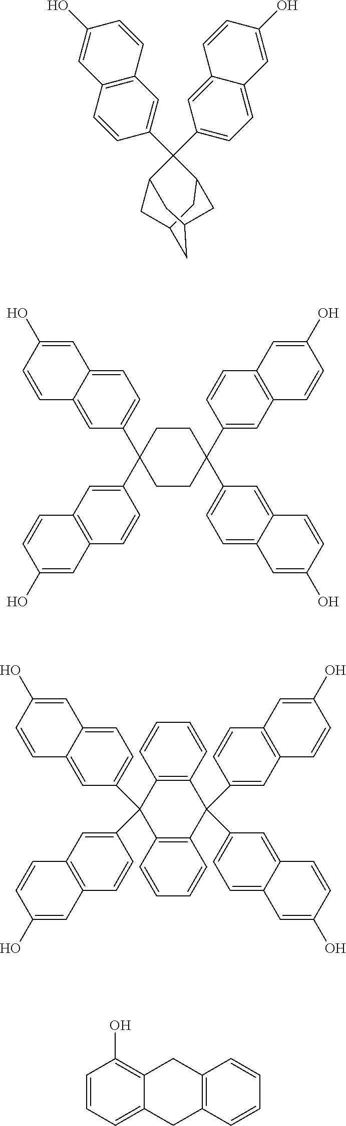 [SMILES: Oc1ccc2cc(C3(c4ccc5cc(O)ccc5c4)C4CC5CC(C4)CC3C5)ccc2c1 ; Oc1ccc2cc(C3(c4ccc5cc(O)ccc5c4)CCC(c4ccc5cc(O)ccc5c4)(c4ccc5cc(O)ccc5c4)CC3)ccc2c1 ; Oc1ccc2cc(C3(c4ccc5cc(O)ccc5c4)c4ccccc4C(c4ccc5cc(O)ccc5c4)(c4ccc5cc(O)ccc5c4)c4ccccc43)ccc2c1 ; Oc1cccc2c1Cc1ccccc1C2]

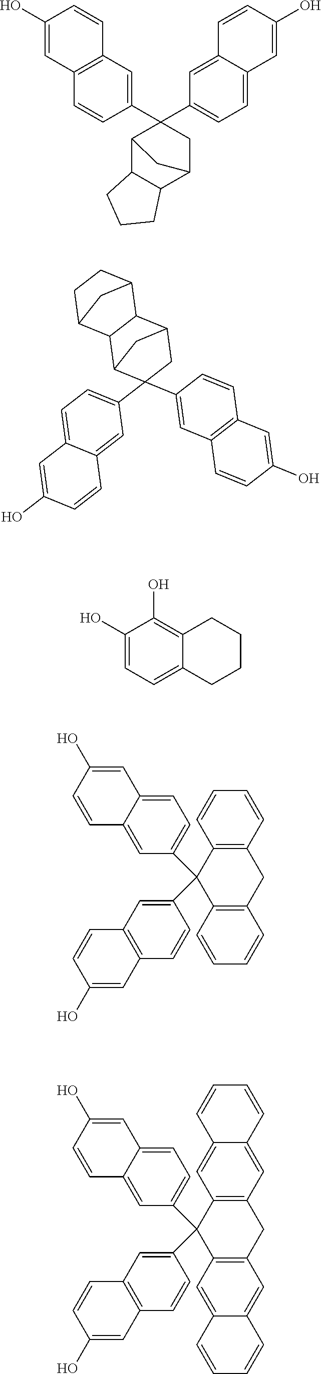 [SMILES: Oc1ccc2cc(C3(c4ccc5cc(O)ccc5c4)CC4CC3C3CCCC43)ccc2c1 ; Oc1ccc2cc(C3(c4ccc5cc(O)ccc5c4)CC4CC3C3C5CCC(C5)C43)ccc2c1 ; Oc1ccc2c(c1O)CCCC2 ; Oc1ccc2cc(C3(c4ccc5cc(O)ccc5c4)c4ccccc4Cc4ccccc43)ccc2c1 ; Oc1ccc2cc(C3(c4ccc5cc(O)ccc5c4)c4cc5ccccc5cc4Cc4cc5ccccc5cc43)ccc2c1]

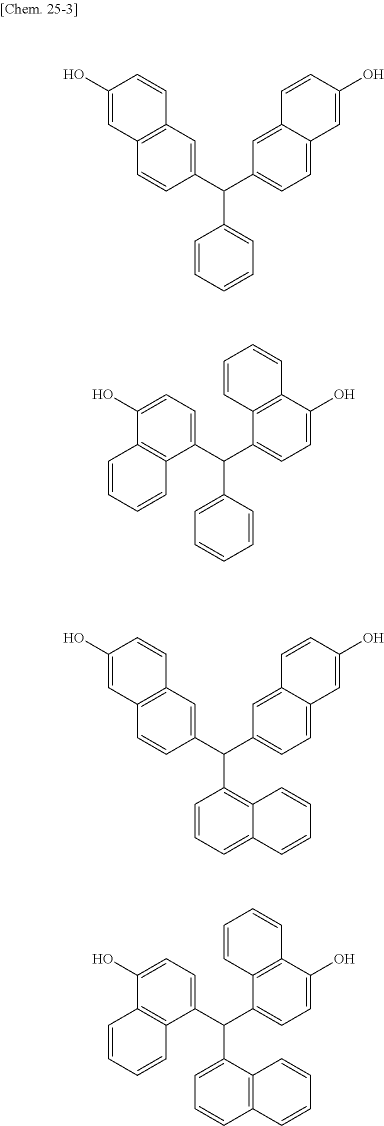 [SMILES: Oc1ccc2cc(C(c3ccccc3)c3ccc4cc(O)ccc4c3)ccc2c1 ; Oc1ccc(C(c2ccccc2)c2ccc(O)c3ccccc23)c2ccccc12 ; Oc1ccc2cc(C(c3ccc4cc(O)ccc4c3)c3cccc4ccccc34)ccc2c1 ; Oc1ccc(C(c2cccc3ccccc23)c2ccc(O)c3ccccc23)c2ccccc12]

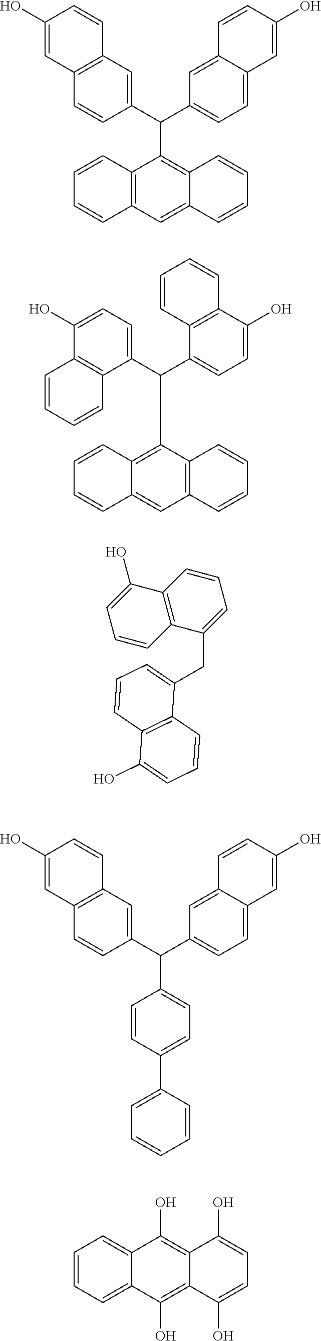

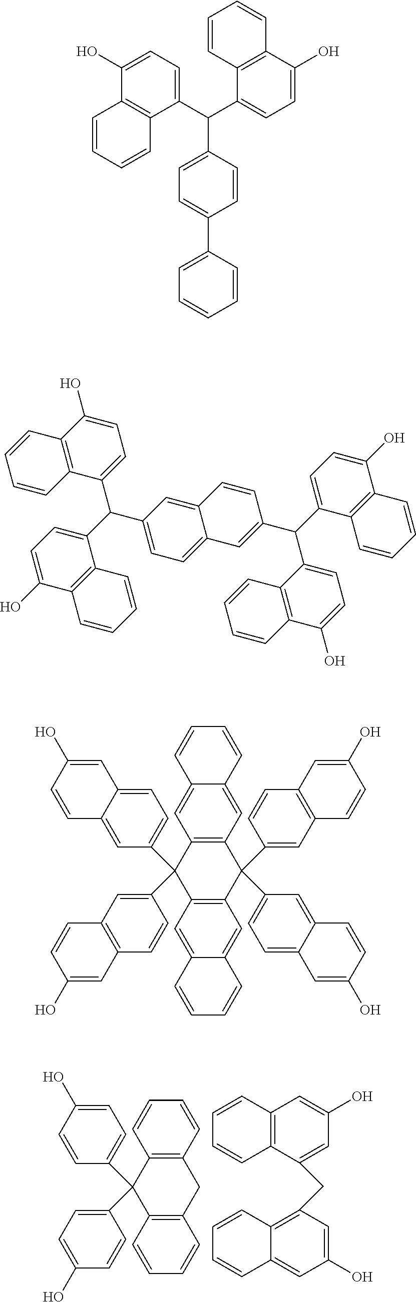

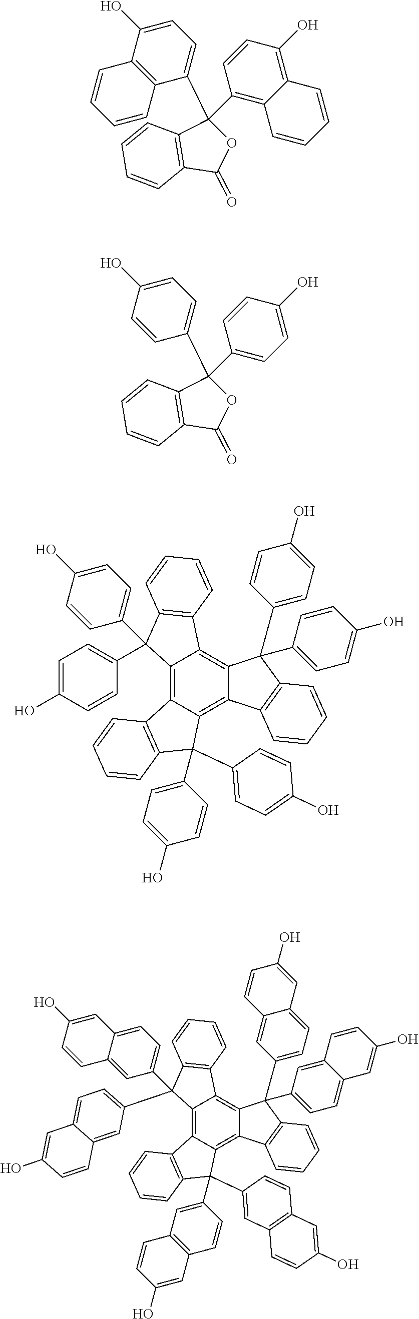 [SMILES: O=C1OC(c2ccc(O)c3ccccc23)(c2ccc(O)c3ccccc23)c2ccccc21 ; O=C1OC(c2ccc(O)cc2)(c2ccc(O)cc2)c2ccccc21 ; Oc1ccc(C2(c3ccc(O)cc3)c3ccccc3-c3c2c2c(c4c3C(c3ccc(O)cc3)(c3ccc(O)cc3)c3ccccc3-4)C(c3ccc(O)cc3)(c3ccc(O)cc3)c3ccccc3-2)cc1 ; Oc1ccc2cc(C3(c4ccc5cc(O)ccc5c4)c4ccccc4-c4c3c3c(c5c4C(c4ccc6cc(O)ccc6c4)(c4ccc6cc(O)ccc6c4)c4ccccc4-5)C(c4ccc5cc(O)ccc5c4)(c4ccc5cc(O)ccc5c4)c4ccccc4-3)ccc2c1]

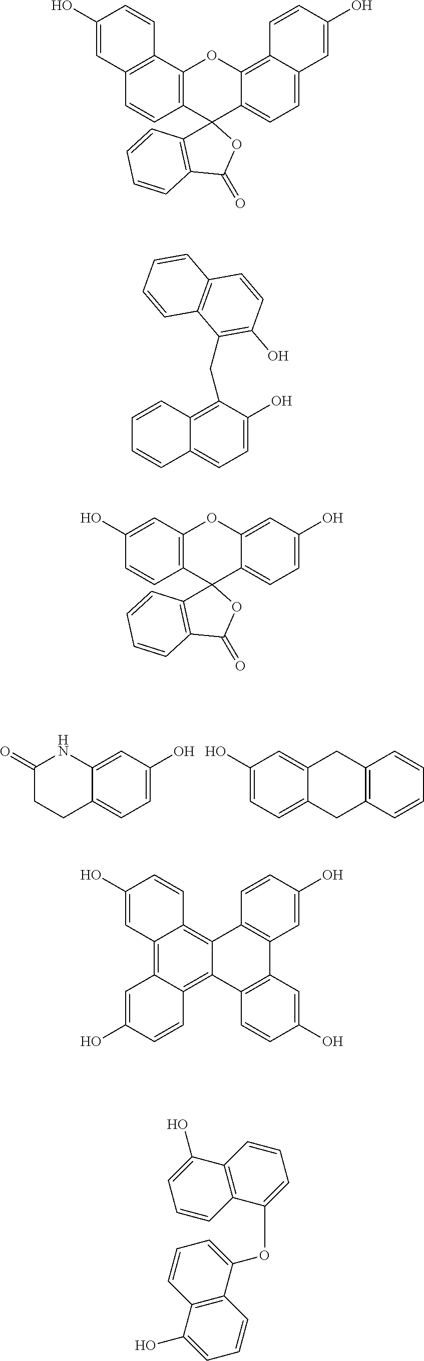

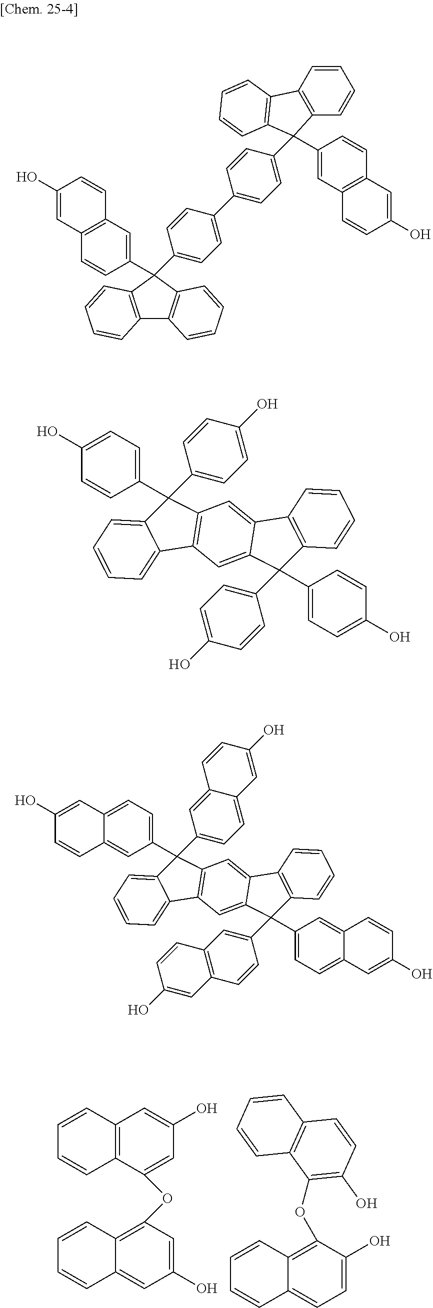

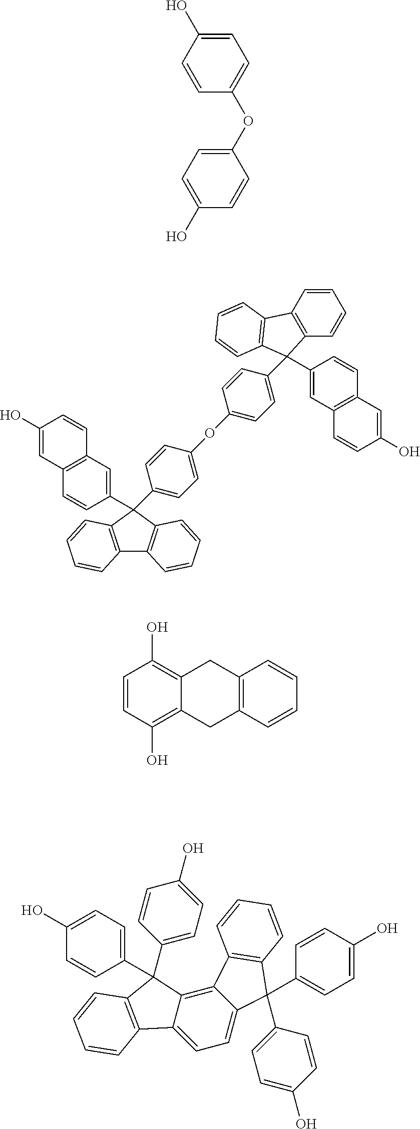 [SMILES: Oc1ccc(Oc2ccc(O)cc2)cc1 ; Oc1ccc2cc(C3(c4ccc(Oc5ccc(C6(c7ccc8cc(O)ccc8c7)c7ccccc7-c7ccccc76)cc5)cc4)c4ccccc4-c4ccccc43)ccc2c1 ; Oc1ccc(O)c2c1Cc1ccccc1C2 ; Oc1ccc(C2(c3ccc(O)cc3)c3ccccc3-c3c2ccc2c3C(c3ccc(O)cc3)(c3ccc(O)cc3)c3ccccc3-2)cc1]

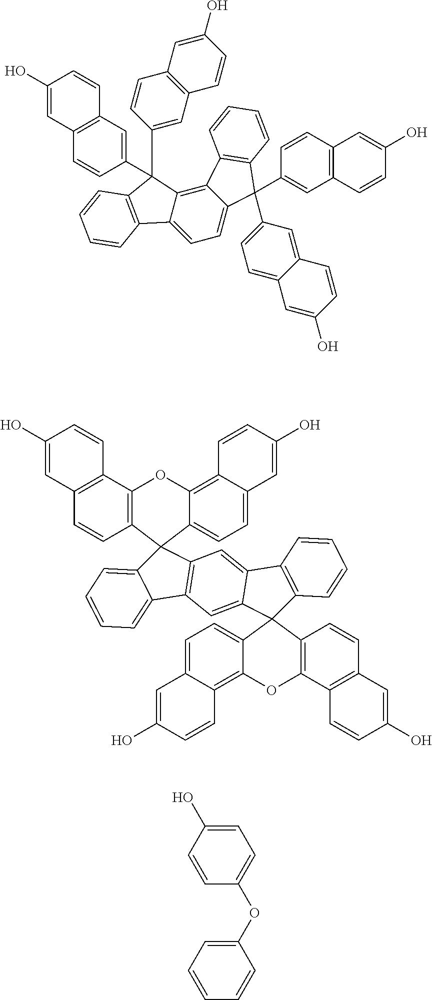 [SMILES: Oc1ccc2cc(C3(c4ccc5cc(O)ccc5c4)c4ccccc4-c4c3ccc3c4C(c4ccc5cc(O)ccc5c4)(c4ccc5cc(O)ccc5c4)c4ccccc4-3)ccc2c1 ; Oc1ccc2c3c(ccc2c1)C1(c2ccccc2-c2cc4c(cc21)-c1ccccc1C41c2ccc4cc(O)ccc4c2Oc2c1ccc1cc(O)ccc21)c1ccc2cc(O)ccc2c1O3 ; Oc1ccc(Oc2ccccc2)cc1]

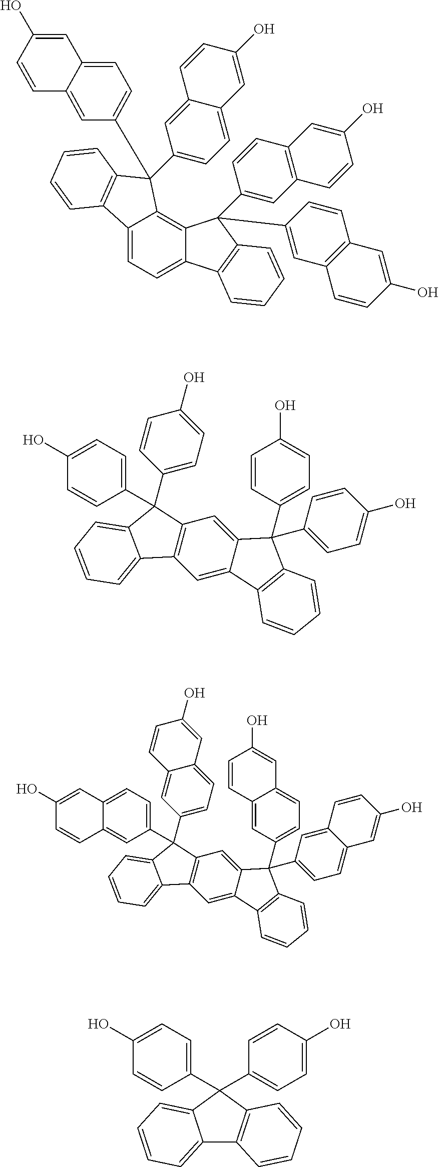 [SMILES: Oc1ccc2cc(C3(c4ccc5cc(O)ccc5c4)c4ccccc4-c4ccc5c(c43)C(c3ccc4cc(O)ccc4c3)(c3ccc4cc(O)ccc4c3)c3ccccc3-5)ccc2c1 ; Oc1ccc(C2(c3ccc(O)cc3)c3ccccc3-c3cc4c(cc32)C(c2ccc(O)cc2)(c2ccc(O)cc2)c2ccccc2-4)cc1 ; Oc1ccc2cc(C3(c4ccc5cc(O)ccc5c4)c4ccccc4-c4cc5c(cc43)C(c3ccc4cc(O)ccc4c3)(c3ccc4cc(O)ccc4c3)c3ccccc3-5)ccc2c1 ; Oc1ccc(C2(c3ccc(O)cc3)c3ccccc3-c3ccccc32)cc1]

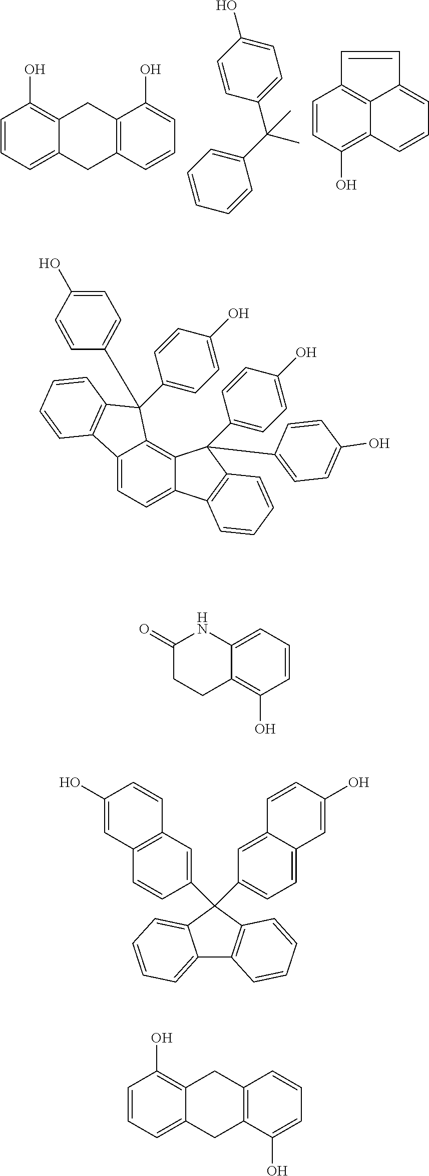

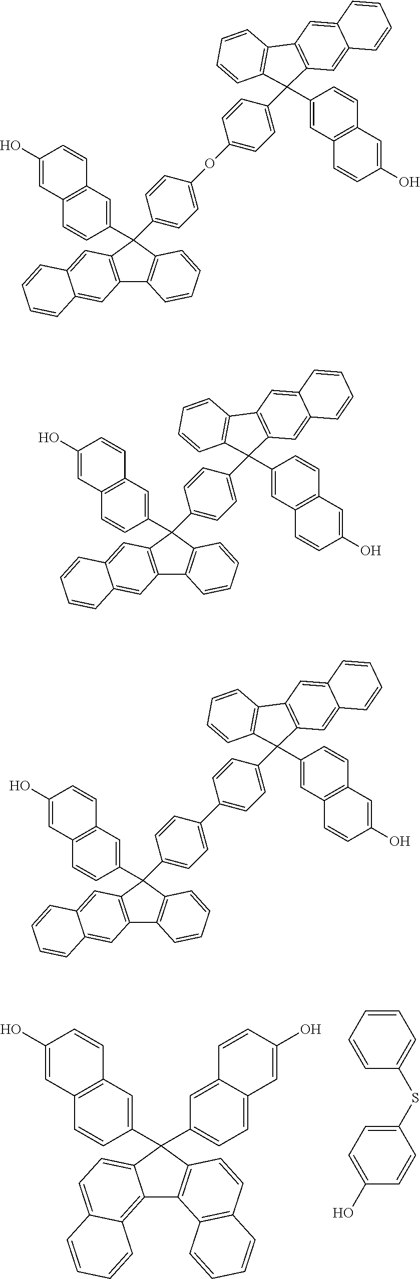

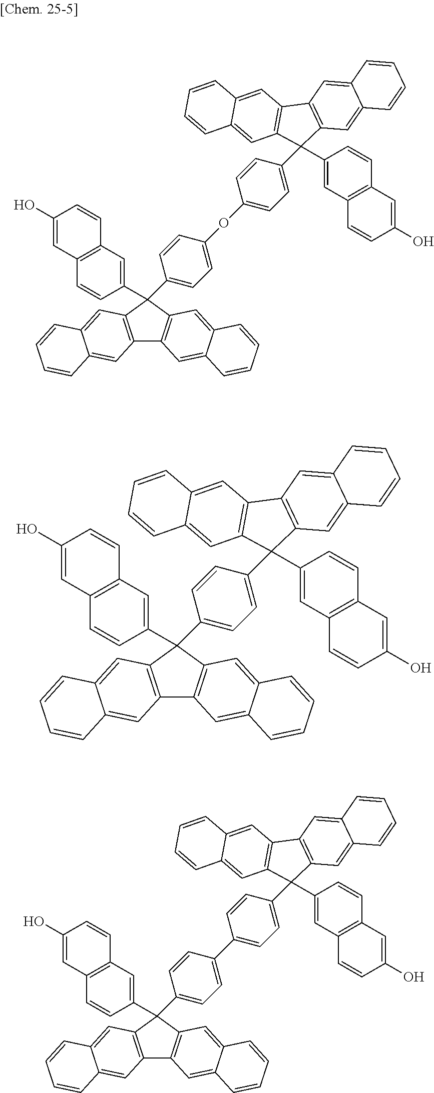 [SMILES: Oc1ccc2cc(C3(c4ccc(Oc5ccc(C6(c7ccc8cc(O)ccc8c7)c7cc8ccccc8cc7-c7cc8ccccc8cc76)cc5)cc4)c4cc5ccccc5cc4-c4cc5ccccc5cc43)ccc2c1 ; Oc1ccc2cc(C3(c4ccc(C5(c6ccc7cc(O)ccc7c6)c6cc7ccccc7cc6-c6cc7ccccc7cc65)cc4)c4cc5ccccc5cc4-c4cc5ccccc5cc43)ccc2c1 ; Oc1ccc2cc(C3(c4ccc(-c5ccc(C6(c7ccc8cc(O)ccc8c7)c7cc8ccccc8cc7-c7cc8ccccc8cc76)cc5)cc4)c4cc5ccccc5cc4-c4cc5ccccc5cc43)ccc2c1]

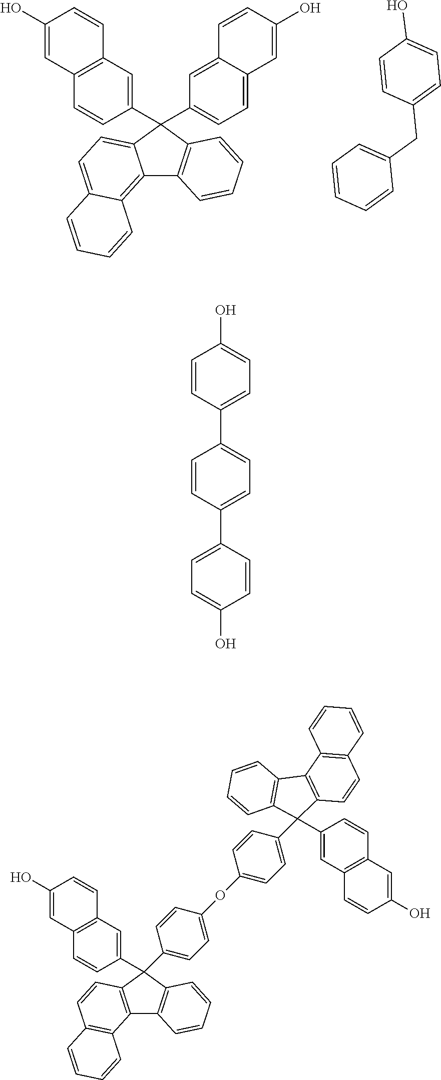 [SMILES: Oc1ccc2cc(C3(c4ccc5cc(O)ccc5c4)c4ccccc4-c4c3ccc3ccccc43)ccc2c1 ; Oc1ccc(Cc2ccccc2)cc1 ; Oc1ccc(-c2ccc(-c3ccc(O)cc3)cc2)cc1 ; Oc1ccc2cc(C3(c4ccc(Oc5ccc(C6(c7ccc8cc(O)ccc8c7)c7ccccc7-c7c6ccc6ccccc76)cc5)cc4)c4ccccc4-c4c3ccc3ccccc43)ccc2c1]

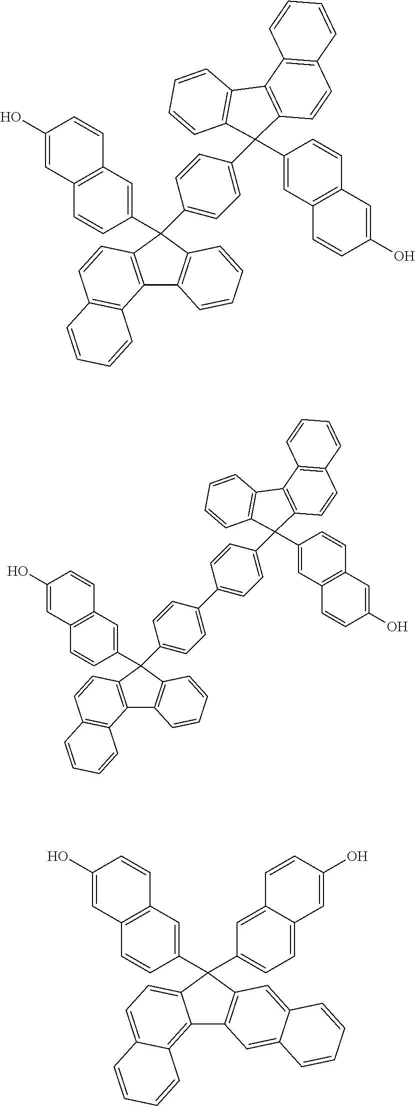 [SMILES: Oc1ccc2cc(C3(c4ccc(C5(c6ccc7cc(O)ccc7c6)c6ccccc6-c6c5ccc5ccccc65)cc4)c4ccccc4-c4c3ccc3ccccc43)ccc2c1 ; Oc1ccc2cc(C3(c4ccc(-c5ccc(C6(c7ccc8cc(O)ccc8c7)c7ccccc7-c7c6ccc6ccccc76)cc5)cc4)c4ccccc4-c4c3ccc3ccccc43)ccc2c1 ; Oc1ccc2cc(C3(c4ccc5cc(O)ccc5c4)c4cc5ccccc5cc4-c4c3ccc3ccccc43)ccc2c1]

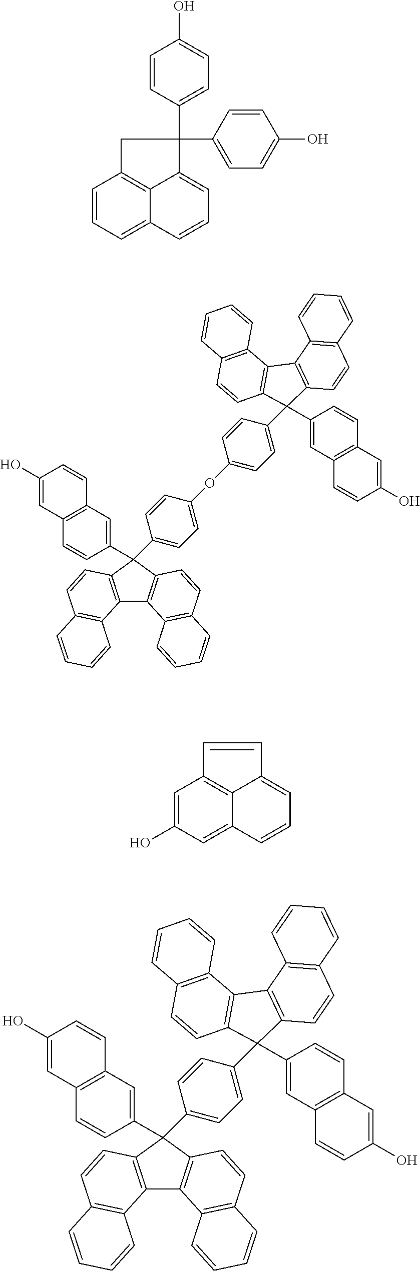 [SMILES: Oc1ccc(C2(c3ccc(O)cc3)Cc3cccc4cccc2c34)cc1 ; Oc1ccc2cc(C3(c4ccc(Oc5ccc(C6(c7ccc8cc(O)ccc8c7)c7ccc8ccccc8c7-c7c6ccc6ccccc76)cc5)cc4)c4ccc5ccccc5c4-c4c3ccc3ccccc43)ccc2c1 ; Oc1cc2c3c(cccc3c1)C=C2 ; Oc1ccc2cc(C3(c4ccc(C5(c6ccc7cc(O)ccc7c6)c6ccc7ccccc7c6-c6c5ccc5ccccc65)cc4)c4ccc5ccccc5c4-c4c3ccc3ccccc43)ccc2c1]

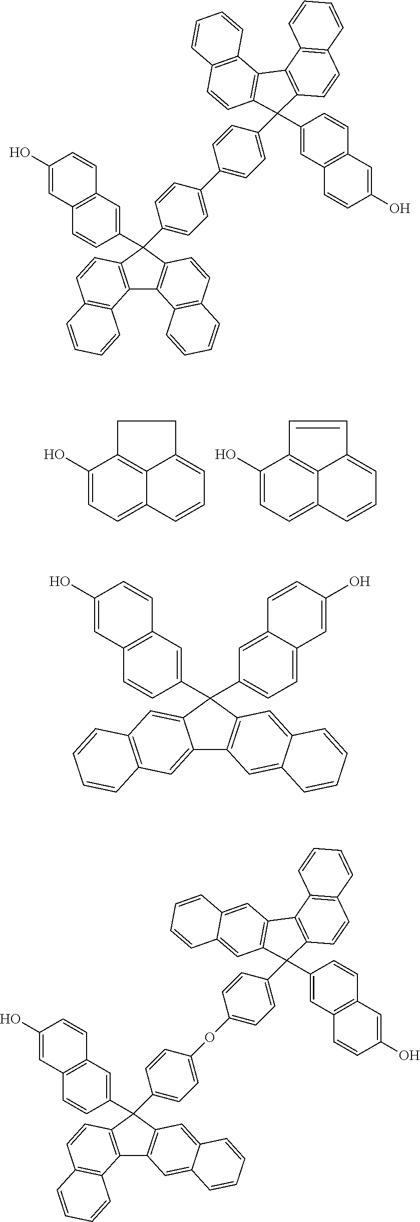

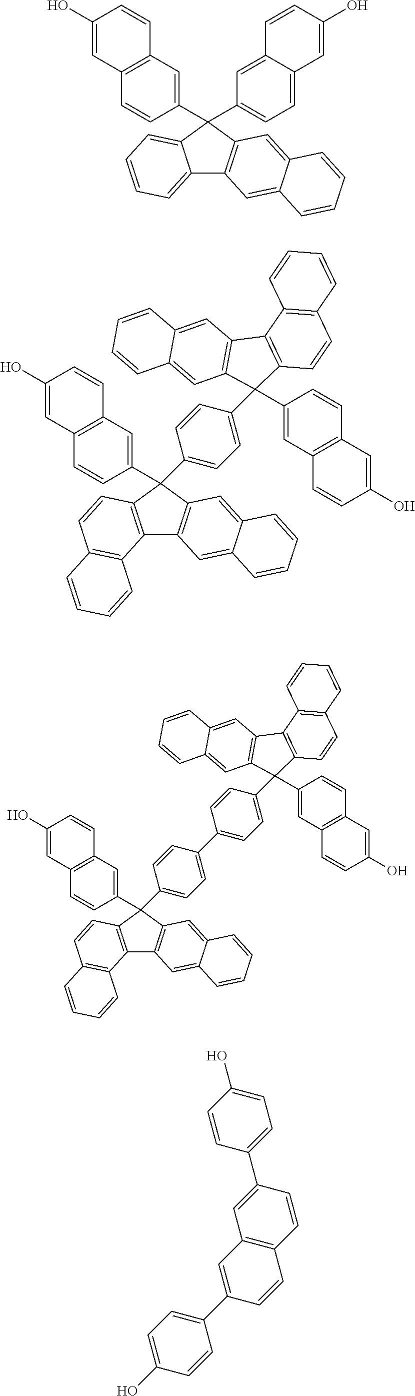

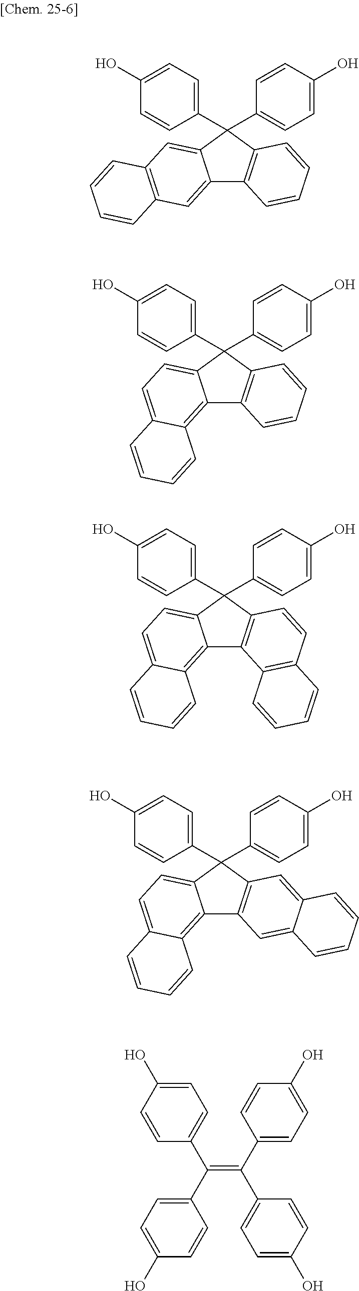

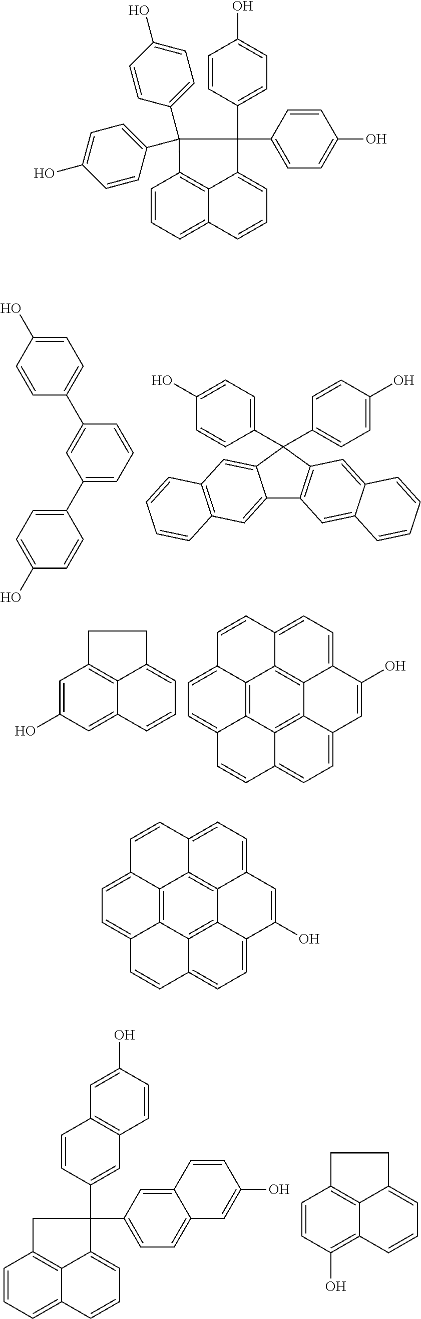

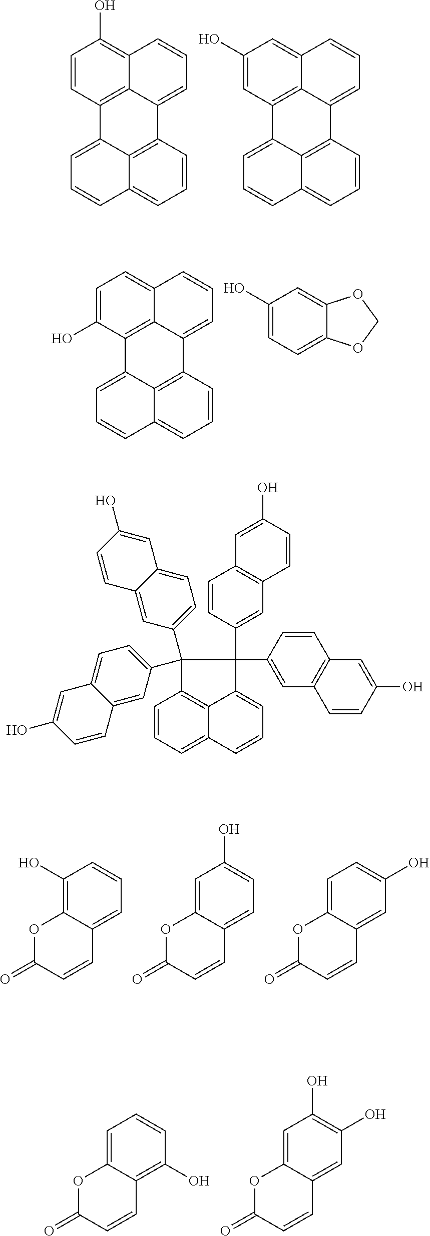

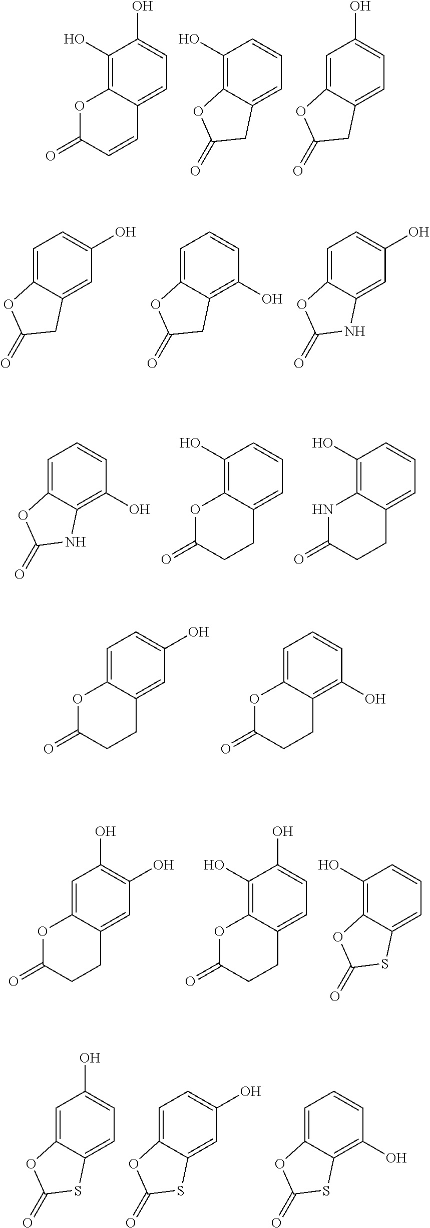

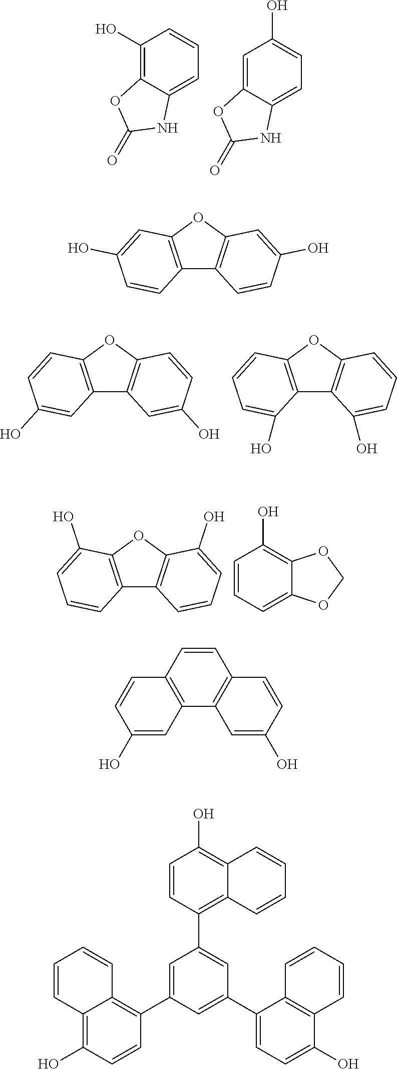

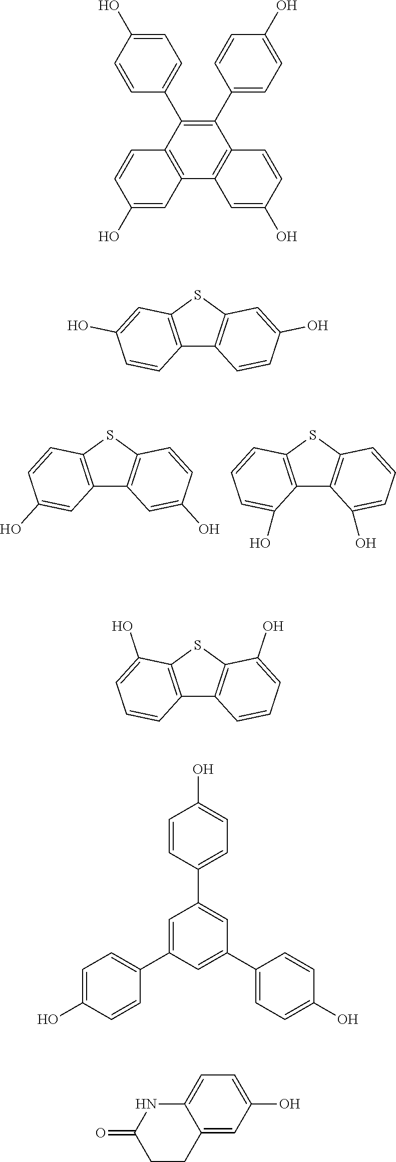

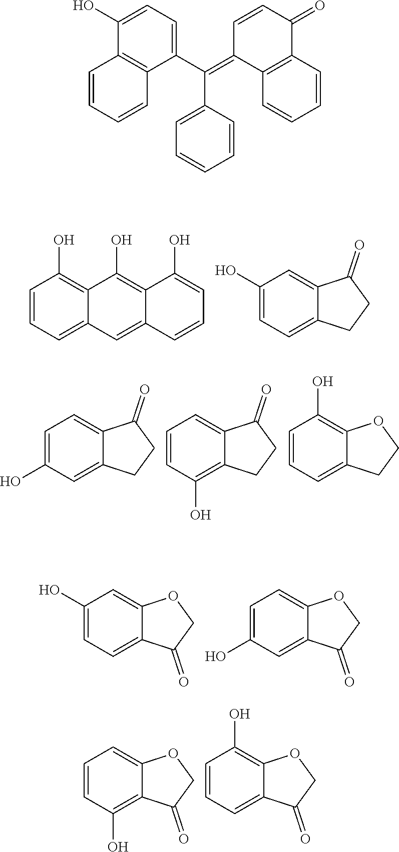

H of NH in the above amine skeletons and H of OH in the above phenol skeletons may be substituted by substituents illustrated below:

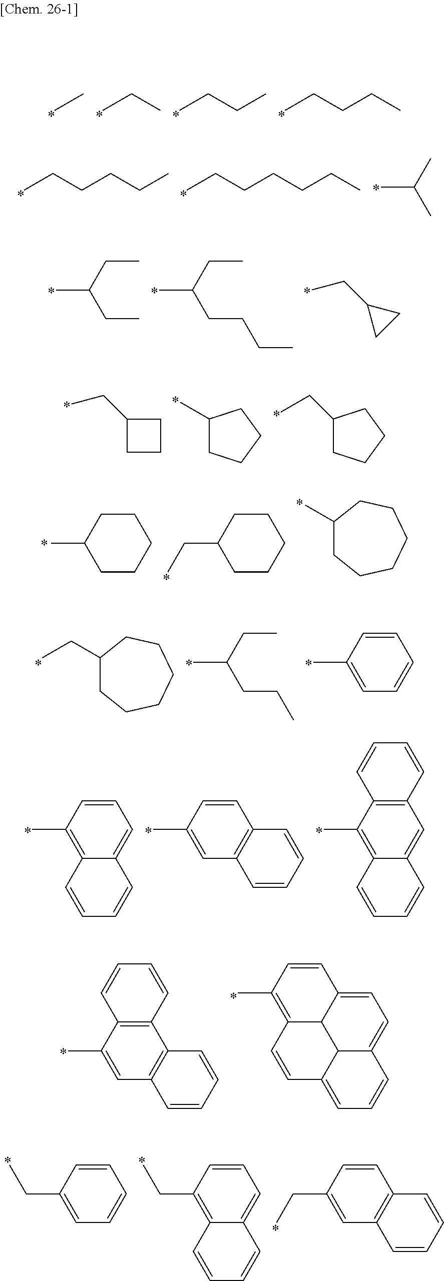

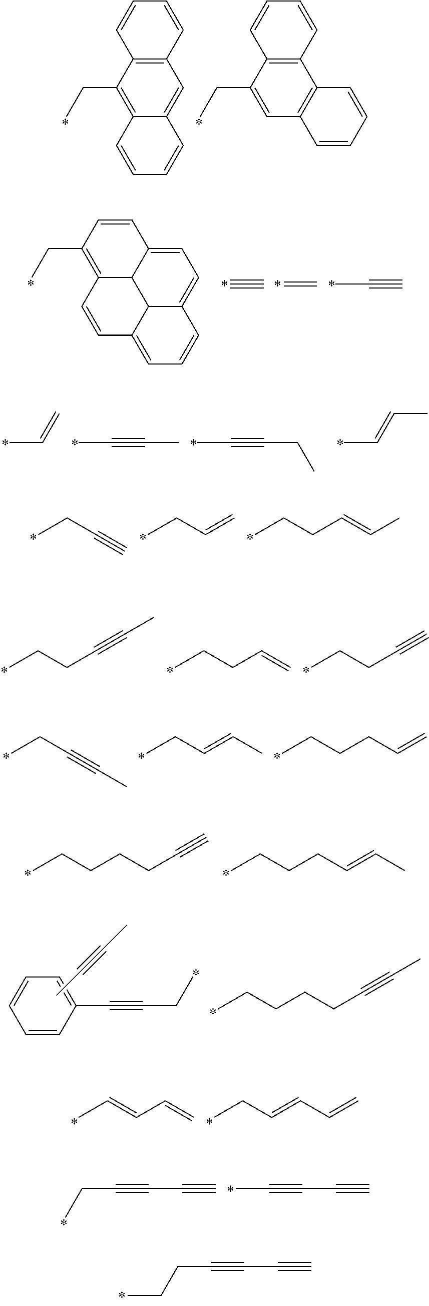

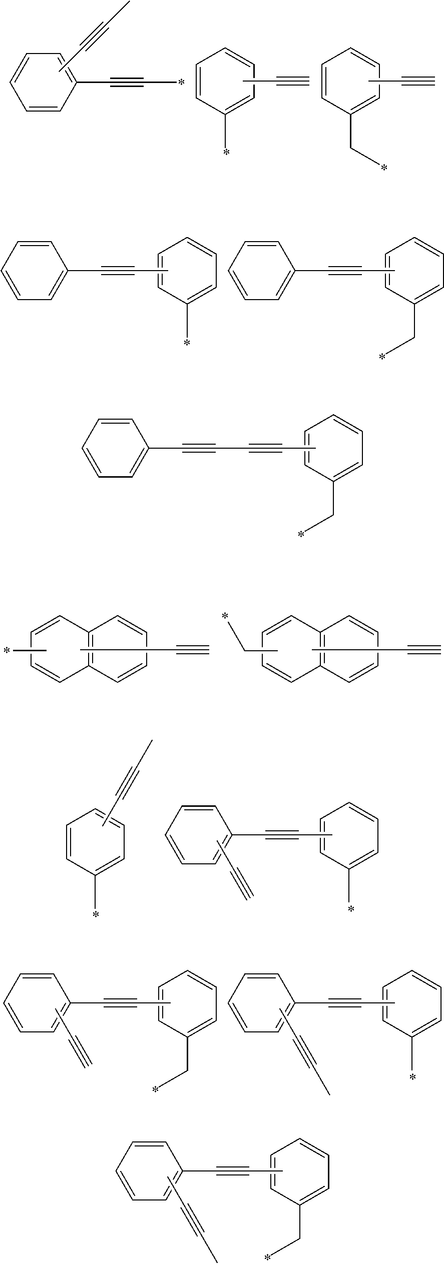

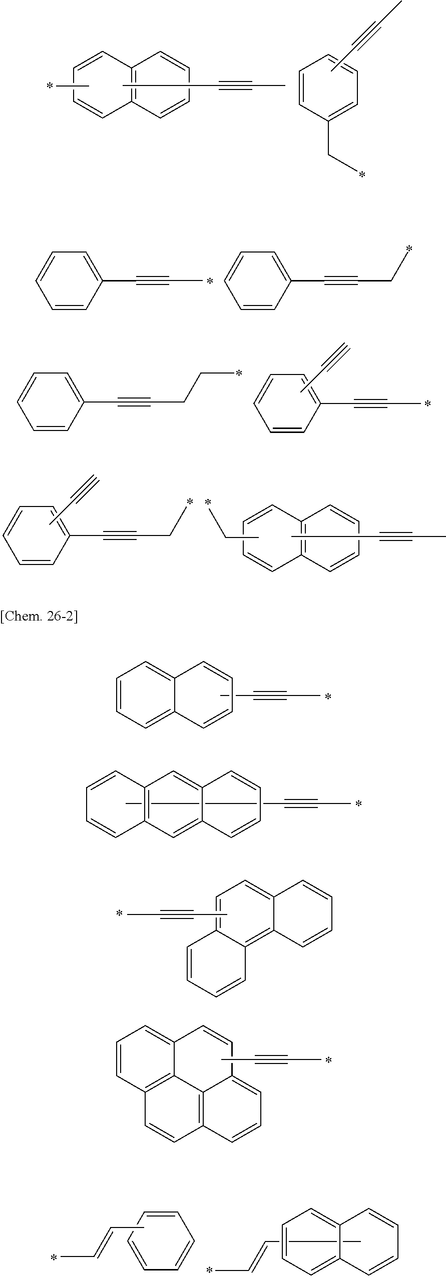

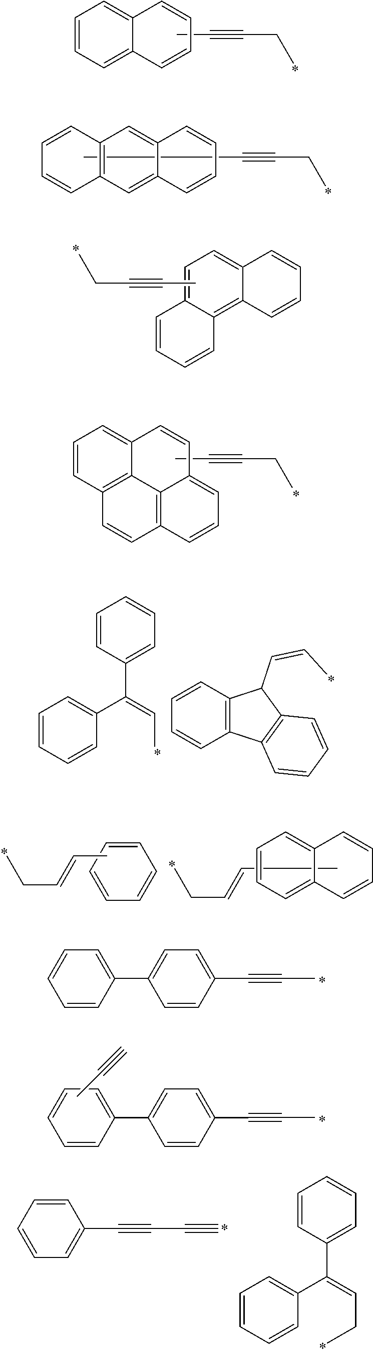

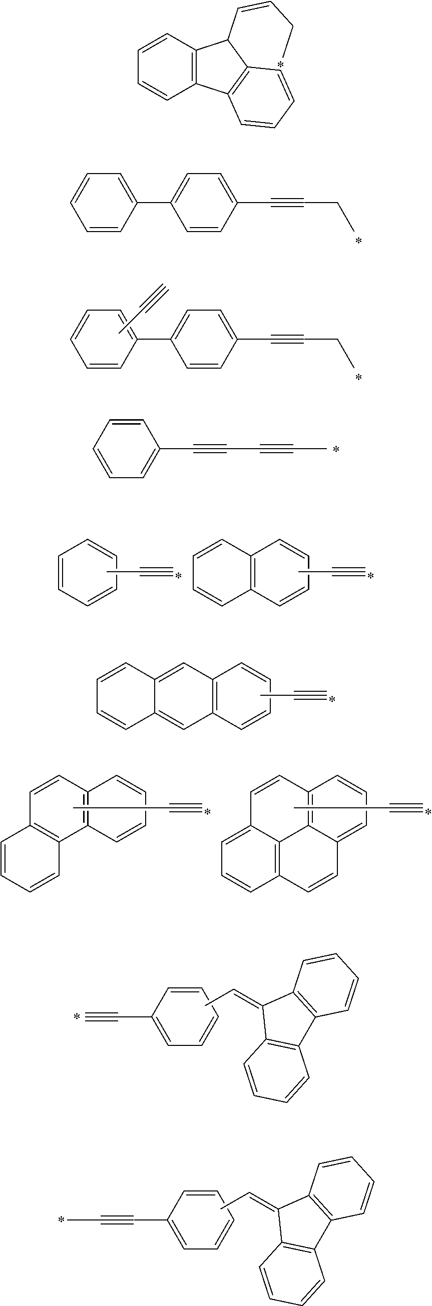

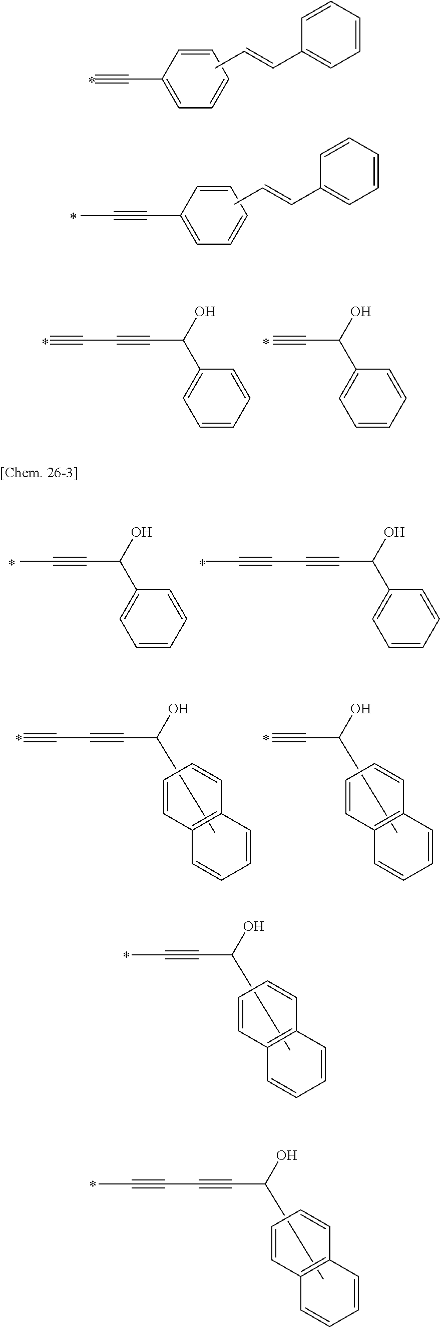

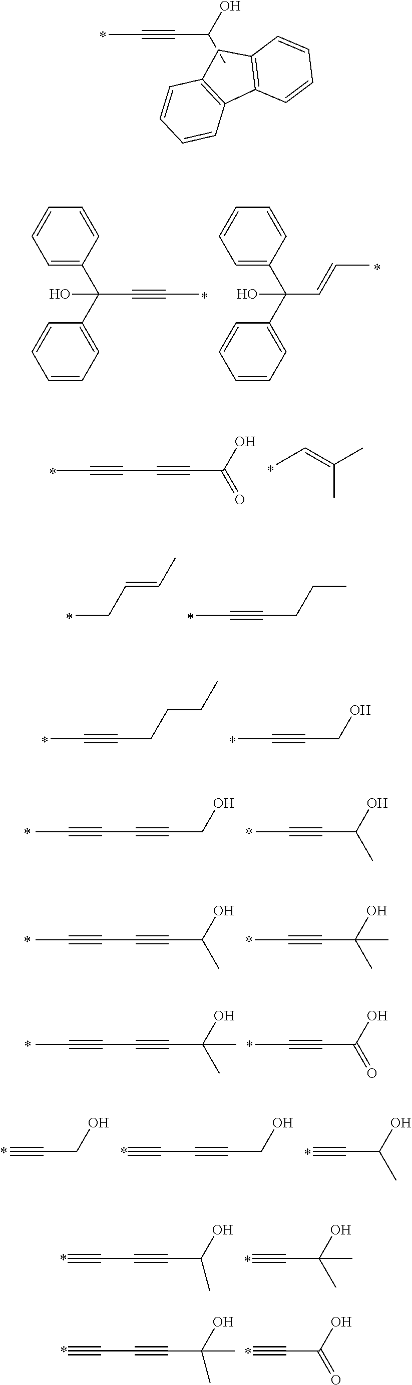

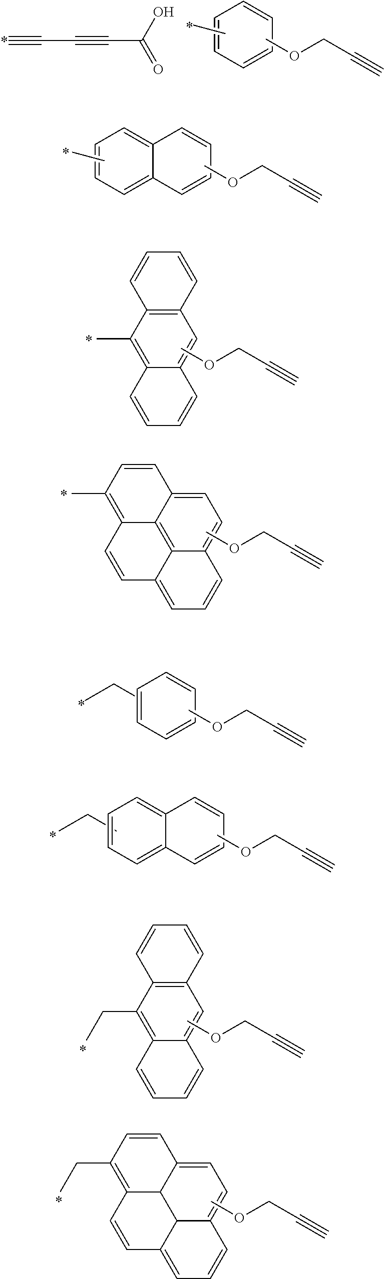

(2-3-4)

Preferably, the unit structure U is at least one member selected from the following:

### Examples of the Unit Structures Derived from a Heterocyclic Ring

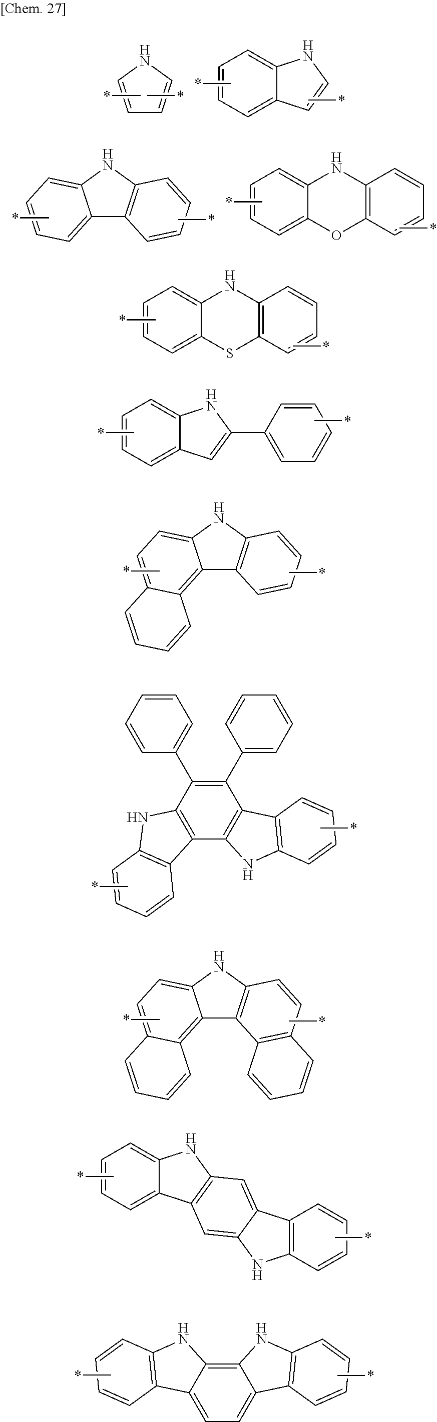

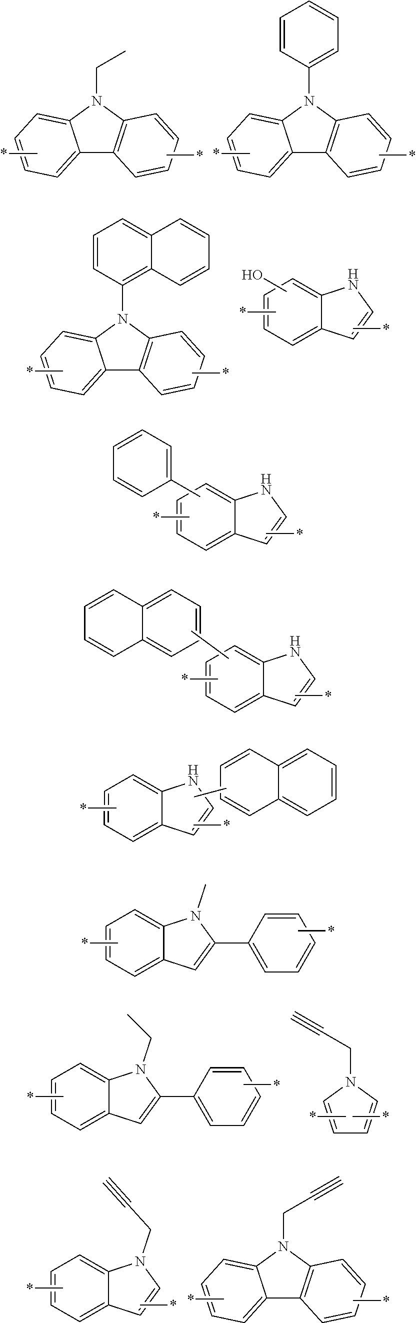

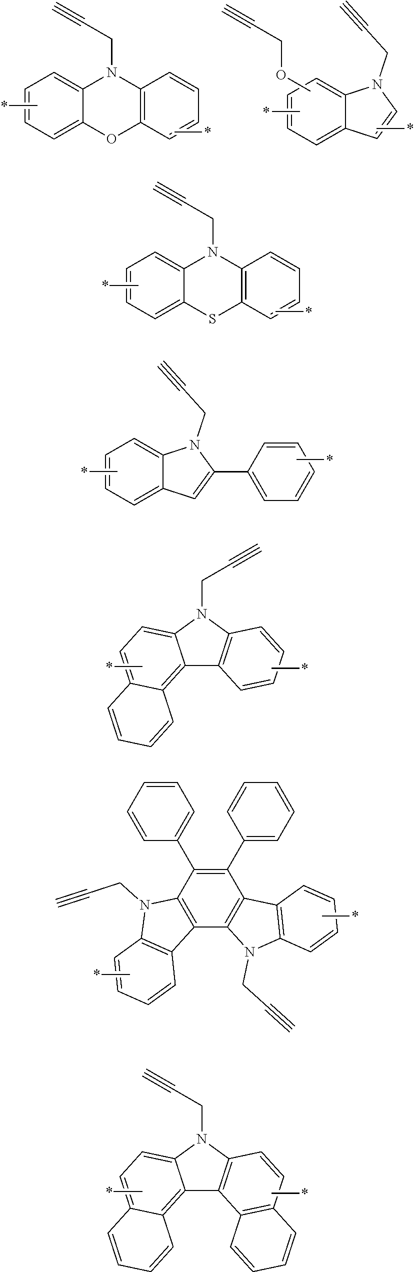

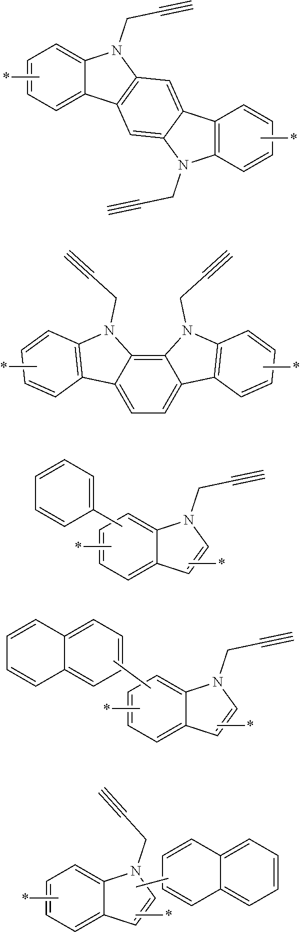

The positions of the two valence bonds * illustrated in each of the unit structures serve as a descriptive purpose and are not limited. The valence bonds may be present on any possible carbon atoms.

More preferred examples of the unit structures are illustrated below:

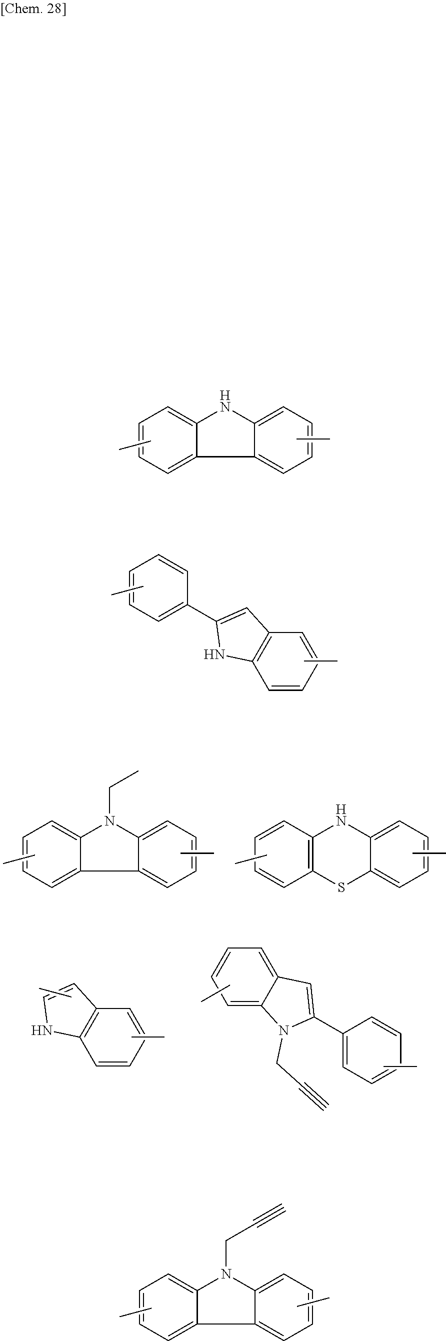

### Examples of the Unit Structures Derived from an Aromatic Hydrocarbon Having an Oxygen-Containing Substituent

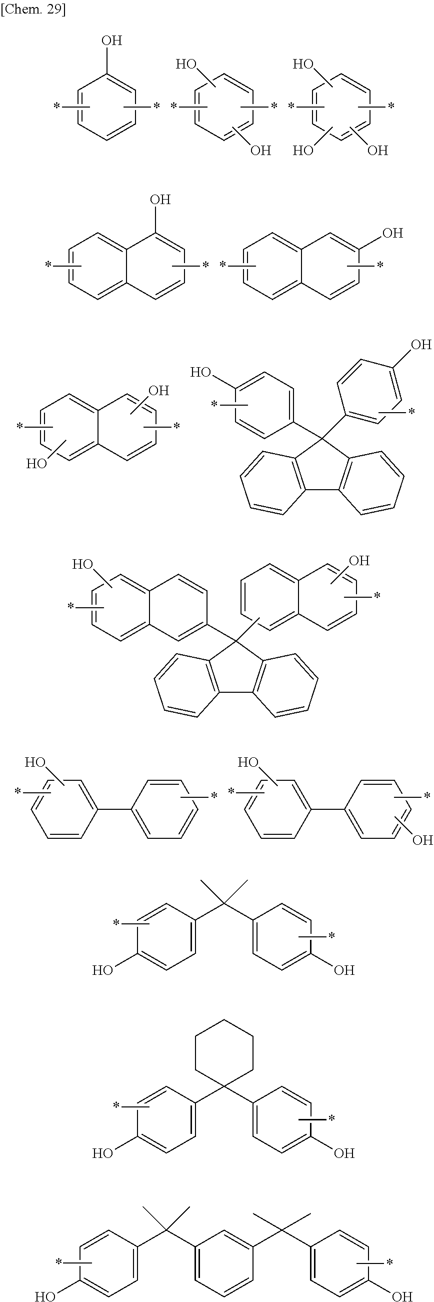

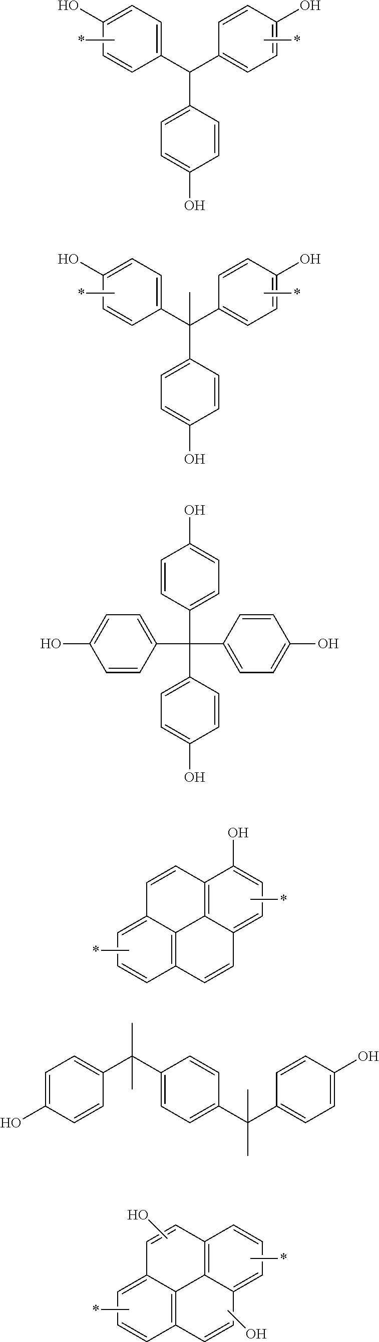

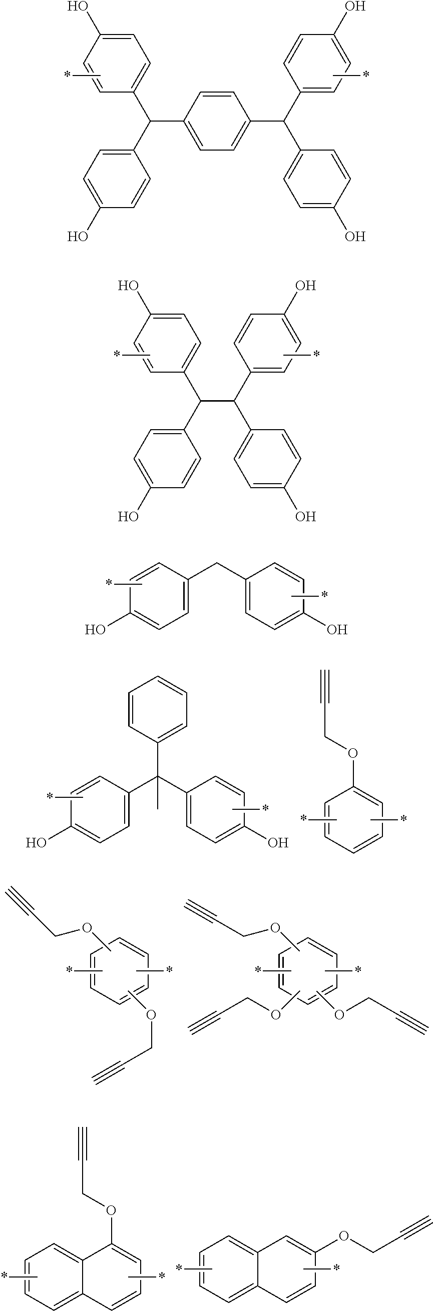

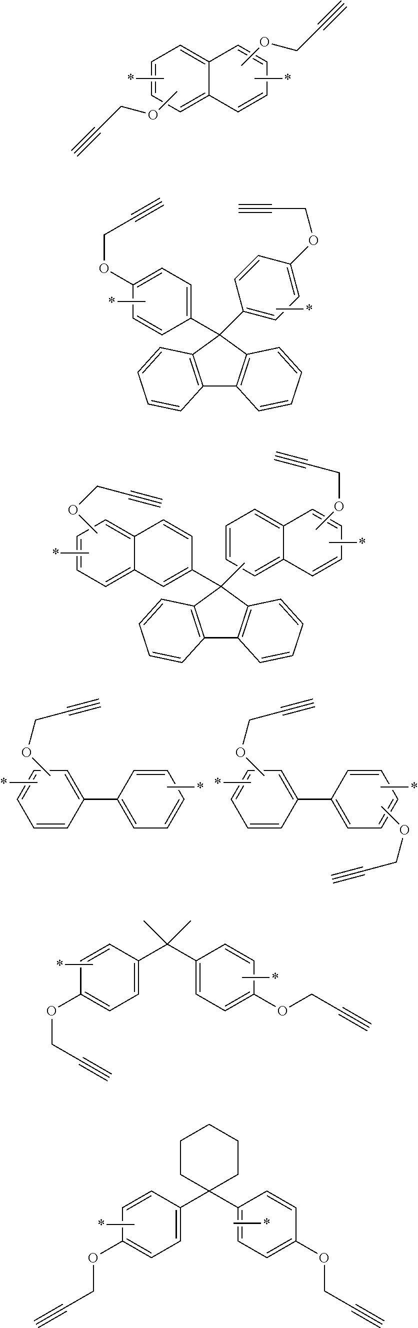

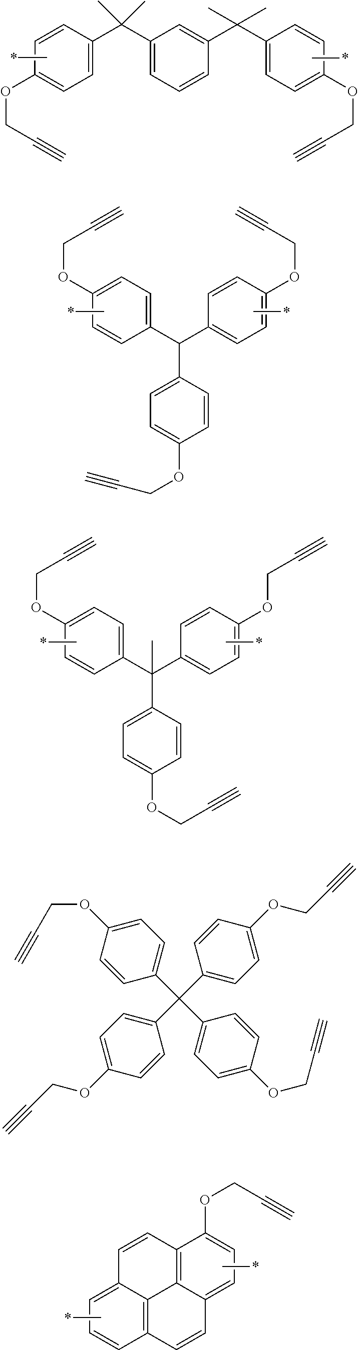

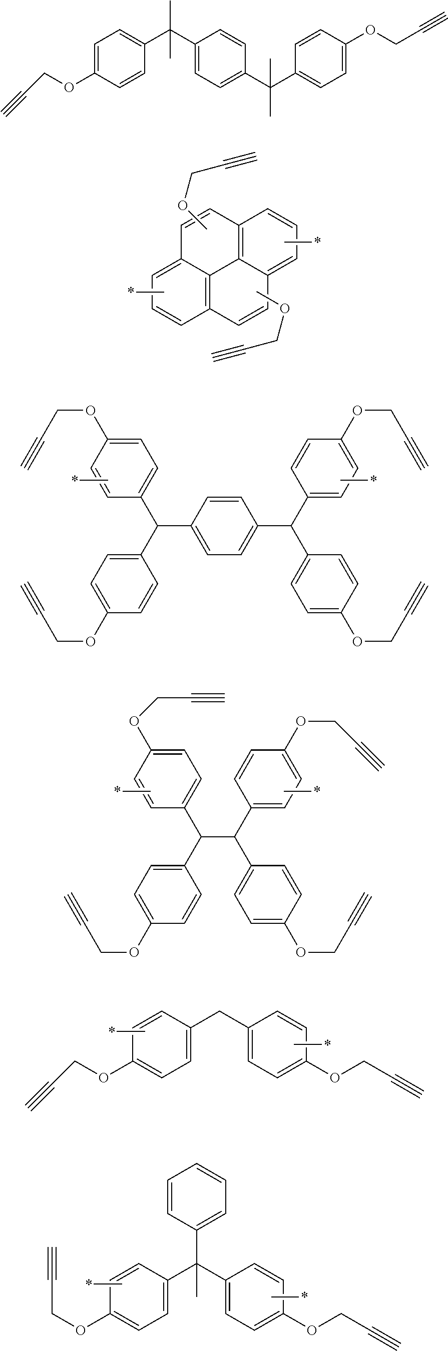

The position of the two valence bonds * illustrated in each of the unit structures serve as a descriptive purpose and are not limited. The valence bonds may be present on any possible carbon atoms.

More preferred examples of the structures are illustrated below:

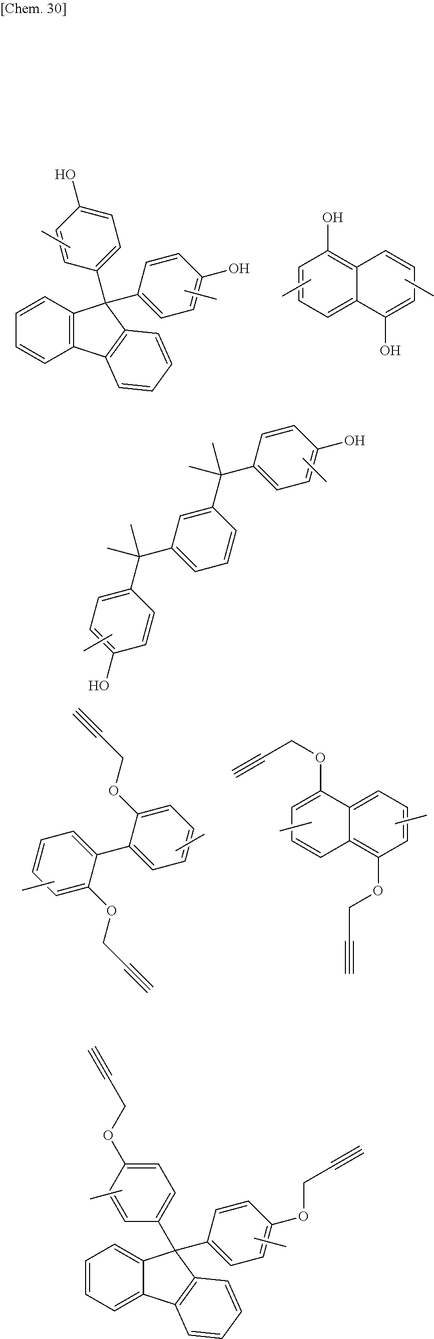

(Examples of the Unit Structures Derived from Aromatic Hydrocarbons Connected by —NH—)

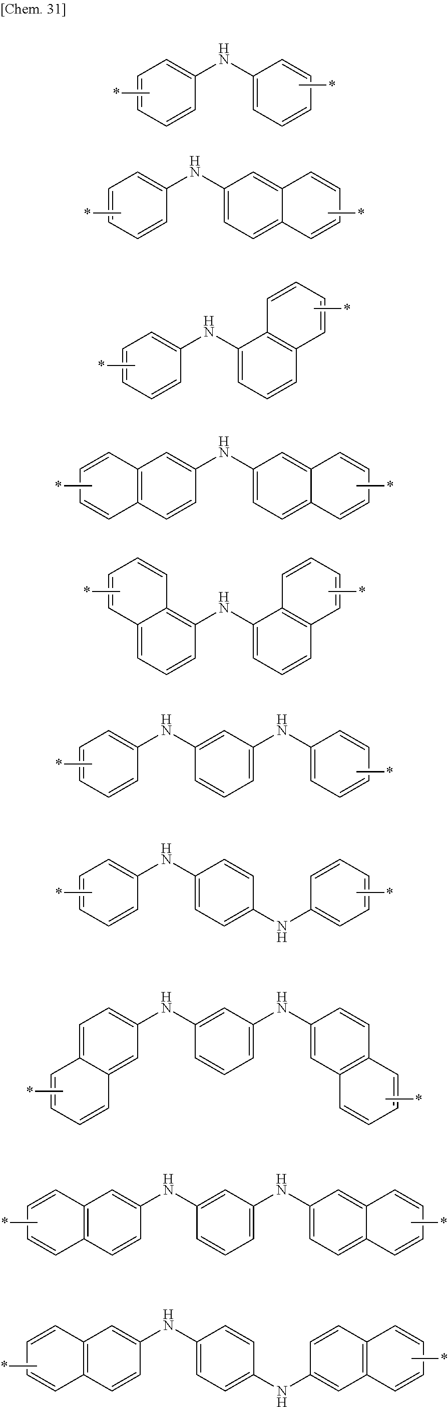

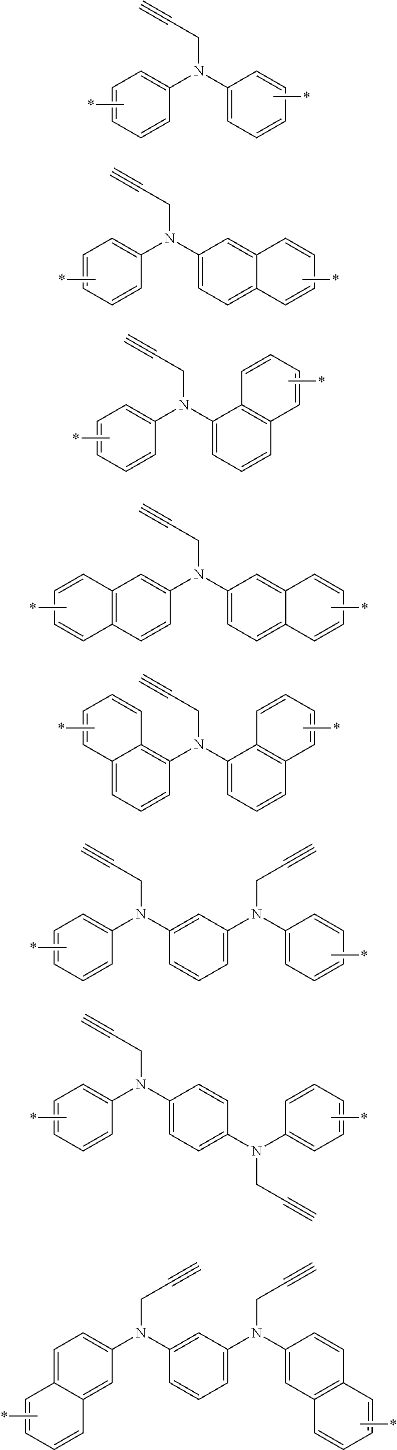

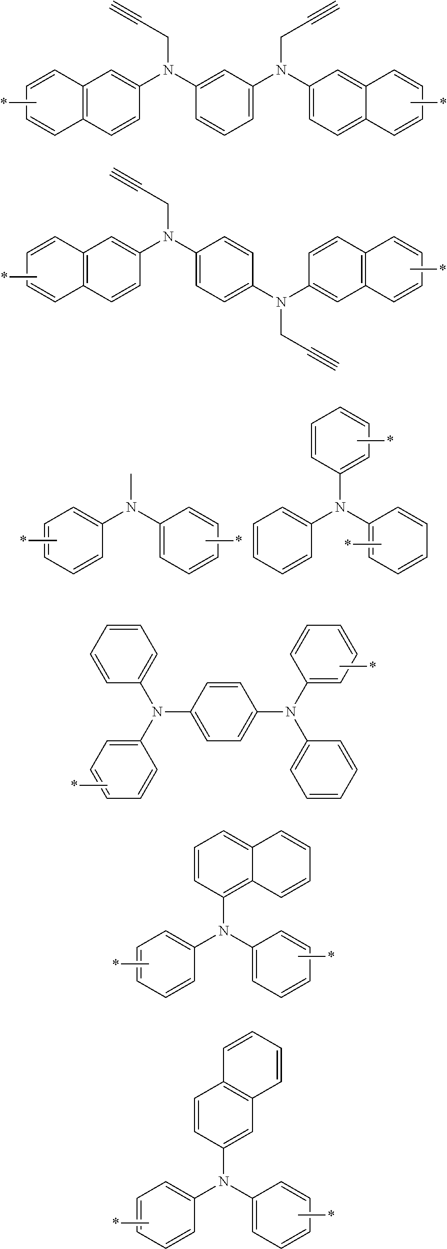

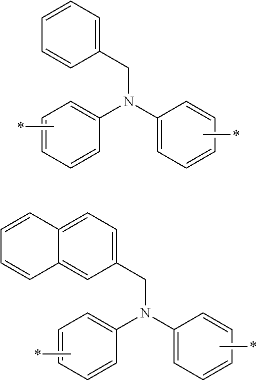

The positions of the two valence bonds * illustrated in each of the unit structures serve as a descriptive purpose and are not limited. The valence bonds may be present on any possible carbon atoms.

More preferred examples of the unit structures are illustrated below.

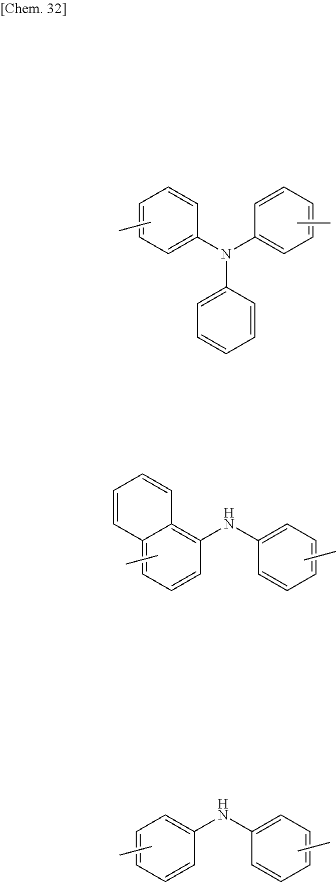

(2-3-5)

The unit structure V comprises one, or two or more types of unit structures including at least one structure selected from formulas (II), (III), or (IV) below. For example, the unit structure may be composed of two or three same or different structures of the following formulas that are connected to one another via a divalent or trivalent linking group.

The unit structures U and V are bonded to each other by a covalent bond formed between the valence bond in formula (II), (III), or (IV) below of the unit structure V and a carbon atom on the aromatic ring in the unit structure U.

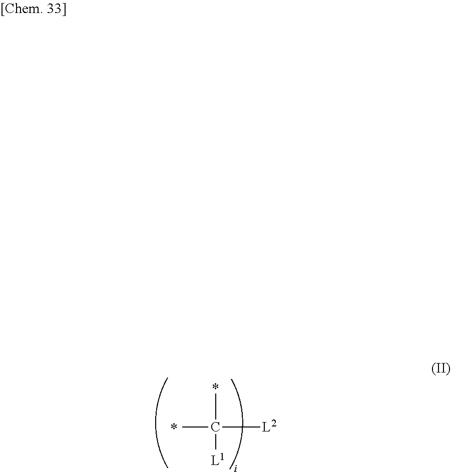

(in formula (II),

* indicates a bonding site to the unit structure U,

L¹ is:
an optionally substituted, saturated or unsaturated, linear, branched, or cyclic, aliphatic hydrocarbon group optionally containing a heteroatom;
an optionally substituted aromatic hydrocarbon group optionally containing a heteroatom;
a group comprising a combination or a condensation product of the above groups; or
a hydrogen atom,

### L² is:

an optionally substituted, saturated or unsaturated, linear, branched, or cyclic, aliphatic hydrocarbon group optionally containing a heteroatom;
an optionally substituted aromatic hydrocarbon group optionally containing a heteroatom;
a group comprising a combination or a condensation product of the above groups;
a direct bond; or
a hydrogen atom,
   L¹ and L² are optionally condensed to each other or are optionally bonded to each other via or without a heteroatom to form a ring,
i is an integer of 1 or more and 8 or less,
when i is 2 or more, L² is not a hydrogen atom, and
when i is 2 or more, the two to i quantity of carbon atoms are optionally connected to one another by the aliphatic hydrocarbon group or the aromatic hydrocarbon group L¹.)

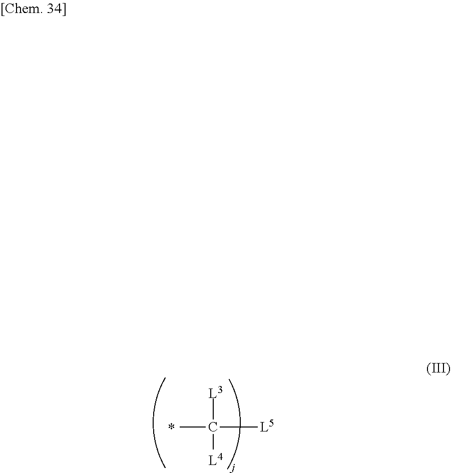

(in formula (III),

* indicates a bonding site to the unit structure U,

### L³ is:

an optionally substituted, saturated or unsaturated, linear, branched, or cyclic, aliphatic hydrocarbon group optionally containing a heteroatom;
an optionally substituted aromatic hydrocarbon group optionally containing a heteroatom;
a group comprising a combination or a condensation product of the above groups;
a hydroxy group; or
a hydrogen atom,

### L⁴ is:

an optionally substituted, saturated or unsaturated, linear, branched, or cyclic, aliphatic hydrocarbon group optionally containing a heteroatom;
an optionally substituted aromatic hydrocarbon group optionally containing a heteroatom;
a group comprising a combination or a condensation product of the above groups;
a hydroxy group; or
a hydrogen atom,

### L⁵ is:

an optionally substituted, saturated or unsaturated, linear, branched, or cyclic, aliphatic hydrocarbon group optionally containing a heteroatom;
an optionally substituted aromatic hydrocarbon group optionally containing a heteroatom;
a group comprising a combination or a condensation product of the above groups; or
a direct bond,

j is an integer of 2 or more and 4 or less, and

L³, L⁴, and L⁵ are optionally condensed to one another or are optionally bonded to one another via or without a heteroatom to form a ring.)

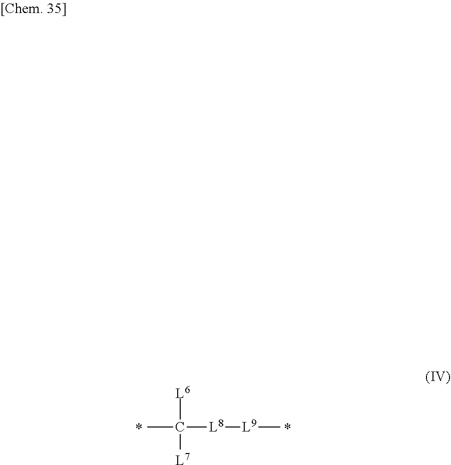

(in formula (IV),

* indicates a bonding site to the unit structure U,

### L⁶ is:

an optionally substituted, saturated or unsaturated, linear, branched, or cyclic, aliphatic hydrocarbon group optionally containing a heteroatom;
an optionally substituted aromatic hydrocarbon group optionally containing a heteroatom;
a group comprising a combination or a condensation product of the above groups; or
a hydrogen atom,

### L⁷ is:

an optionally substituted, saturated or unsaturated, linear, branched, or cyclic, aliphatic hydrocarbon group optionally containing a heteroatom;
an optionally substituted aromatic hydrocarbon group optionally containing a heteroatom;
a group comprising a combination or a condensation product of the above groups; or
a hydrogen atom,

L⁶, L⁸, and L⁹ are optionally condensed to one another or are optionally bonded to one another via or without a heteroatom to form a ring,

### L⁸ is:

a direct bond;
an optionally substituted, saturated or unsaturated, linear or branched hydrocarbon group; or
an aromatic ring optionally containing a heteroatom, and

### L⁹ is:

an aromatic ring optionally containing a heteroatom.)
   (2-3-6)

In formulas (II), (III), and (IV), the term “heteroatom” refers to an atom other than a carbon atom and a hydrogen atom, with examples including oxygen atom, nitrogen atom, and sulfur atom.

Examples of the “substituents” include halogen atoms, saturated or unsaturated, linear, branched, or cyclic hydrocarbon groups optionally containing a heteroatom, hydroxy group, amino group, carboxyl group, cyano group, nitro group, alkoxy groups, aldehyde groups, ester groups, amide groups, sulfonyl group, sulfide group, ether groups, ketone groups, aryl groups, and combinations thereof. The substituents are not limited to those mentioned above as long as the advantageous effects of the present invention are not impaired.

Examples of the “saturated, linear, branched, or cyclic, aliphatic hydrocarbon group” include methyl group, ethyl group, n-propyl group, i-propyl group, cyclopropyl group, n-butyl group, i-butyl group, s-butyl group, t-butyl group, cyclobutyl group, 1-methyl-cyclopropyl group, 2-methyl-cyclopropyl group, n-pentyl group, 1-methyl-n-butyl group, 2-methyl-n-butyl group, 3-methyl-n-butyl group, 1,1-dimethyl-n-propyl group, 1,2-dimethyl-n-propyl group, 2,2-dimethyl-n-propyl group, 1-ethyl-n-propyl group, cyclopentyl group, 1-methyl-cyclobutyl group, 2-methyl-cyclobutyl group, 3-methyl-cyclobutyl group, 1,2-dimethyl-cyclopropyl group, 2,3-dimethyl-cyclopropyl group, 1-ethyl-cyclopropyl group, 2-ethyl-cyclopropyl group, n-hexyl group, 1-methyl-n-pentyl group, 2-methyl-n-pentyl group, 3-methyl-n-pentyl group, 4-methyl-n-pentyl group, 1,1-dimethyl-n-butyl group, 1,2-dimethyl-n-butyl group, 1,3-dimethyl-n-butyl group, 2,2-dimethyl-n-butyl group, 2,3-dimethyl-n-butyl group, 3,3-dimethyl-n-butyl group, 1-ethyl-n-butyl group, 2-ethyl-n-butyl group, 1,1,2-trimethyl-n-propyl group, 1,2,2-trimethyl-n-propyl group, 1-ethyl-I-methyl-n-propyl group, 1-ethyl-2-methyl-n-propyl group, cyclohexyl group, 1-methyl-cyclopentyl group, 2-methyl-cyclopentyl group, 3-methyl-cyclopentyl group, 1-ethyl-cyclobutyl group, 2-ethyl-cyclobutyl group, 3-ethyl-cyclobutyl group, 1,2-dimethyl-cyclobutyl group, 1,3-dimethyl-cyclobutyl group, 2,2-dimethyl-cyclobutyl group, 2,3-dimethyl-cyclobutyl group, 2,4-dimethyl-cyclobutyl group, 3,3-dimethyl-cyclobutyl group, 1-n-propyl-cyclopropyl group, 2-n-propyl-cyclopropyl group, 1-i-propyl-cyclopropyl group, 2-i-propyl-cyclopropyl group, 1,2,2-trimethyl-cyclopropyl group, 1,2,3-trimethyl-cyclopropyl group, 2,2,3-trimethyl-cyclopropyl group, 1-ethyl-2-methyl-cyclopropyl group, 2-ethyl-1-methyl-cyclopropyl group, 2-ethyl-2-methyl-cyclopropyl group, and 2-ethyl-3-methyl-cyclopropyl group.

Examples of the “unsaturated, linear, branched, or cyclic, aliphatic hydrocarbon group” include ethenyl group, 1-propenyl group, 2-propenyl group, 1-methyl-1-ethenyl group, 1-butenyl group, 2-butenyl group, 3-butenyl group, 2-methyl-1-propenyl group, 2-methyl-2-propenyl group, 1-ethylethenyl group, 1-methyl-1-propenyl group, 1-methyl-2-propenyl group, 1-pentenyl group, 2-pentenyl group, 3-pentenyl group, 4-pentenyl group, 1-n-propylethenyl group, 1-methyl-1-butenyl group, 1-methyl-2-butenyl group, 1-methyl-3-butenyl group, 2-ethyl-2-propenyl group, 2-methyl-1-butenyl group, 2-methyl-2-butenyl group, 2-methyl-3-butenyl group, 3-methyl-1-butenyl group, 3-methyl-2-butenyl group, 3-methyl-3-butenyl group, 1,1-dimethyl-2-propenyl group, 1-i-propylethenyl group, 1,2-dimethyl-1-propenyl group, 1,2-dimethyl-2-propenyl group, 1-cyclopentenyl group, 2-cyclopentenyl group, 3-cyclopentenyl group, 1-hexenyl group, 2-hexenyl group, 3-hexenyl group, 4-hexenyl group, 5-hexenyl group, 1-methyl-1-pentenyl group, 1-methyl-2-pentenyl group, 1-methyl-3-pentenyl group, 1-methyl-4-pentenyl group, 1-n-butylethenyl group, 2-methyl-1-pentenyl group, 2-methyl-2-pentenyl group, 2-methyl-3-pentenyl group, 2-methyl-4-pentenyl group, 2-n-propyl-2-propenyl group, 3-methyl-1-pentenyl group, 3-methyl-2-pentenyl group, 3-methyl-3-pentenyl group, 3-methyl-4-pentenyl group, 3-ethyl-3-butenyl group, 4-methyl-1-pentenyl group, 4-methyl-2-pentenyl group, 4-methyl-3-pentenyl group, 4-methyl-4-pentenyl group, 1,1-dimethyl-2-butenyl group, 1,1-dimethyl-3-butenyl group, 1,2-dimethyl-1-butenyl group, 1,2-dimethyl-2-butenyl group, 1,2-dimethyl-3-butenyl group, 1-methyl-2-ethyl-2-propenyl group, 1-s-butylethenyl group, 1,3-dimethyl-1-butenyl group, 1,3-dimethyl-2-butenyl group, 1,3-dimethyl-3-butenyl group, 1-i-butylethenyl group, 2,2-dimethyl-3-butenyl group, 2,3-dimethyl-1-butenyl group, 2,3-dimethyl-2-butenyl group, 2,3-dimethyl-3-butenyl group, 2-i-propyl-2-propenyl group, 3,3-dimethyl-1-butenyl group, 1-ethyl-I-butenyl group, 1-ethyl-2-butenyl group, 1-ethyl-3-butenyl group, 1-n-propyl-1-propenyl group, 1-n-propyl-2-propenyl group, 2-ethyl-1-butenyl group, 2-ethyl-2-butenyl group, 2-ethyl-3-butenyl group, 1,1,2-trimethyl-2-propenyl group, 1-t-butylethenyl group, 1-methyl-1-ethyl-2-propenyl group, 1-ethyl-2-methyl-1-propenyl group, 1-ethyl-2-methyl-2-propenyl group, 1-i-propyl-1-propenyl group, 1-i-propyl-2-propenyl group, 1-methyl-2-cyclopentenyl group, 1-methyl-3-cyclopentenyl group, 2-methyl-1-cyclopentenyl group, 2-methyl-2-cyclopentenyl group, 2-methyl-3-cyclopentenyl group, 2-methyl-4-cyclopentenyl group, 2-methyl-1-cyclopentenyl group, 2-methylene-cyclopentyl group, 3-methyl-1-cyclopentenyl group, 3-methyl-2-cyclopentenyl group, 3-methyl-3-cyclopentenyl group, 3-methyl-4-cyclopentenyl group, 3-methyl-5-cyclopentenyl group, 3-methylene-cyclopentyl group, 1-cyclohexenyl group, 2-cyclohexenyl group, and 3-cyclohexenyl group.

The term “aromatic hydrocarbon group” refers to a hydrocarbon group that exhibits aromaticity. The aromatic groups include aryl groups and heteroaryl groups.

Examples of the aryl groups include phenyl group, o-methylphenyl group, m-methylphenyl group, p-methylphenyl group, 2,3-dimethylphenyl group, 2,4-dimethylphenyl group, 2,5-dimethylphenyl group, 2,6-dimethylphenyl group, 3,4-dimethylphenyl group, 3,5-dimethylphenyl group, o-chlorophenyl group, m-chlorophenyl group, p-chlorophenyl group, o-fluorophenyl group, p-fluorophenyl group, o-methoxyphenyl group, p-methoxyphenyl group, p-nitrophenyl group, p-cyanophenyl group, a-naphthyl group, P-naphthyl group, o-biphenylyl group, m-biphenylyl group, p-biphenylyl group, 1-anthryl group, 2-anthryl group, 9-anthryl group, 1-phenanthryl group, 2-phenanthryl group, 3-phenanthryl group, 4-phenanthryl group, 9-phenanthryl group, 1-naphthacenyl group, 2-naphthacenyl group, 3-naphthacenyl group, 1-pyrenyl group, 2-pyrenyl group, and 3-pyrenyl group.

Examples of the heteroaryl groups include furanyl group, thiophenyl group, pyrrolyl group, imidazolyl group, pyranyl group, pyridinyl group, pyrimidinyl group, pyrazinyl group, pyrrolidinyl group, piperidinyl group, piperazinyl group, morpholinyl group, quinuclidinyl group, indolyl group, purinyl group, quinolinyl group, isoquinolinyl group, chromenyl group, thianthrenyl group, phenothiazinyl group, phenoxazinyl group, xanthenyl group, acridinyl group, phenazinyl group, and carbazolyl group.

Examples of the groups comprising a combination or a condensation product of the above groups include organic groups, in which two aromatic ring residues or aliphatic ring residues are connected to each other by a single bond, for example, divalent residues, such as biphenyl, cyclohexylphenyl, and bicyclohexyl.

When L², L⁵, and L⁸ defined above are “divalent organic groups”, they are preferably C1-C6 linear or branched alkylene groups optionally substituted with a hydroxy group or a halo group (for example, fluorine). Examples of the linear alkylene groups include methylene group, ethylene group, propylene group, butylene group, pentylene group, and hexylene group.

(2-3-7-1)

Some specific examples of the organic groups including a structure represented by formula (II) are illustrated below. * indicates a bonding site to the unit structure U. It is needless to say that the illustrated structures may be part of the whole structure.

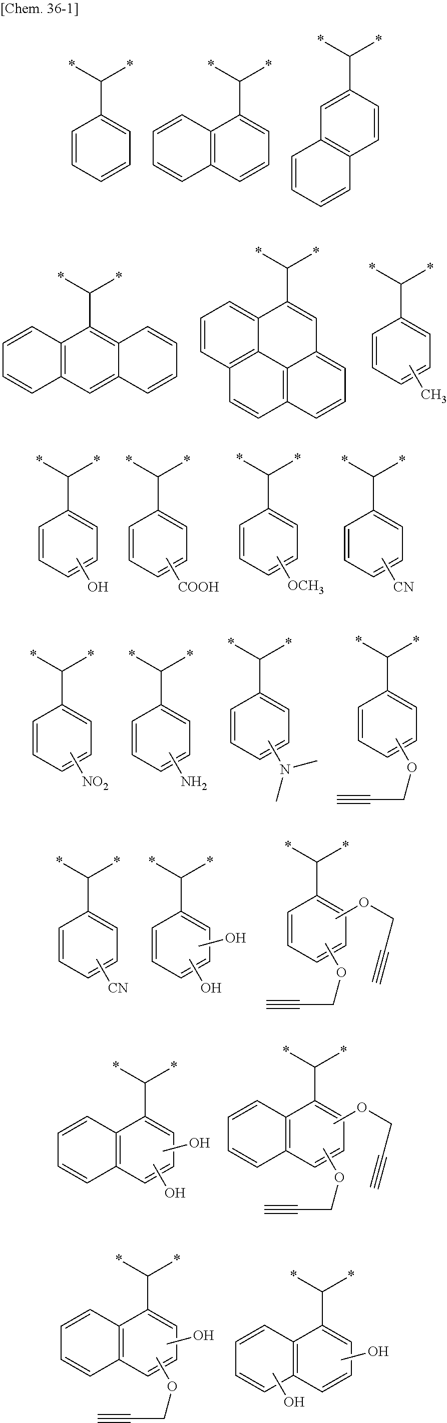

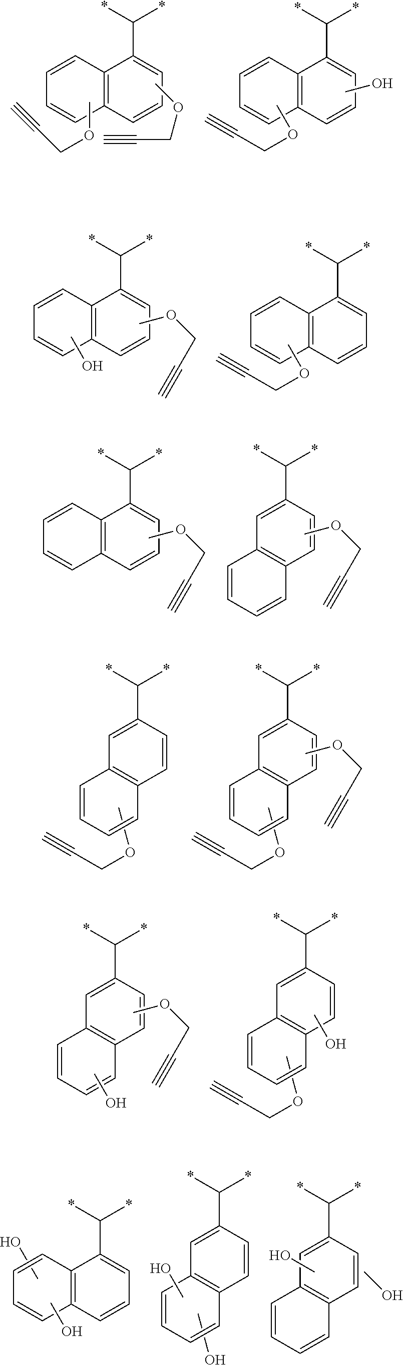

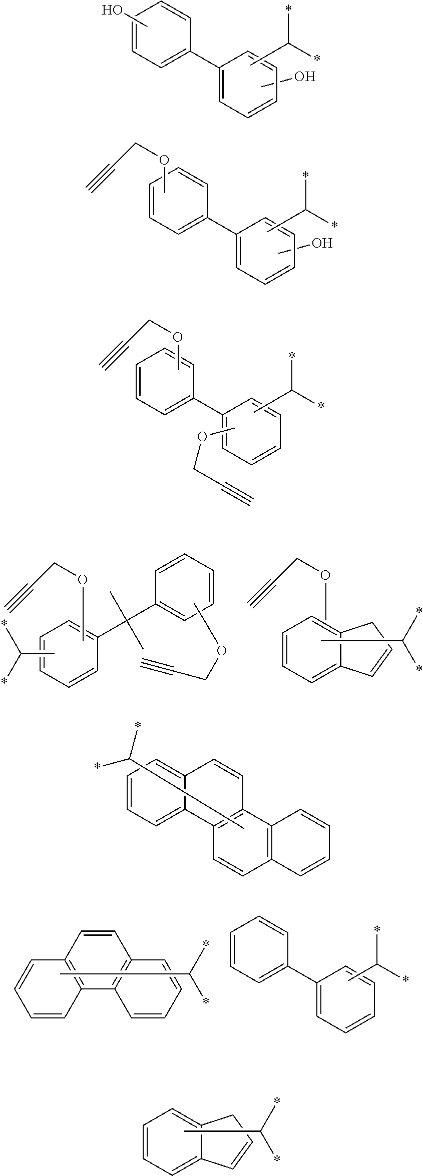

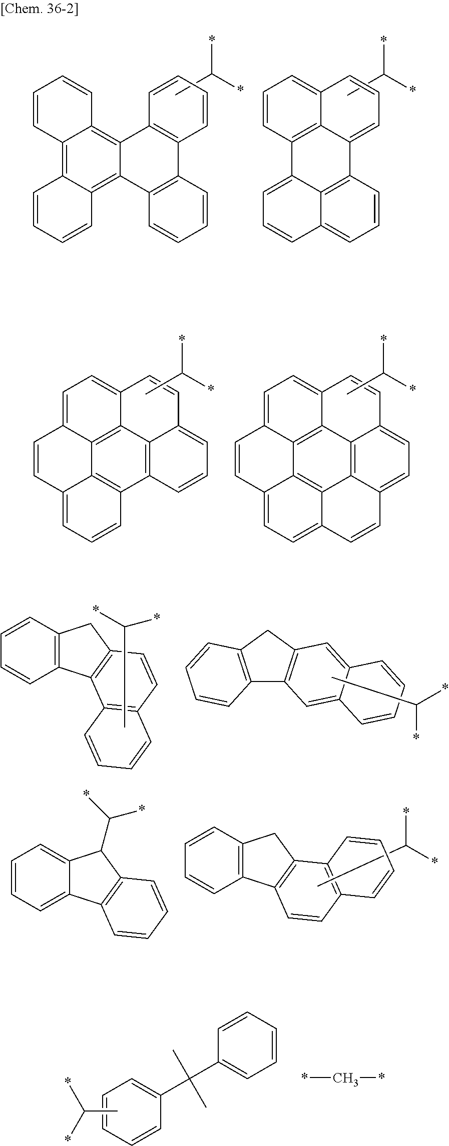

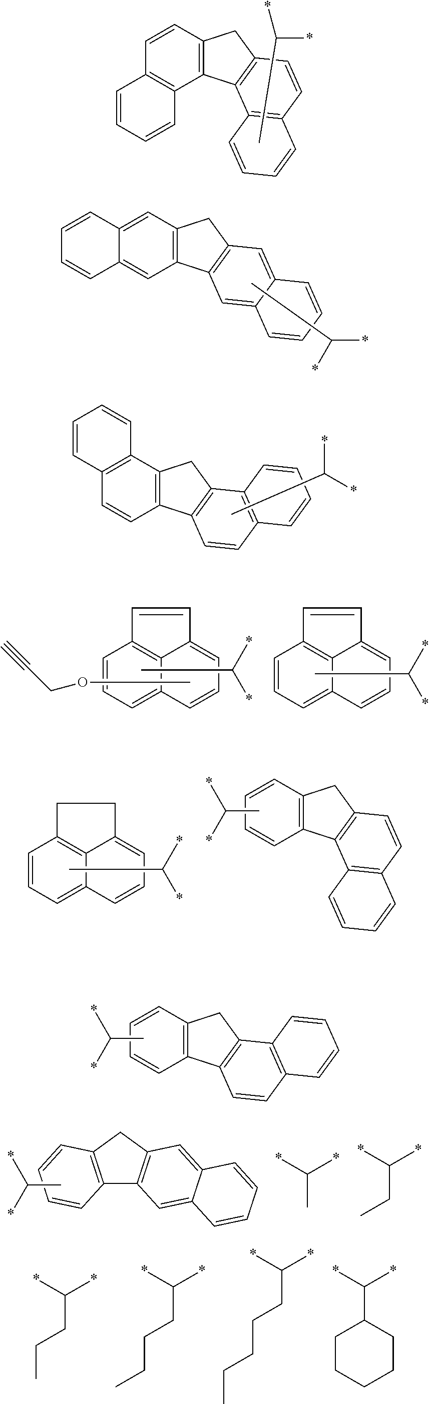

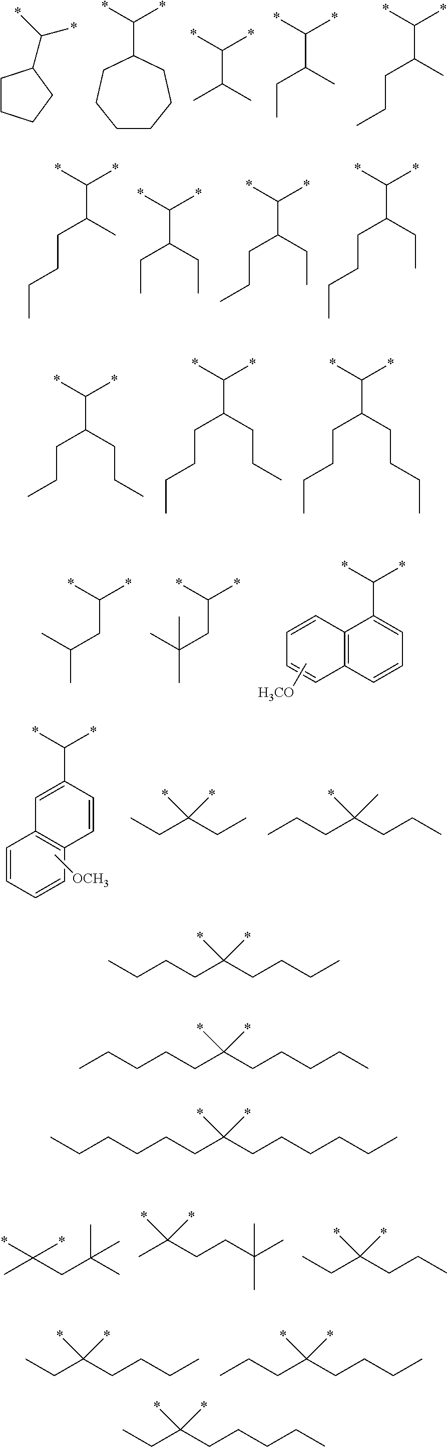

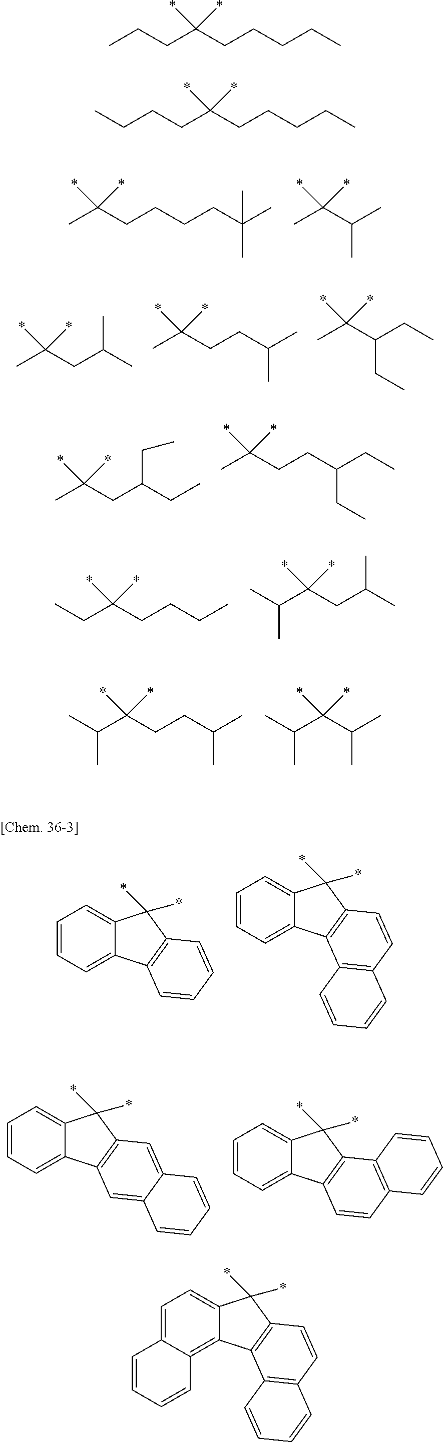

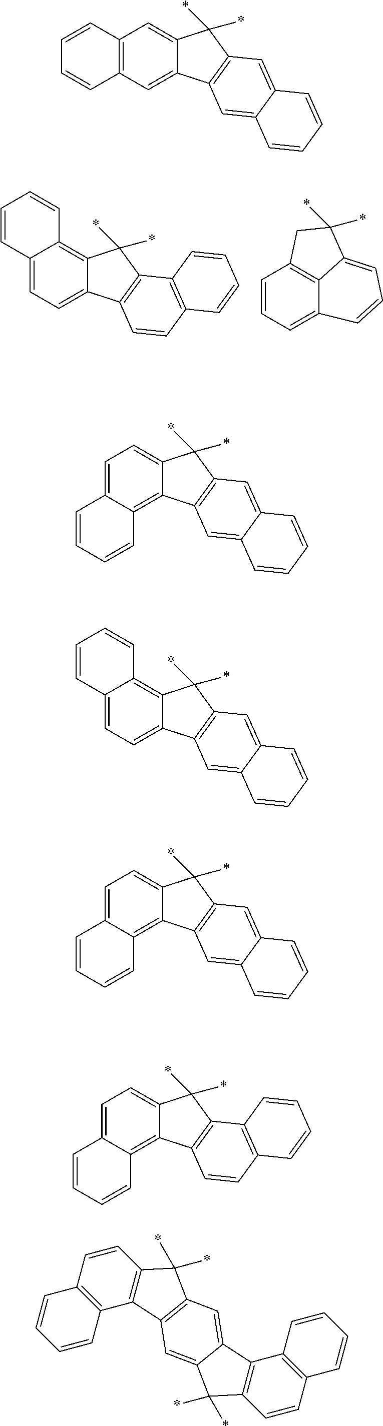

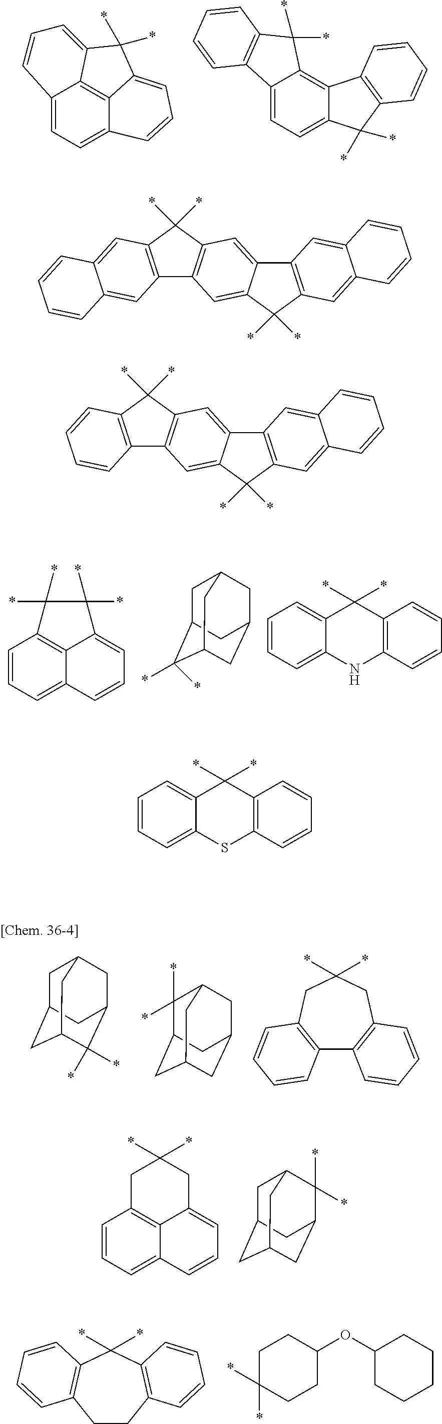

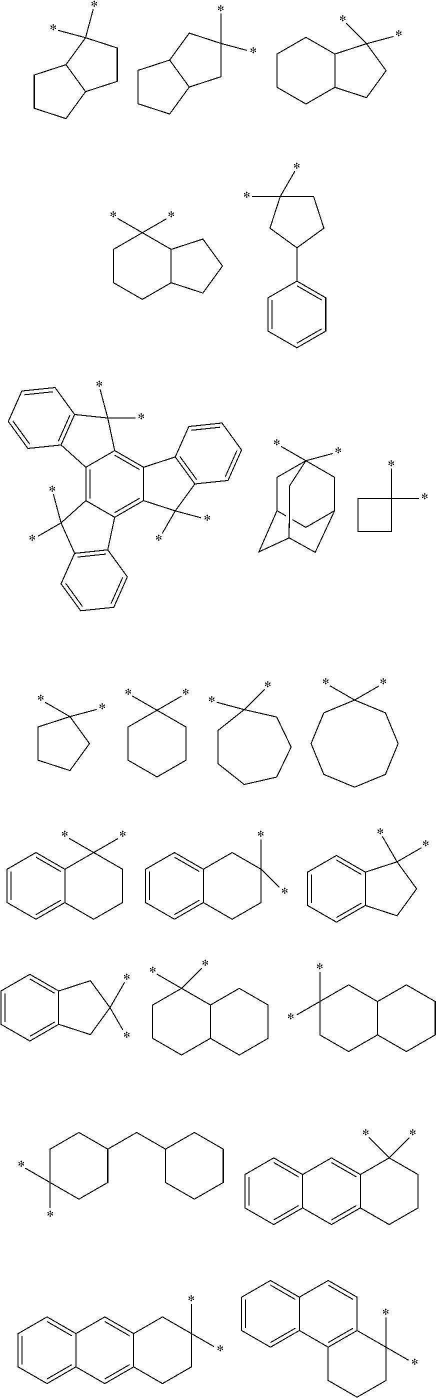

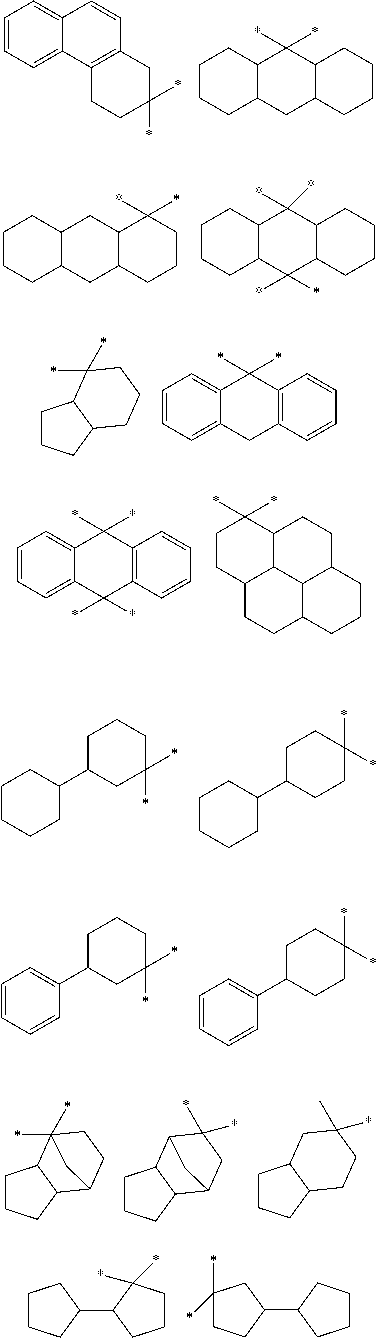

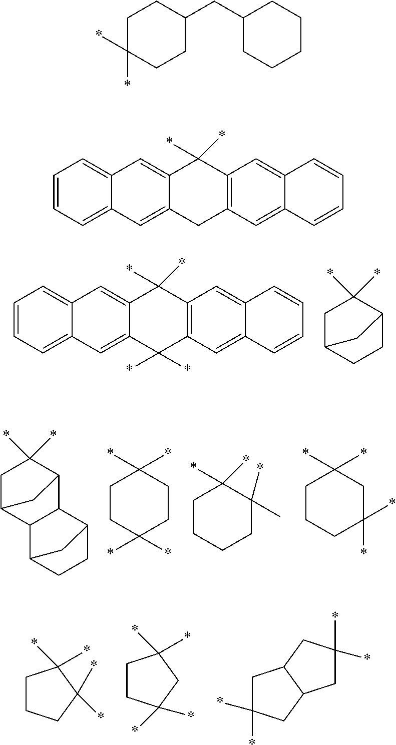

(2-3-7-2)

In principle, the two valence bonds in formula (II) are bonded to aromatic rings of the other structure having an aromatic ring (corresponding to the unit structures U). At a polymer terminal, however, the valence bond is bonded to a polymer terminal group [see (2-3-11) below].

For example, the unit structure including a structure represented by formula (II) may be in the form of a dimer or trimer structure, in which two or three same or different structures of formula (II) are connected to one another via a divalent or trivalent linking group. In this case, one of the two valence bonds in each of the structures of formula (II) is bonded to the linking group. Examples of such linking groups include linking groups having two or three aromatic rings (corresponding to the unit structures U). Refer to (2-3-8) for specific examples of the divalent or trivalent linking groups.

(2-3-8)

Some specific examples of the organic groups including a structure represented by formula (III) are illustrated below. The bonding sites to the unit structures U are not particularly limited. It is needless to say that the illustrated structures may be part of the whole structure.

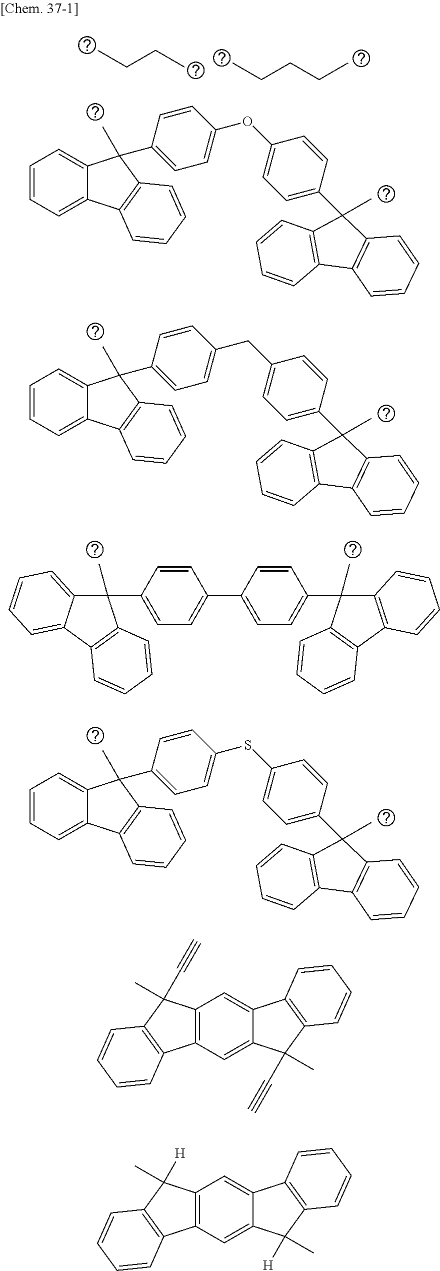

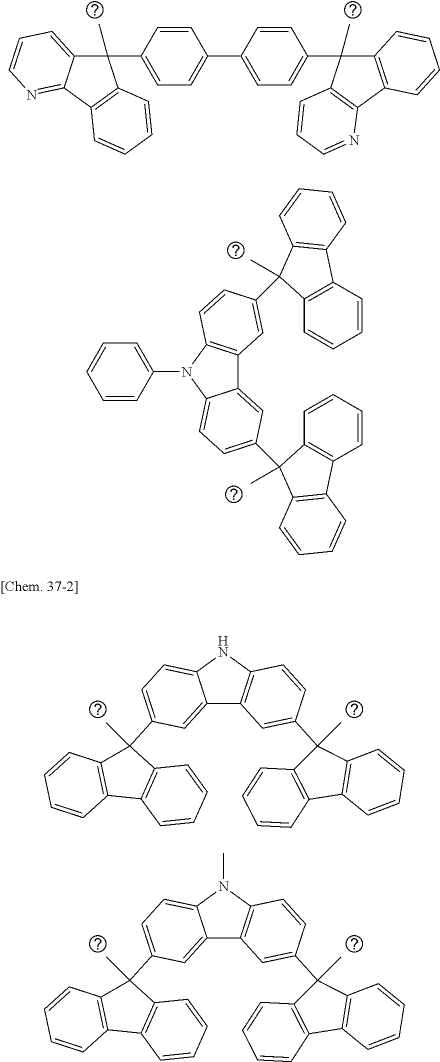

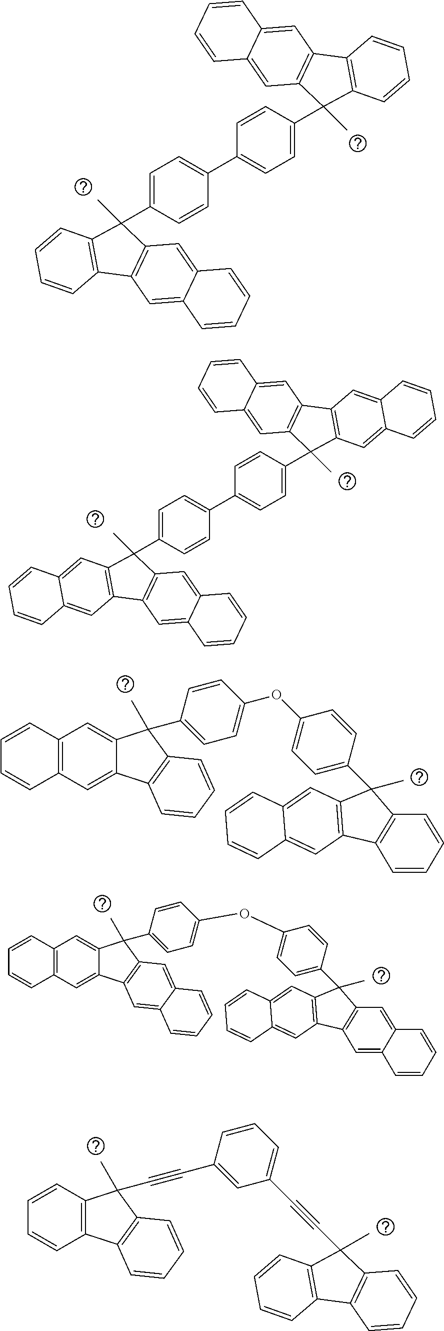

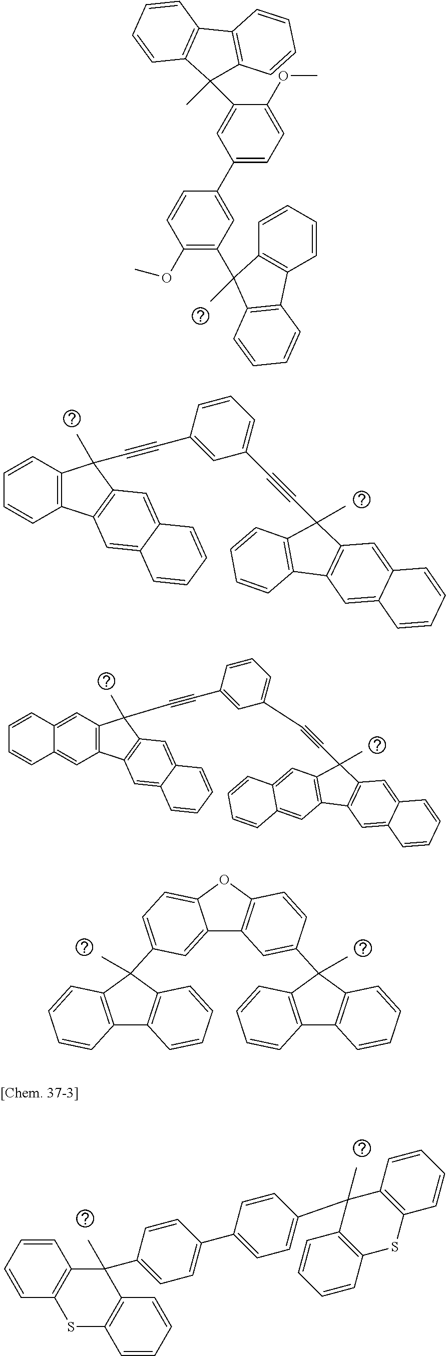

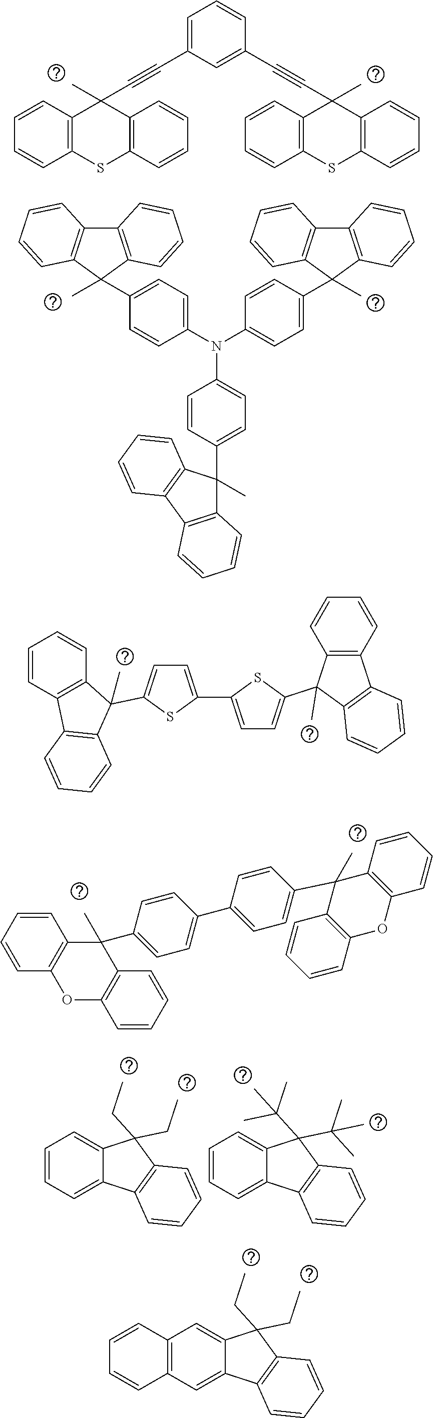

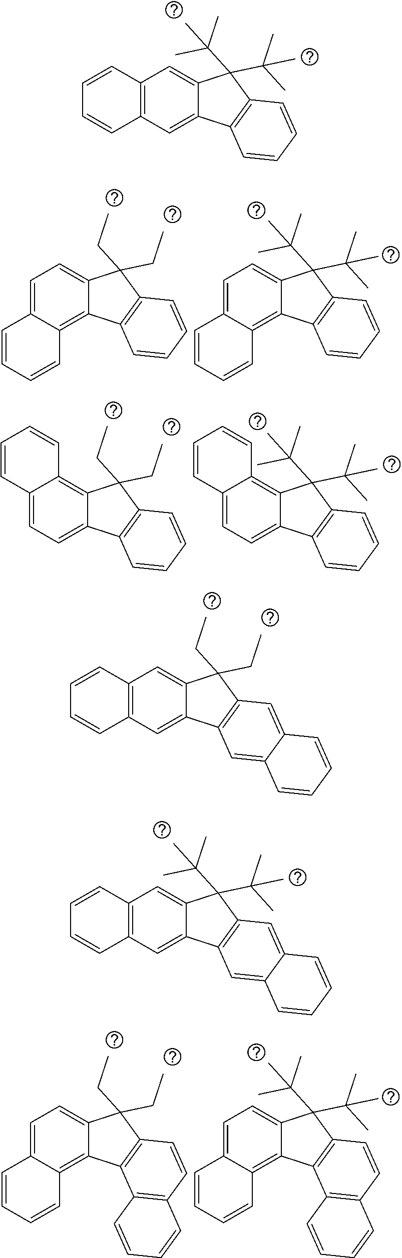

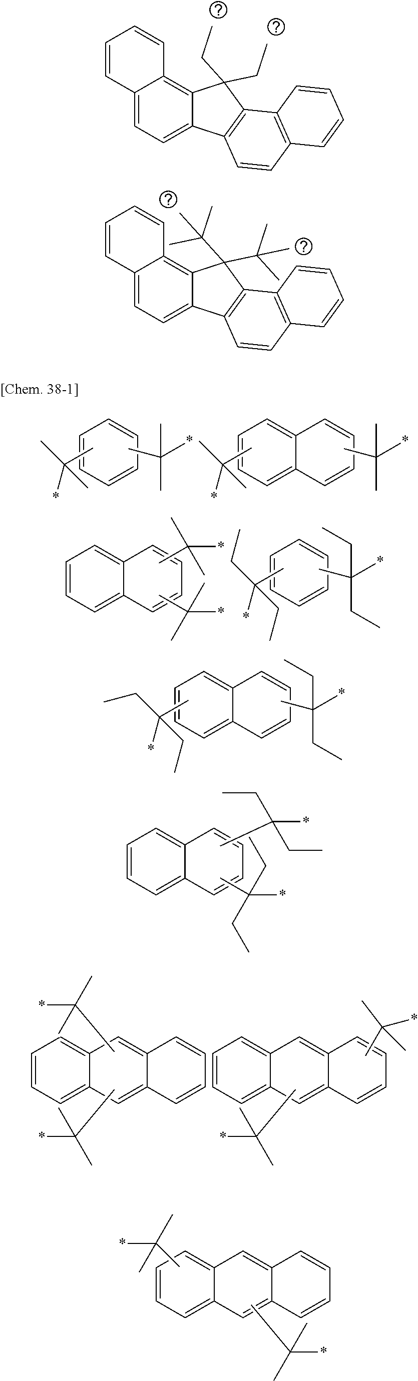

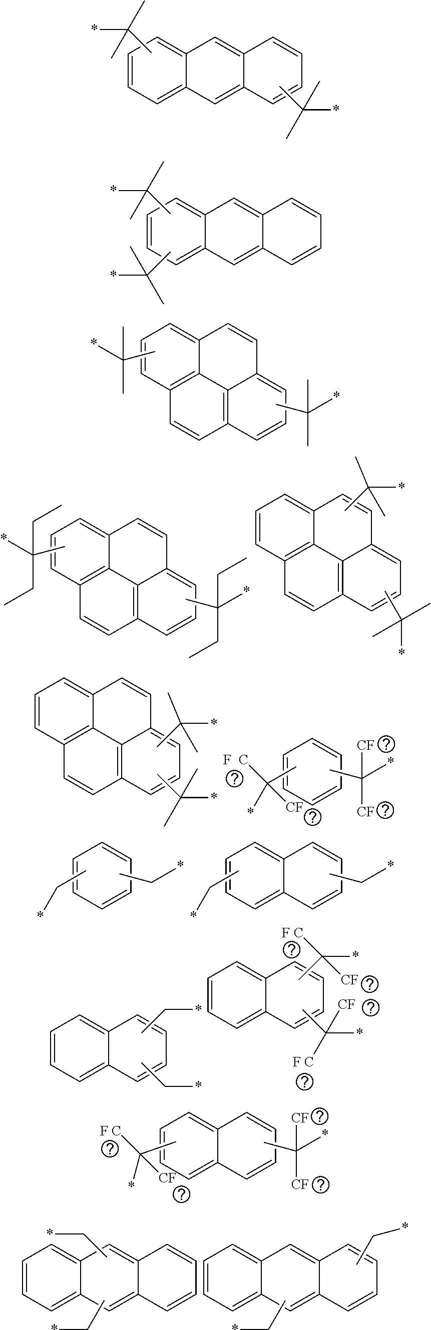

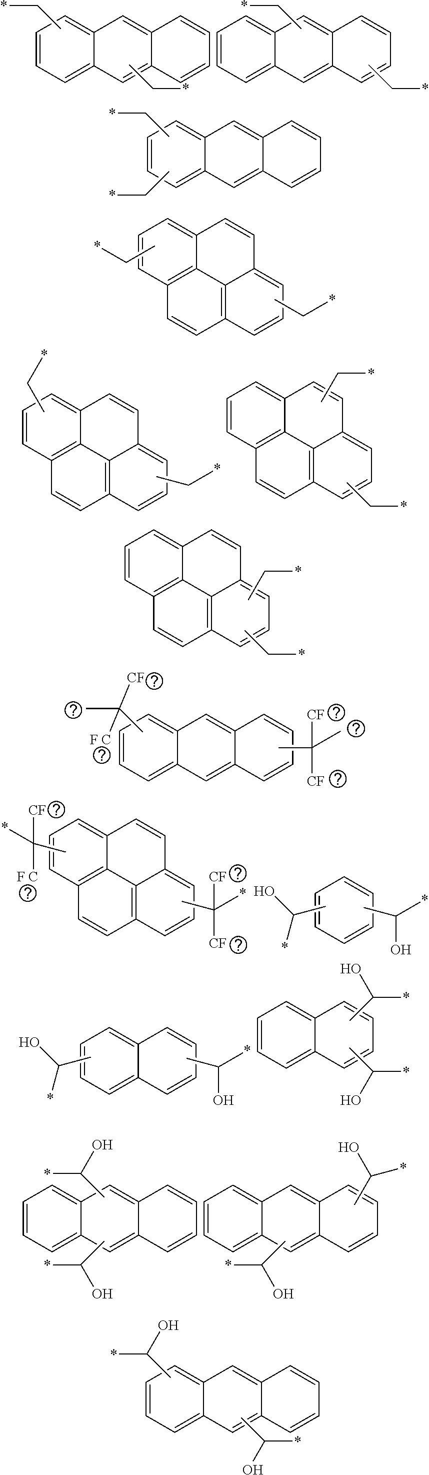

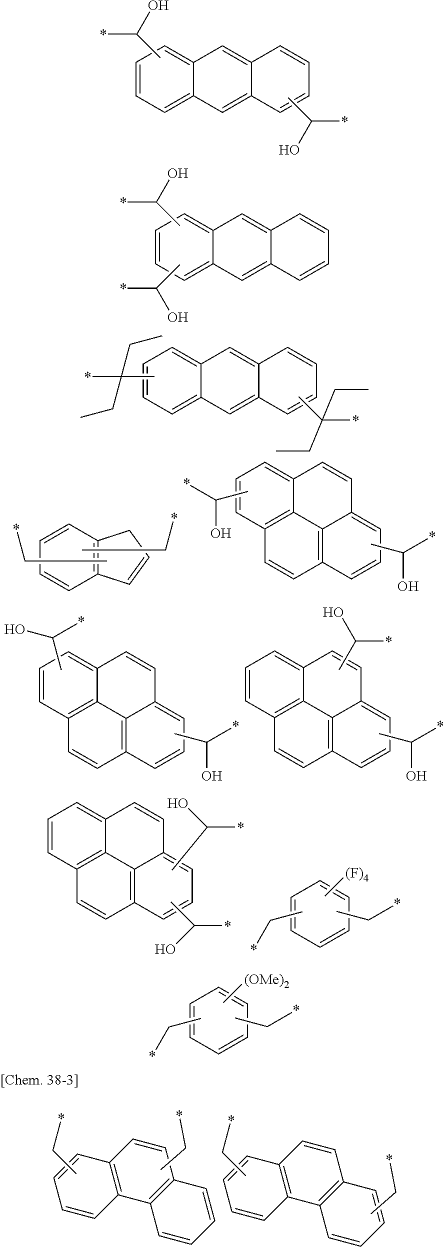

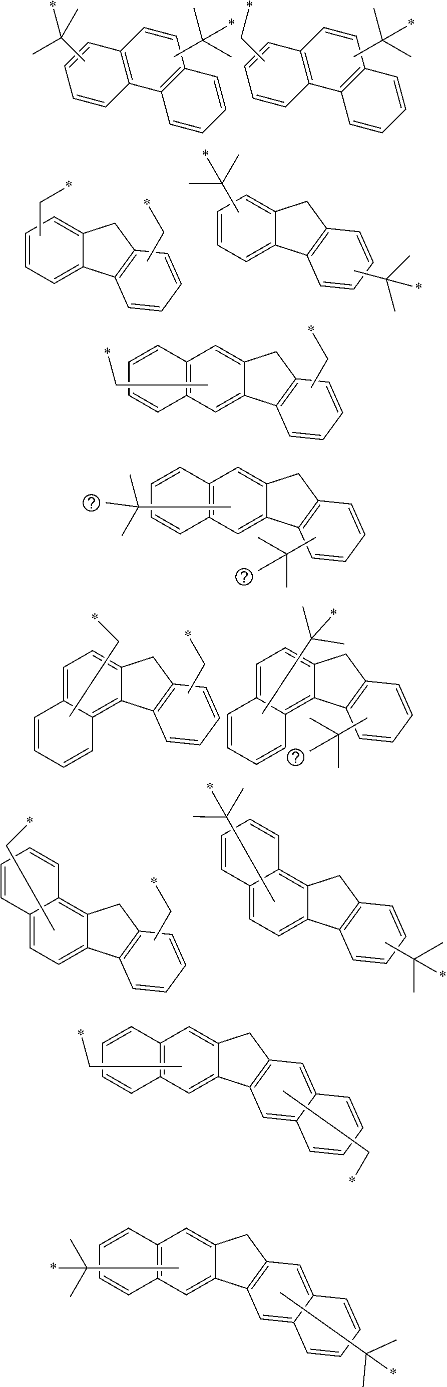

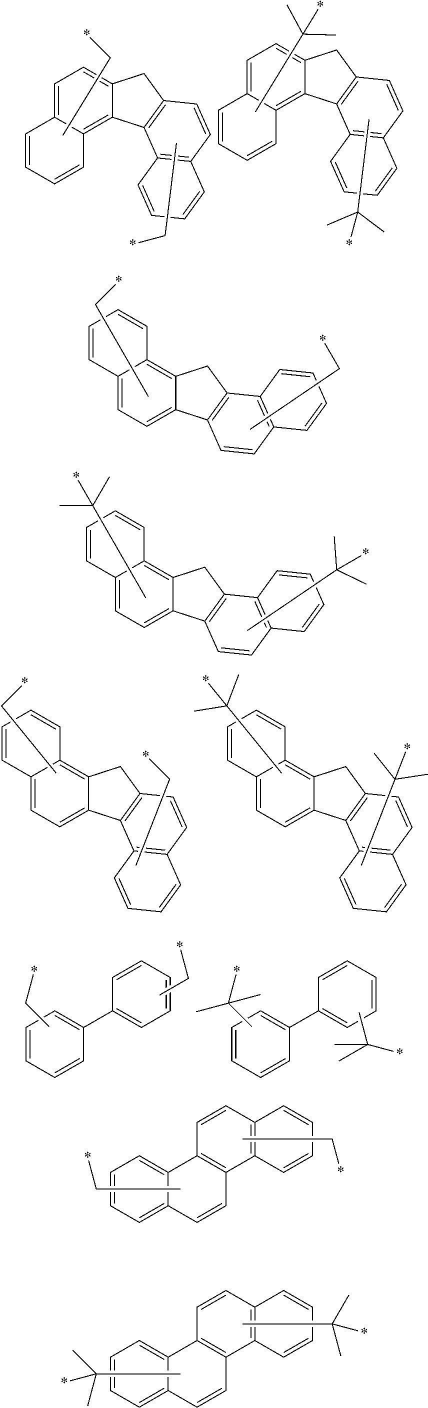

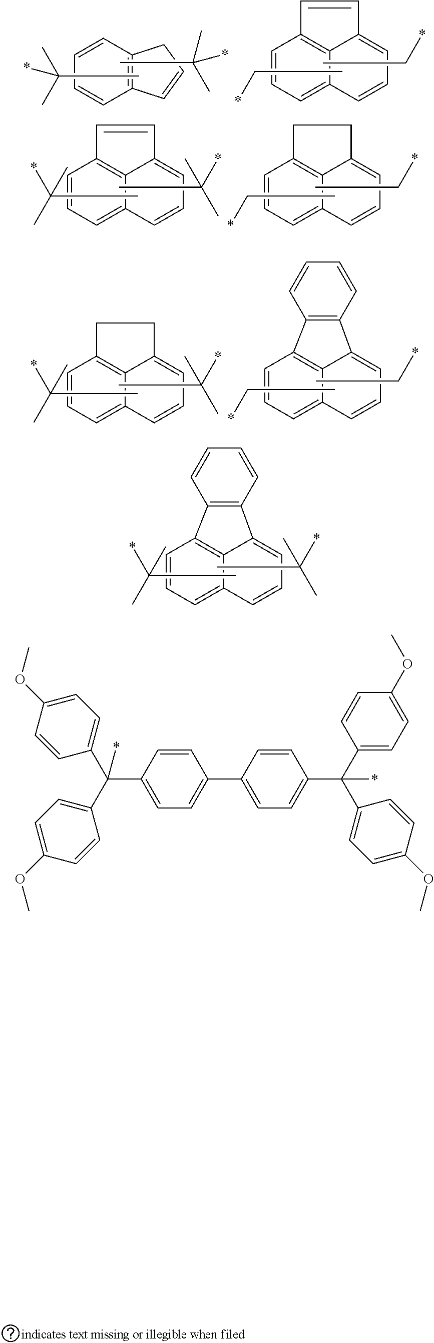

Examples of the linking groups corresponding to in formula (III) include linking groups that can be used as the unit structures U and have two or three aromatic rings. Examples of such linking groups include divalent or trivalent linking groups of

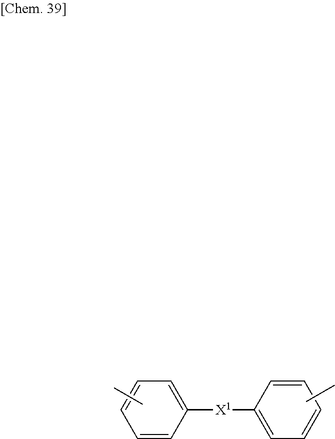

[X¹ denotes a single bond, a methylene group, an oxygen atom, a sulfur atom, or —N(R¹)—, and R¹ denotes a hydrogen atom or a C1-C20 hydrocarbon group (that may be a chain hydrocarbon or a cyclic hydrocarbon (that may be aromatic or non-aromatic)].

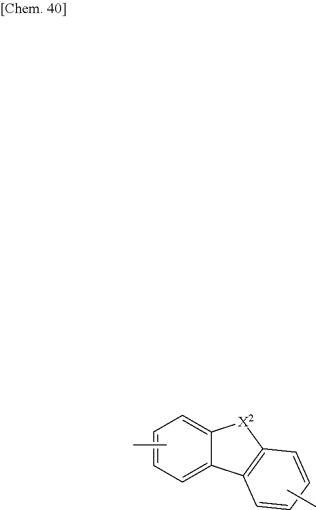

[X² denotes a methylene group, an oxygen atom, or —N(R²)—, and R² denotes a hydrogen atom, a C1-C10 aliphatic hydrocarbon group, or a C5-C20 aromatic hydrocarbon group].

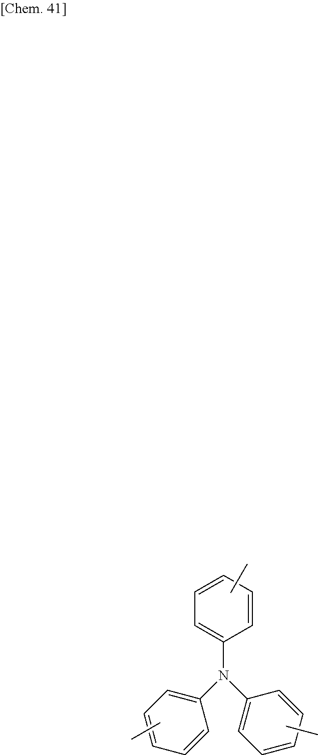

Examples further include a divalent linking group of the following formula that can undergo an addition reaction of the acetylide with a ketone to form a covalent bond with the linking carbon atom.

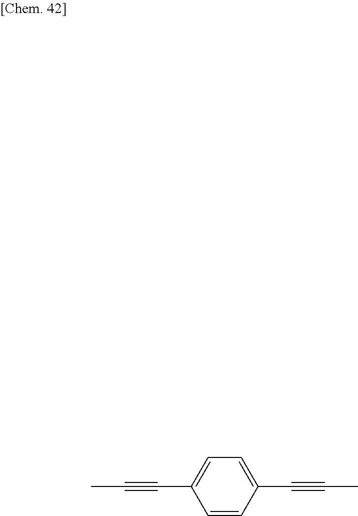

(2-3-9-1)

Some specific examples of the unit structures including a structure represented by formula (IV) are illustrated below. * indicates a bonding site to the unit structure U. It is needless to say that the illustrated structures may be part of the whole unit structure.

While the unit structures including a structure represented by formula (IV) have a valence bond on any of the aromatic rings in the structure that bonds to the unit structure V, the specific examples below omit such a valence bond. It is needless to say that the illustrated structures may be part of the whole unit structure. The illustrations without a valence bond on an aromatic ring can serve as specific examples of the polymer terminals.

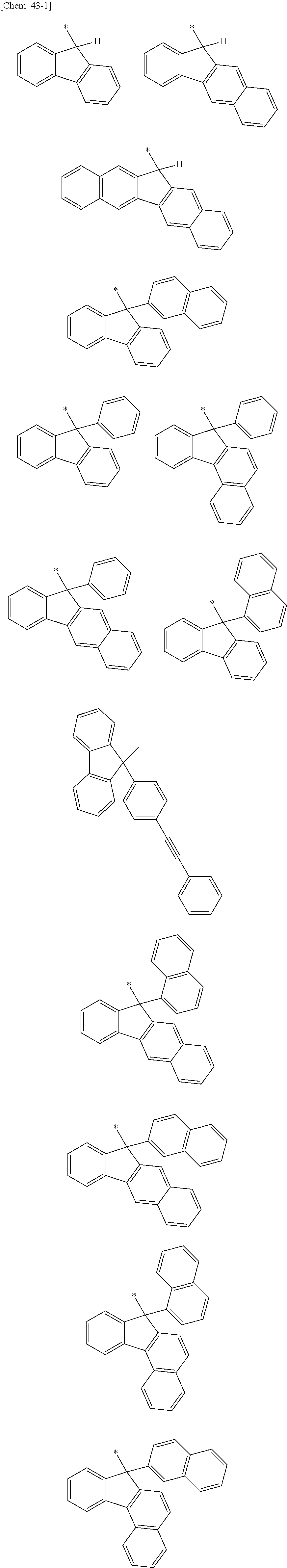

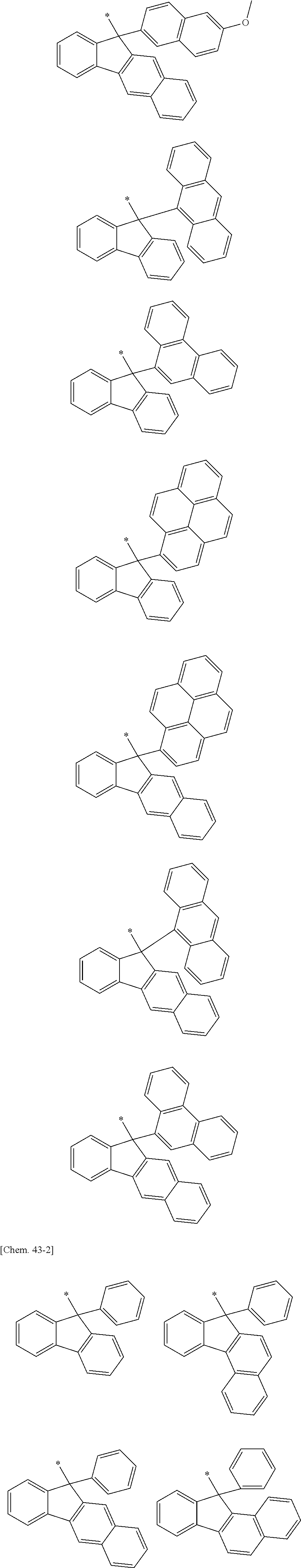

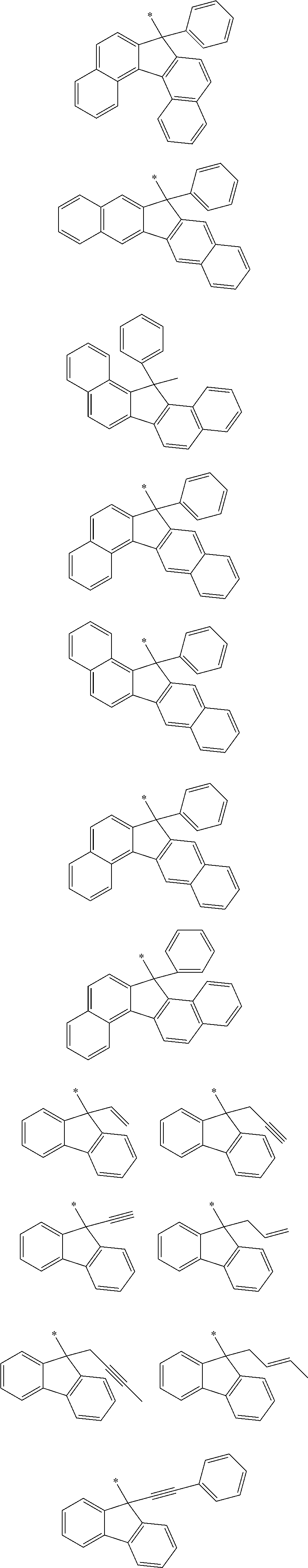

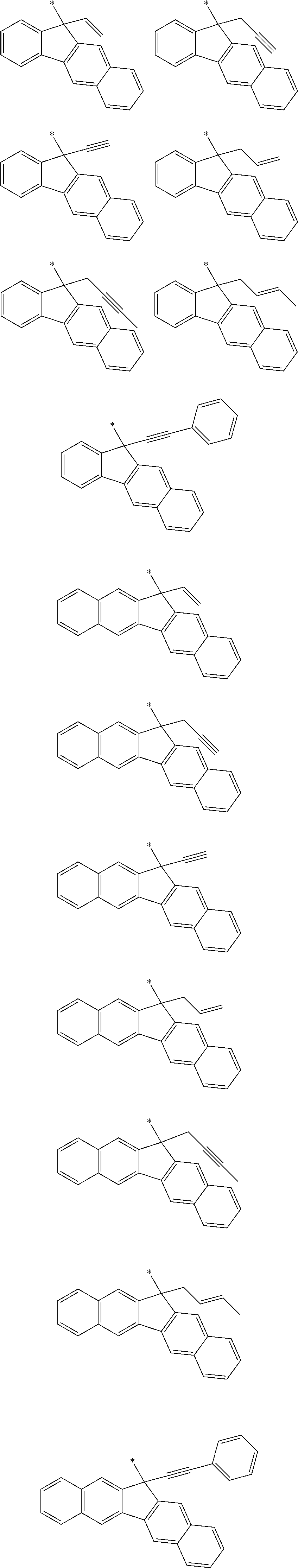

(2-3-9-2)

Two or three same or different structures of formula (IV) may be bonded to a divalent or trivalent linking group to form a dimer or trimer structure.

In such a case. one of the two valence bonds in each of the structures of formula (IV) is bonded to the linking group.

Examples of such linking groups include linking groups that can be used as the unit structures U and have two or three aromatic rings.

Refer to (2-3-8) for specific examples of the divalent or trivalent linking groups.

(2-3-9-3)

Formula (IV) may include an aromatic ring. In such a case, the aromatic ring in formula (IV) may be bonded to another of the unit structure V, and one of the valence bonds in formula (IV) may be bonded to the aromatic ring in the unit structure U. The unit structure resulting from this manner of bonding is equivalent to the composite unit structure U-V and may be used in place of at least one of the composite unit structures U-V.

The unit structure described above may be regarded as a type of the unit structures including a structure represented by formula (IV). In this case, the other of the valence bonds in formula (IV) may be bonded to, for example, a polymer terminal group or may be bonded to an aromatic ring in other polymer chain to form a crosslink.

(2-3-10)

The unit structure described above will be described based on more specific structures.

For example, the structure below may serve as a single unit structure equivalent to the composite unit structure U-V by bonding via p and k₁ or via p and k₂.

Alternatively, the structure below may also function as the unit structure U by bonding via k₁ and k₂.

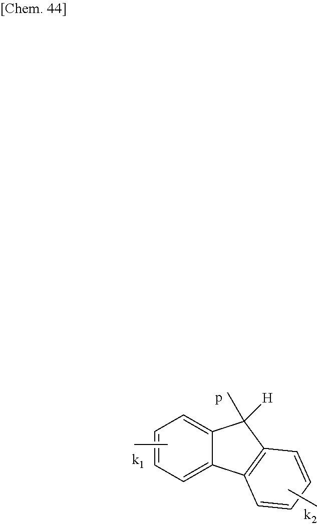

Furthermore, the exemplary structure below may serve as a single unit structure equivalent to the composite unit structure U-V by bonding via p and k₁, via p and k₂, or via p and m.

Alternatively, the structure below may also function as the unit structure U by bonding via k₁ and k₂, via k₁ and m, or via k₂ and m.

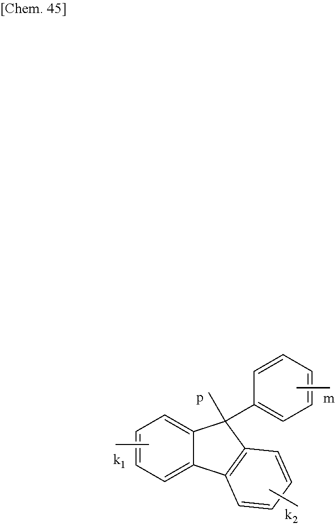

(2-3-11)

At a polymer terminal, the unit structure V forms a covalent bond with a terminal group (a polymer terminal group). The polymer terminal group may or may not be an aromatic ring derived from the unit structure U.

Examples of the polymer terminal groups include organic groups that contain a hydrogen atom, an optionally substituted aromatic ring residue, and an optionally substituted unsaturated aliphatic hydrocarbon residue [see substituents corresponding to the specific examples in (2-3-10)].

(2-3-12)

### Synthesis Methods

The novolac resin having a structure represented by formula (X) may be prepared by a known method. For example, such a novolac resin may be prepared by condensing a ring-containing compound represented by H-U-H with an oxygen-containing compound represented by, for example, OHC-V, O═C-V, HO-V-OH, or RO-V-OR. In the above formulas, U and V are the same as defined hereinabove, and R denotes a halogen or an alkyl group having about 1 to 3 carbon atoms.

The ring-containing compound and the oxygen-containing compound may be each a single compound or a combination of two or more compounds. In the condensation reaction, the oxygen-containing compound may be used in an amount of 0.1 to 10 mol, preferably 0.1 to 2 mol, per mol of the ring-containing compound.

A catalyst is used in the condensation reaction, with examples including mineral acids, such as sulfuric acid, phosphoric acid, and perchloric acid, organic sulfonic acids, such as p-toluenesulfonic acid, p-toluenesulfonic acid monohydrate, methanesulfonic acid, and trifluoromethanesulfonic acid, and carboxylic acids, such as formic acid and oxalic acid. The amount in which the catalyst is used varies depending on the type of the catalyst used, but is usually within the range of 0.001 to 10,000 parts by mass, preferably 0.01 to 1,000 parts by mass, and more preferably 0.05 to 100 parts by mass with respect to 100 parts by mass of the ring-containing compound (when more than one ring-containing compound is used, the total of the ring-containing compounds).

The condensation reaction may be carried out without a solvent, but is usually performed using a solvent. The solvent is not particularly limited as long as it can dissolve the reaction substrates and does not inhibit the reaction. Examples include 1,2-dimethoxyethane, diethylene glycol dimethyl ether, propylene glycol monomethyl ether, propylene glycol monomethyl ether acetate, tetrahydrofuran, dioxane, 1,2-dichloromethane, 1,2-dichloroethane, toluene, N-methylpyrrolidone, and dimethylformamide. The condensation reaction temperature is usually within the range of 40° C. to 200° C., and preferably 100° C. to 180° C. The reaction time varies depending on the reaction temperature, but is usually within the range of 5 minutes to 50 hours, and preferably 5 minutes to 24 hours.

The weight average molecular weight of the novolac resin according to an aspect of the present invention is usually within the range of 500 to 100,000, preferably 600 to 50,000, 700 to 10,000, or 800 to 8,000.

Examples of the polymer (G) include polymers disclosed in JP 2019-41059 A that have a repeating unit of general formula (1) below.

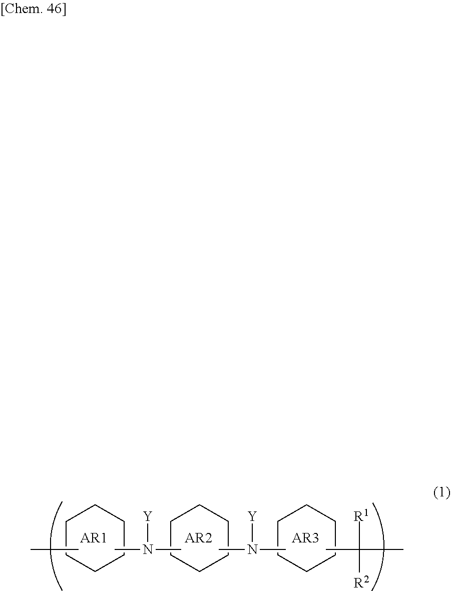

(In formula (1), AR1, AR2, and AR3 are each an optionally substituted benzene, naphthalene, or anthracene ring; AR1 and AR2, or AR2 and AR3 may form a bridged structure by bonding of carbon atoms on their aromatic rings directly or via a linking group; R¹ and R² are each independently a hydrogen atom or a C1-C30 organic group; when R¹ and R² are any of the organic groups, R¹ and R² may form a cyclic organic group by intramolecular bonding; and Y is a group represented by formula (2) below.)

[Chem. 47]

—R³—C═C—R⁴ (2)

(In formula (2), R³ is a single bond or a C1-C20 divalent organic group, R⁴ is a hydrogen atom or a C1-C20 monovalent organic group, and the broken line indicates a valence bond.)

Examples of the polymer (G) include polymers disclosed in JP 2019-44022 A that have a repeating unit of general formula (1) below.

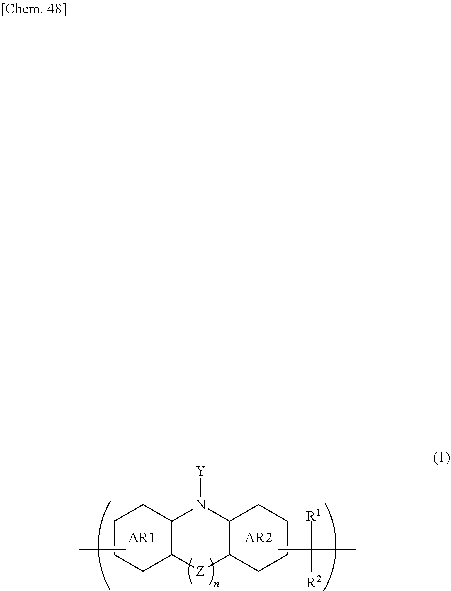

(In formula (1), AR1 and AR2 are each an optionally substituted benzene or naphthalene ring; R¹ and R² are each independently a hydrogen atom or a C1-C30 organic group; when R¹ and R² are any of the organic groups, R¹ and R² may form a cyclic organic group by intramolecular bonding; n is 0 or 1; when n=0, AR1 and AR2 do not form a bridged structure between the aromatic rings AR1 and AR2 via Z; when n=1, AR1 and AR2 form a bridged structure between the aromatic rings AR1 and AR2 via Z; Z is a single bond or any of formula (2) below; and Y is a group represented by formula (3) below.)

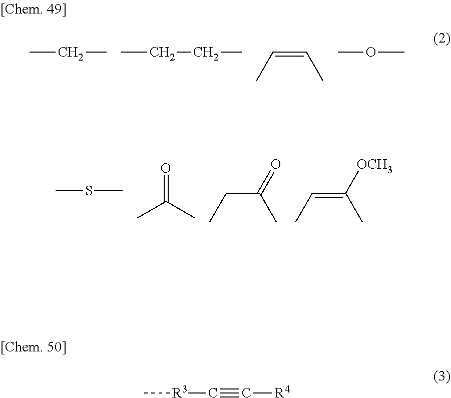

(In formula (3), R³ is a single bond or a C1-C20 divalent organic group, R⁴ is a hydrogen atom or a C1-C20 monovalent organic group, and the broken line indicates a valence bond.)

Examples of the polymer (G) include polymers disclosed in JP 2018-168375 A. The polymers include a unit structure of formula (5) below:

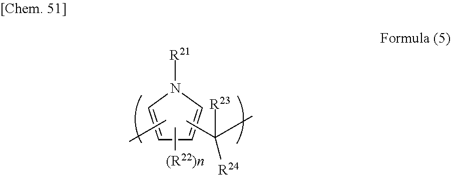

(In formula (5), R²¹ is selected from the group consisting of a hydrogen atom, a C1-C10 alkyl group, a C2-C10 alkenyl group, a C6-C40 aryl group, and a combination of these groups, and the alkyl group, the alkenyl group, or the aryl group may contain an ether bond, a ketone bond, or an ester bond; R²² is selected from the group consisting of a halogen group, a nitro group, an amino group, a hydroxy group, a C1-C10 alkyl group, a C2-C10 alkenyl group, a C6-C40 aryl group, and a combination of these groups, and the alkyl group, the alkenyl group, or the aryl group may contain an ether bond, a ketone bond, or an ester bond; R²³ is a hydrogen atom, or a C6-C40 aryl group or a heterocyclic group optionally substituted with a halogen group, a nitro group, an amino group, a carbonyl group, a C6-C40 aryl group, or a hydroxy group; R²⁴ is a C1-C10 alkyl group, a C6-C40 aryl group, or a heterocyclic group optionally substituted with a halogen group, a nitro group, an amino group, or a hydroxy group; R²³ and R²⁴ may form a ring together with the carbon atom to which they are bonded; and n denotes an integer of 0 to 2.)

Examples of the polymer (G) include polymers disclosed in Japanese Patent No. 5641253. The polymers include a unit structure represented by formula (1) below:

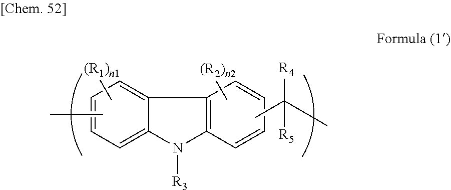

(In formula (1′),
R₁, R₂, and R₃ each denote a hydrogen atom,
R₄ and Rₛ together with the carbon atom to which they are bonded form a fluorene ring, in which the carbon atom is in the 9-position of the fluorene ring, and
n1 and n2 are each an integer of 3). The polymers have a weight average molecular weight of 1000 to 6400.

Examples of the polymer (G) include polymers disclosed in Japanese Patent No. 6041104 that include a unit structure composed of a reaction product of a carbazole compound or a substituted carbazole compound with a bicyclic ring compound.

Examples of the polymer (G) include polymers disclosed in Japanese Patent No. 6066092. The polymers include a unit structure (A) represented by formula (1) below:

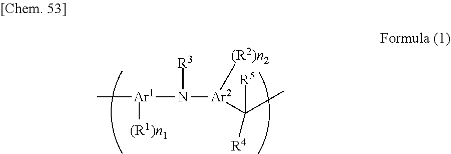

(In formula (1), Ar^{l} and Ar² each denote a benzene ring or a naphthalene ring; R¹ and R² are each a substituent for a hydrogen atom on the ring and are each selected from the group consisting of a halogen group, a nitro group, an amino group, a hydroxy group, a C1-C10 alkyl group, a C2-C10 alkenyl group, a C6-C40 aryl group, and a combination thereof, and the alkyl group, the alkenyl group, and the aryl group are organic groups optionally containing an ether bond, a ketone bond, or an ester bond;

R³ is selected from the group consisting of a hydrogen atom, a C1-C10 alkyl group, a C2-C10 alkenyl group, a C6-C40 aryl group, and a combination thereof, and the alkyl group, the alkenyl group, and the aryl group are organic groups optionally containing an ether bond, a ketone bond, or an ester bond;

R⁴ is selected from the group consisting of a C6-C40 aryl group and a heterocyclic group, and the aryl group and the heterocyclic group are organic groups optionally substituted with a halogen group, a nitro group, an amino group, a C1-C10 alkyl group, a C1-C10 alkoxy group, a C6-C40 aryl group, a formyl group, a carboxyl group, or a hydroxy group; and

R⁵ is selected from the group consisting of a hydrogen atom, a C1-C10 alkyl group, a C6-C40 aryl group, and a heterocyclic group, and the alkyl group, the aryl group, and the heterocyclic group are organic groups optionally substituted with a halogen group, a nitro group, an amino group, or a hydroxy group; R⁴ and R⁵ may form a ring together with the carbon atom to which they are bonded; and n₁ and n₂ are each an integer of 0 to 3.)

The unit structure (A) is a unit structure (al) in which either Ar^{l} or Ar² is a benzene ring and the other is a naphthalene ring, and R³ is selected from the group consisting of a hydrogen atom, a C1-C10 alkyl group, a C2-C10 alkenyl group, and a combination thereof, and the alkyl group and the alkenyl group are organic groups optionally containing an ether bond, a ketone bond, or an ester bond.

Examples of the polymer (G) include polymers disclosed in Japanese Patent No. 6094767. The polymers have a unit structure represented by formula (1) below:

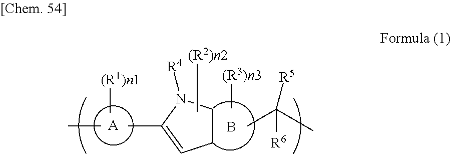

(In formula (1), R¹, R², and R³ are each a substituent for a hydrogen atom on the ring and are each independently a halogen group, a nitro group, an amino group, a hydroxy group, a C1-C10 alkyl group, a C2-C10 alkenyl group, a C6-C40 aryl group, or a combination thereof optionally containing an ether bond, a ketone bond, or an ester bond; R⁴ is a hydrogen atom, a C1-C10 alkyl group, a C2-C10 alkenyl group, a C6-C40 aryl group, or a combination thereof optionally containing an ether bond, a ketone bond, or an ester bond; R⁵ is a hydrogen atom, or a C6-C40 aryl group or a heterocyclic group optionally substituted with a halogen group, a nitro group, an amino group, a formyl group, a carboxyl group, a carboxylic acid alkyl ester group, a phenyl group, a C1-C10 alkoxy group, or a hydroxy group; R⁶ is a hydrogen atom, or a C1-C10 alkyl group, a C6-C40 aryl group, or a heterocyclic group optionally substituted with a halogen group, a nitro group, an amino group, a formyl group, a carboxyl group, a carboxylic acid alkyl ester group, or a hydroxy group; R⁵ and R⁶ may form a ring together with the carbon atom to which they are bonded; the ring A and the ring B each denote a benzene ring, a naphthalene ring, or an anthracene ring; and n1, n2, and n3 are each an integer from 0 up to the number of the maximum substitution that is possible on the ring).

Examples of the polymer (G) include polymers disclosed in Japanese Patent No. 6137486. The polymers include a unit structure represented by formula (1) below:

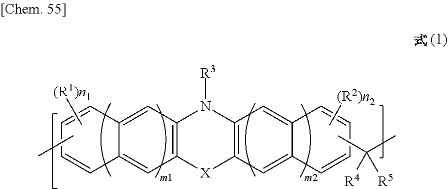

(In formula (1), R¹ and R² are each a substituent for a hydrogen atom on the aromatic ring and are each independently a halogen group, a nitro group, an amino group, a carboxylic acid group, a hydroxy group, a C1-C10 alkyl group, a C2-C10 alkenyl group, a C6-C40 aryl group, an ether bond-containing organic group, a ketone bond-containing organic group, an ester bond-containing organic group, or a combination thereof,
R³ is a hydrogen atom, a C1-C10 alkyl group, a C2-C10 alkenyl group, a C6-C40 aryl group, an ether bond-containing organic group, a ketone bond-containing organic group, an ester bond-containing organic group, or a combination thereof,
R⁴ is a C6-C40 aryl group or a heterocyclic group, and the aryl group and the heterocyclic group are each optionally substituted with a halogen group, a nitro group, an amino group, a C1-C10 alkyl group, a C1-C10 alkoxy group, a C6-C40 aryl group, a formyl group, a carboxyl group, a carboxylic acid ester group, or a hydroxy group; R⁵ is a hydrogen atom, a C1-C10 alkyl group, a C6-C40 aryl group, or a heterocyclic group, and the alkyl group, the aryl group, and the heterocyclic group are each optionally substituted with a halogen group, a nitro group, an amino group, or a hydroxy group; R⁴ and R⁵ may form a ring together with the carbon atom to which they are bonded;
X is an O atom, a S atom, a CH₂ group, a C═O group, a CH═CH group, or a CH₂—CH₂ group; n₁ and n₂ are each an integer of 0 to 3; and m1 and m2 are each an integer of 0 to 3.)

Examples of the polymer (G) include novolac resins disclosed in Japanese Patent No. 6583636 that have an additional structural group (C) obtained by the reaction of an aromatic ring structure in an aromatic ring-containing compound (A) with a vinyl group in an aromatic vinyl compound (B) having one vinyl group in the molecule. In the novolac resins, the aromatic ring-containing compound (A) is an aromatic amine compound.

Examples of the polymer (G) include novolac resins disclosed in WO 2017/069063 that are obtained by reacting an aromatic compound (A) with an aldehyde (B) having a formyl group bonded to a secondary carbon atom or a tertiary carbon atom of a C2-C26 alkyl group.

Examples of the polymer (G) include polymers disclosed in WO 2017/094780. The polymers include a unit structure represented by formula (1) below:

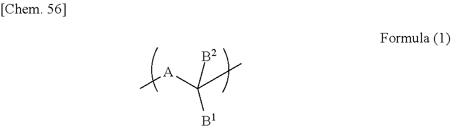

(In formula (1), A is a divalent group having at least two amino groups, this group is derived from a compound having a condensed ring structure and an aromatic group substituting for a hydrogen atom on the condensed ring, and B¹ and B² are each independently a hydrogen atom, an alkyl group, a benzene ring group, a condensed ring group, or a combination thereof, or B¹ and B² may form a ring together with the carbon atom to which they are bonded.)

Examples of the polymer (G) include polymers disclosed in WO 2018/043410. The polymers include a unit structure represented by formula (1) below:

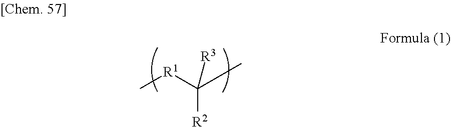

(In formula (1), R¹ is an organic group containing at least two amines and at least three C6-C40 aromatic rings, and
R² and R³ are each a hydrogen atom, a C1-C10 alkyl group, a C6-C40 aryl group, a heterocyclic group, or a combination thereof, and the alkyl group, the aryl group, and the heterocyclic group are optionally substituted with a halogen group, a nitro group, an amino group, a formyl group, an alkoxy group, or a hydroxy group, or
R² and R³ may form a ring together.)

Examples of the polymer (G) include resins disclosed in Japanese Patent No. 4877101 that have a group represented by general formula (1) below and an aromatic hydrocarbon group.

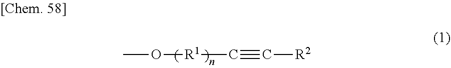

(In general formula (1), n denotes 0 or 1; R¹ denotes an optionally substituted methylene group, an optionally substituted C2-C20 alkylene group, or an optionally substituted C6-C20 arylene group; and R² denotes a hydrogen atom, an optionally substituted C1-C20 alkyl group, or an optionally substituted C6-C20 aryl group.)

Examples of the polymer (G) include compounds disclosed in Japanese Patent No. 4662063 that have a bisphenol group represented by general formula (1) below:

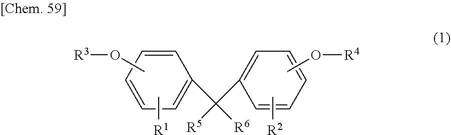

(wherein R¹ and R² are identical to or different from each other and are each a hydrogen atom, a C1-C10 linear, branched, or cyclic alkyl group, a C6-C10 aryl group, or a C2-C10 alkenyl group; R³ and R⁴ are each a hydrogen atom, a C1-C6 linear, branched, or cyclic alkyl group, a C2-C6 linear, branched, or cyclic alkenyl group, a C6-C10 aryl group, a C2-C6 acetal group, a C2-C6 acyl group, or a glycidyl group; and R⁵ and R⁶ are each a C5-C30 alkyl group having a ring structure and may have a double bond or may be interrupted with a heteroatom; or R⁵ and R⁶ may be bonded to each other and the group represented by:

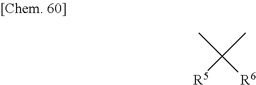

may be any of the following formulas:

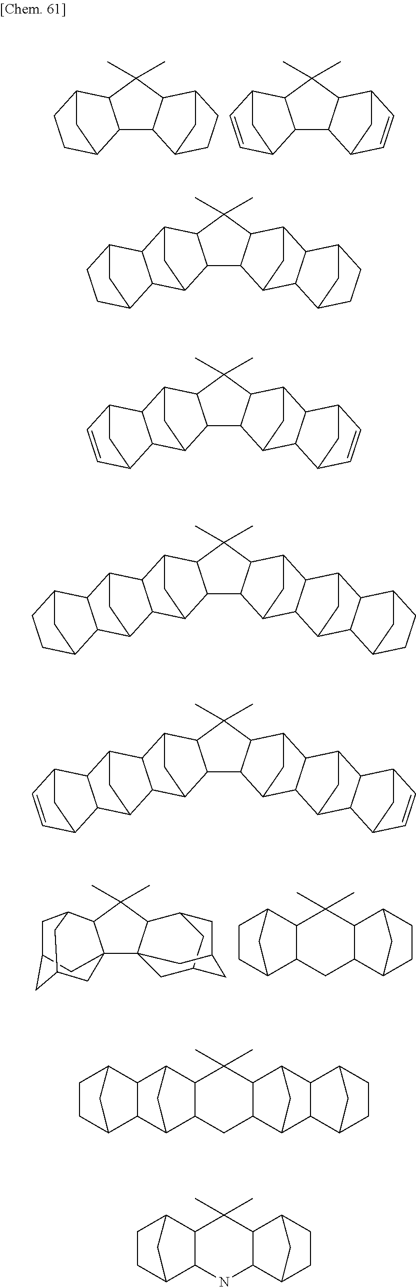

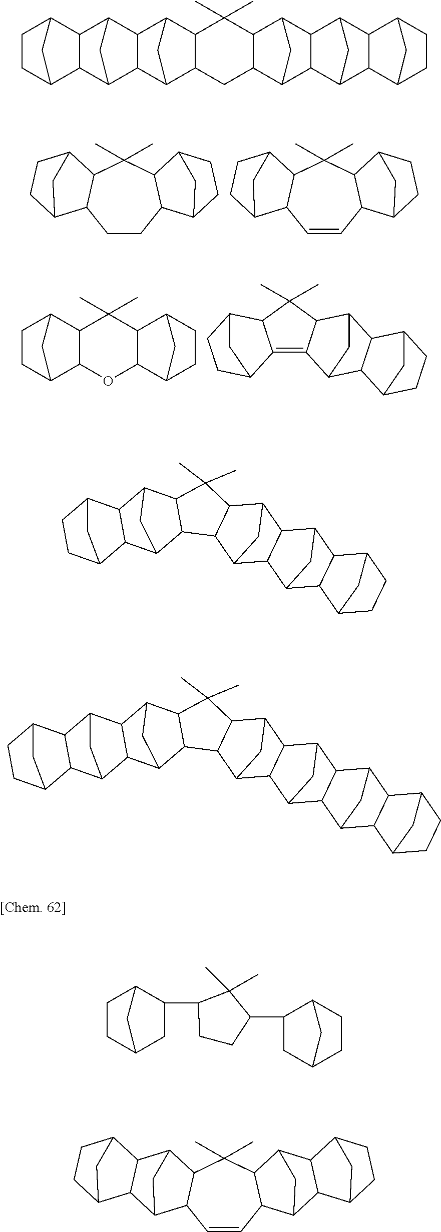

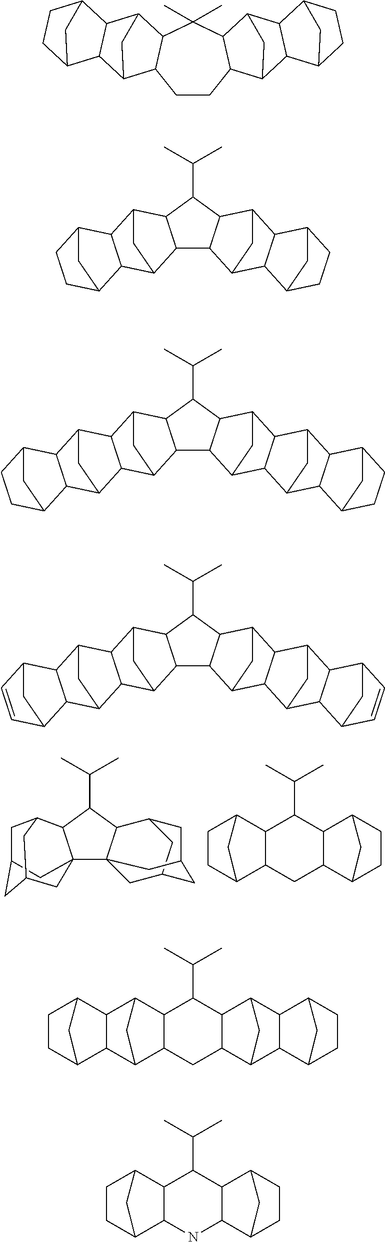

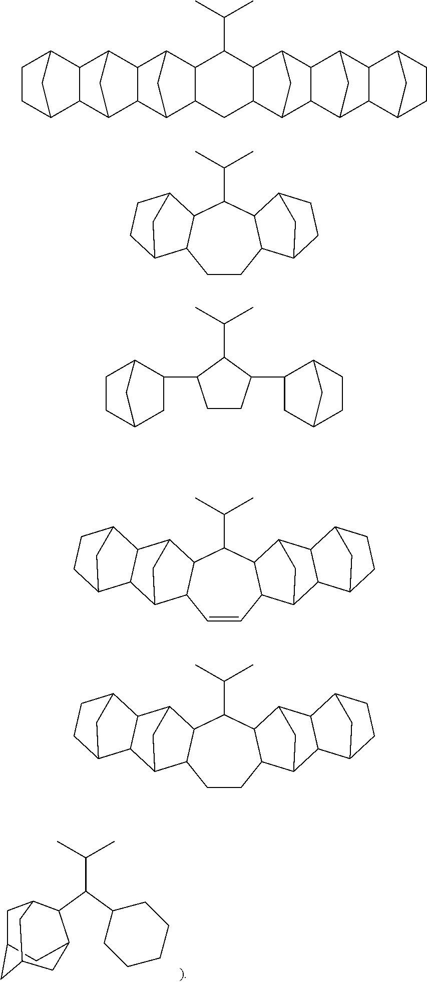

Examples of the polymer (G) include novolac resins disclosed in Japanese Patent No. 6196190 that are obtained from a compound with a bisnaphthol group represented by general formula (1) below:

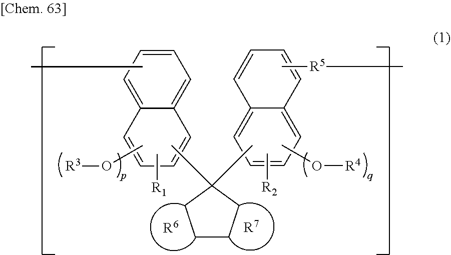

(wherein R₁ and R₂ are each independently a hydrogen atom, a C1-C10 linear, branched, or cyclic alkyl group, a C6-C20 aryl group, or a C2-C20 alkenyl group; R³ and R⁴ are each independently a hydrogen atom or a glycidyl group; R⁵ is a C1-C10 linear or branched alkylene group; R⁶ and R⁷ are each independently a benzene ring or a naphthalene ring, and the benzene ring or the naphthalene ring may be substituted with a C1-C6 hydrocarbon group in place of a hydrogen atom; and p and q are each independently 1 or 2.)

Examples of the polymer (G) include reaction products disclosed in Japanese Patent Application No. 2020-106318 that are obtained by reaction of a C6-C120 aromatic compound (A) with a compound represented by formula (1) below:

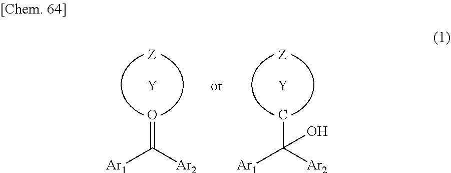

[In formula (1), Z denotes —(C═O)— or —C(—OH)—; Ar₁ and Ar₂ each independently denote an optionally substituted phenyl, naphthyl, anthracenyl, or pyrenyl group; and the ring Y denotes an optionally substituted cyclic aliphatic ring, an optionally substituted aromatic, or an optionally substituted cyclic aliphatic-aromatic condensed ring.]

Examples of the polymer (G) include polymers disclosed in Japanese Patent No. 6191831. The polymers have one, or two or more of repeating structural units represented by formula (Ia), formula (Ib), and formula (Ic) below:

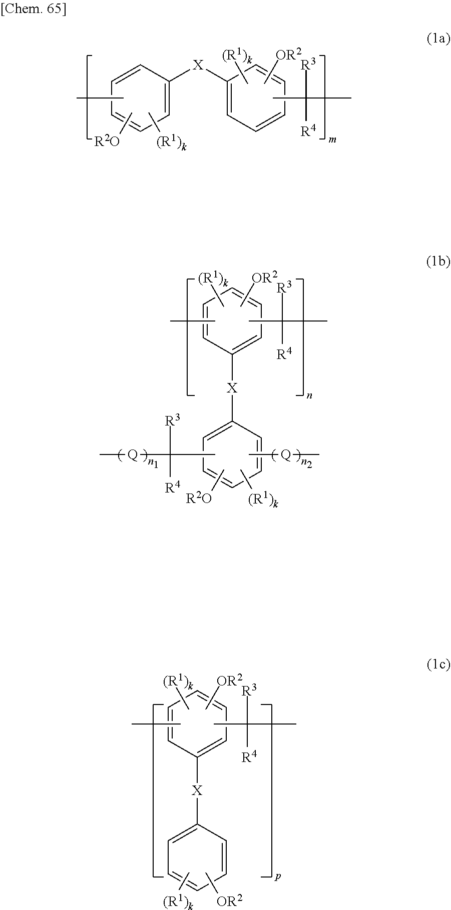

[in the formulas, R¹ independently at each of the two occurrences denotes a C1-C10 alkyl group, a C2-C6 alkenyl group, an aromatic hydrocarbon group, a halogen atom, a nitro group, or an amino group; R² independently at each of the two occurrences denotes a hydrogen atom, a C1-C10 alkyl group, a C2-C6 alkenyl group, an acetal group, an acyl group, or a glycidyl group; R³ denotes an optionally substituted aromatic hydrocarbon group; R⁴ denotes a hydrogen atom, a phenyl group, or a naphthyl group; when R³ and R⁴ bonded to the same carbon atom each denote a phenyl group, they may be bonded to each other to form a fluorene ring; in formula (Ib), the groups represented by two R³'s may be different from each other, and the atoms or the groups represented by two R⁴'s may be different from each other; k independently at each of the two occurrences denotes 0 or 1; m denotes an integer of 3 to 500; n, n₁, and n₂ each denote an integer of 2 to 500; p denotes an integer of 3 to 500; X denotes a single bond or a heteroatom; and Q independently at each of the two occurrences denotes a structural unit represented by formula (2) below:

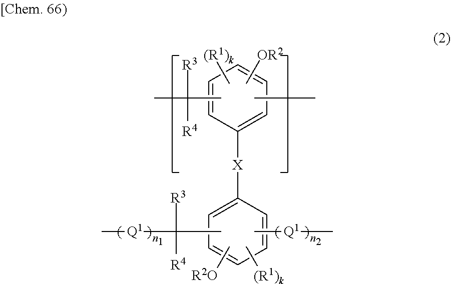

(wherein R at two occurrences, R² at two occurrences, R³ at two occurrences, R⁴ at two occurrences, k at two occurrences, n¹, n₂, and X are the same as defined in formula (Ib), and Q¹ independently at each of the two occurrences denotes a structural unit represented by formula (2))].

Examples of the polymer (G) include polymers disclosed in WO 2017/199768. The polymers have a repeating structural unit represented by formula (Ia) and/or formula (Ib) below:

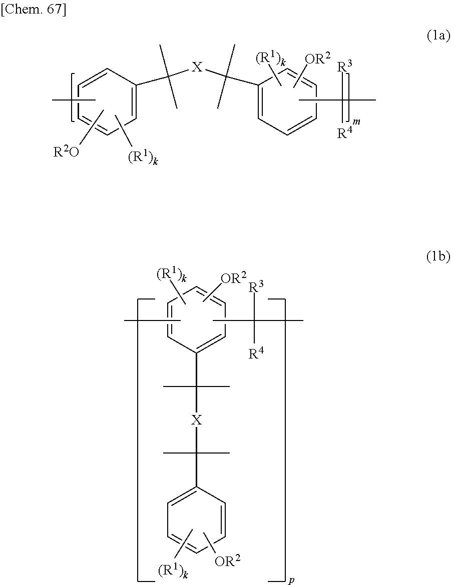

[In formulas (1a) and (1b), R¹ independently at each of the two occurrences denotes a C1-C10 alkyl group, a C2-C6 alkenyl group, an aromatic hydrocarbon group, a halogen atom, a nitro group, or an amino group; R² independently at each of the two occurrences denotes a hydrogen atom, a C1-C10 alkyl group, a C2-C6 alkenyl group, an acetal group, an acyl group, or a glycidyl group; R³ denotes an optionally substituted, aromatic hydrocarbon or heterocyclic group; R⁴ denotes a hydrogen atom, a phenyl group, or a naphthyl group; when R³ and R⁴ bonded to the same carbon atom each denote a phenyl group, they may be bonded to each other to form a fluorene ring; k independently at each of the two occurrences denotes 0 or 1; m denotes an integer of 3 to 500; p denotes an integer of 3 to 500; X denotes a benzene ring; and the two —C(CH₃)₂— groups bonded to the benzene ring are in meta-positions or para-positions relative to each other.]

Some preferred specific examples of the compound (D) are illustrated below:

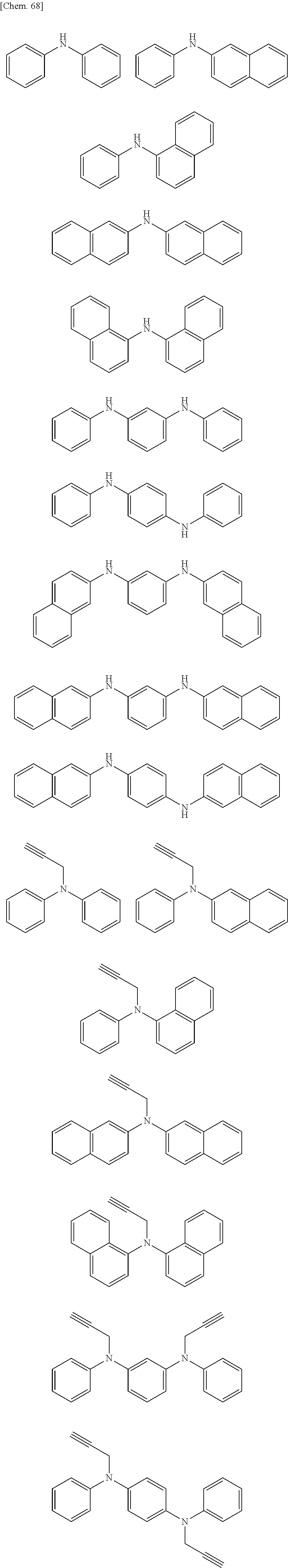

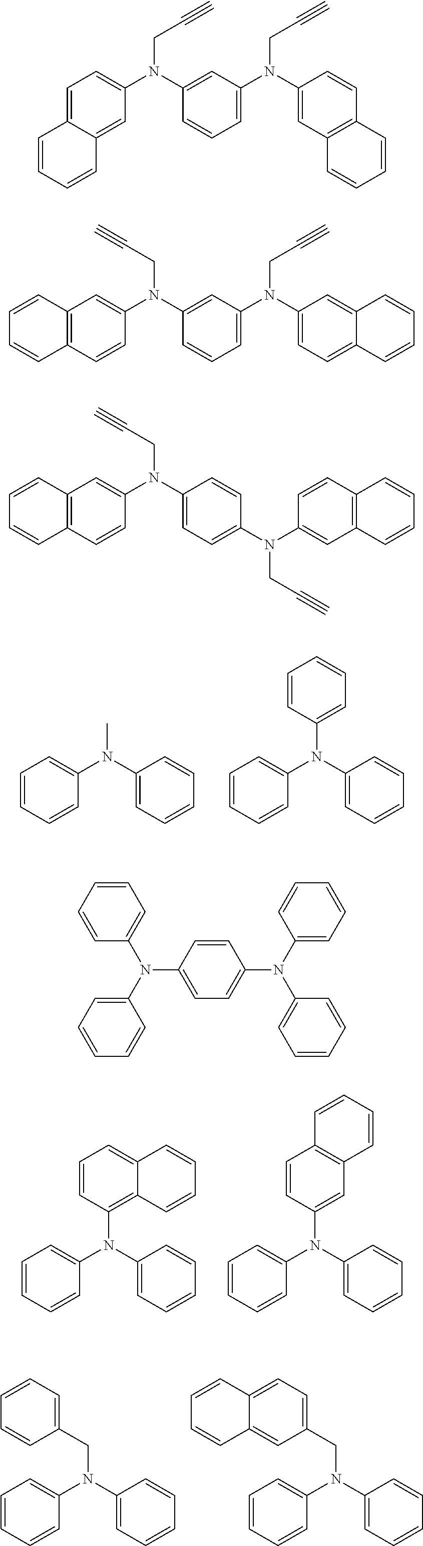

More preferred compounds are illustrated below.

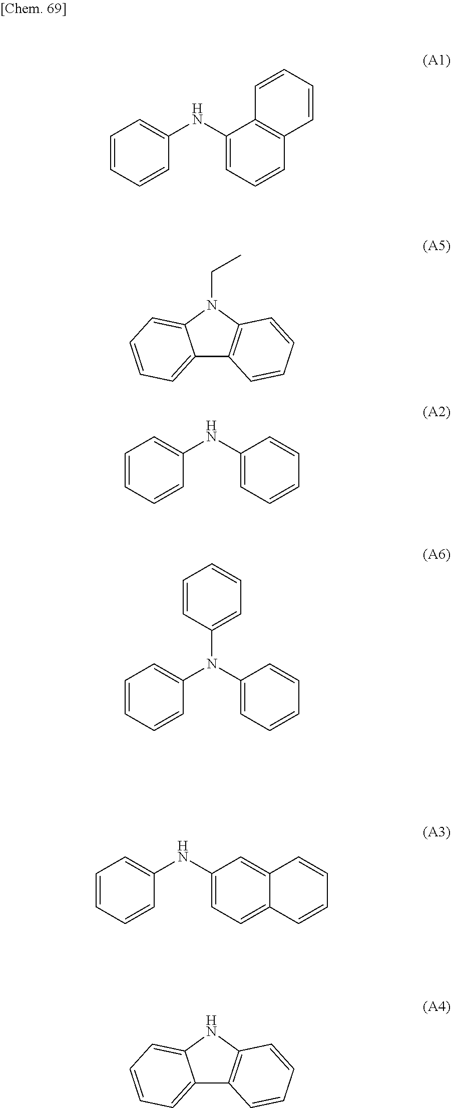 [SMILES: c1ccc(Nc2cccc3ccccc23)cc1 ; CCn1c2ccccc2c2ccccc21 ; c1ccc(Nc2ccccc2)cc1 ; c1ccc(N(c2ccccc2)c2ccccc2)cc1 ; c1ccc(Nc2ccc3ccccc3c2)cc1 ; c1ccc2c(c1)[nH]c1ccccc12]

Some preferred specific examples of the compound (D) are illustrated below.

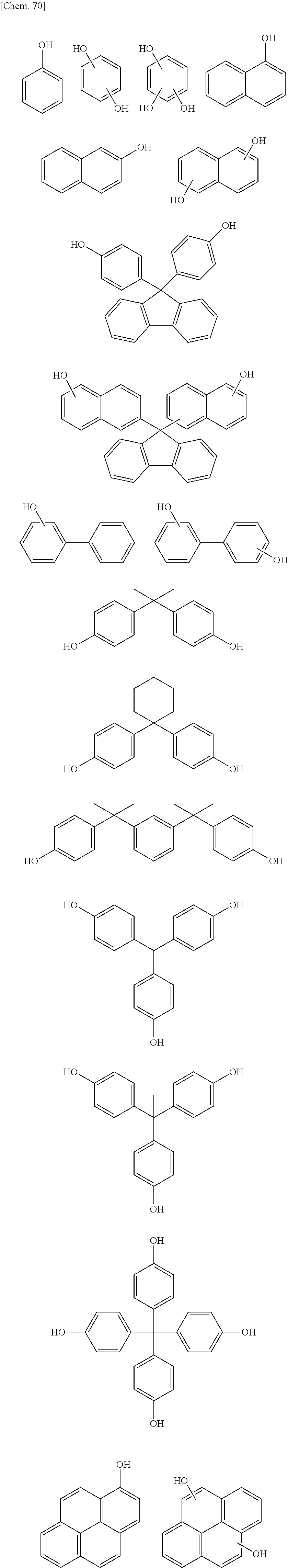

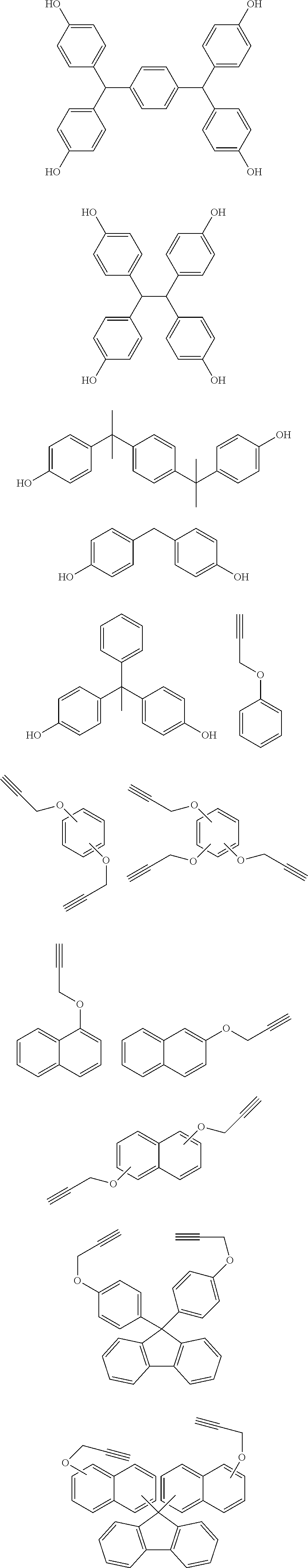

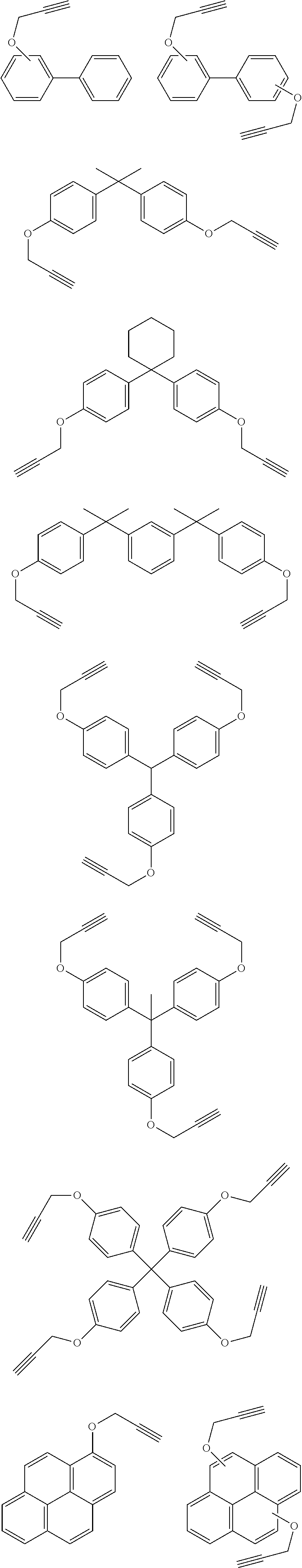

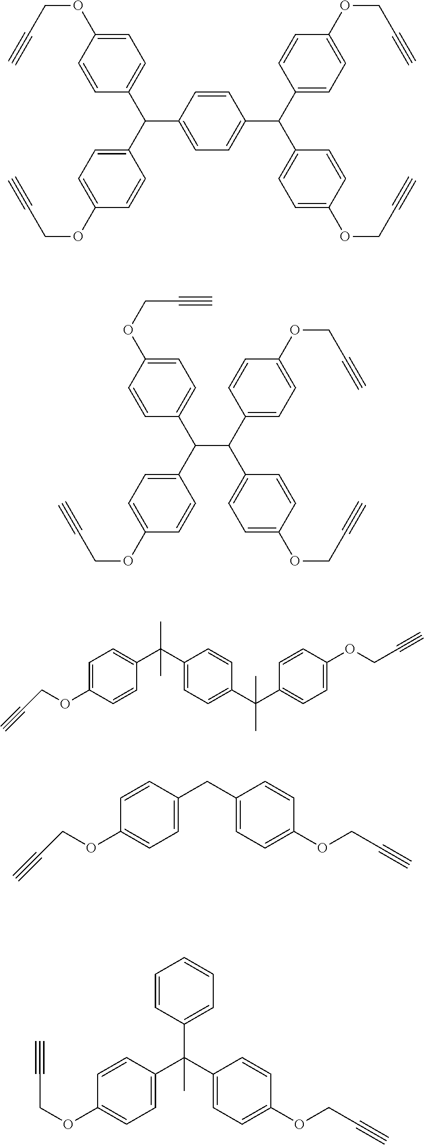

More preferred compounds are illustrated below:

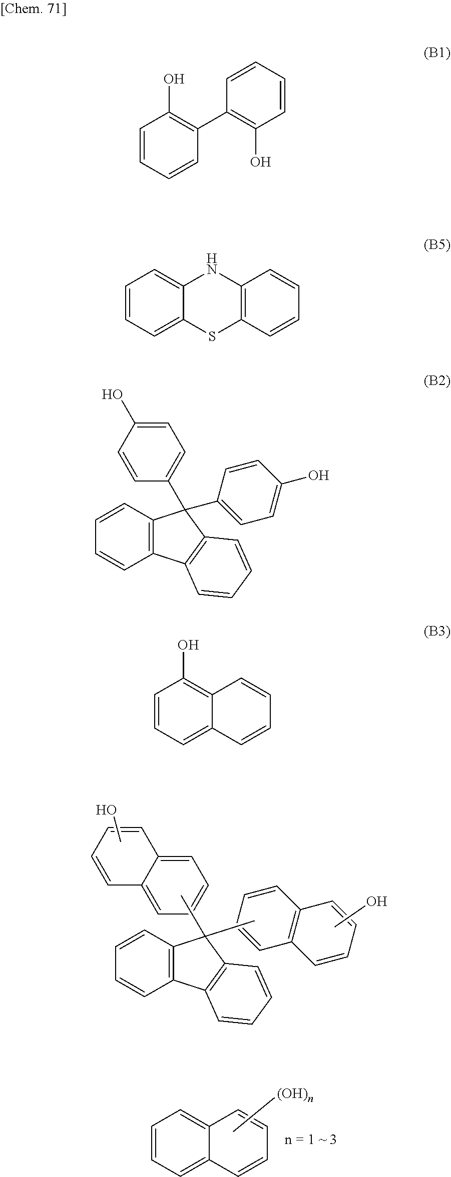

Some preferred specific examples of the aldehyde compound and aldehyde equivalent (E) are illustrated below.

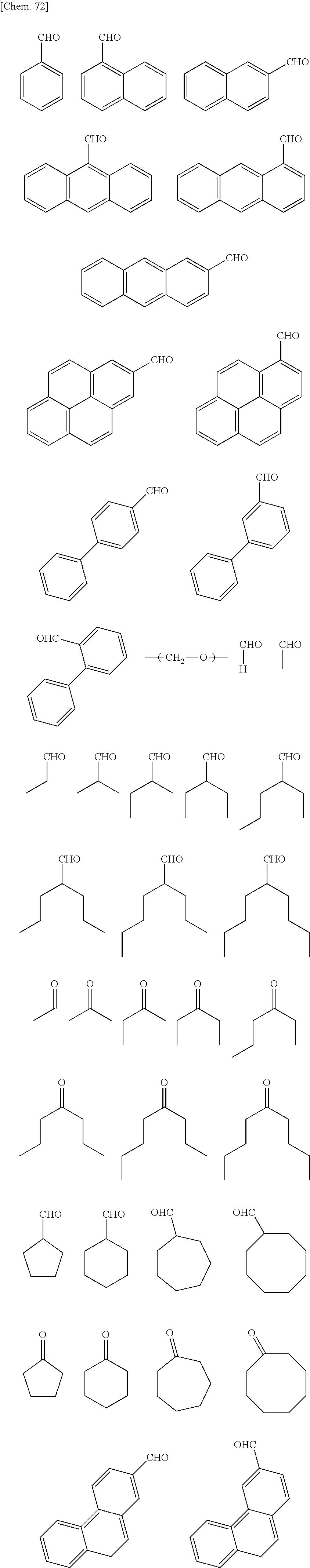

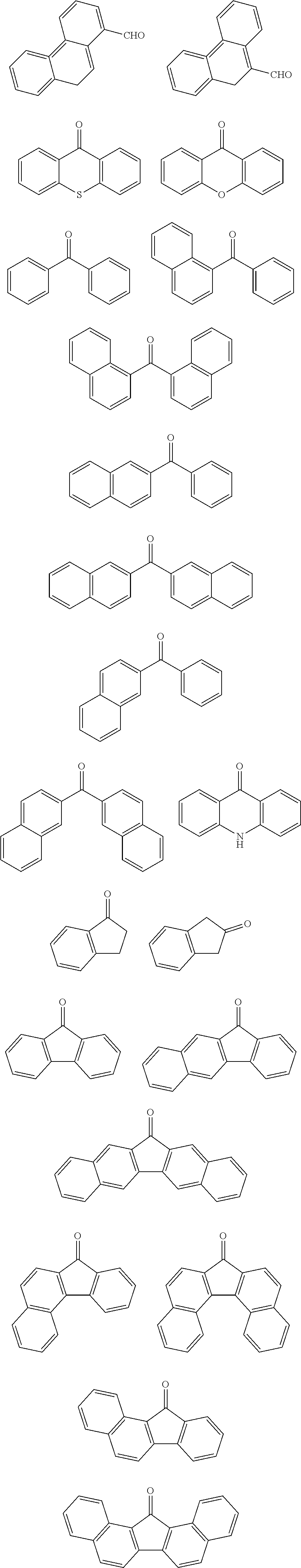

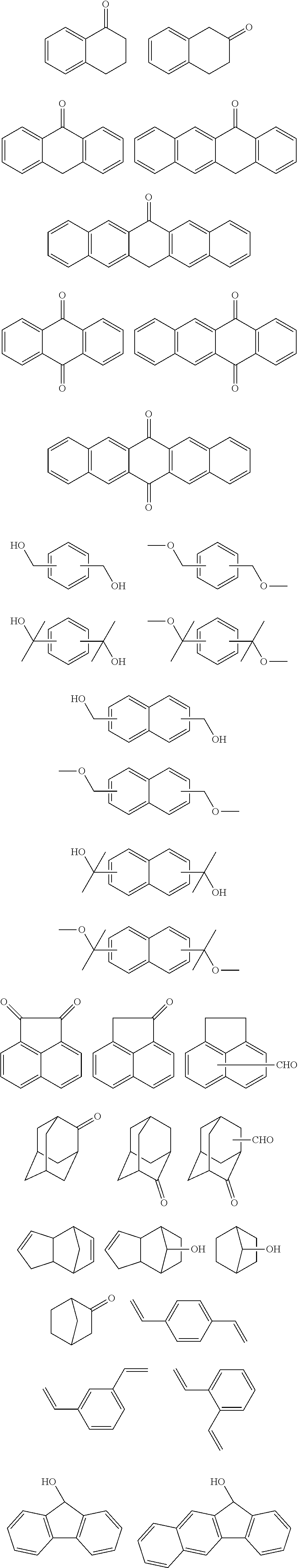

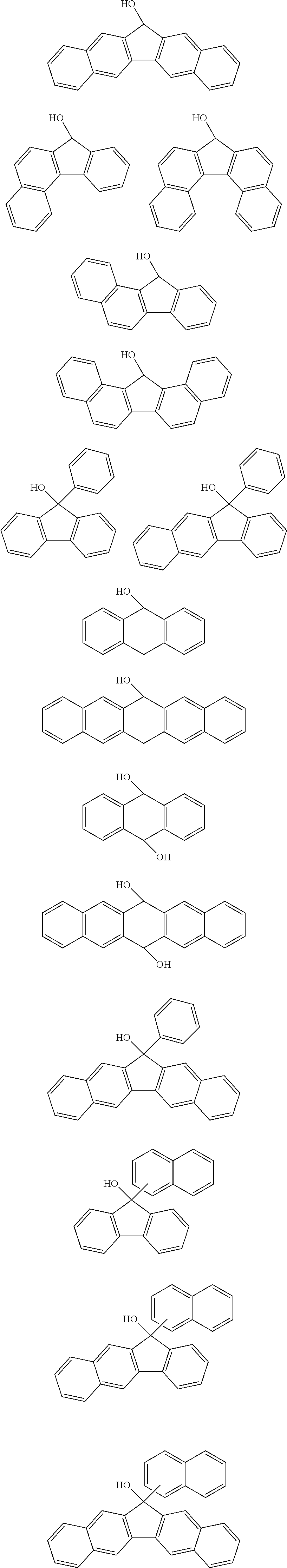

Some more preferred specific examples of the aldehyde compound and aldehyde equivalent (E) are illustrated below:

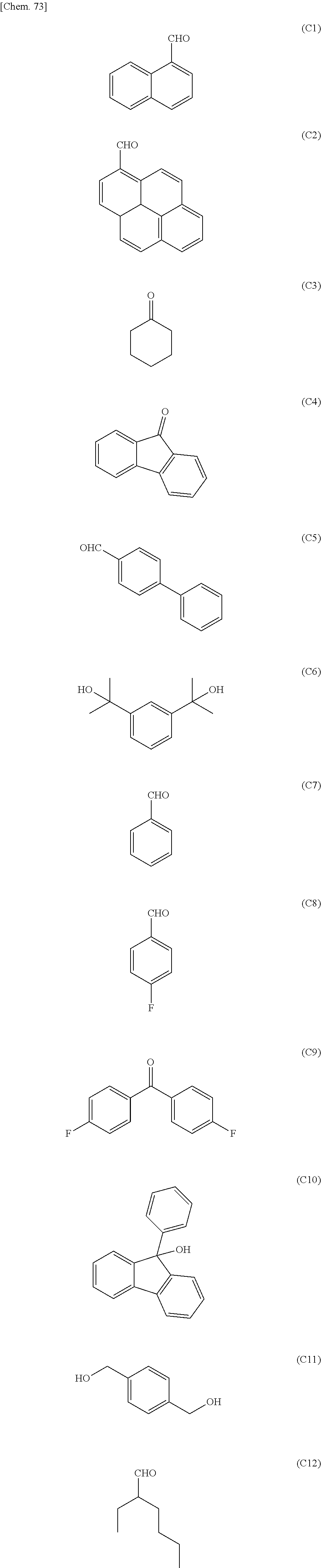

Some preferred specific Examples of the polymer (G) (repeating unit structures) are illustrated below:

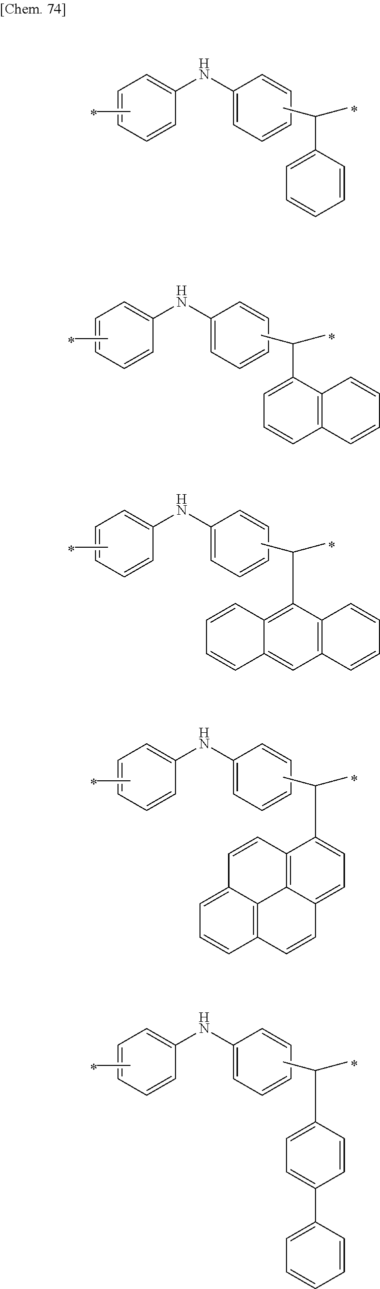

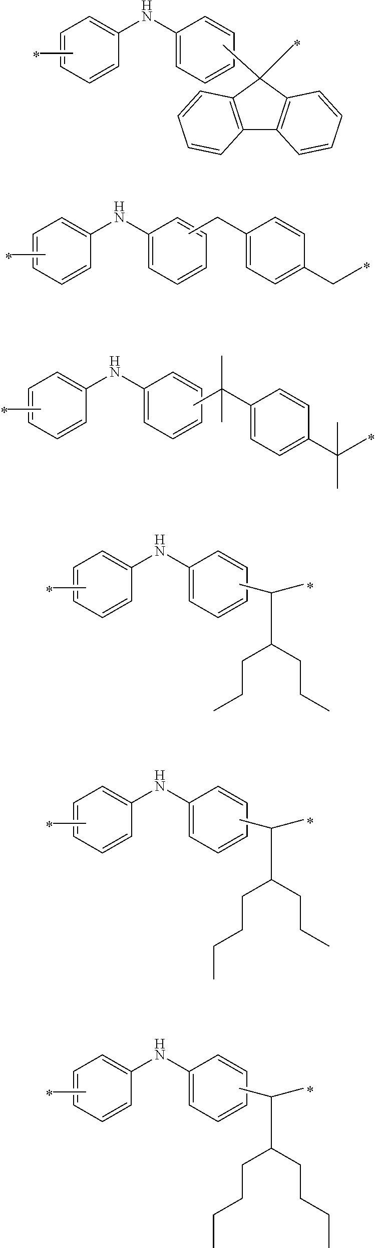

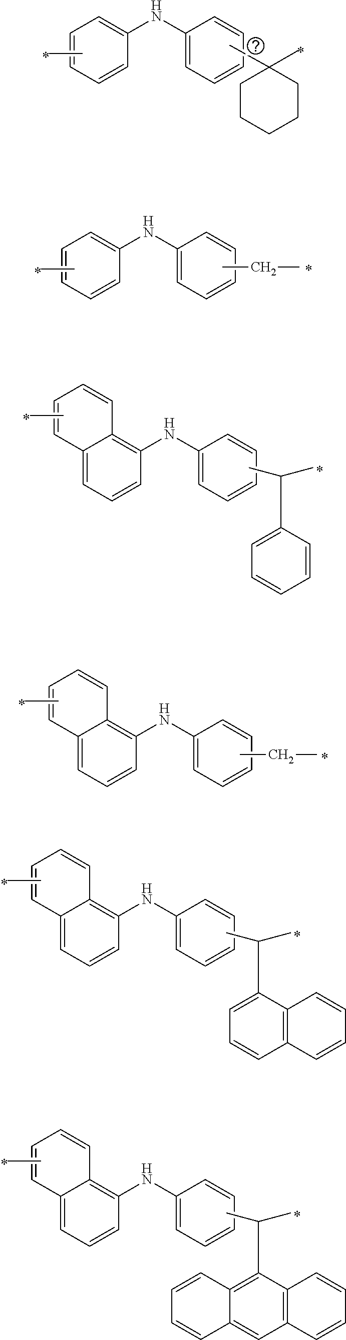

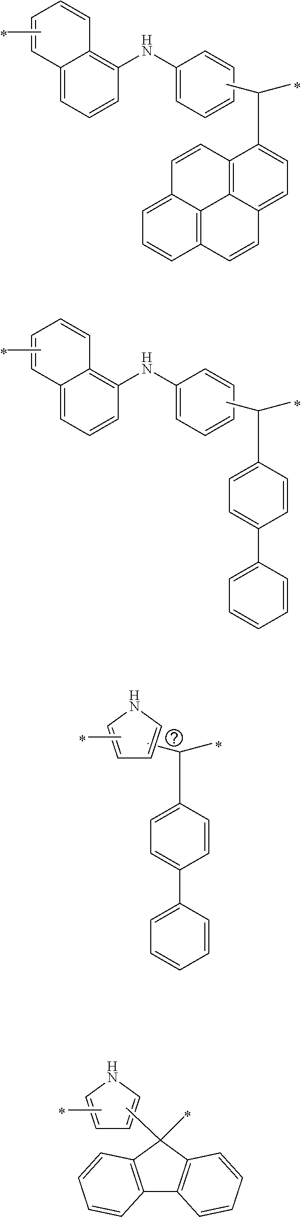

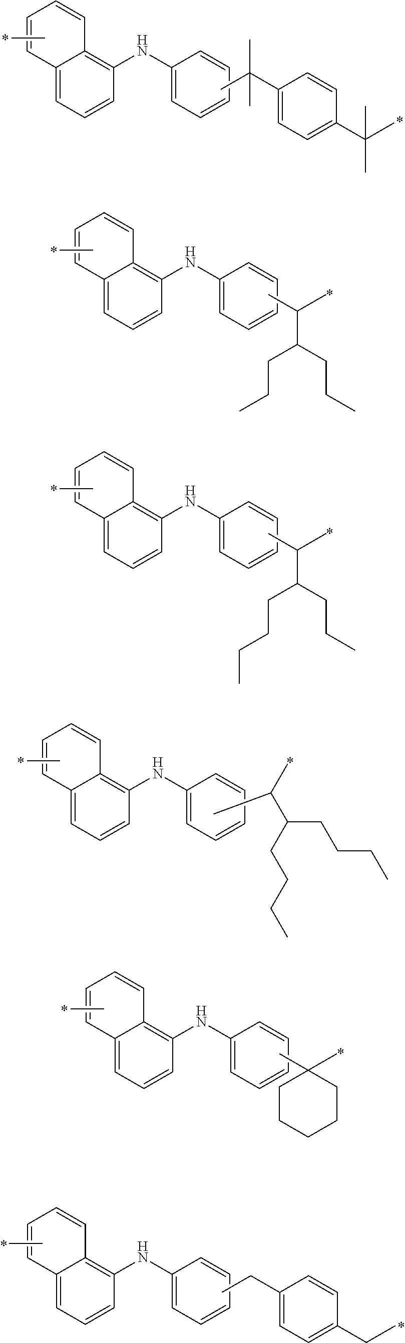

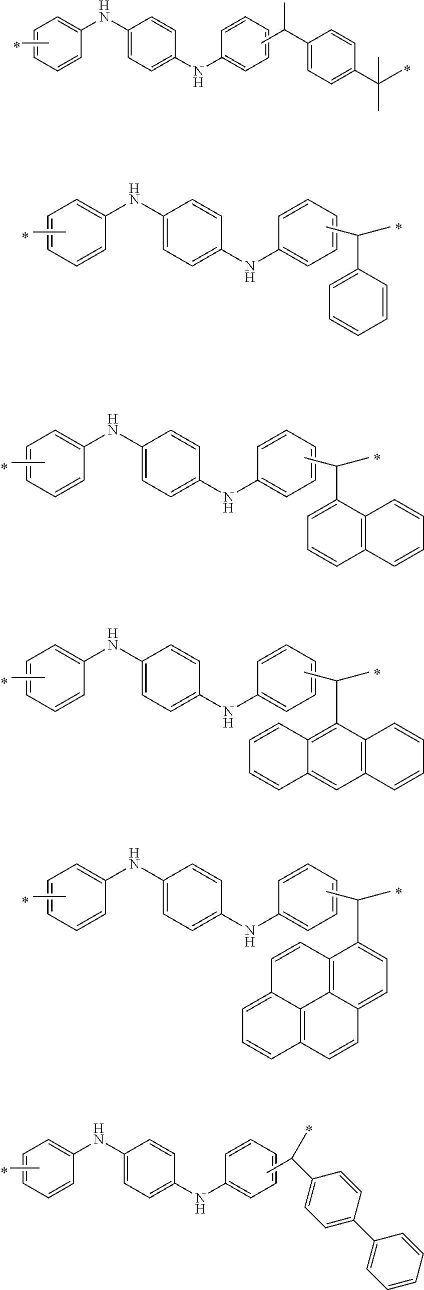

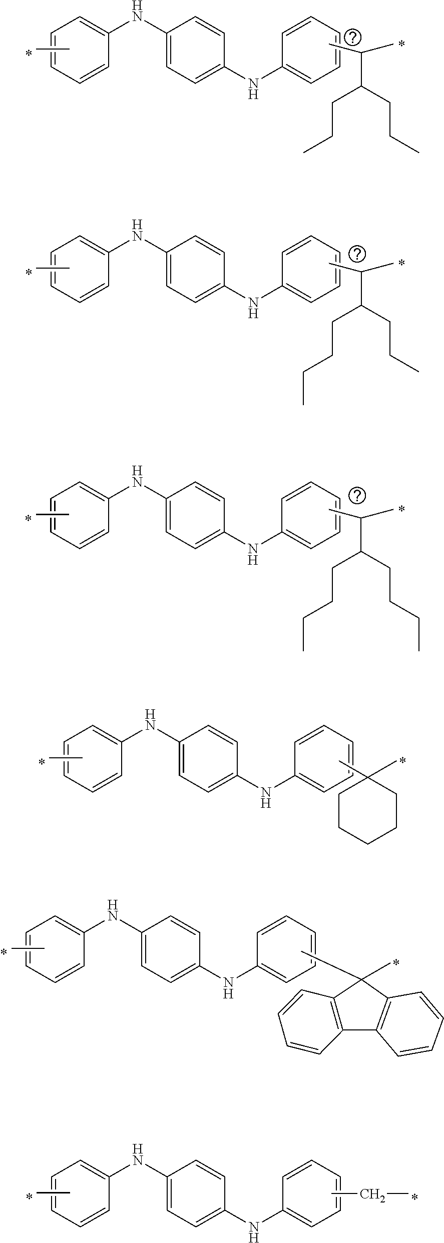

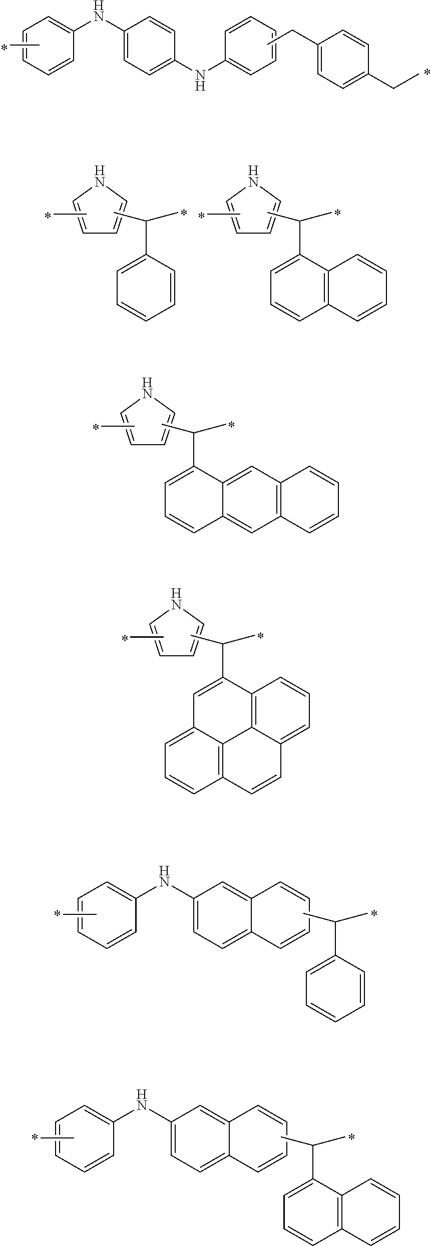

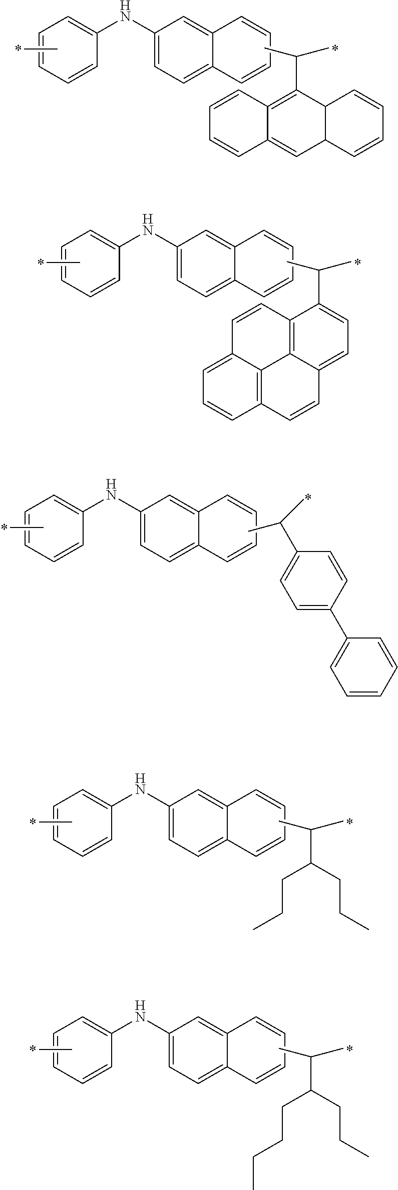

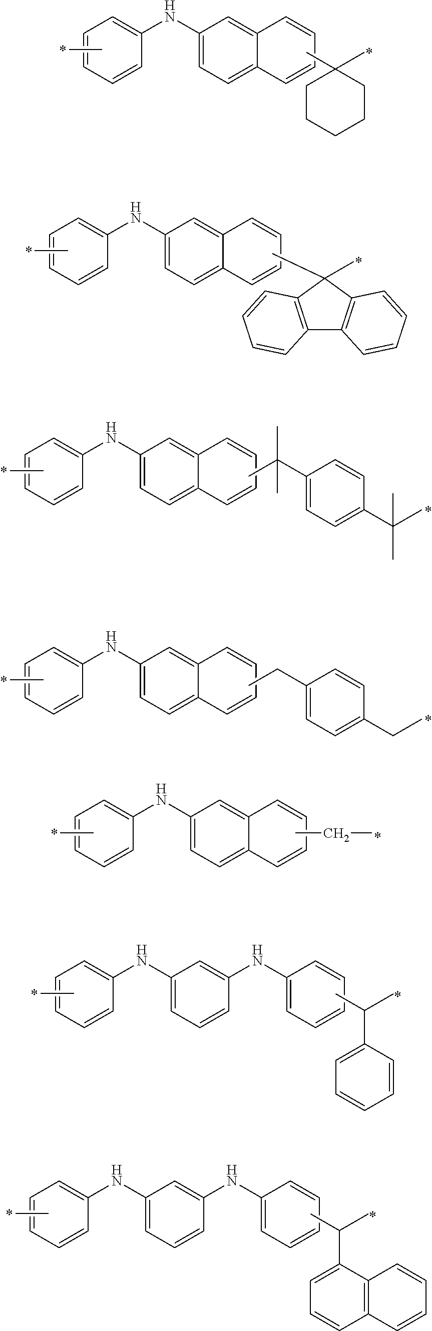

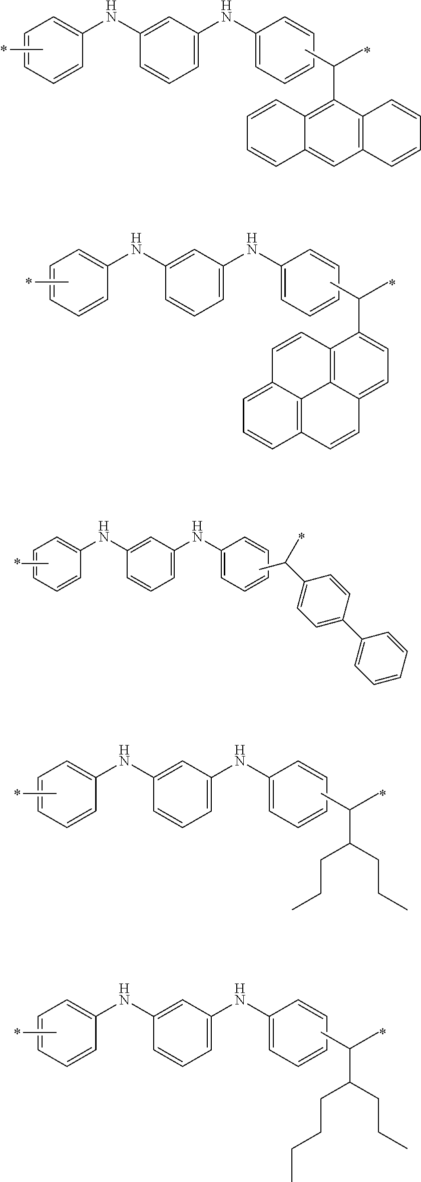

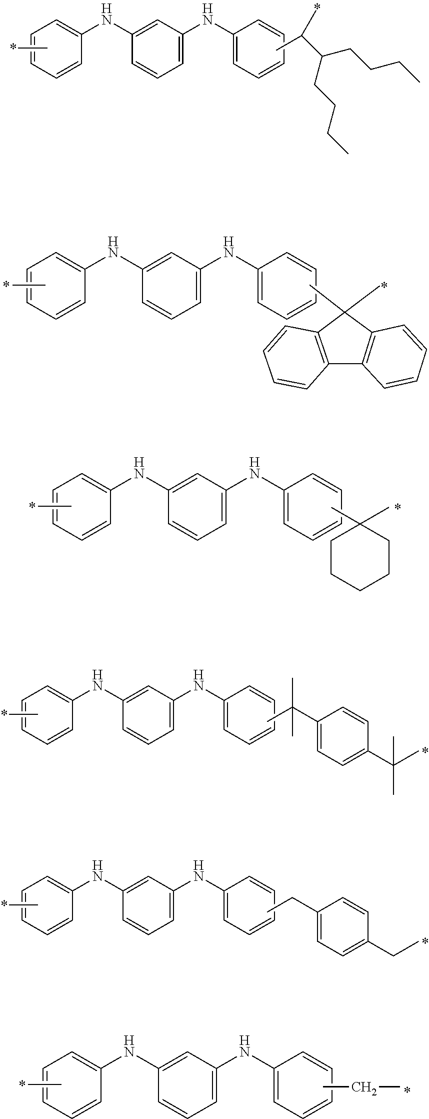

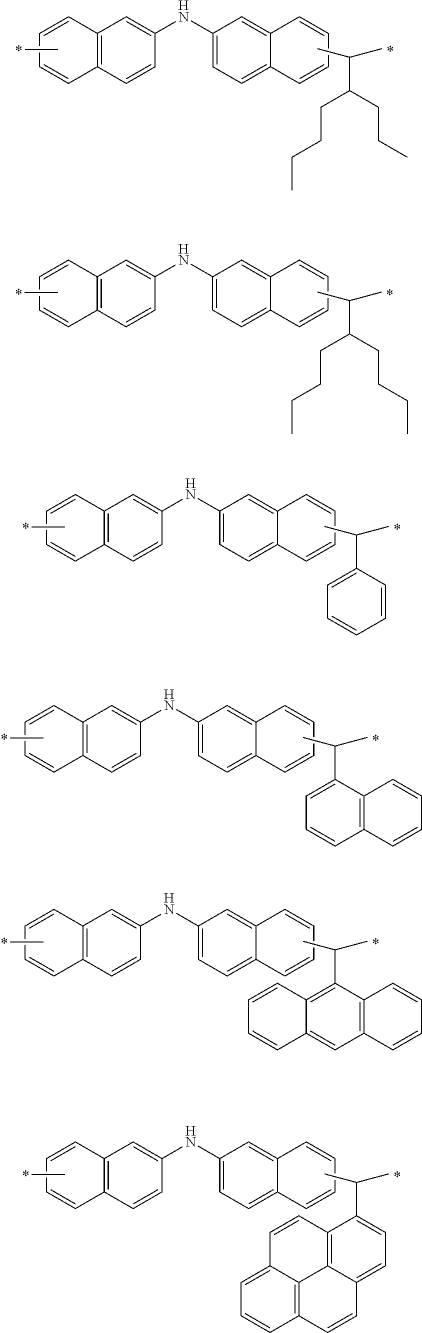

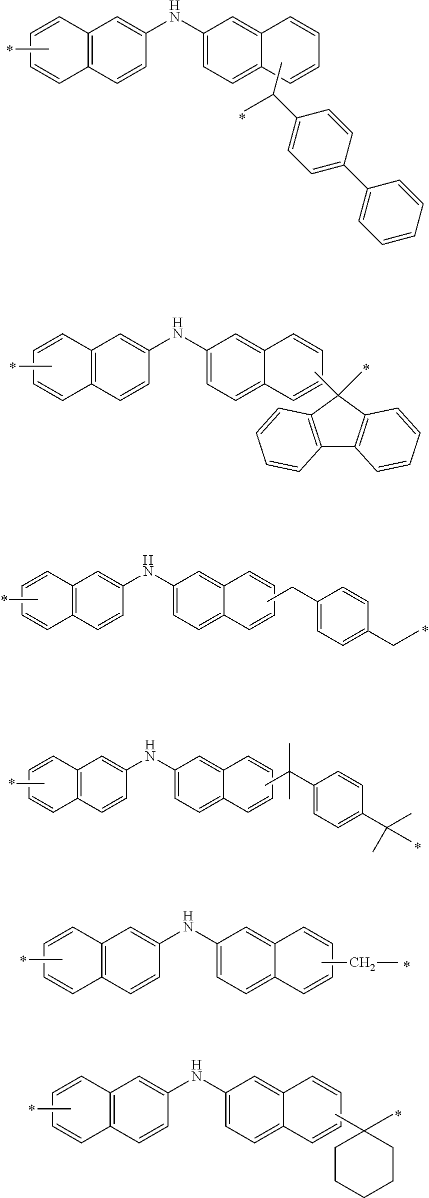

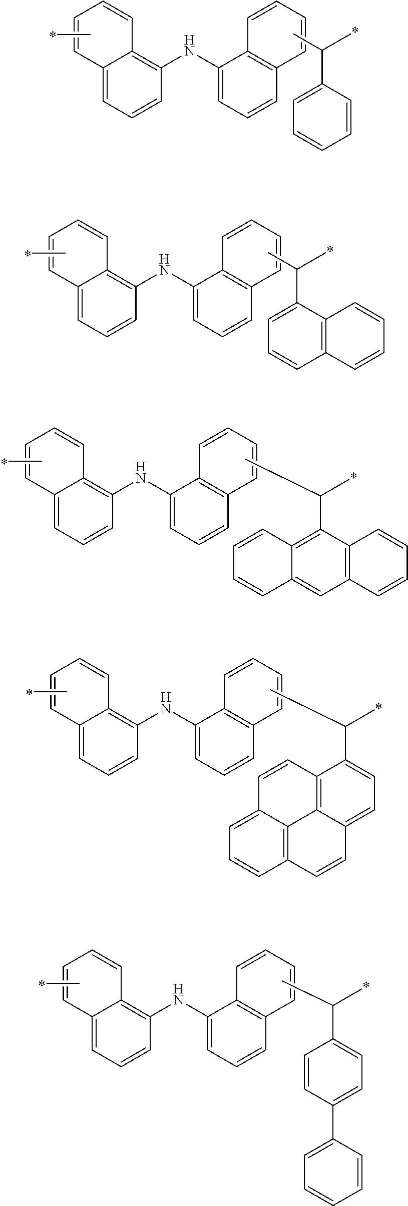

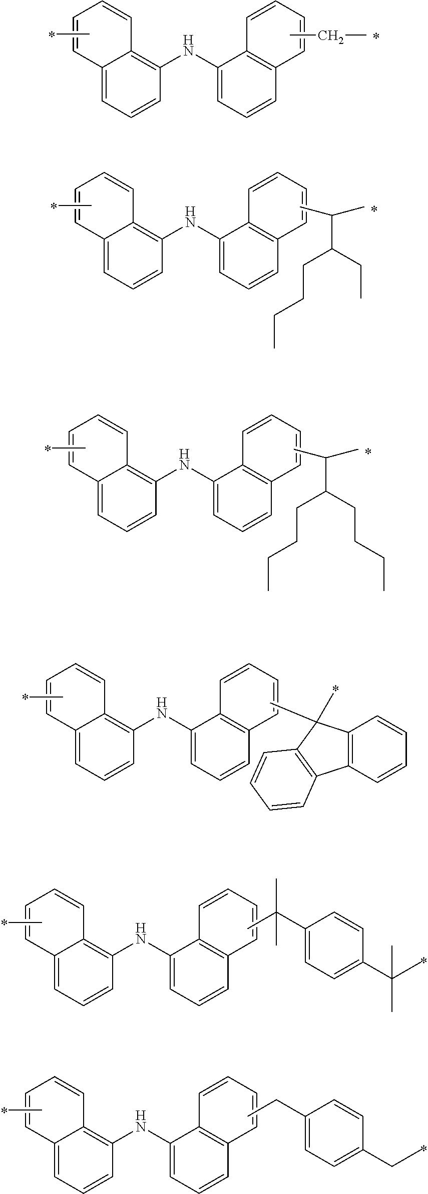

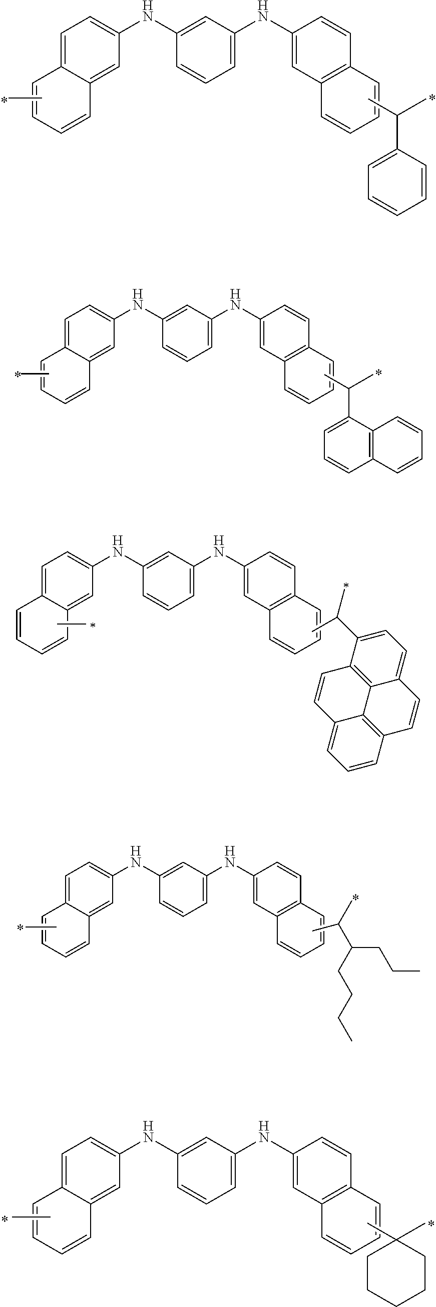

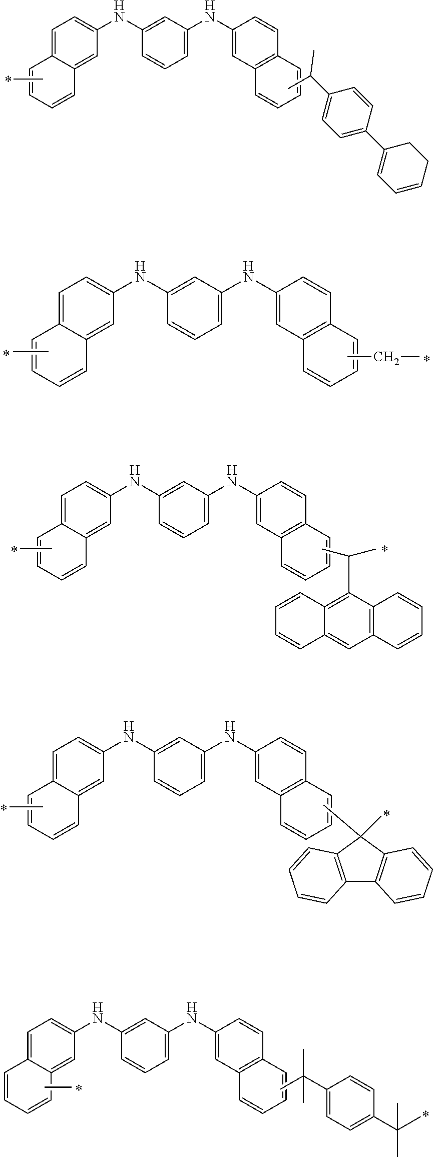

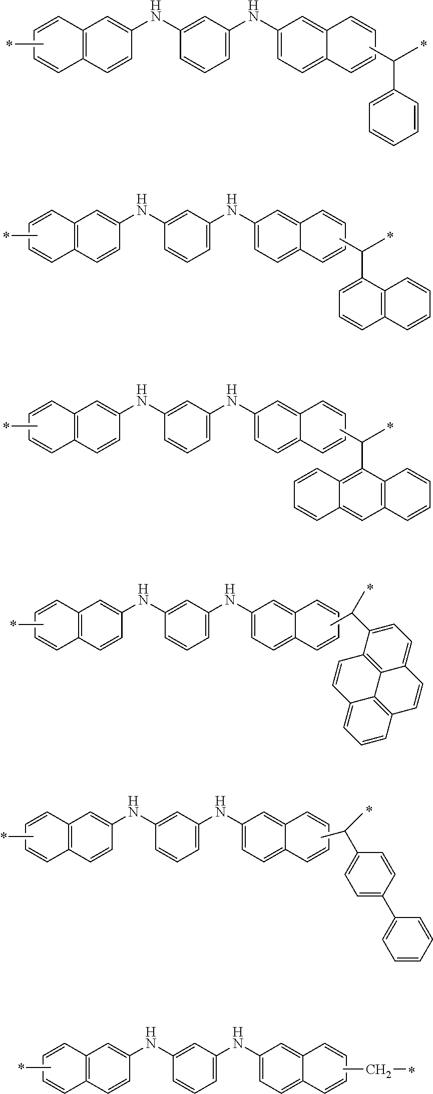

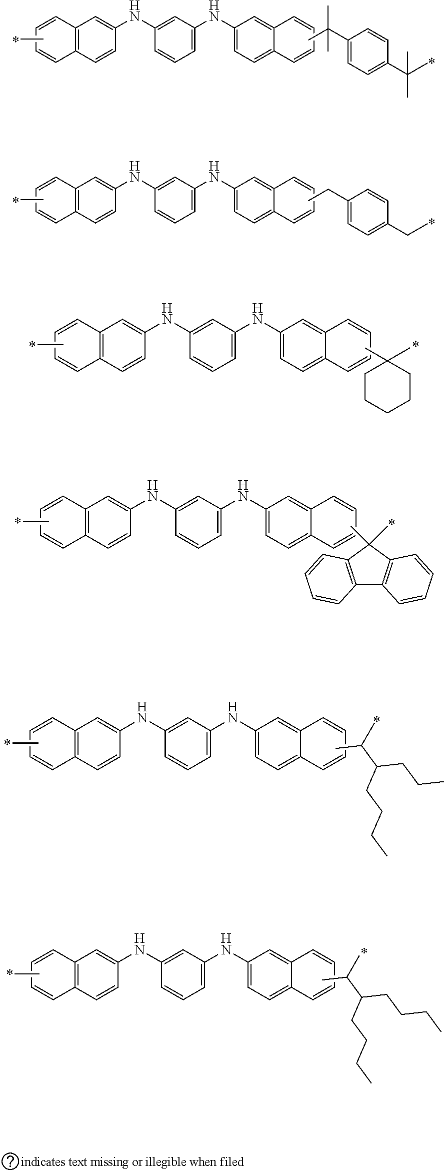

Some preferred specific Examples of the polymer (G) (repeating unit structures) are illustrated below:

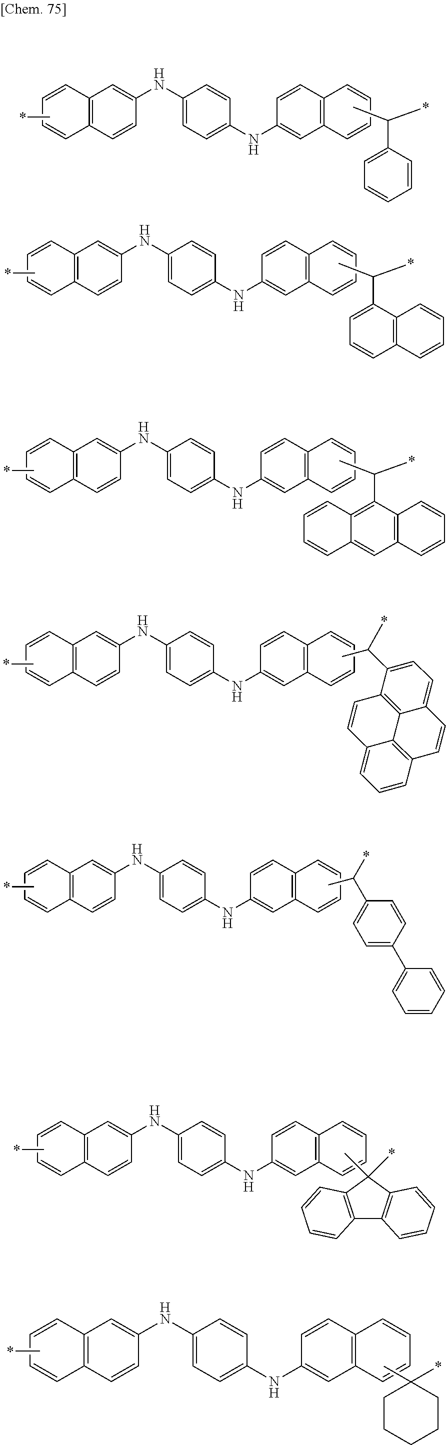

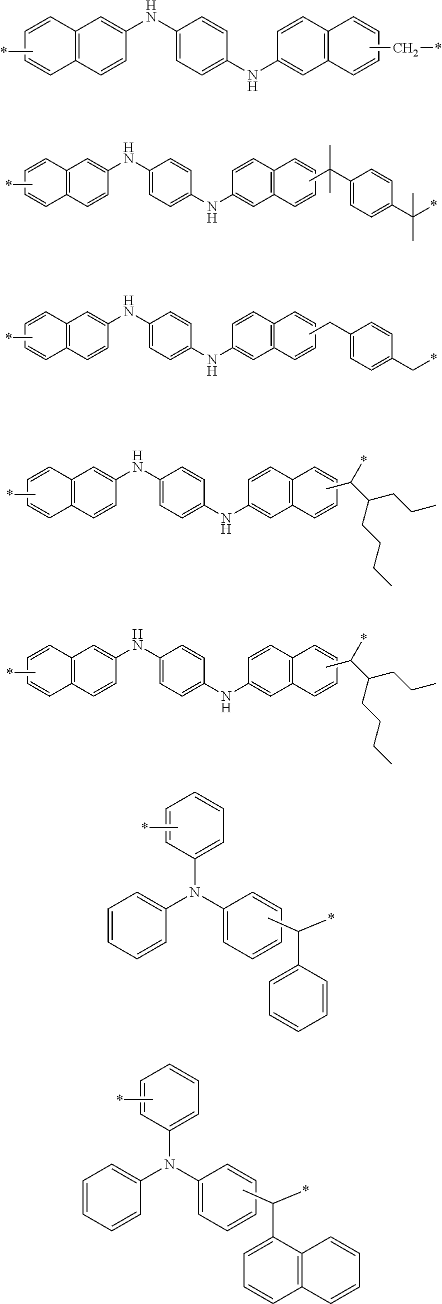

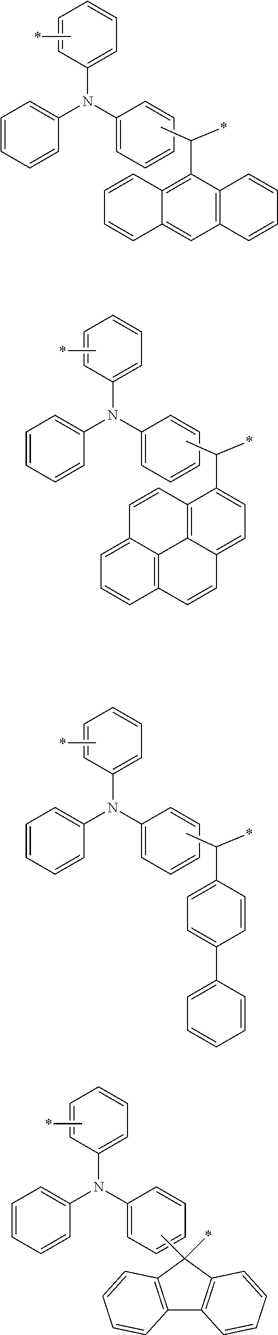

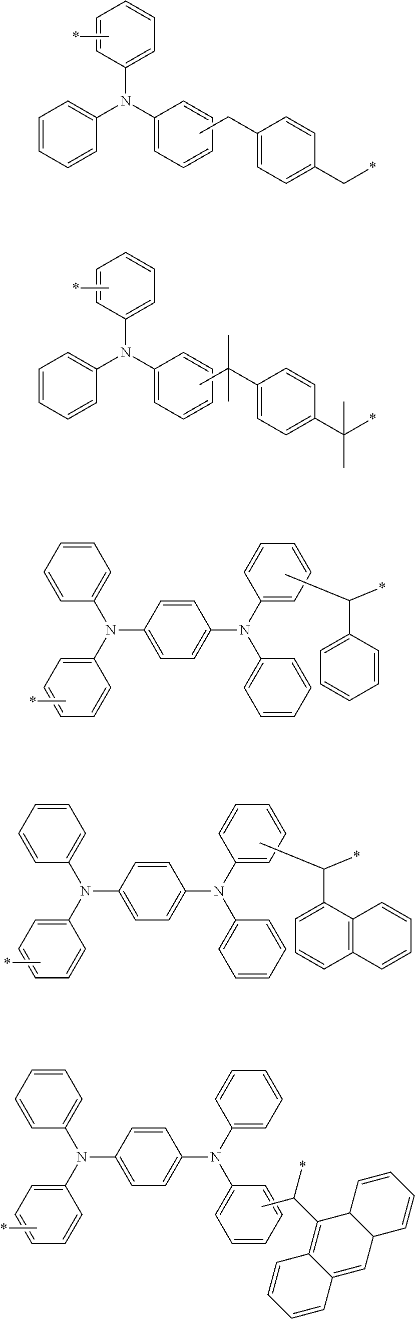

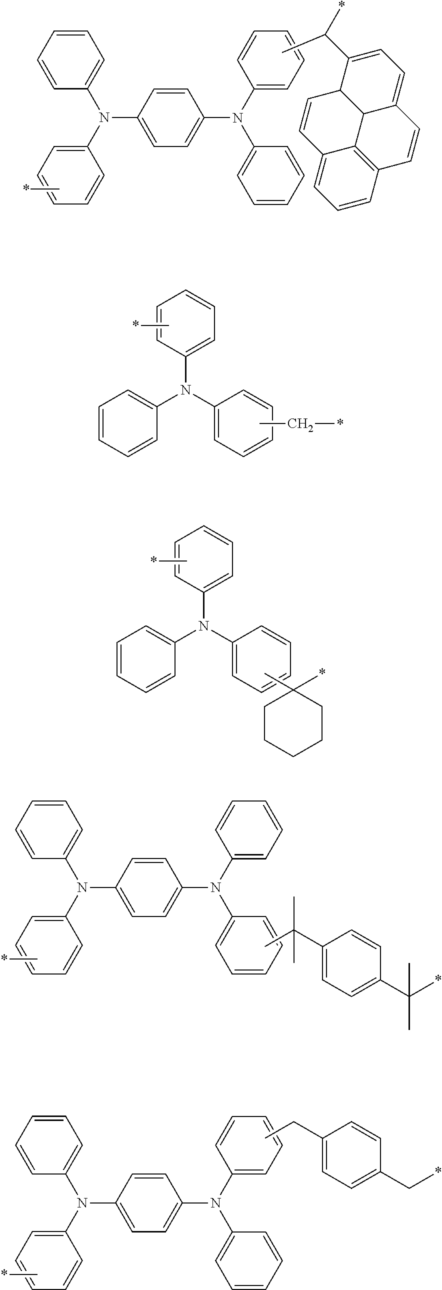

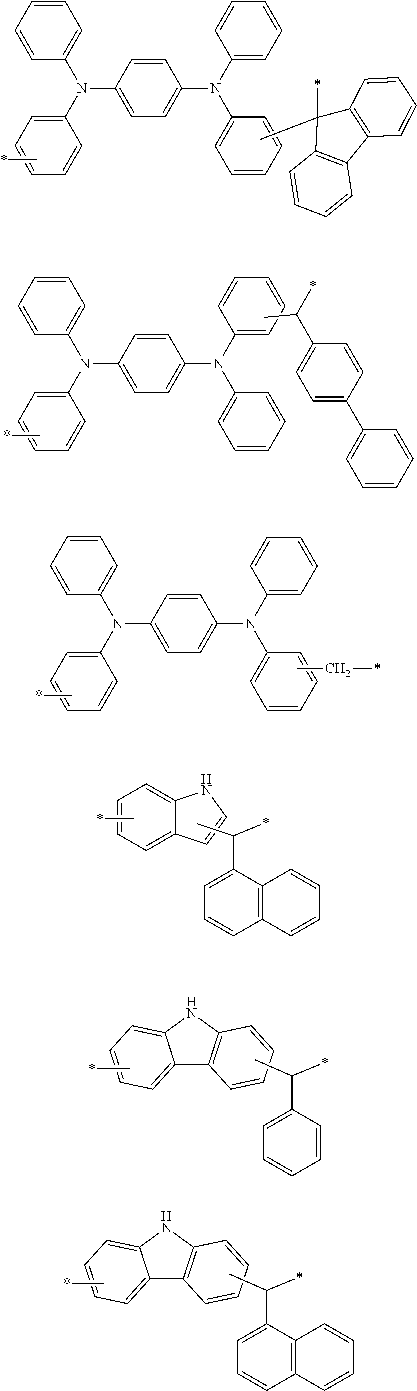

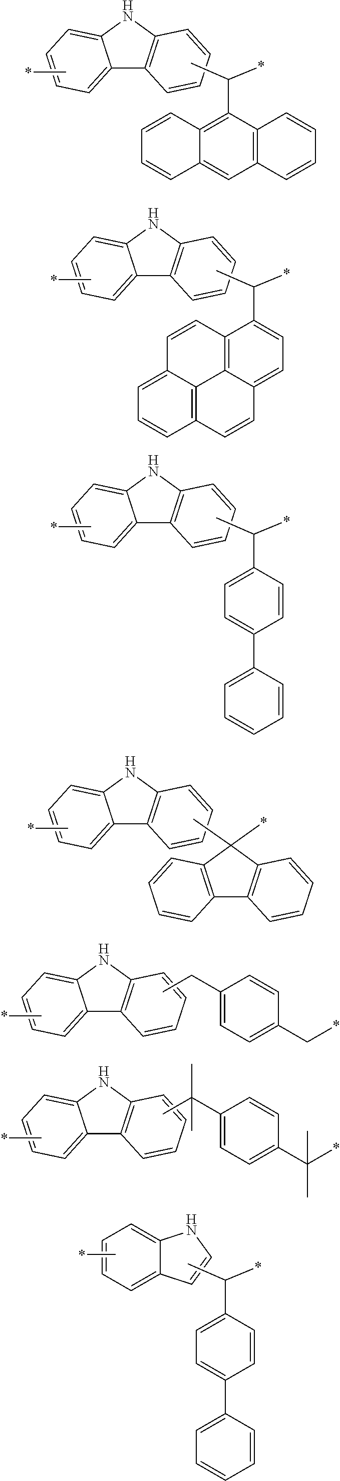

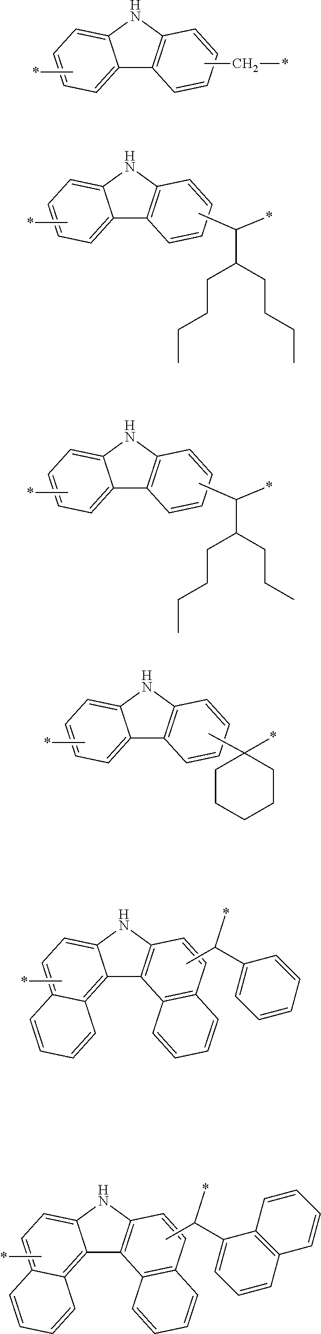

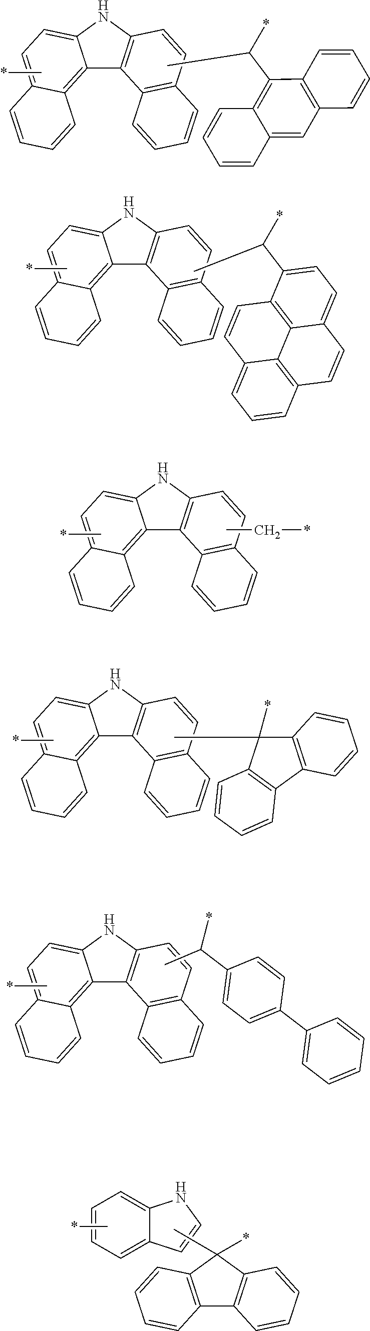

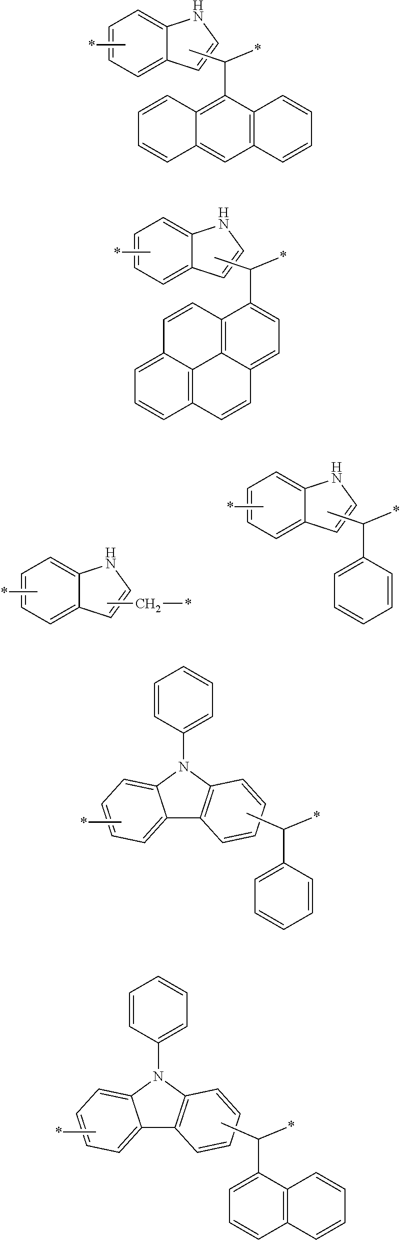

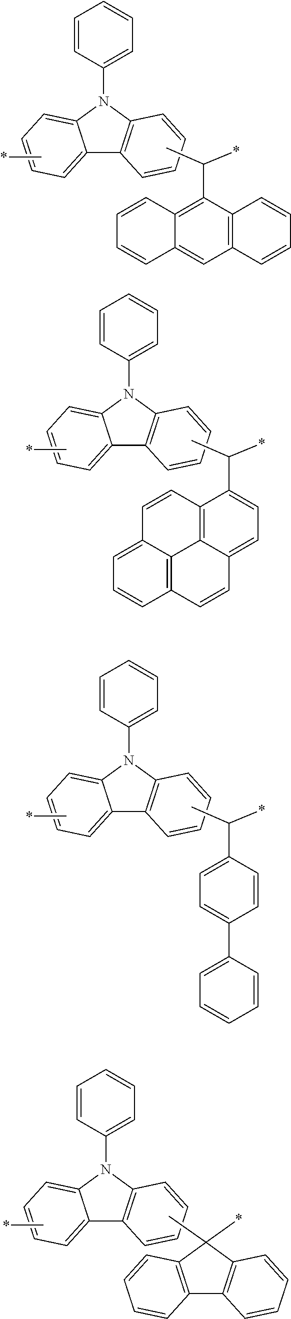

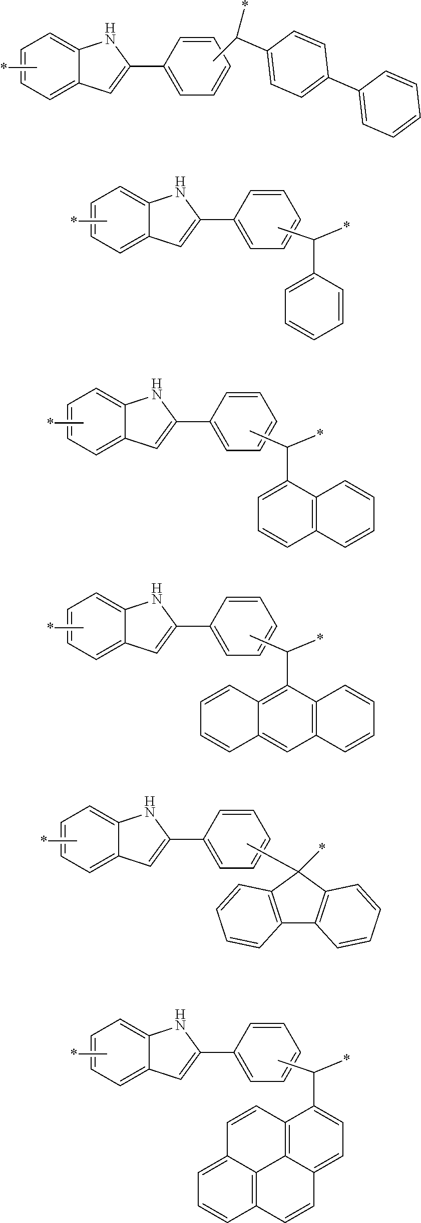

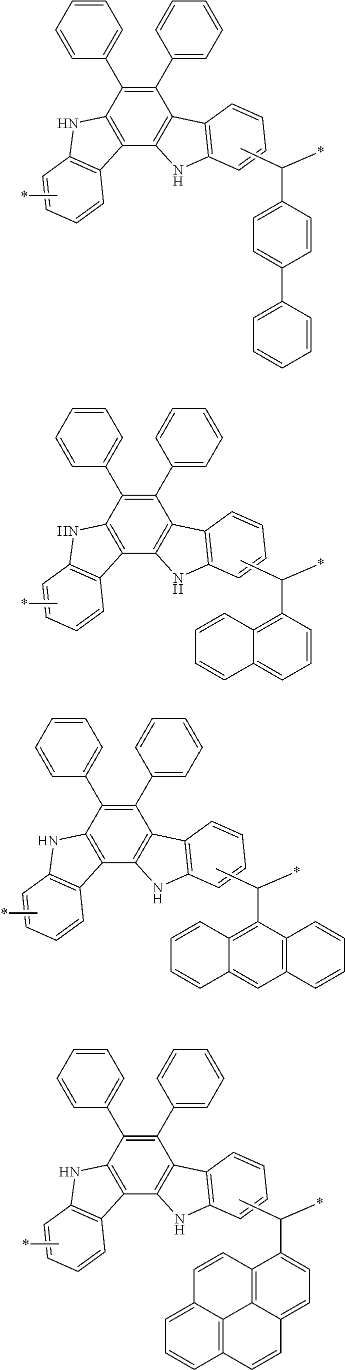

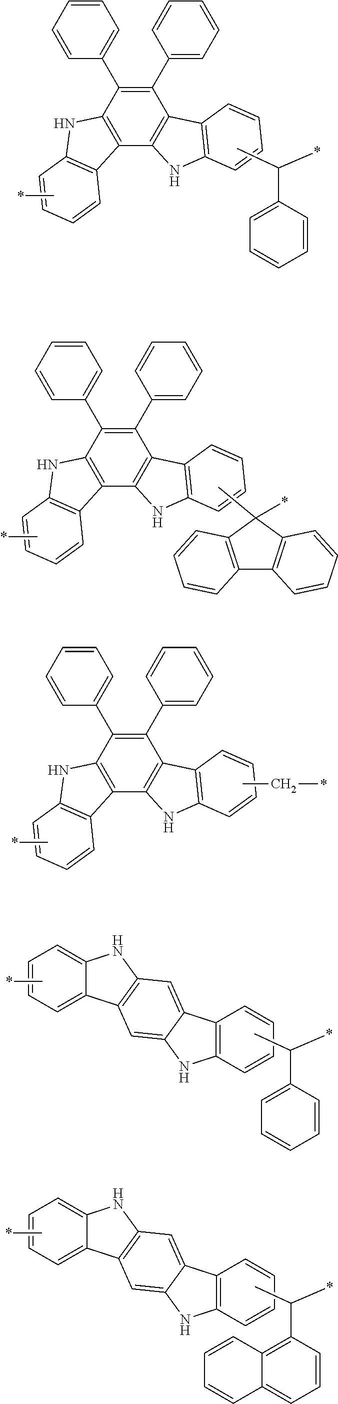

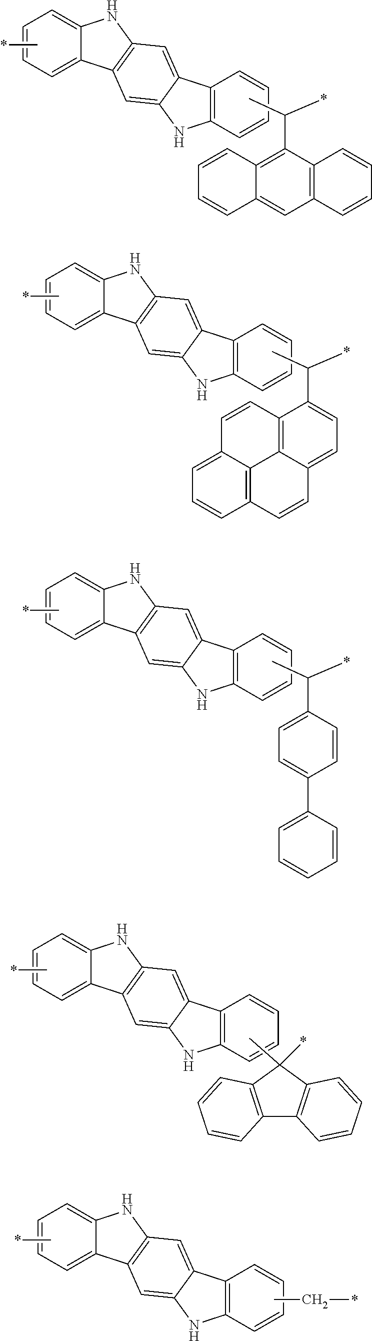

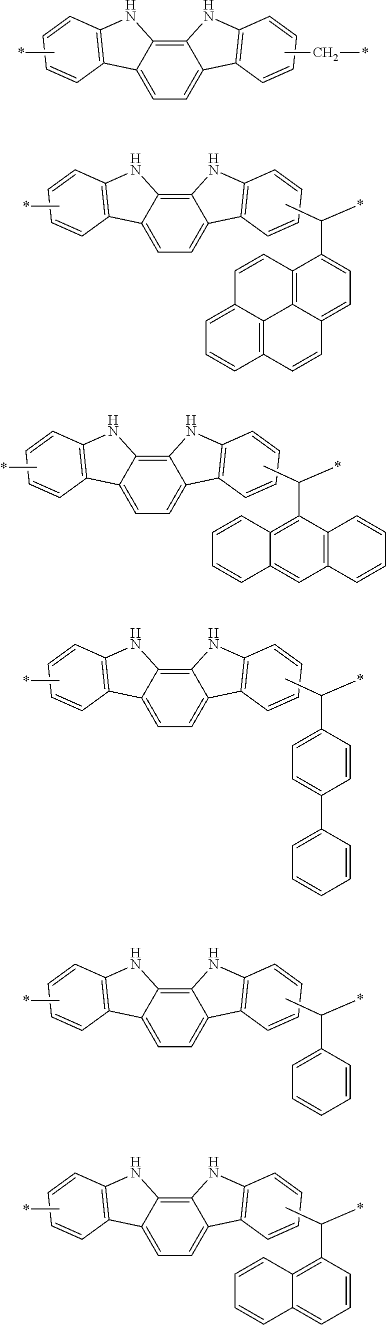

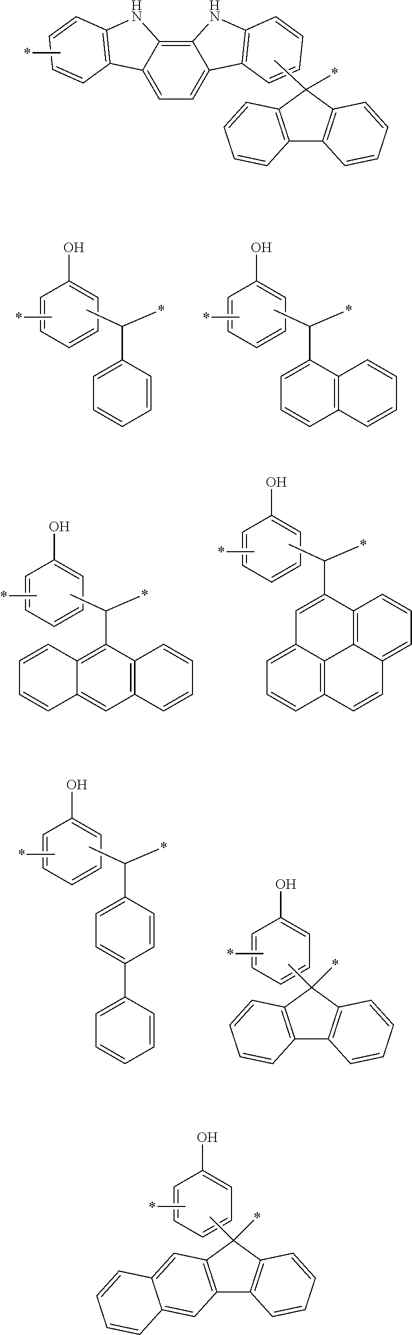

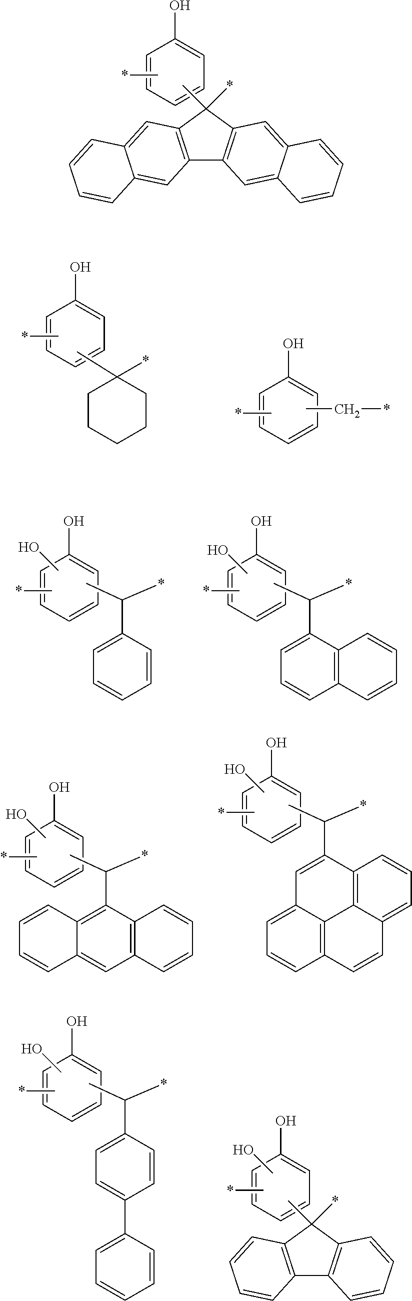

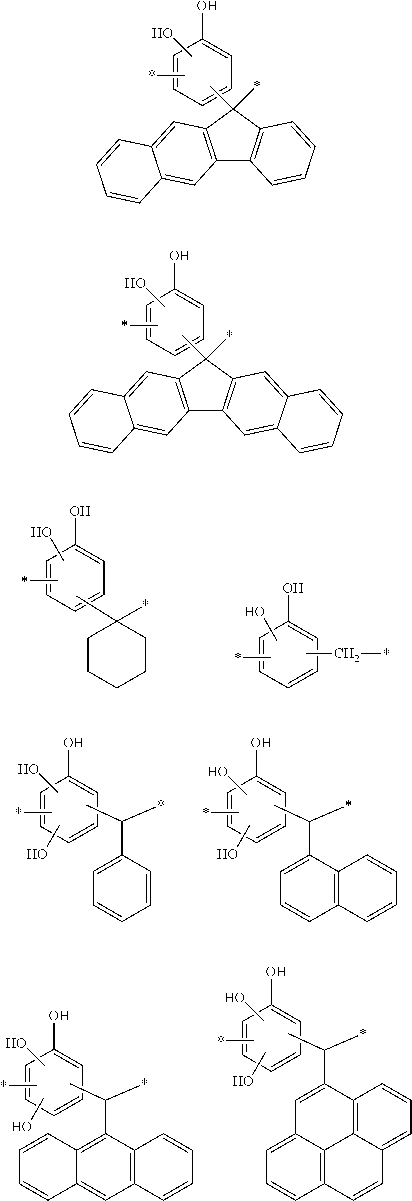

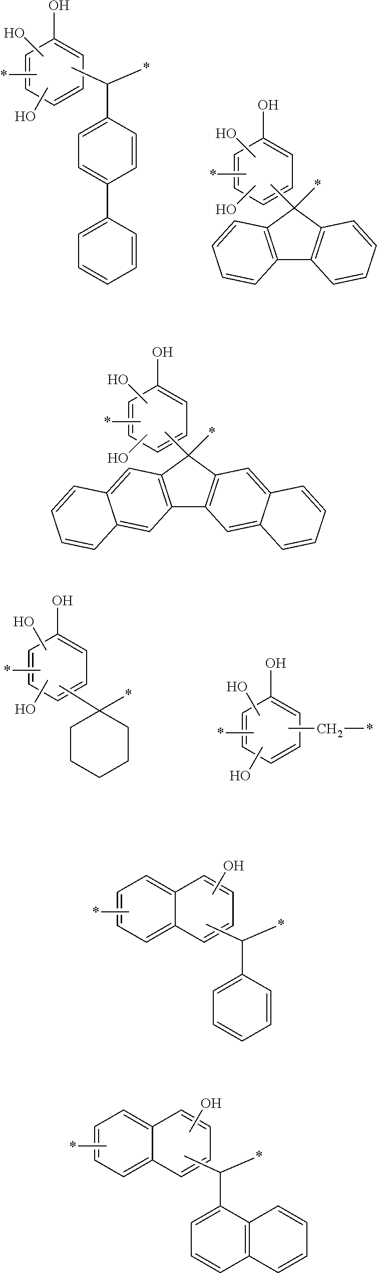

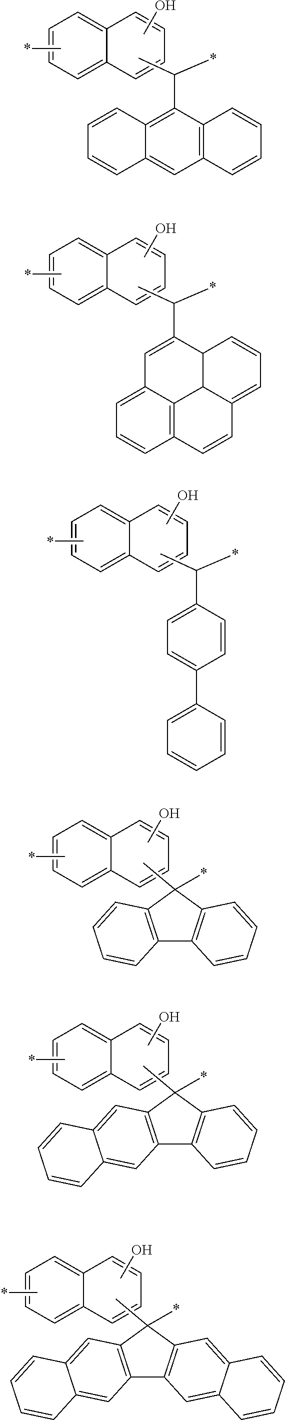

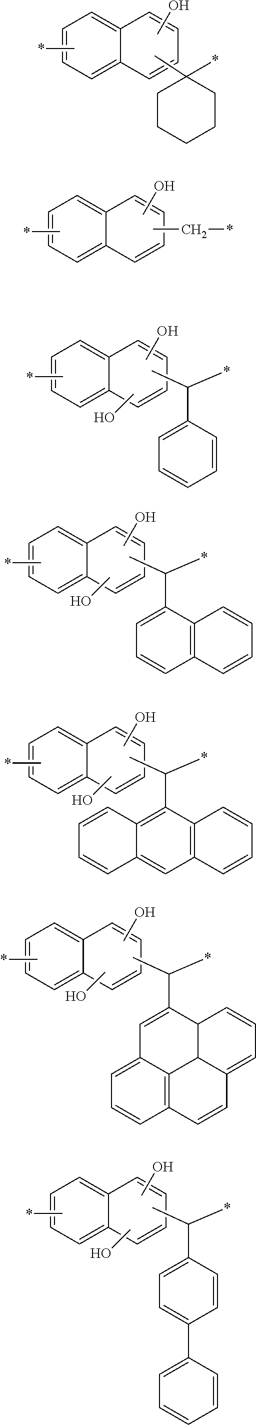

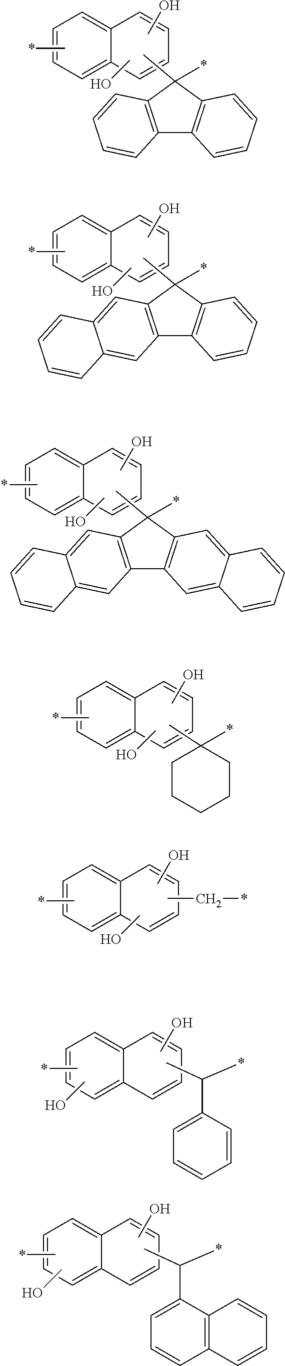

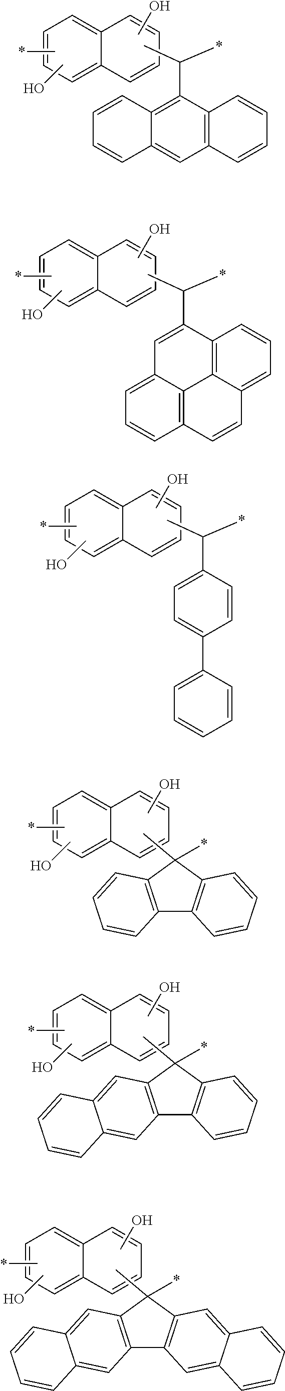

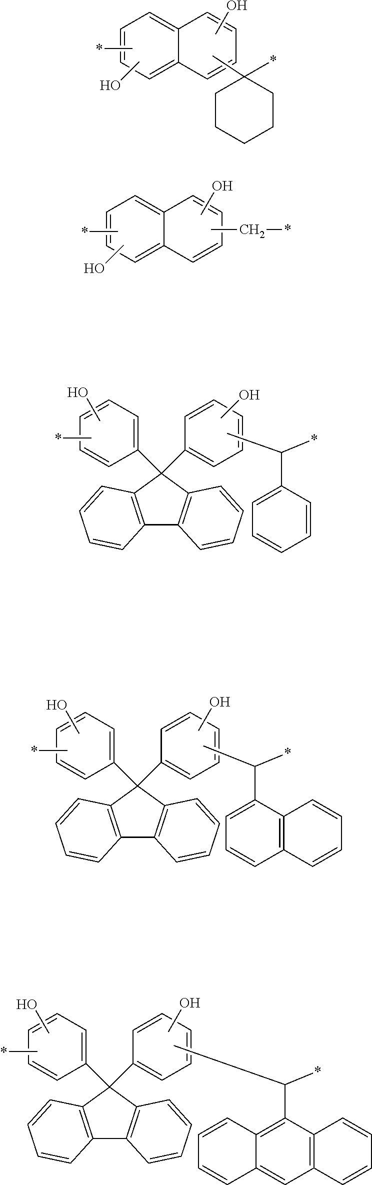

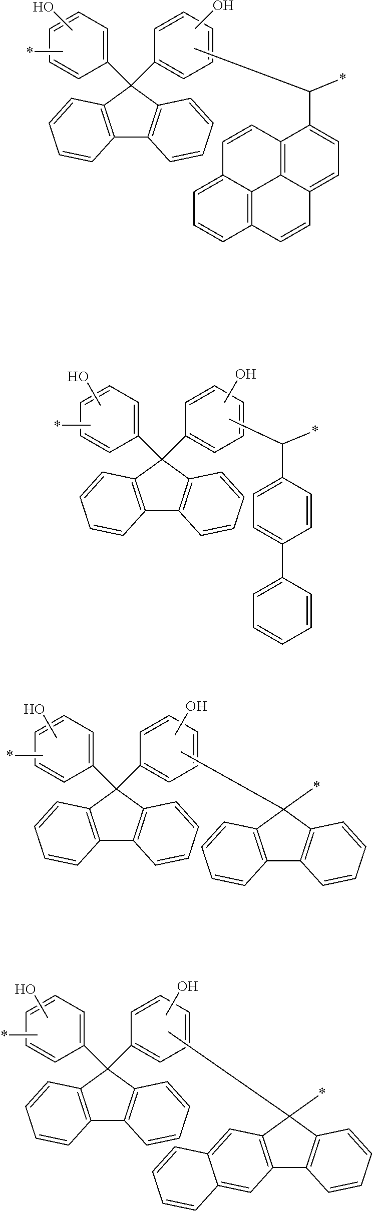

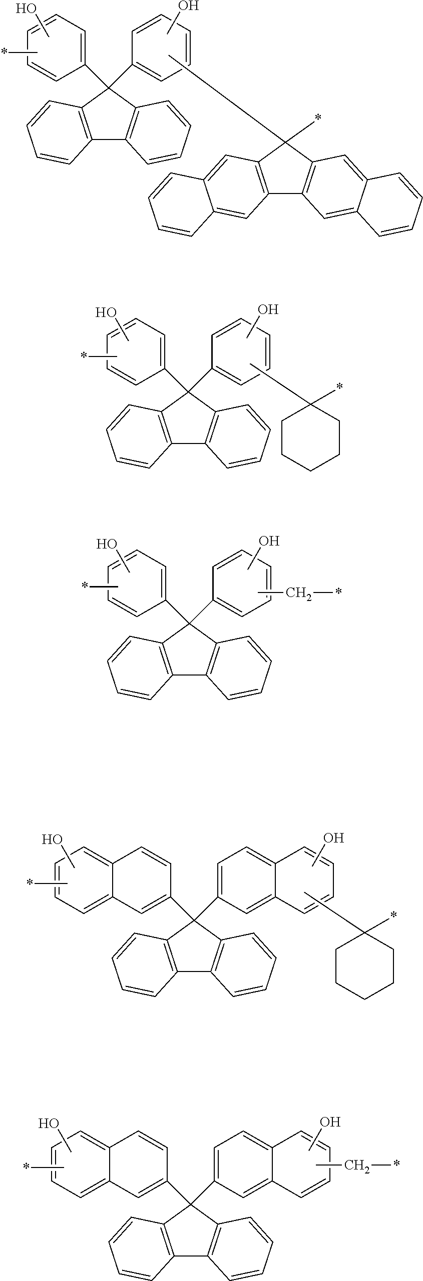

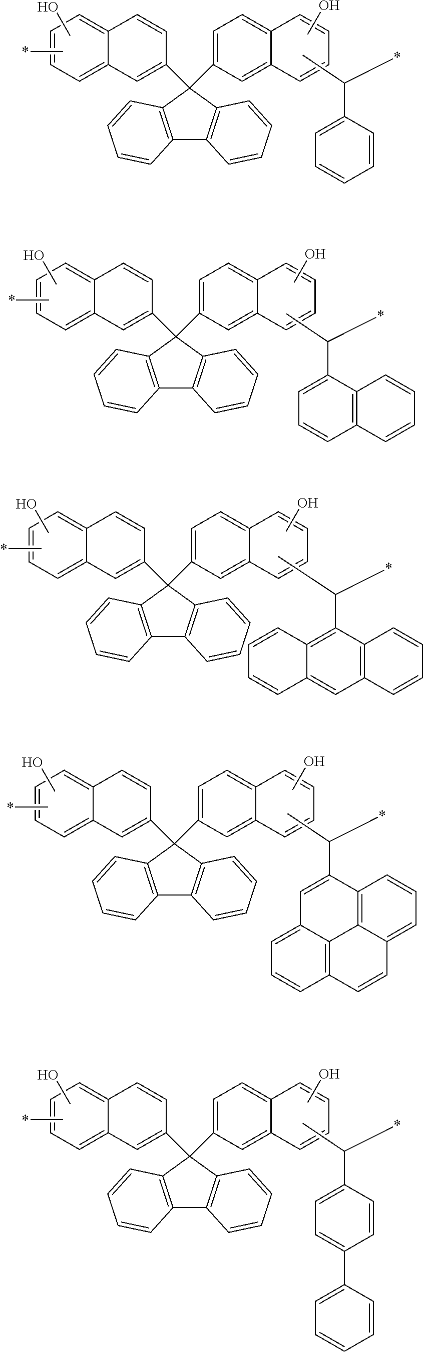

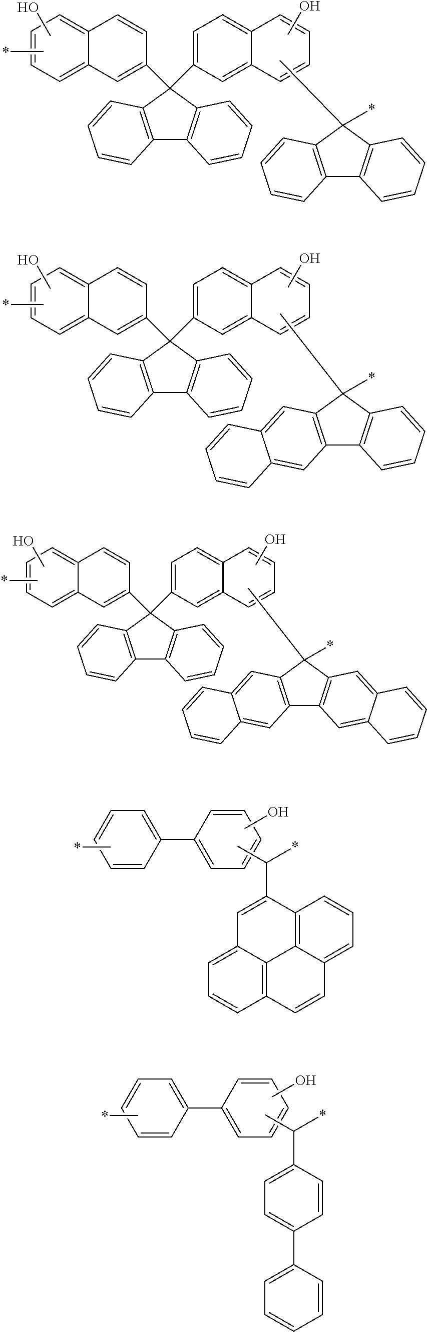

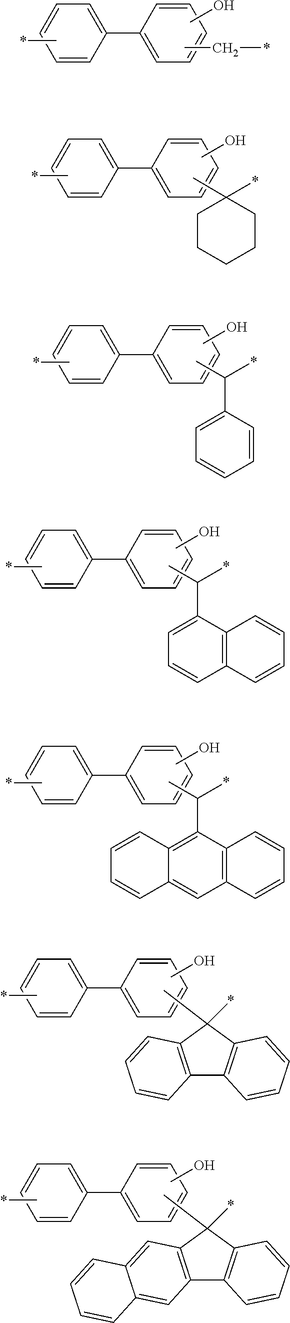

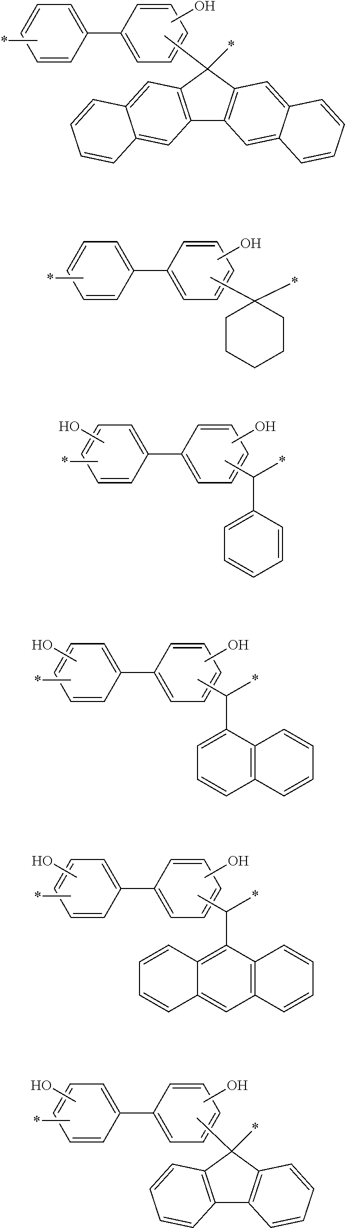

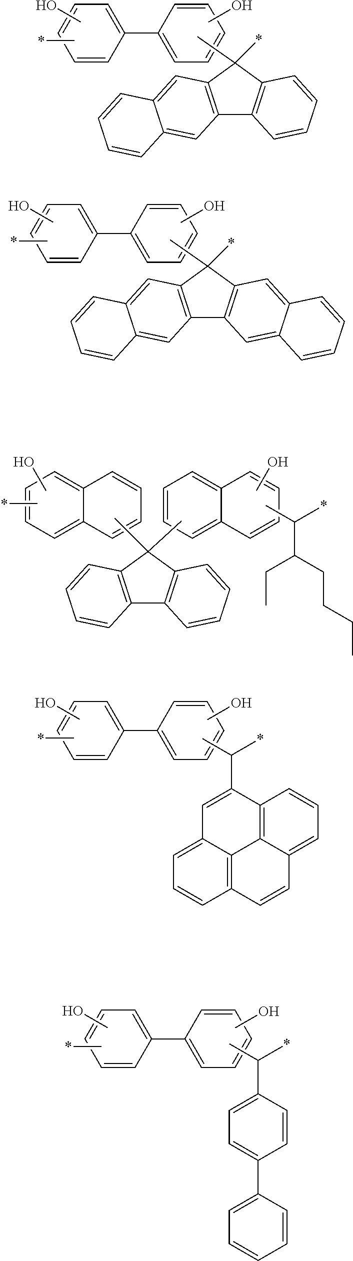

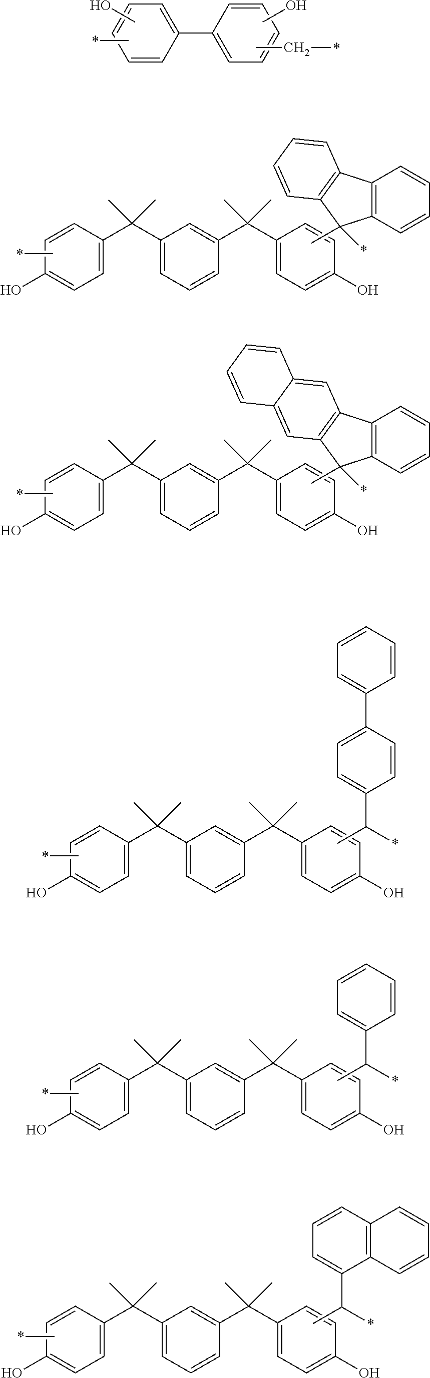

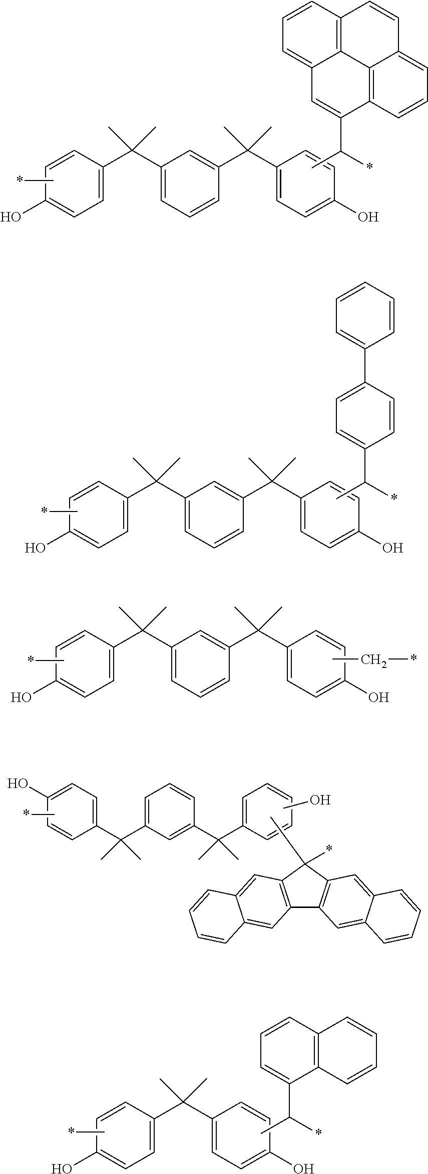

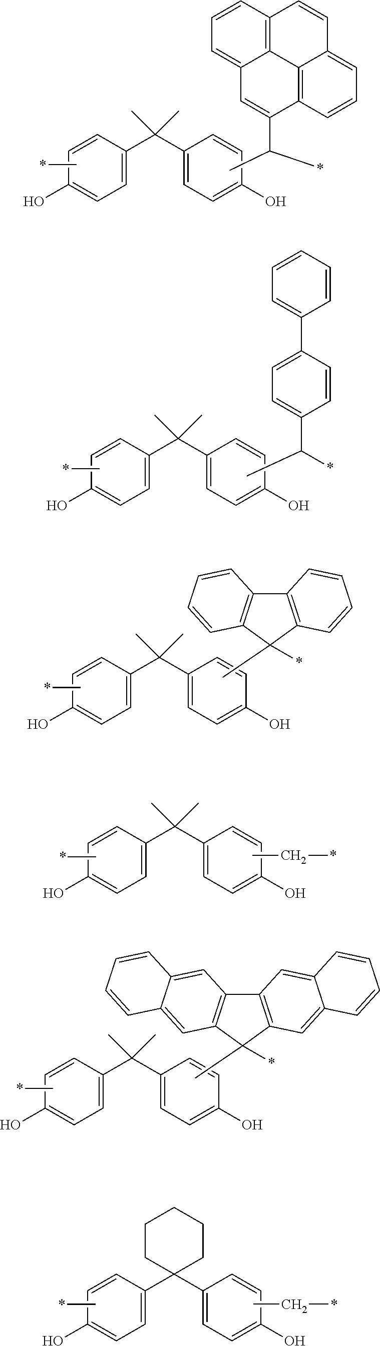

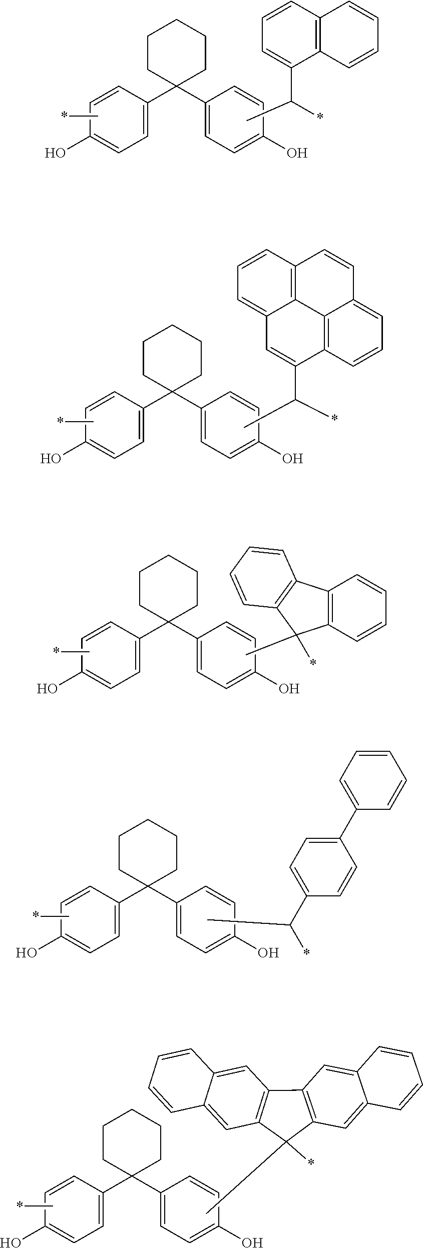

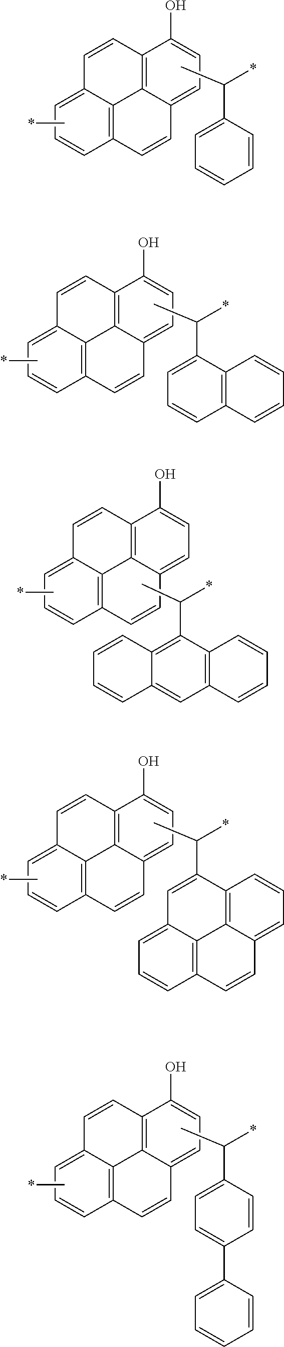

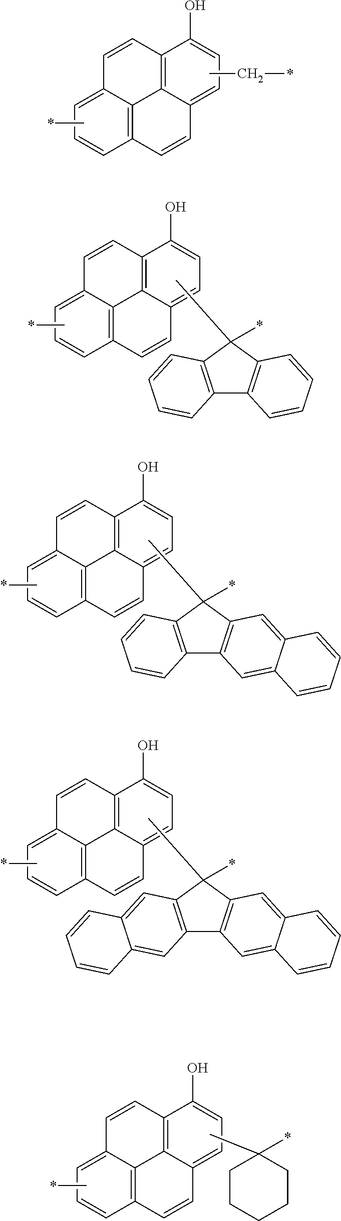

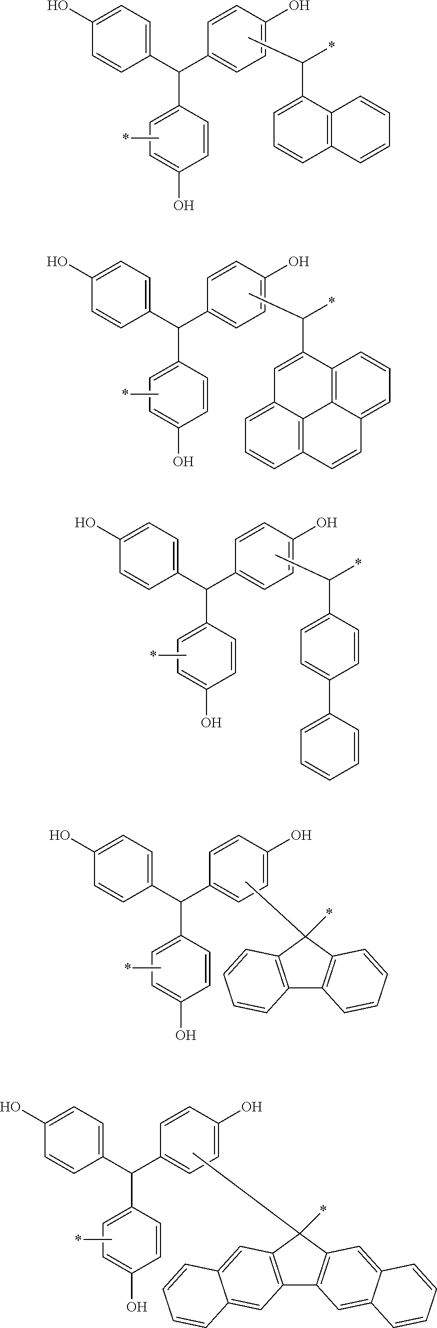

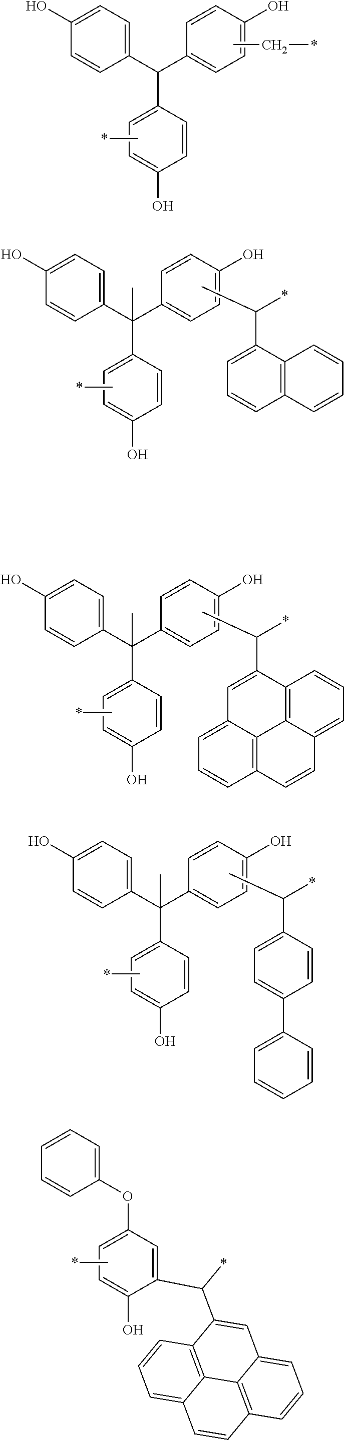

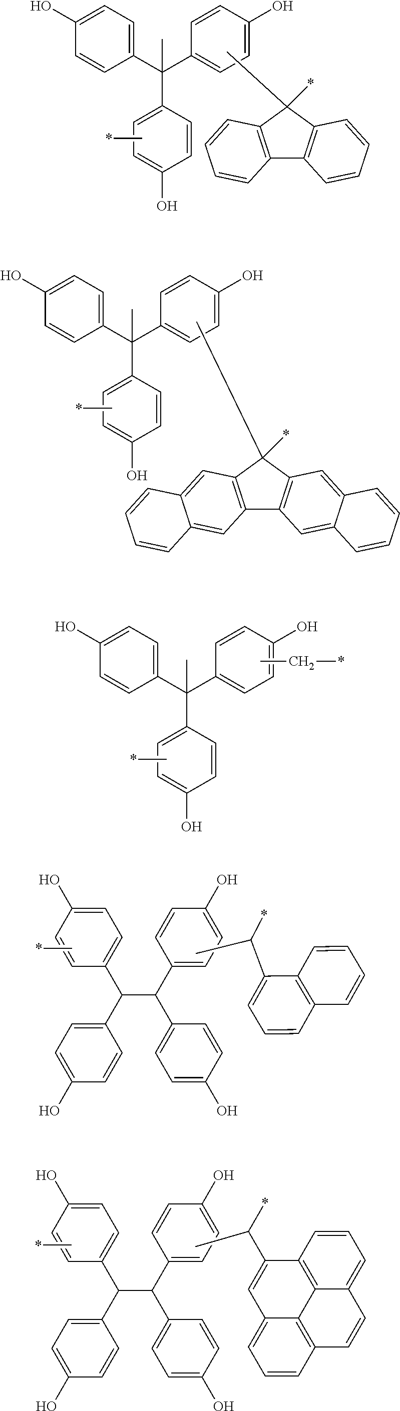

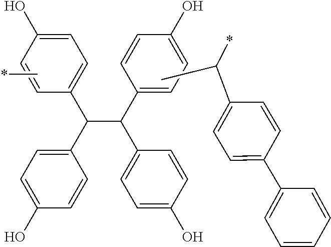

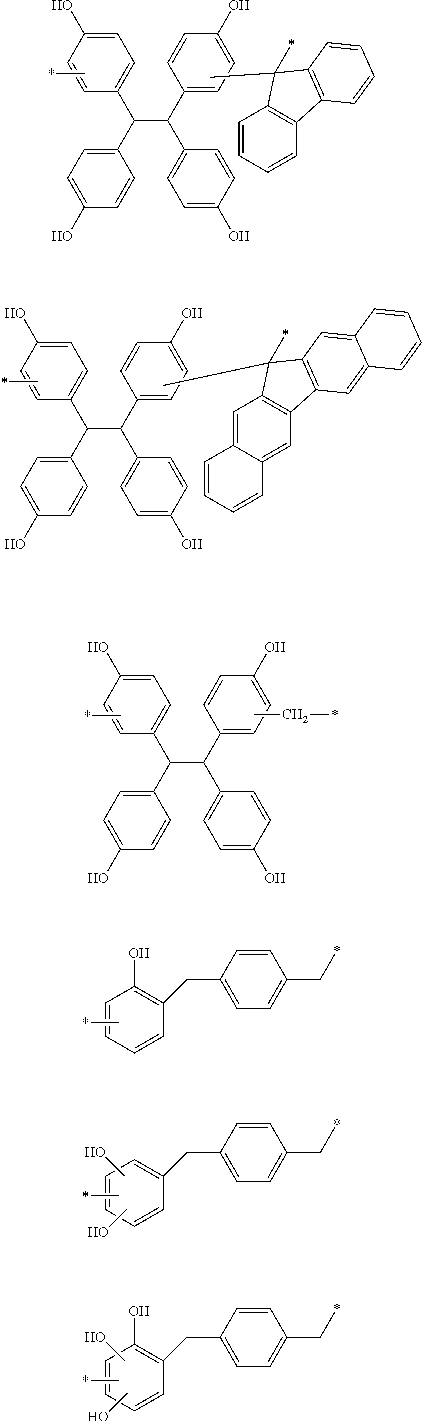

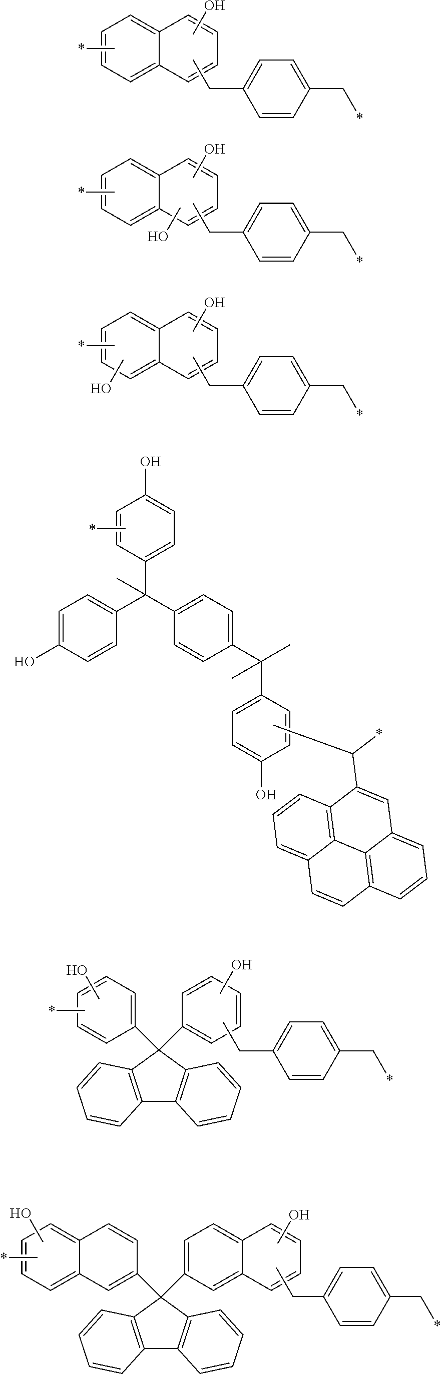

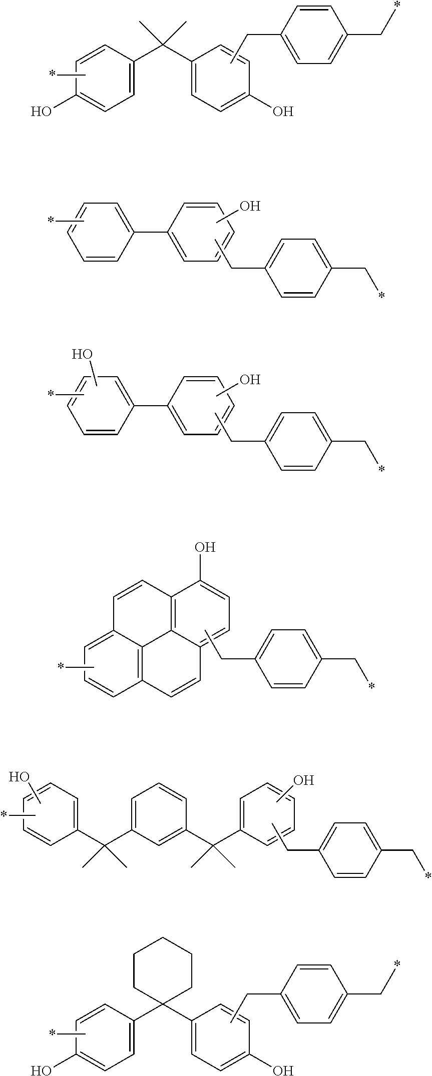

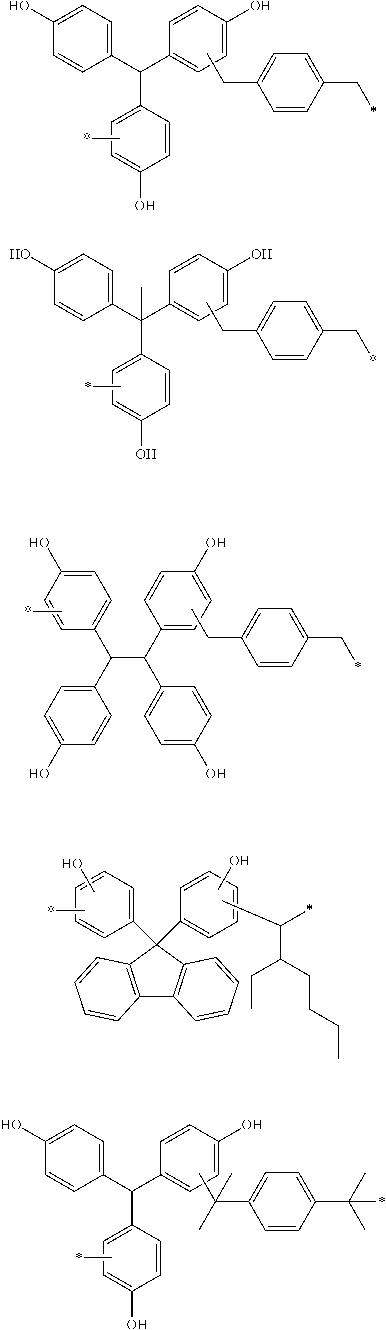

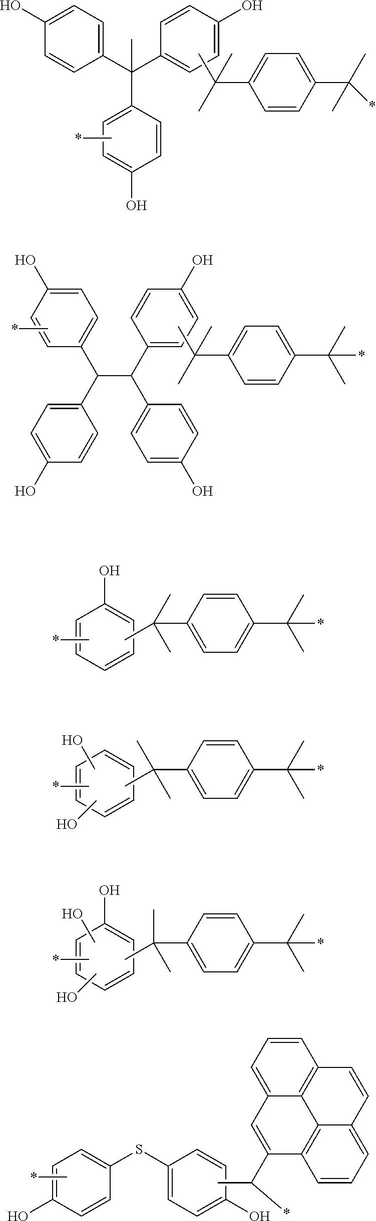

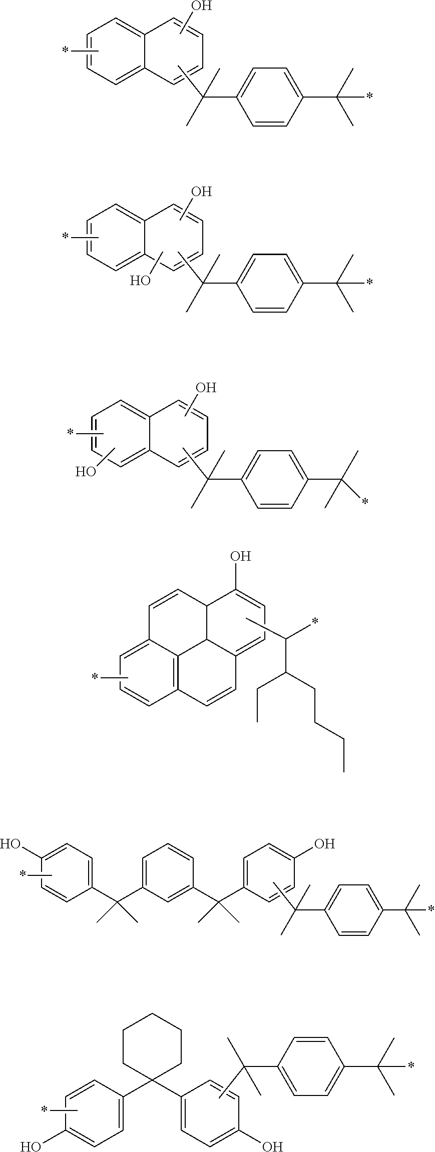

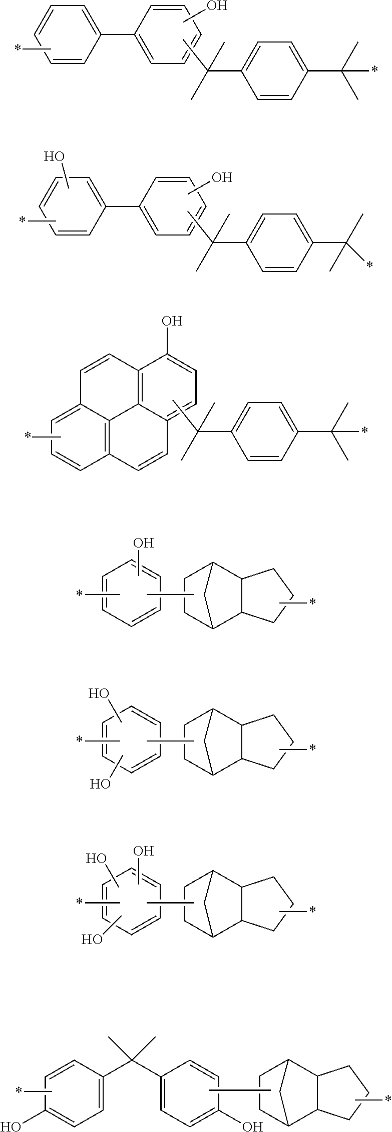

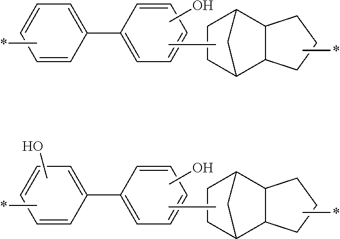

### [Solvents]

The resist underlayer film-forming composition according to the present invention may further include, as a solvent, a compound having an alcoholic hydroxy group or a compound having a group capable of forming an alcoholic hydroxy group. The amount thereof is usually such that the solvent uniformly dissolves the crosslinkable resin, aminoplast crosslinking agent or a phenoplast crosslinking agent, and the crosslinking represented by formula (I).

Examples of the compounds having an alcoholic hydroxy group or the compounds having a group capable of forming an alcoholic hydroxy group include ethylene glycol monomethyl ether, ethylene glycol monoethyl ether, methyl cellosolve acetate, ethyl cellosolvate acetate, diethylene glycol monomethyl ether, diethylene glycol acetate, ethyl cellosolve acetate, diethylene glycol monomethyl ether, diethylene glycol monoethyl ether, propylene glycol, propylene glycol monomethyl ether, propylene glycol monomethyl ether acetate, propylene glycol monoethyl ether, propylene glycol monoethyl ether acetate, propylene glycol propyl ether acetate, cyclopentanone, cyclohexanone, ethyl 2-hydroxypropionate, methyl 2-hydroxy-2-methylpropionate, ethyl 2-hydroxy-2-methylpropionate, ethyl ethoxyacetate, ethyl hydroxyacetate, methyl 2-hydroxy-3-methylbutanoate, methyl 3-methoxypropionate, ethyl 3-methoxypropionate, ethyl 3-ethoxypropionate, methyl 3-ethoxypropionate, methyl pyruvate, ethyl pyruvate, ethyl acetate, butyl acetate, ethyl lactate, and butyl lactate.

Of those mentioned above, propylene glycol solvents, cycloaliphatic ketone solvents, oxyisobutyric acid ester solvents, and butylene glycol solvents are preferable.

The compounds having an alcoholic hydroxy group or the compounds having a group capable of forming an alcoholic hydroxy group may be used each alone or in combination of two or more thereof.

Furthermore, high-boiling solvents, such as propylene glycol monobutyl ether and propylene glycol monobutyl ether acetate, may be mixed.

Preferred examples include propylene glycol monomethyl ether, propylene glycol monomethyl ether acetate, ethyl lactate, butyl lactate, and cyclohexanone. More preferred examples include propylene glycol monomethyl ether and propylene glycol monomethyl ether acetate.

Where necessary, the resist underlayer film-forming composition according to the present invention may include additives, such as crosslinking agents, surfactants, light absorbers, rheology modifiers, and adhesion aids, in addition to the above components.

### [Aminoplast Crosslinking Agent]

Exemplary aminoplast crosslinking agent includes highly alkylated, alkoxylated, or alkoxyalkylated melamines, benzoguanamines, glycolurils, ureas, and polymers thereof. Preferably, the crosslinking agent has at least two crosslinking substituents, with examples including methoxymethylated glycoluril, butoxymethylated glycoluril, methoxymethylated melamine, butoxymethylated melamine, methoxymethylated benzoguanamine, butoxymethylated benzoguanamine, methoxymethylated urea, butoxymethylated urea, methoxymethylated thiourea, and methoxymethylated thiourea. Condensates of these compounds may also be used.

Furthermore, highly heat-resistant crosslinking agents may be used as the crosslinking agent. Compounds that contain a crosslinking substituent having an aromatic ring (for example, a benzene ring or a naphthalene ring) in the molecule may be preferably used as the highly heat-resistant crosslinking agent.

Preferably, the crosslinking agent is at least one member selected from the group consisting of tetramethoxymethylglycoluril and hexamethoxymethylmelamine.

The aminoplast crosslinking agents may be used each alone or in combination of two or more thereof. The aminoplast crosslinking agent may be produced by a method known per se or that deemed as known or may be purchased from the market.

The amount in which the aminoplast crosslinking agent is used varies depending on such factors as the coating solvent that is used, the base substrate that is used, the solution viscosity that is required, and the film shape that is required; however, it is 0.001% by mass or more, 0.01% by mass or more, 0.05% by mass or more, 0.5% by mass or more, or 1.0% by mass or more, and is 80% by mass or less, 50% by mass or less, 40% by mass or less, 20% by mass or less, or 10% by mass or less, based on the total solid content in the resist underlayer film-forming composition according to the present invention.

Some specific examples are illustrated below:

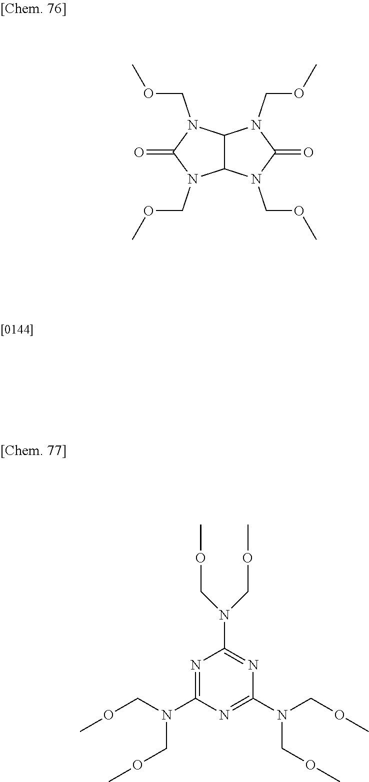

### [Phenoplast Crosslinking Agent]

Exemplary phenoplast crosslinking agent includes highly alkylated, alkoxylated, or alkoxyalkylated aromatics, and polymers thereof. Preferably, the crosslinking agent has at least two crosslinking substituents in the molecule, with examples including 2,6-dihydroxymethyl-4-methylphenol,
2,4-dihydroxymethyl-6-methylphenol,
bis(2-hydroxy-3-hydroxymethyl-5-methylphenyl)methane, bis(4-hydroxy-3-hydroxymethyl-5-methylphenyl)methane,
2,2-bis(4-hydroxy-3, 5-dihydroxymethylphenyl)propane,
bis(3-formyl-4-hydroxyphenyl)methane,
bis(4-hydroxy-2,5-dimethylphenyl)formylmethane, and
α,α-bis(4-hydroxy-2,5-dimethylphenyl)-4-formyltoluene. Condensates of these compounds may also be used.

Furthermore, highly heat-resistant crosslinking agents may be used as the crosslinking agent. Compounds that contain a crosslinking substituent having an aromatic ring (for example, a benzene ring or a naphthalene ring) in the molecule may be preferably used as the highly heat-resistant crosslinking agent.

The phenoplast crosslinking agents may be used each alone or in combination of two or more thereof. The phenoplast crosslinking agent may be produced by a method known per se or that deemed as known or may be purchased from the market.

The amount in which the phenoplast crosslinking agent is used varies depending on such factors as the coating solvent that is used, the base substrate that is used, the solution viscosity that is required, and the film shape that is required; however, it is 0.001% by mass or more, 0.01% by mass or more, 0.05% by mass or more, 0.5% by mass or more, or 1.0% by mass or more, and is 80% by mass or less, 50% by mass or less, 40% by mass or less, 20% by mass or less, or 10% by mass or less, based on the total solid content in the resist underlayer film-forming composition according to the present invention.

Examples of such compounds, in addition to those described above, include compounds that have a partial structure of formula (4) below, and polymers or oligomers that have a repeating unit of formula (5) below.

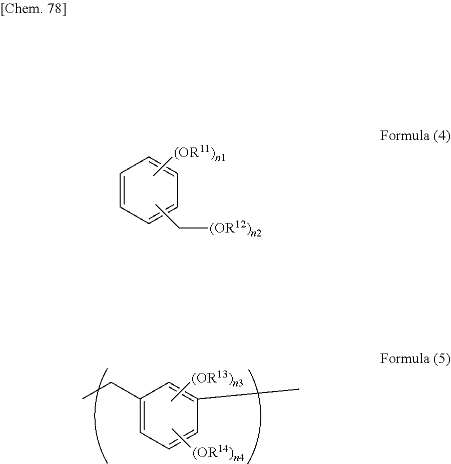

R¹¹, R¹², R¹³, and R¹⁴ are each a hydrogen atom or a C1-C10 alkyl group. Examples of the alkyl group includes those mentioned hereinabove. n1 is an integer of 1 to 4, n2 is an integer of 1 to (5−n1), and (n1+n2) is an integer of 2 to 5. n3 is an integer of 1 to 4, n4 is 0 to (4−n3), and (n3+n4) is an integer of 1 to 4. The number of the repeating unit structures in the oligomers and polymers may be within the range of 2 to 100, or 2 to 50.

Some specific examples are illustrated below:

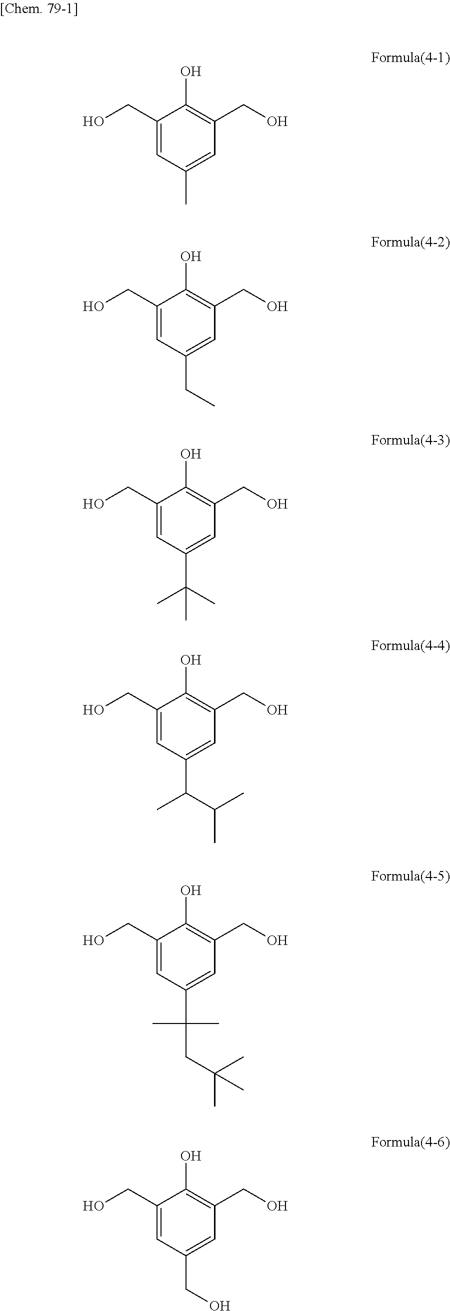

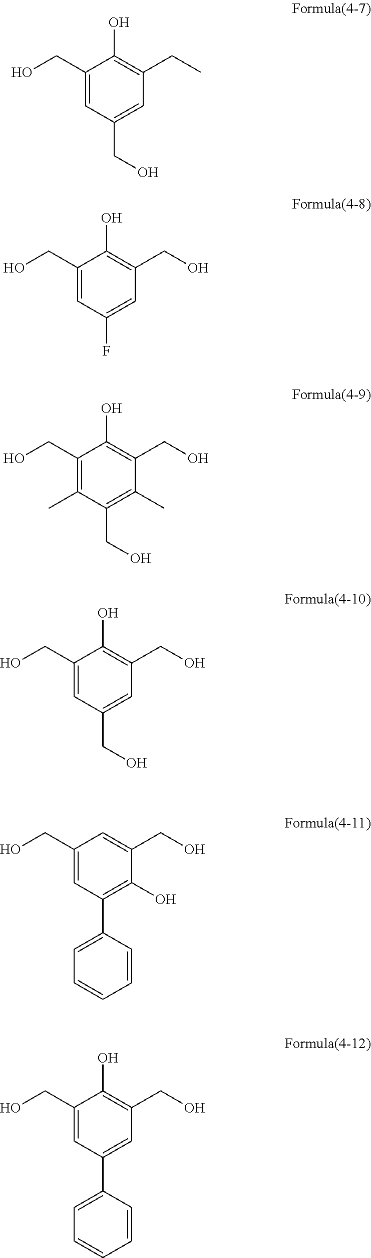 [SMILES: CCc1cc(CO)cc(CO)c1O ; OCc1cc(F)cc(CO)c1O ; Cc1c(CO)c(C)c(CO)c(O)c1CO ; OCc1cc(CO)c(O)c(CO)c1 ; OCc1cc(CO)c(O)c(-c2ccccc2)c1 ; OCc1cc(-c2ccccc2)cc(CO)c1O]

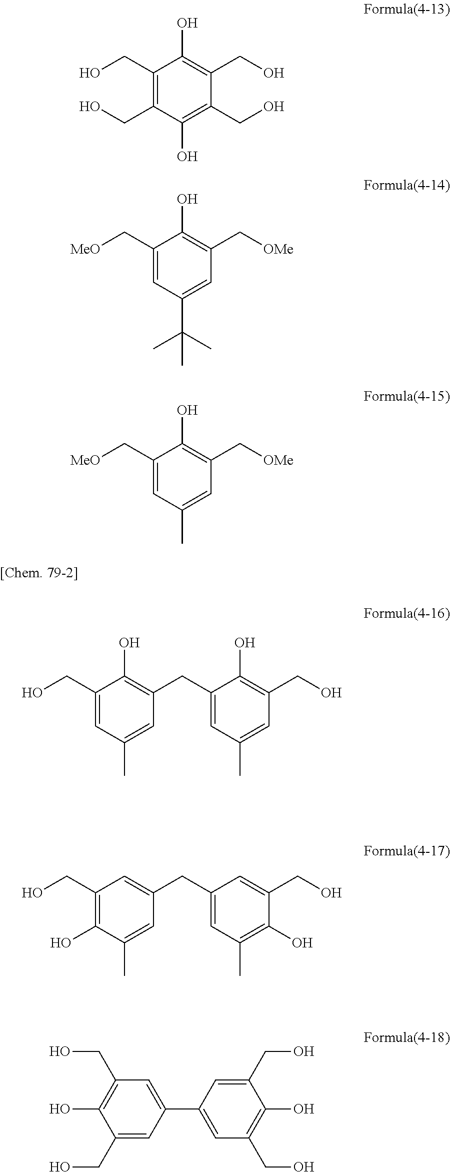

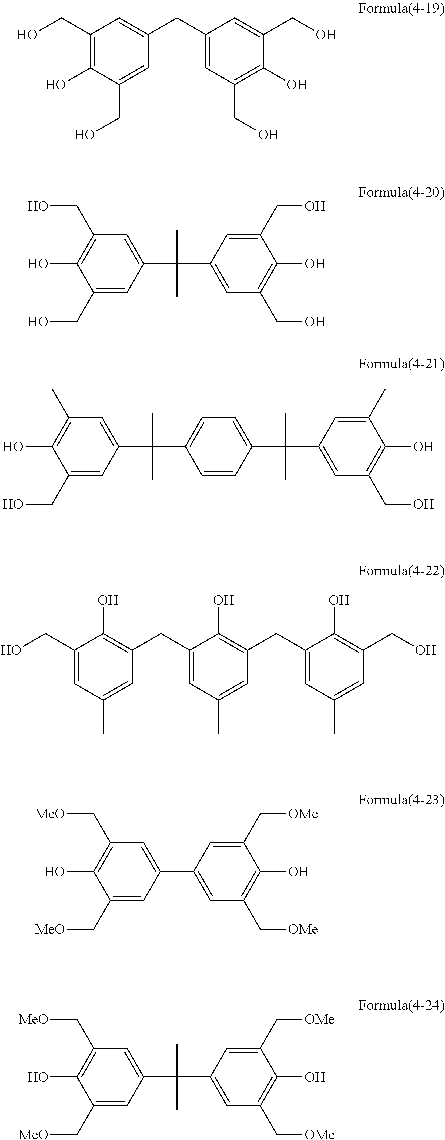 [SMILES: OCc1cc(Cc2cc(CO)c(O)c(CO)c2)cc(CO)c1O ; CC(C)(c1cc(CO)c(O)c(CO)c1)c1cc(CO)c(O)c(CO)c1 ; Cc1cc(C(C)(C)c2ccc(C(C)(C)c3cc(C)c(O)c(CO)c3)cc2)cc(CO)c1O ; Cc1cc(CO)c(O)c(Cc2cc(C)cc(Cc3cc(C)cc(CO)c3O)c2O)c1 ; COCc1cc(-c2cc(COC)c(O)c(COC)c2)cc(COC)c1O ; COCc1cc(C(C)(C)c2cc(COC)c(O)c(COC)c2)cc(COC)c1O]

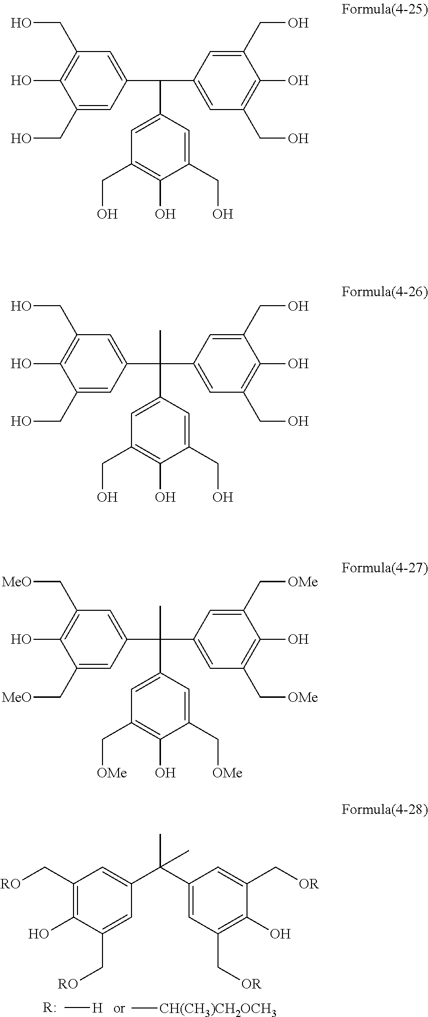

### [Surfactant]

To reduce the occurrence of defects, such as pinholes or striation, and to further enhance the applicability to surface unevenness, the resist underlayer film-forming composition according to the present invention may include a surfactant.

Examples of the surfactant include nonionic surfactants, for example, polyoxyethylene alkyl ethers, such as polyoxyethylene lauryl ether, polyoxyethylene stearyl ether, polyoxyethylene cetyl ether, and polyoxyethylene oleyl ether, polyoxyethylene alkyl aryl ethers, such as polyoxyethylene octyl phenol ether and polyoxyethylene nonyl phenol ether, polyoxyethylene/polyoxypropylene block copolymers, sorbitan fatty acid esters, such as sorbitan monolaurate, sorbitan monopalmitate, sorbitan monostearate, sorbitan monooleate, sorbitan trioleate, and sorbitan tristearate, and polyoxyethylene sorbitan fatty acid esters, such as polyoxyethylene sorbitan monolaurate, polyoxyethylene sorbitan monopalmitate, polyoxyethylene sorbitan monostearate, polyoxyethylene sorbitan trioleate, and polyoxyethylene sorbitan tristearate; fluorine surfactants, such as EFTOP series EF301, EF303, and EF352 (product names, manufactured by Tohkem Products Corp.), MEGAFACE series F171, F173, R-30, and R-40 (product names, manufactured by DIC CORPORATION), FLUORAD series FC430 and FC431 (product names, manufactured by Sumitomo 3M Ltd.), ASAHI GUARD AG710, and SURFLON series S-382, SC101, SC102, SC103, SC104, SC105, and SC106 (product names, manufactured by AGC Inc.); and organosiloxane polymer KP341 (manufactured by Shin-Etsu Chemical Co., Ltd.). The amount in which the surfactant is incorporated is usually 2.0% by mass or less, and preferably 1.0% by mass or less relative to the total solid content in the resist underlayer film-forming composition according to the present invention. The surfactants may be added each alone or in combination of two or more thereof.

### [Other Additives]

In order to promote the crosslinking reaction, the resist underlayer film-forming composition according to the present invention may contain a catalyst in addition to the crosslinking catalyst of formula (I). Examples of such additional catalysts include acidic compounds, such as citric acid; thermal acid generators, such as 2,4,4,6-tetrabromocyclohexadienone, benzoin tosylate, 2-nitrobenzyl tosylate, and other organic sulfonic acid alkyl esters; onium salt photoacid generators, such as bis(4-t-butylphenyl)iodonium trifluoromethanesulfonate and triphenylsulfonium trifluoromethanesulfonate; halogen-containing compound-based photoacid generators, such as phenyl-bis(trichloromethyl)-s-triazine; and sulfonic acid-based photoacid generators, such as benzoin tosylate and N-hydroxysuccinimide trifluoromethanesulfonate.

Some example light absorbers that may be suitably used are commercially available light absorbers described in “Kougyouyou Shikiso no Gijutsu to Shijou (Technology and Market of Industrial Dyes)” (CMC Publishing Co., Ltd.) and “Senryou Binran (Dye Handbook)” (edited by The Society of Synthetic Organic Chemistry, Japan), such as, for example, C. I. Disperse Yellow 1, 3, 4, 5, 7, 8, 13, 23, 31, 49, 50, 51, 54, 60, 64, 66, 68, 79, 82, 88, 90, 93, 102, 114, and 124; C. I. Disperse Orange 1, 5, 13, 25, 29, 30, 31, 44, 57, 72, and 73; C. I. Disperse Red 1, 5, 7, 13, 17, 19, 43, 50, 54, 58, 65, 72, 73, 88, 117, 137, 143, 199, and 210; C. I. Disperse Violet 43; C. I. Disperse Blue 96; C. I. Fluorescent Brightening Agent 112, 135, and 163; C. I. Solvent Orange 2 and 45; C. I. Solvent Red 1, 3, 8, 23, 24, 25, 27, and 49; C. I. Pigment Green 10; and C. I. Pigment Brown 2. The light absorber is usually added in a proportion of 10% by mass or less, preferably 5% by mass or less relative to the total solid content in the resist underlayer film-forming composition according to the present invention.

A rheology modifier may be added mainly to enhance the fluidity of the resist underlayer film-forming composition and thereby, particularly in the baking step, to enhance the uniformity in thickness of a resist underlayer film and to increase the filling performance of the resist underlayer film-forming composition with respect to the inside of holes. Specific examples thereof include phthalic acid derivatives, such as dimethyl phthalate, diethyl phthalate, diisobutyl phthalate, dihexyl phthalate, and butyl isodecyl phthalate; adipic acid derivatives, such as di-n-butyl adipate, diisobutyl adipate, diisooctyl adipate, and octyl decyl adipate; maleic acid derivatives, such as di-n-butyl maleate, diethyl maleate, and dinonyl maleate; oleic acid derivatives, such as methyl oleate, butyl oleate, and tetrahydrofurfuryl oleate; and stearic acid derivatives, such as n-butyl stearate and glyceryl stearate. The rheology modifier is usually added in a proportion of less than 30% by mass relative to the total solid content in the resist underlayer film-forming composition according to the present invention.

An adhesion aid may be added mainly to enhance the adhesion between the resist underlayer film-forming composition and a substrate or a resist and thereby to prevent the detachment of the resist particularly during development. Specific examples thereof include chlorosilanes, such as trimethylchlorosilane, dimethylvinylchlorosilane, methyldiphenylchlorosilane, and chloromethyldimethylchlorosilane; alkoxysilanes, such as trimethylmethoxysilane, dimethyldiethoxysilane, methyldimethoxysilane, dimethylvinylethoxysilane, diphenyldimethoxysilane, and phenyltriethoxysilane; silazanes, such as hexamethyldisilazane, N,N′-bis(trimethylsilyl)urea, dimethyltrimethylsilylamine, and trimethylsilylimidazole; silanes, such as vinyltrichlorosilane, γ-chloropropyltrimethoxysilane, γ-aminopropyltriethoxysilane, and γ-glycidoxypropyltrimethoxysilane; heterocyclic compounds, such as benzotriazole, benzimidazole, indazole, imidazole, 2-mercaptobenzimidazole, 2-mercaptobenzothiazole, 2-mercaptobenzoxazole, urazole, thiouracil, mercaptoimidazole, and mercaptopyrimidine; and urea or thiourea compounds, such as 1,1-dimethylurea and 1,3-dimethylurea. The adhesion aid is usually added in a proportion of less than 5% by mass, preferably less than 2% by mass relative to the total solid content in the resist underlayer film-forming composition according to the present invention.

The solid content in the resist underlayer film-forming composition according to the present invention is 0.1 to 70% by mass or 0.1 to 60% by mass. The solid content is the content of all the components except the solvent in the resist underlayer film-forming composition. The proportion of the crosslinkable resin in the solid content may be within the range of 1 to 99.9% by mass, or 50 to 99.9% by mass, or 50 to 95% by mass, or 50 to 90% by mass.

### [Resist Underlayer Film]

A resist underlayer film may be formed as described below using the resist underlayer film-forming composition according to the present invention.

The resist underlayer film-forming composition of the present invention is applied with an appropriate technique, such as a spinner or a coater, onto a semiconductor device substrate (such as, for example, a silicon wafer substrate, a silicon dioxide-coated substrate (a SiO₂ substrate), a silicon nitride substrate (a SiN substrate), a silicon oxynitride substrate (a SiON substrate), a titanium nitride substrate (a TiN substrate), a tungsten substrate (a W substrate), a glass substrate, an ITO substrate, a polyimide substrate, or a low-dielectric constant material (low-k material)-coated substrate), and the coating is baked using a heating device, such as a hot plate, to form a resist underlayer film. The baking conditions are appropriately selected from baking temperatures of 80° C. to 600° C. and amounts of baking time of 0.3 to 60 minutes. The baking temperature is preferably 150° C. to 400° C., and the baking time is preferably 0.5 to 2 minutes. The atmosphere gas at the time of baking may be air or an inert gas, such as nitrogen or argon. Here, the film thickness of the underlayer film that is formed is, for example, 10 to 1000 nm, or 20 to 500 nm, or 30 to 400 nm, or 50 to 300 nm. Furthermore, replicas (mold replicas) of a quartz imprinting mold may be produced by using quartz substrates as the substrates.

Furthermore, an adhesion layer and/or a silicone layer containing 99% by mass or less, or 50% by mass or less of Si may be formed on the resist underlayer film according to the present invention by application or deposition. For example, an adhesion layer described in JP 2013-202982 A or Japanese Patent No. 5827180 may be formed, or a silicon-containing resist underlayer film (inorganic resist underlayer film)-forming composition described in WO 2009/104552 A1 may be applied by spin coating. Furthermore, a Si-based inorganic material film may be formed by such a method as a CVD method.

The resist underlayer film-forming composition according to the present invention may be applied onto a semiconductor substrate having a stepped region and a stepless region (the so-called non-planar substrate) and may be baked to reduce the difference in height between the stepped region and the stepless region.

### [Method for Manufacturing Semiconductor Device]

(1) A method for manufacturing a semiconductor device according to the present invention comprises the steps of:
forming a resist underlayer film from the resist underlayer film-forming composition according to the present invention;
forming a resist film on the resist underlayer film;
forming a resist pattern by irradiation with a light or electron beam followed by development;
etching the resist underlayer film through the resist pattern; and
processing the semiconductor substrate through the patterned resist underlayer film.

(2) A method for manufacturing a semiconductor device according to another aspect of the present invention comprises the steps of:
forming a resist underlayer film from the resist underlayer film-forming composition according to the present invention;
forming a hard mask on the resist underlayer film;
forming a resist film on the hard mask;
forming a resist pattern by irradiation with a light or electron beam followed by development;
etching the hard mask through the resist pattern;
etching the resist underlayer film through the patterned hard mask; and processing the semiconductor substrate through the patterned resist underlayer film.

(3) A method for manufacturing a semiconductor device according to another aspect of the present invention comprises the steps of:
forming a resist underlayer film from the resist underlayer film-forming composition according to the present invention;
forming a hard mask on the resist underlayer film;
forming a resist film on the hard mask;
forming a resist pattern by irradiation with a light or electron beam followed by development;
etching the hard mask through the resist pattern;
etching the resist underlayer film through the patterned hard mask;
removing the hard mask; and
processing the semiconductor substrate through the patterned resist underlayer film.

(4) A method for manufacturing a semiconductor device according to another aspect of the present invention comprises the steps of:
forming a resist underlayer film from the resist underlayer film-forming composition according to the present invention;
forming a hard mask on the resist underlayer film;
forming a resist film on the hard mask;
forming a resist pattern by irradiation with a light or electron beam followed by development;
etching the hard mask through the resist pattern formed;
etching the resist underlayer film through the patterned hard mask; removing the hard mask;
forming a deposited film or spacer on the hard mask-removed, resist underlayer film;
processing the deposited film or spacer by etching;
removing the patterned resist underlayer film to leave the patterned deposited film or spacer; and
processing the semiconductor substrate through the patterned deposited film or spacer.

The step of forming a resist underlayer film from the resist underlayer film-forming composition according to the present invention is as described hereinabove.

As a second resist underlayer film, an organopolysiloxane film may be formed on the resist underlayer film resulting from the above step, and a resist pattern may be formed thereon. The second resist underlayer film may be a SiON film or a SiN film formed by a deposition method, such as CVD or PVD. Furthermore, a bottom anti-reflective coating (BARC) as a third resist underlayer film may be formed on the second resist underlayer film. The third resist underlayer film may be a resist shape correction film having no antireflection function.

In the step of forming a resist pattern, the exposure is performed through a mask (a reticle) for forming a predetermined pattern or is carried out by direct drawing. For example, g-ray, i-ray, KrF excimer laser, ArF excimer laser, EUV, or electron beam may be used as the exposure source. After the exposure, post exposure baking is performed as required. Subsequently, the latent image is developed with a developing solution (for example, a 2.38% by mass aqueous tetramethylammonium hydroxide solution), and the pattern is further rinsed with a rinsing solution or pure water to remove the developing solution used. Subsequently, post-baking is performed to dry the resist pattern and to enhance the adhesion with respect to the base.

The hard mask may be formed by applying a composition containing an inorganic substance or by depositing an inorganic substance. Examples of the inorganic substances include silicon oxynitride.

The etching steps after the resist pattern formation are performed by dry etching. The etching gas used for dry etching may be CF₄, CHF₃, CH₂F2, CH₃F, C₄F₆, C₄F₈, O₂, N₂O, NO₂, He, or H₂ for the processing of the second resist underlayer film (the organopolysiloxane film), the first resist underlayer film formed from the resist underlayer film-forming composition of the present invention, and the substrate. These gases may be used each alone or in combination of two or more thereof. Furthermore, argon, nitrogen, carbon dioxide, carbonyl sulfide, sulfur dioxide, neon, or nitrogen trifluoride may be mixed with the above gases.

Wet etching is sometimes performed for the purposes of simplifying the process step and reducing the damage to the workpiece substrate. This leads to smaller variations in processing dimensions and smaller pattern roughness and enables processing of substrates with a high yield. Thus, the hard mask may be removed by either etching or using an alkaline chemical solution in (3) and (4) of [Method for manufacturing semiconductor device]. When, in particular, an alkaline chemical solution is used, the components are not particularly limited but the solution preferably includes any of the following alkaline components.

Examples of the alkaline components include tetramethylammonium hydroxide, tetraethylammonium hydroxide, tetrapropylammonium hydroxide, tetrabutylammonium hydroxide, methyltripropylammonium hydroxide, methyltributylammonium hydroxide, ethyltrimethylammonium hydroxide, dimethyldiethylammonium hydroxide, benzyltrimethylammonium hydroxide, hexadecyltrimethylammonium hydroxide, and (2-hydroxyethyl)trimethylammonium hydroxide, monoethanolamine, diethanolamine, triethanolamine, 2-(2-aminoethoxy)ethanol, N,N-dimethylethanolamine, N,N-diethylethanolamine, N,N-dibutylethanolamine, N-methylethanolamine, N-ethylethanolamine, N-butylethanolamine, N-methyldiethanolamine, monoisopropanolamine, diisopropanolamine, triisopropanolamine, tetrahydrofurfurylamine, N-(2-aminoethyl)piperazine, 1,8-diazabicyclo[5.4.0]undecene-7, 1,4-diazabicyclo[2.2.2]octane, hydroxyethylpiperazine, piperazine, 2-methylpiperazine, trans-2,5-dimethylpiperazine, cis-2,6-dimethylpiperazine, 2-piperidinemethanol, cyclohexylamine, and 1,5-diazabicyclo[4.3.0]nonene-5. From the point of view of handling in particular, tetramethylammonium hydroxide and tetraethylammonium hydroxide are particularly preferable, and an inorganic base may be used in combination with the quaternary ammonium hydroxide. Preferred inorganic bases are alkali metal hydroxides, such as potassium hydroxide, sodium hydroxide, and rubidium hydroxide, with potassium hydroxide being more preferable.

### [Formation of Resist Underlayer Film by Nanoimprinting Method]

The step of forming a resist underlayer film may also be performed by a nanoimprinting method. This method comprises the steps of:
applying a curable composition onto a resist underlayer film formed; bringing the curable composition into contact with a mold;
irradiating the curable composition with a light or electron beam to form a cured film; and
separating the cured film and the mold from each other.

### [Formation of Resist Underlayer Film by Self-Assembling Method]

The step of forming a resist underlayer film may also be performed by a self-assembling method. In the self-assembling method, a pattern is formed using a self-assembled film that naturally forms a regular structure on the order of nanometers, for example, a diblock polymer (such as polystyrene-polymethyl methacrylate).

The polymer (G) according to the present invention is expected to show good permeability to gases, such as He, H₂, N₂, and air, exhibits good gap-filling properties, hardness, and bend resistance, and can be controlled to appropriate optical constants and etching rates suited for the process by altering the molecular skeleton. For example, the details are as disclosed in the section [Formation of resist underlayer films by nanoimprinting method] in the specification of Japanese Patent Application No. 2020-033333.

The thermal acid generator used in the resist underlayer film-forming composition according to the present invention is characterized in that an amine compound having a higher basicity than pyridine is selected as the base paired with sulfonic acid.

Although not intending to be bound by theory, the above thermal acid generator offers high storage stability of a polymer that is the main component of a resist underlayer film and, as a result, the polymer can form with high productivity a film that is resistant to dissolution into a photoresist solvent. When, in particular, a polymer has an amine skeleton, the amine moiety of the polymer will be subjected to action by sulfonic acid derived from a thermal acid generator to cause over-time coloration of a resist underlayer film-forming composition during storage. The above thermal acid generator is also expected to be effective in suppressing this coloration.

### EXAMPLES

Hereinbelow, the contents of the present invention will be described in detail by presenting Examples without limiting the scope of the present invention to such Examples.

The following are the apparatus and conditions used in the measurement of the weight average molecular weight of reaction products obtained in Synthesis Examples below.
Apparatus: HLC-8320 GPC manufactured by TOSOH CORPORATION
GPC columns: TSKgel Super-Multipore HZ-N(two columns)
Column temperature: 40° C.
Flow rate: 0.35 mL/min
Eluent: THF
Standard samples: polystyrene

### [Synthesis of Polymers]

Compounds A, compounds B, compounds C, catalysts D, solvents E, and reprecipitation solvent F shown below were used for the synthesis of polymers with structural formulas (S1) to (S15) for use in resist underlayer films.

### (Compounds A to C)

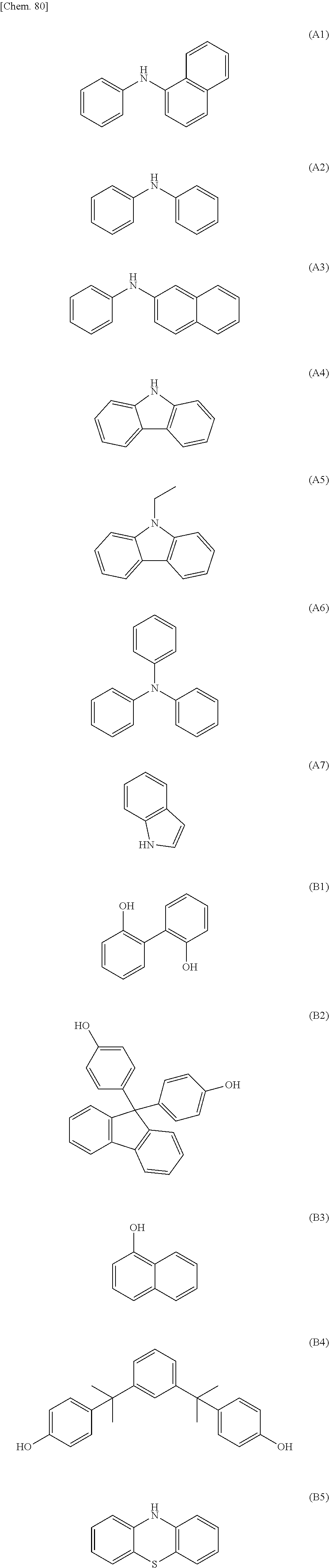

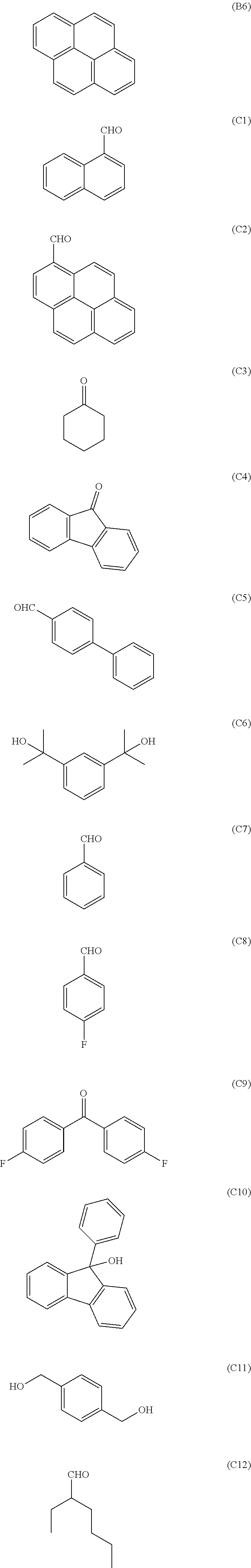

### (Catalysts D)

p-Toluenesulfonic acid monohydrate: D1
Methanesulfonic acid: D2

### (Solvents E)

1,4-Dioxane: E1
Toluene: E2
Propylene glycol monomethyl ether acetate (=PGMEA): E3

### (Reprecipitation Solvent F)

Methanol: F1

### Synthesis Example 1

A flask was charged with 10.0 g of phenylnaphthylamine, 7.1 g of 1-naphthaldehyde, 0.9 g of p-toluenesulfonic acid monohydrate, and 21.0 g of 1,4-dioxane. Subsequently, the mixture was heated to 110° C. under nitrogen and allowed to react for about 12 hours. After the reaction was discontinued, the product was reprecipitated from methanol and was dried to give a resin (S1). The polystyrene-equivalent weight average molecular weight Mw measured by GPC was about 1,400. The resin obtained was dissolved into PGMEA, and ion exchange was performed for 4 hours using a cation exchange resin and an anion exchange resin. A target compound solution was thus obtained.

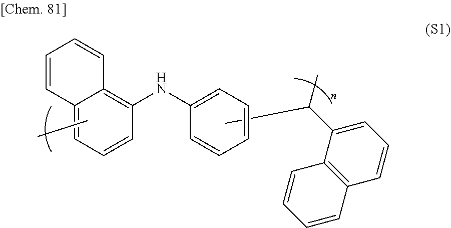

### Synthesis Examples 2 to 15

Polymers for use in resist underlayer films were synthesized while changing the type of the compounds A, the compounds B, the compounds C, the catalysts D, the solvents E, and the reprecipitation solvent F. The experimental procedures were the same as in Synthesis Example 1. The conditions adapted in the synthesis of polymers (S1) to (S15) are shown below.

TABLE 1

| | Structural | | | | | Re- |
|---|---|---|---|---|---|---|
| Syn. Ex. | formula | Compounds | Catalysts | Solvents | Temp./time | precipitation |
| 1 | S1 | A1/C1 | D1 | E1 | 110° C./ | F1 |
| | | 10.0 g/7.1 g | 0.9 g | 21.0 g | 12 hr | |
| 2 | S2 | A1/C2 | D1 | E1/E2 | Reflux/ | F1 |
| | | 8.0 g/8.5 g | 1.0 g | 16.3 g/ | 20 hr | |
| | | | | 16.3 g | | |
| 3 | S3 | A2/C3/C4 | D2 | — | Reflux/ | F1 |
| | | 10.0 g/10.7 g/ | 2.8 g | | 2.5 hr | |
| | | 23.5 g | | | | |
| 4 | S4 | A2/C5 | D2 | E3 | 115° C. | F1 |
| | | 10.0 g/10.8 g | 0.3 g | 63.2 g | 4 hr | |
| 5 | S5 | A3/C6 | D2 | E3 | Reflux/ | F1 |
| | | 65.0 g/55.3 g | 8.2 g | 192.7 g | 5 hr | |
| 6 | S6 | A4/C1 | D2 | E3 | 120° C./ | F1 |
| | | 35.0 g/32.7 g | 2.0 g | 162.7 g | 7 hr | |
| 7 | S7 | A3/C4 | D2 | E3 | Reflux/ | F1 |
| | | 8.0 g/8.6 g | 2.3 g | 18.9 g | 1.5 hr | |
| 8 | S8 | A5/C7 | D2 | E3 | Reflux/ | F1 |
| | | 8.0 g/4.4 g | 0.6 g | 30.2 g | 3 hr | |
| 9 | S9 | A6/C5 | D2 | E3 | 100° C./ | F1 |
| | | 10.0 g/7.4 g | 0.4 g | 53.5 g | 15 hr | |
| 10 | S10 | A2/B3/B4/C8 | D2 | E3 | Reflux/ | F1 |
| | | 15.0 g/1.8 g/ | 0.6 g | 168.0 g | 4 hr | |
| | | 8.8 g/15.7 g | | | | |
| 11 | S11 | A2/B1/B2/C8 | D2 | E3 | Reflux/ | F1 |
| | | 15.0 g/2.4 g/ | 0.6 g | 170.3 g | 4 hr | |
| | | 8.9 g/15.7 g | | | | |
| 12 | S12 | A2/B5/C8/C9 | D2 | E3 | Reflux/ | F1 |
| | | 60.0 g/10.1 g/ | 2.4 g | 630.2 g | 4 hr | |
| | | 62.9 g/22.1 g | | | | |
| 13 | S13 | A2/A7/B6/C8 | D2 | E3 | Reflux/ | F1 |
| | | 70.0 g/13.9 g/ | 2.8 g | 53.5 g | 4 hr | |
| | | 112.0 g/73.4 g | | | | |
| 14 | S14 | A2/C8/C10/C11 | D2 | E3 | Reflux/ | F1 |
| | | 70.0 g/41.1 g/ | 1.7 g | 688.4 g | 5 hr | |
| | | 5.3 g/8.6 g | | | | |
| 15 | S15 | A2/C1/C8/C12 | D2 | E3 | Reflux/ | F1 |
| | | 100.0 g/9.2 g/ | 2.8 g | 675.6 g | 4 hr | |
| | | 66.1 g/7.6 g | | | | |

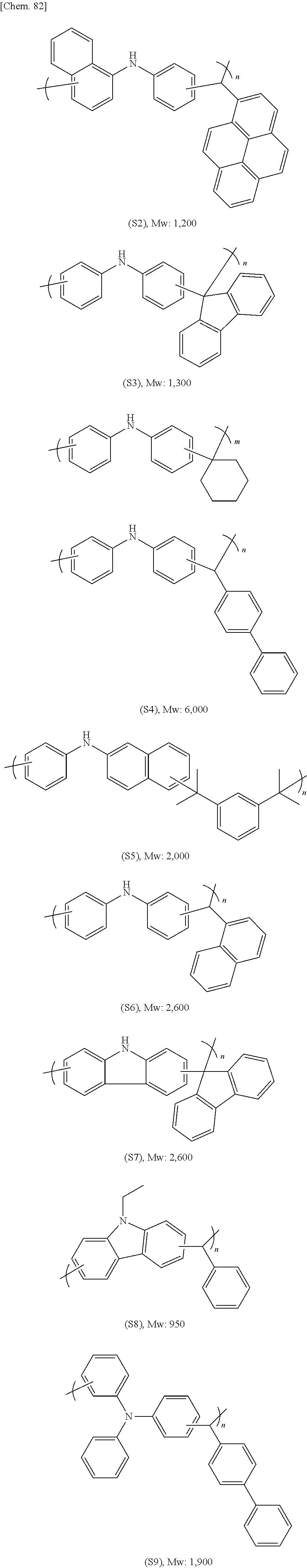

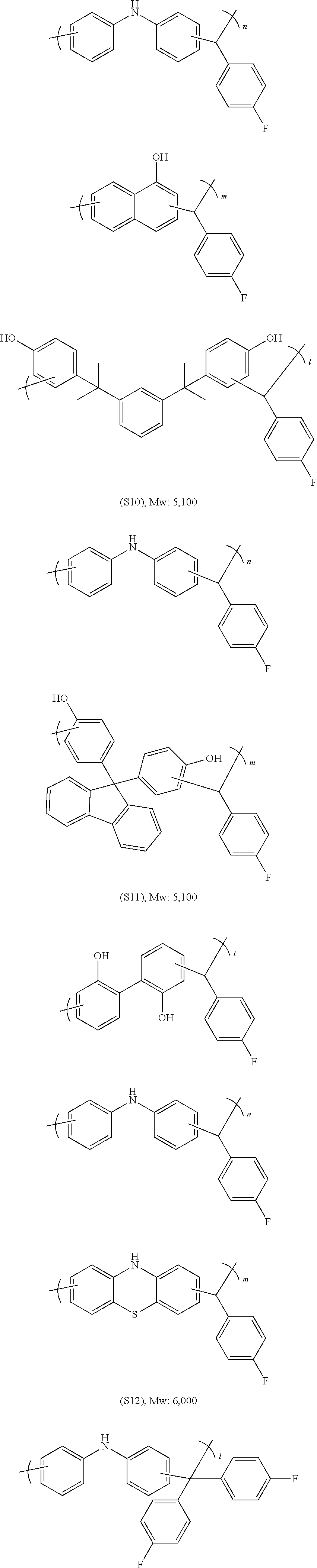

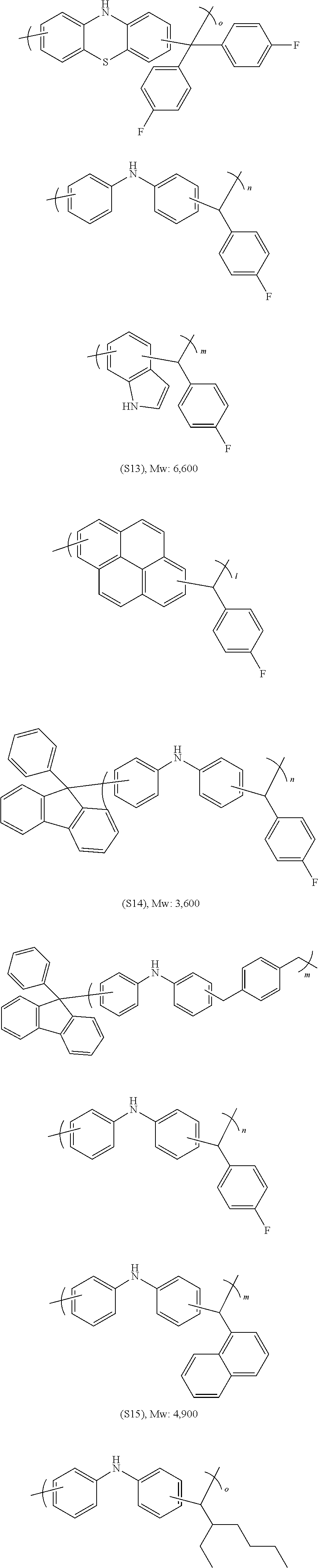

### Synthesis of Acid Generators

Acid compounds, base compounds, and solvents E shown below were used for the synthesis of acid generators with structural formulas (S16) to (S43) for use in resist underlayer films.

### (Acid Compounds)

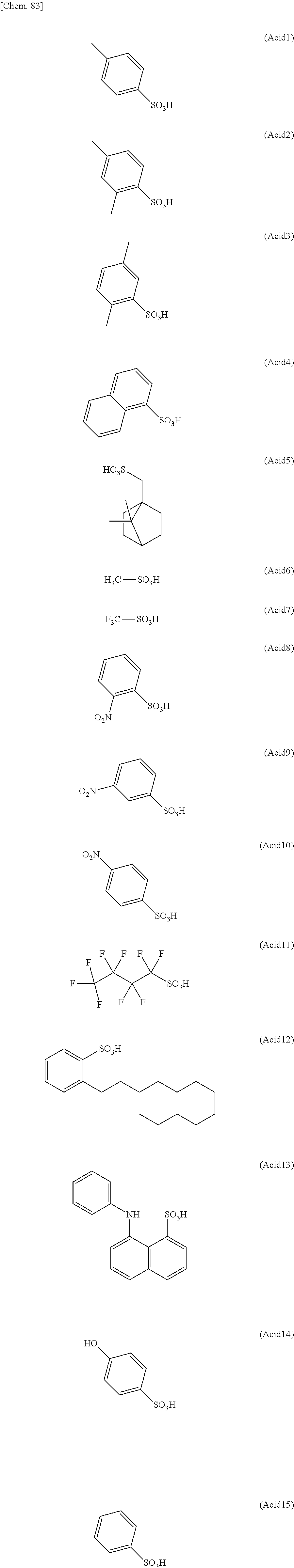

### (Base Compounds)

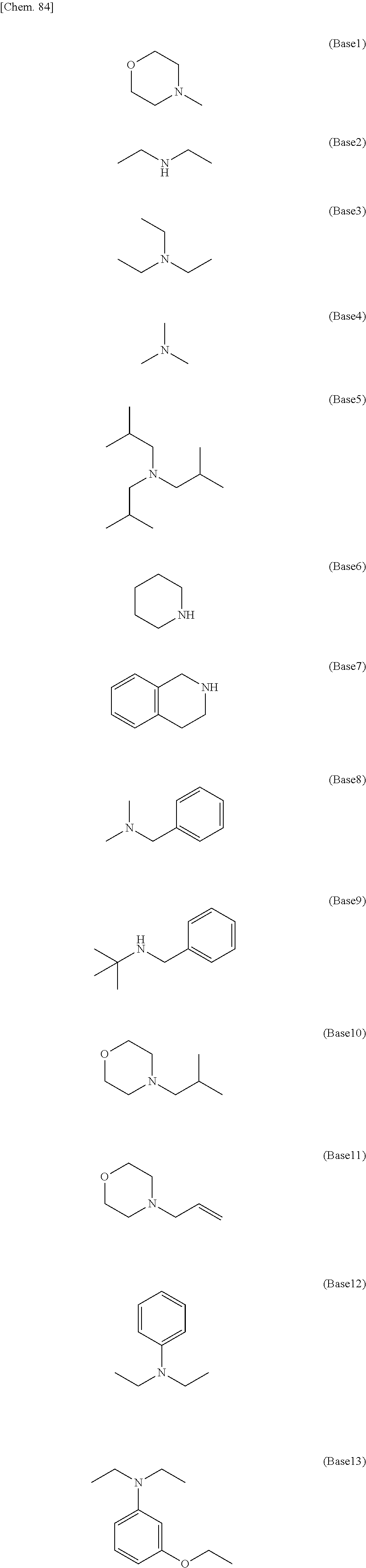

### (Solvents E)

Isopropyl alcohol: E4
Pure water: E5
Methanol: E6
Propylene glycol monomethyl ether (=PGME): E7

### Synthesis Example 16

A flask was charged with 2.9 g of N-methylmorpholine, 5.0 g of p-toluenesulfonic acid monohydrate, and 18.5 g of isopropyl alcohol. Subsequently, the mixture was heated to 40° C. and allowed to react for about 12 hours. After the reaction was discontinued, the product was distilled with an evaporator under reduced pressure at 50° C. until a constant weight was reached. The crystal of the target product was thus obtained.

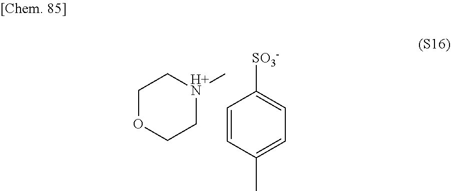

### Synthesis Example 16 to Synthesis Example 43

Acid generators for use in resist underlayer films were synthesized while changing the type of the acid compounds, the base compounds, and the solvents E. The experimental procedures were the same as in Synthesis Example 16. The acid generators that did not crystallize during the distillation under reduced pressure were recovered in a form of acid generator solution. The conditions adapted in the synthesis of acid generators (S16) to (S43) are shown below.

TABLE 2

| Syn. | Structural | | | |
|---|---|---|---|---|
| Ex. | formula | Compounds | Solvents | Temp./time |
| 16 | S16 | Acid1/Base1 | E4 | 40° C./12 hr |
| | | 5.0 g/2.9 g | 18.5 g | |
| 17 | S17 | Acid2/Base1 | E4 | 40° C./12 hr |
| | | 5.0 g/2.7 g | 18.0 g | |
| 18 | S18 | Acid3/Base1 | E4 | 40° C./12 hr |
| | | 5.0 g /2.7 g | 18.0 g | |
| 19 | S19 | Acid4/Base1 | E4 | 40° C./12 hr |
| | | 5.0 g/2.4 g | 17.3 g | |
| 20 | S20 | Acid5/Base1 | E 4 | 40° C./12 hr |
| | | 5.0 g/2.2 g | 16.7 g | |
| 21 | S21 | Acid6/Base1 | E 4 | 40° C./12 hr |
| | | 5.0 g/5.3 g | 23.9 g | |
| 22 | S22 | Acid7/Base1 | E4 | Room temp./12 hr |
| | | 5.0 g/3.4 g | 19.5 g | |
| 23 | S23 | Acid8/Base1 | E5 | 40° C./12 hr |
| | | 5.0 g/2.5 g | 30.0 g | |
| 24 | S24 | Acid9/Base1 | E5 | 40° C./12 hr |
| | | 5.0 g/2.5 g | 30.0 g | |
| 25 | S25 | Acid10/Base1 | E5 | 40° C./12 hr |
| | | 5.0 g /2.5 g | 30.0 g | |
| 26 | S26 | Acid11/Base1 | E 4 | Room temp./12 hr |
| | | 5.0 g/1.9 g | 61.7 g | |
| 27 | S27 | Acid12/Basa1 | E4 | 40° C./12 hr |
| | | 5.0 g/1.6 g | 26.2 g | |
| 28 | S28 | Acid13/Base1 | E4 | 40° C./12 hr |
| | | 5.0 g/1.6 g | 60.2 g | |
| 29 | S29 | Acid14/Base1 | E 4 | 40° C./12 hr |
| | | 5.0 g/2.9 g | 18.4 g | |
| 30 | S30 | Acid15/Base1 | E4 | 40° C./12 hr |
| | | 5.0 g/3.2 g | 19.1 g | |
| 31 | S31 | Acid1/Base2 | E4 | 40° C./6 hr |
| | | 5.0 g/2.2 g | 16.7 g | |
| 32 | S32 | Acid1/Base3 | E4 | 40° C./12 hr |
| | | 5.0 g/3.0 g | 18.7 g | |
| 33 | S33 | Acid1/Base4 | E6 | 40° C./12 hr |
| | | 5.0 g/1.8 g | 15.8 g | |
| 34 | S34 | Acid1/Base5 | E4 | 40° C./12 hr |
| | | 5.0 g/5.5 g | 24.5 g | |
| 35 | S35 | Acid1/Base6 | E4 | 40° C./7 hr |
| | | 5.0 g/2.5 g | 17.6 g | |
| 36 | S36 | Acid1/Base7 | E4/E7 | 40° C./6 hr |
| | | 5.0 g/3.9 g | 10.4 g/10.4 g | |
| 37 | S37 | Acid1/Base8 | E4 | 40° C./6 hr |
| | | 5.0 g/4.0 g | 21.0 g | |
| 38 | S38 | Acid1/Base8 | E6 | 40° C./6 hr |
| | | 5.0 g/4.8 g | 23.0 g | |
| 39 | S39 | Acid1/Base9 | E4 | 40° C./7 hr |
| | | 5.0 g/2.6 g | 17.7 g | |
| 40 | S40 | Acid1/Base10 | E4 | 40° C./12 hr |
| | | 5.0 g/4.2 g | 21.6 g | |
| 41 | S41 | Acid1/Base11 | E4 | 40° C./7 hr |
| | | 5.0 g/3.8 g | 20.5 g | |
| 42 | S42 | Acid1/Base12 | E4 | 40° C./6 hr |
| | | 5.0 g/4.4 g | 22.0 g | |
| 43 | S43 | Acid1/Base13 | E4 | 40° C./6 hr |
| | | 5.0 g/5.7 g | 25.0 g | |

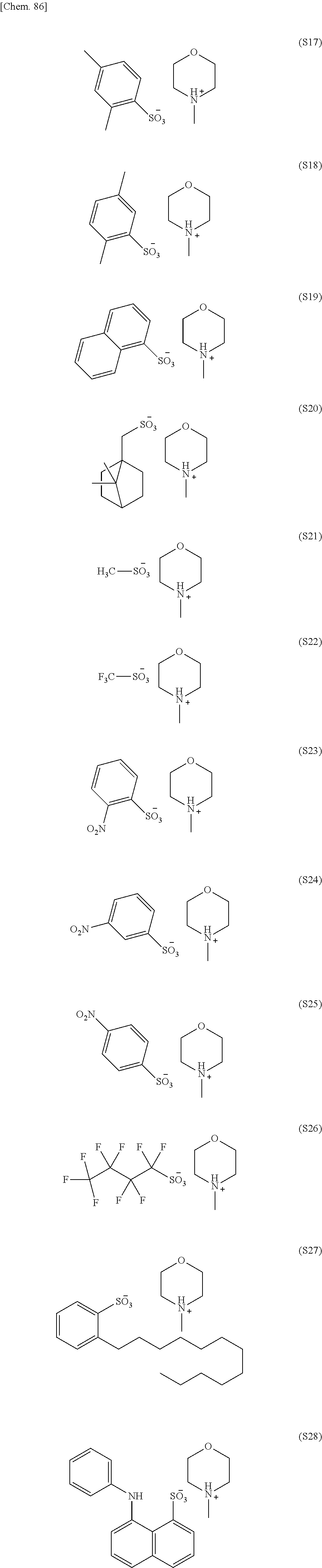

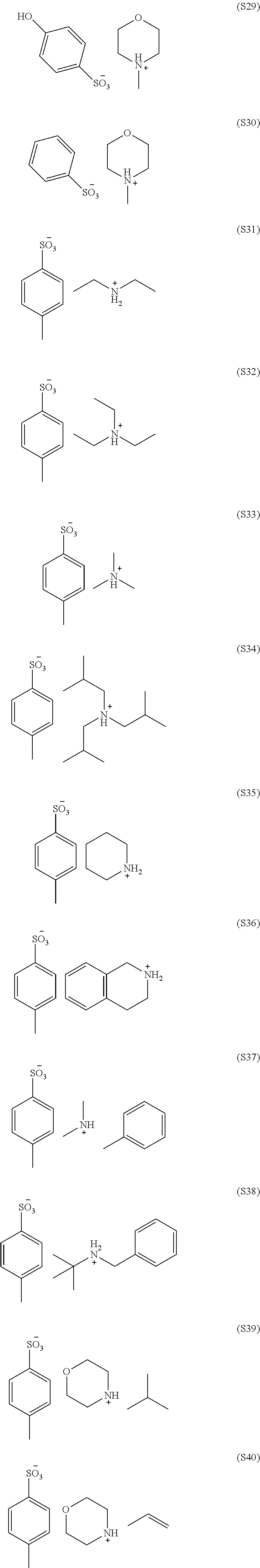

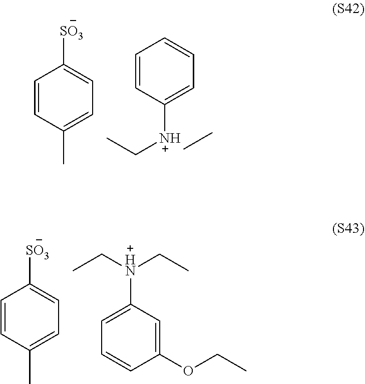

### [Preparation of Resist Underlayer Films]

The polymers (S1) to (S15), crosslinking agents (CR1 and CR2), the acid generators (Ad1 to Ad3, and S16 to S43), solvents (propylene glycol monomethyl ether acetate (PGMEA), propylene glycol monomethyl ether (PGME), cyclohexanone (CYH)), and MEGAFACE R-40 (manufactured by DIC CORPORATION, G1) as a surfactant were mixed in proportions shown in the tables below (the values are expressed by part by mass). The mixtures were filtered through a 0.1 m polytetrafluoroethylene microfilter. Resist underlayer film materials (M1 to M43 and Comparative M1 to Comparative M18) were thus prepared.

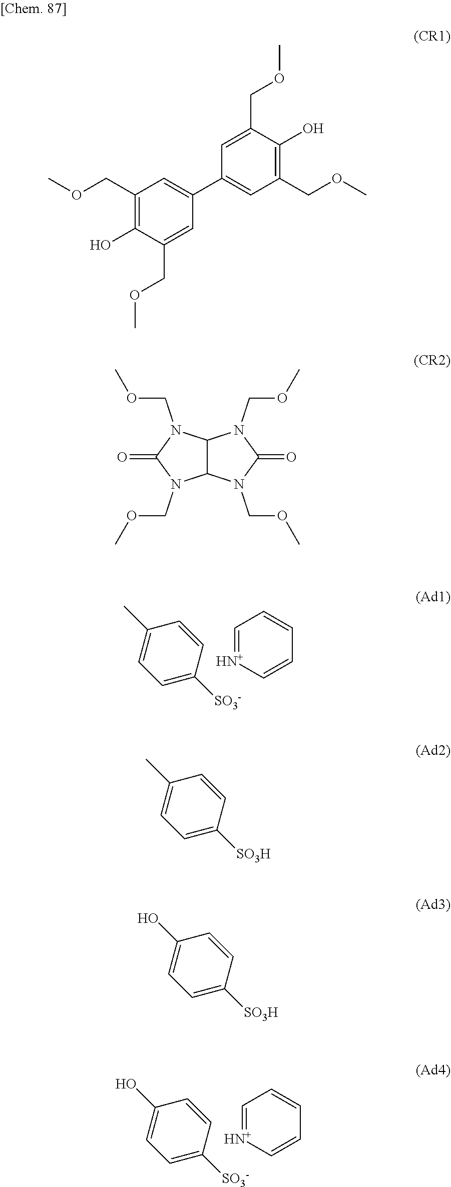

TABLE 3

| | | Crosslinking | Acid | | Solvents | | |
|---|---|---|---|---|---|---|---|
| Composition | Polymer | agent | generator | Surfactant | (100 parts by mass in total) | | |
| M1 | Syn. Ex. 1 | CR1 | S16 | G1 | PGMEA | PGME | CYH |
| | 100 | 35 | 3 | 0.1 | 40 | 40 | 20 |
| M2 | Syn. Ex. 1 | CR1 | S31 | G1 | PGMEA | PGME | CYH |
| | 100 | 35 | 3 | 0.1 | 40 | 40 | 20 |
| M3 | Syn. Ex. 1 | CR1 | S32 | G1 | PGMEA | PGME | CYH |
| | 100 | 35 | 3 | 0.1 | 40 | 40 | 20 |
| M4 | Syn. Ex. 1 | CR1 | S33 | G1 | PGMEA | PGME | CYH |
| | 100 | 35 | 3 | 0.1 | 40 | 40 | 20 |
| M5 | Syn. Ex. 1 | CR1 | S34 | G1 | PGMEA | PGME | CYN |
| | 100 | 35 | 3 | 0.1 | 40 | 40 | 20 |
| M6 | Syn. Ex. 1 | CR1 | S35 | G1 | PGMEA | PGME | CYH |
| | 100 | 35 | 3 | 0.1 | 40 | 40 | 20 |
| M7 | Syn. Ex. 1 | CR1 | S36 | G1 | PGMEA | PGME | CYH |
| | 100 | 35 | 3 | 0.1 | 40 | 40 | 20 |
| M8 | Syn. Ex. 1 | CR1 | S37 | G1 | PGMEA | PGME | CYH |
| | 100 | 35 | 3 | 0.1 | 40 | 40 | 20 |
| M9 | Syn. Ex. 1 | CR1 | S38 | G1 | PGMEA | PGME | CYH |
| | 100 | 35 | 3 | 0.1 | 40 | 40 | 20 |
| M10 | Syn. Ex. 1 | CR1 | S39 | G1 | PGMEA | PGME | CYN |
| | 100 | 35 | 3 | 0.1 | 40 | 40 | 20 |
| M11 | Syn. Ex. 1 | CR1 | S40 | G1 | PGMEA | PGME | CYH |
| | 100 | 35 | 3 | 0.1 | 40 | 40 | 20 |
| M12 | Syn. Ex. 1 | CR1 | S41 | G1 | PGMEA | PGME | CYH |
| | 100 | 35 | 3 | 0.1 | 40 | 40 | 20 |
| M13 | Syn. Ex. 1 | CR1 | S42 | G1 | PGMEA | PGME | CYH |
| | 100 | 35 | 3 | 0.1 | 40 | 40 | 20 |
| M14 | Syn. Ex. 1 | CR1 | S43 | G1 | PGMEA | PGME | CYH |
| | 100 | 35 | 3 | 0.1 | 40 | 40 | 20 |
| M15 | Syn. Ex. 2 | CR2 | S16 | G1 | PGMEA | PGME | CYH |
| | 100 | 20 | 3 | 0.1 | 40 | 30 | 50 |
| M16 | Syn. Ex. 3 | CR1 | S29 | G1 | PGMEA | PGME | CYH |
| | 100 | 35 | 3.8 | 0.1 | 40 | 30 | 30 |
| M17 | Syn. Ex. 4 | CR2 | S16 | G1 | PGMEA | PGME | CYH |
| | 100 | 25 | 3.75 | 0.1 | 40 | 30 | 30 |
| M18 | Syn. Ex. 4 | CR2 | S17 | G1 | PGMEA | PGME | CYH |
| | 100 | 25 | 3.75 | 0.1 | 40 | 30 | 30 |
| M19 | Syn. Ex. 4 | CR2 | S18 | G1 | PGMEA | PGME | CYH |
| | 100 | 25 | 3.75 | 0.1 | 40 | 30 | 30 |
| M20 | Syn. Ex. 4 | CR2 | S19 | G1 | PGMEA | PGME | CYH |
| | 100 | 25 | 3.75 | 0.1 | 40 | 30 | 30 |
| M21 | Syn. Ex. 4 | CR2 | S20 | G1 | PGMEA | PGME | CYH |
| | 100 | 25 | 3.75 | 0.1 | 40 | 30 | 30 |
| M22 | Syn. Ex. 4 | CR2 | S21 | G1 | PGMEA | PGME | CYH |
| | 100 | 25 | 3.75 | 0.1 | 40 | 30 | 30 |
| M23 | Syn. Ex. 4 | CR2 | S22 | G1 | PGMEA | PGME | CYH |
| | 100 | 25 | 3.75 | 0.1 | 40 | 30 | 30 |
| M24 | Syn. Ex. 4 | CR2 | S23 | G1 | PGMEA | PGME | CYH |
| | 100 | 25 | 3.75 | 0.1 | 40 | 30 | 30 |
| M25 | Syn. Ex. 4 | CR2 | S24 | G1 | PGMEA | PGME | CYH |
| | 100 | 25 | 3.75 | 0.1 | 40 | 30 | 30 |
| M26 | Syn. Ex. 4 | CR2 | S25 | G1 | PGMEA | PGME | CYH |
| | 100 | 25 | 3.75 | 0.1 | 40 | 30 | 30 |
| M27 | Syn. Ex. 4 | CR2 | S26 | G1 | PGMEA | PGME | CYH |
| | 100 | 25 | 3.75 | 0.1 | 40 | 30 | 30 |
| M28 | Syn. Ex. 4 | CR2 | S27 | G1 | PGMEA | PGME | CYH |
| | 100 | 25 | 3.75 | 0.1 | 40 | 30 | 30 |
| M29 | Syn. Ex. 4 | CR2 | S28 | G1 | PGMEA | PGME | CYH |
| | 100 | 25 | 3.75 | 0.1 | 40 | 30 | 30 |
| M30 | Syn. Ex. 4 | CR2 | S29 | G1 | PGMEA | PGME | CYH |
| | 100 | 25 | 3.75 | 0.1 | 40 | 30 | 30 |

TABLE 4

| | | Crosslinking | Acid | | Solvents | | |
|---|---|---|---|---|---|---|---|
| Composition | Polymer | agent | generator | Surfactant | (100 parts by mass in total) | | |
| M31 | Syn. Ex. 4 | CR2 | S30 | G1 | PGMEA | PGME | CYH |
| | 100 | 25 | 3.75 | 0.1 | 40 | 30 | 30 |
| M32 | Syn. Ex. 5 | CR2 | S16 | G1 | PGMEA | PGME | CYH |
| | 100 | 25 | 3.75 | 0.1 | 40 | 30 | 30 |
| M33 | Syn. Ex 6 | CR2 | S16 | G1 | PGMEA | PGME | CYH |
| | 100 | 20 | 3 | 0.1 | 70 | 30 | 0 |
| M34 | Syn. Ex. 7 | CR2 | S16 | G1 | PGMEA | PGME | CYH |
| | 100 | 20 | 3 | 0.1 | 40 | 40 | 20 |
| M35 | Syn. Ex. 8 | CR1 | S29 | G1 | PGMEA | PGME | CYH |
| | 100 | 30 | 3.75 | 0.1 | 20 | 20 | 60 |
| M36 | Syn. Ex. 9 | CR1 | S29 | G1 | PGMEA | PGME | CYH |
| | 100 | 30 | 3.75 | 0.1 | 10 | 20 | 70 |
| M37 | Syn. Ex. 1 | CR1 | S16 | G1 | PGMEA | PGME | CYH |
| | 100 | 20 | 3 | 0.1 | 40 | 40 | 20 |
| M38 | Syn. Ex. 10 | CR1 | S16 | G1 | PGMEA | PGME | CYH |
| | 100 | 20 | 3 | 0.1 | 70 | 30 | 0 |
| M39 | Syn. Ex. 11 | CR1 | S16 | G1 | PGMEA | PGME | CYH |
| | 100 | 20 | 3 | 0.7 | 70 | 30 | 0 |
| M40 | Syn. Ex. 12 | CR1 | S16 | G1 | PGMEA | PGME | CYH |
| | 100 | 20 | 3 | 0.1 | 70 | 30 | 0 |
| M41 | Syn. Ex. 13 | CR1 | S16 | G1 | PGMEA | PGME | CYH |
| | 100 | 20 | 3 | 0.1 | 70 | 30 | 0 |
| M42 | Syn. Ex. 14 | CR1 | S16 | G1 | PGMEA | PGME | CYH |
| | 100 | 20 | 3 | 0.1 | 70 | 30 | 0 |
| M43 | Syn. Ex. 15 | CR1 | S16 | G1 | PGMEA | PGME | CYH |
| | 100 | 20 | 3 | 0.1 | 70 | 30 | 0 |

TABLE 5

| | | Crosslinking | Acid | | Solvents | | |
|---|---|---|---|---|---|---|---|
| Composition | Polymer | agent | generator | Surfactant | (100 parts by mass in total) | | |
| Comp. M1 | Syn. Ex. 1 | CR1 | Ad1 | G1 | PGMEA | PGME | CYH |
| | 100 | 35 | 3 | 0.1 | 40 | 40 | 20 |
| Comp. M2 | Syn. Ex. 2 | CR2 | Ad1 | G1 | PGMEA | PGME | CYH |
| | 100 | 20 | 3 | 0.1 | 40 | 10 | 50 |
| Comp. M3 | Syn. Ex. 3 | CR1 | Ad4 | G1 | PGMEA | PGME | CYH |
| | 100 | 35 | 3.8 | 0.1 | 40 | 30 | 30 |
| Comp. M4 | Syn. Ex. 4 | CR2 | Ad1 | G1 | PGMEA | PGME | CYH |
| | 100 | 25 | 3.75 | 0.1 | 40 | 30 | 30 |
| Comp. M5 | Syn. Ex. 4 | CR2 | Ad2 | G1 | PGMEA | PGME | CYH |
| | 100 | 25 | 3.75 | 0.1 | 40 | 30 | 30 |
| Comp. M6 | Syn. Ex. 4 | CR2 | Ad3 | G1 | PGMEA | PGME | CYH |
| | 100 | 25 | 3.75 | 0.1 | 40 | 30 | 30 |
| Comp. M7 | Syn. Ex. 5 | CR2 | Ad1 | G1 | PGMEA | PGME | CYH |
| | 100 | 25 | 3.75 | 0.1 | 40 | 30 | 30 |
| Comp. M8 | Syn. Ex. 6 | CR2 | Ad1 | G1 | PGMEA | PGME | CYH |
| | 100 | 20 | 3 | 0.1 | 70 | 30 | 0 |
| Comp. M9 | Syn. Ex. 7 | CR2 | Ad1 | G1 | PGMEA | PGME | CYH |
| | 100 | 20 | 3 | 0.1 | 40 | 40 | 20 |
| Comp. M10 | Syn. Ex. 8 | CR1 | Ad4 | G1 | PGMEA | PGME | CYH |
| | 100 | 30 | 3.75 | 0.1 | 20 | 20 | 60 |
| Comp. M11 | Syn. Ex. 9 | CR1 | Ad4 | G1 | PGMEA | PGME | CYH |
| | 100 | 30 | 3.75 | 0.1 | 10 | 20 | 70 |
| Comp. M12 | Syn. Ex. 1 | CR1 | Ad1 | G1 | PGMEA | PGME | CYH |
| | 100 | 20 | 3 | 0.1 | 40 | 40 | 20 |
| Comp. M13 | Syn. Ex. 10 | CR1 | Ad1 | G1 | PGMEA | PGME | CYH |
| | 100 | 20 | 3 | 0.1 | 70 | 30 | 0 |
| Comp. M14 | Syn. Ex. 11 | CR1 | Ad1 | G1 | PGMEA | PGME | CYH |
| | 100 | 20 | 3 | 0.1 | 70 | 30 | 0 |
| Comp. M15 | Syn. Ex. 12 | CR1 | Ad1 | G1 | PGMEA | PGME | CYH |
| | 100 | 20 | 3 | 0.1 | 70 | 30 | 0 |

TABLE 6

| | | Crosslinking | Acid | | Solvents | | |
|---|---|---|---|---|---|---|---|
| Composition | Polymer | agent | generator | Surfactant | (100 parts by mass in total) | | |
| Comp. M16 | Syn. Ex. 13 | CR1 | Ad1 | G1 | PGMEA | PGME | CYH |
| | 100 | 20 | 3 | 0.1 | 70 | 30 | 0 |
| Comp. M17 | Syn. Ex. 14 | CR1 | Ad1 | G1 | PGMEA | PGME | CYH |
| | 100 | 20 | 3 | 0.1 | 70 | 30 | 0 |
| Comp. M18 | Syn. Ex. 15 | CR1 | Ad1 | G1 | PGMEA | PGME | CYH |
| | 100 | 20 | 3 | 0.1 | 70 | 30 | 0 |

[Test of Dissolution into Resist Solvent]

Each of the resist underlayer film materials prepared in Comparative Examples 1 to 18 and Examples 1 to 43 was applied onto a silicon wafer using a spin coater, and each of the resultant coatings was baked on a hot plate at 240° C. for 60 seconds to form a resist underlayer film with a film thickness of about 120 nm. The resist underlayer films formed were soaked in a general-purpose thinner, specifically, PGME/PGMEA=7/3, for 60 seconds to examine the resistance to the solvent. The evaluation was made as o when the loss in film thickness after the thinner immersion was 1% or less, and as x when the loss in film thickness was more than 1%.

### [Test of Storage Stability of Resist Underlayer Film Materials]

Samples were prepared so that the total solid content in each of the resist underlayer film materials of Comparative Examples 1 to 18 and Examples 1 to 43 would be 3%. Each of the samples was placed in a screw tube and was stored in a thermostatic chamber at 35° C. under dark conditions for one week. After one week, the color of the samples was visually compared to that before the storage. The storage stability was evaluated as x when the color had changed, and as o when the color remained unchanged.

TABLE 7

| | | | | Change in |
|---|---|---|---|---|
| Ex./Comp. | | | Solvent | solution |
| Ex. | Composition | Baking temp. | resistance | color |
| Ex. 1 | M1 | 240° C./60 sec | ∘ | ∘ |
| Ex. 2 | M2 | 240° C./60 sec | ∘ | ∘ |
| Ex. 3 | M3 | 240° C./60 sec | ∘ | ∘ |
| Ex. 4 | M4 | 240° C./60 sec | ∘ | ∘ |
| Ex. 5 | M5 | 240° C./60 sec | ∘ | ∘ |
| Ex. 6 | M6 | 240° C./60 sec | ∘ | ∘ |
| Ex. 7 | M7 | 240° C./60 sec | ∘ | ∘ |
| Ex. 8 | M8 | 240° C./60 sec | ∘ | ∘ |
| Ex. 9 | M9 | 240° C./60 sec | ∘ | ∘ |
| Ex. 10 | M10 | 240° C./60 sec | ∘ | ∘ |
| Ex. 11 | M11 | 240° C./60 sec | ∘ | ∘ |
| Ex. 12 | M12 | 240° C./60 sec | ∘ | ∘ |
| Ex. 13 | M13 | 240° C./60 sec | ∘ | ∘ |
| Ex. 14 | M14 | 240° C./60 sec | ∘ | ∘ |
| Ex. 15 | M15 | 240° C./60 sec | ∘ | ∘ |
| Ex. 16 | M16 | 240° C./60 sec | ∘ | ∘ |
| Ex. 17 | M17 | 240° C./60 sec | ∘ | ∘ |
| Ex. 18 | M18 | 240° C./60 sec | ∘ | ∘ |
| Ex. 19 | M19 | 240° C./60 sec | ∘ | ∘ |
| Ex. 20 | M20 | 240° C./60 sec | ∘ | ∘ |
| Ex. 21 | M21 | 240° C./60 sec | ∘ | ∘ |
| Ex. 22 | M22 | 240° C./60 sec | ∘ | ∘ |
| Ex. 23 | M23 | 240° C./60 sec | ∘ | ∘ |
| Ex. 24 | M24 | 240° C./60 sec | ∘ | ∘ |
| Ex. 25 | M25 | 240° C./60 sec | ∘ | ∘ |
| Ex. 26 | M26 | 240° C./60 sec | ∘ | ∘ |
| Ex. 27 | M27 | 240° C./60 sec | ∘ | ∘ |
| Ex. 28 | M28 | 240° C./60 sec | ∘ | ∘ |
| Ex. 29 | M29 | 240° C./60 sec | ∘ | ∘ |
| Ex. 30 | M30 | 240° C./60 sec | ∘ | ∘ |
| Ex. 31 | M31 | 240° C./60 sec | ∘ | ∘ |
| Ex. 32 | M32 | 240° C./60 sec | ∘ | ∘ |
| Ex. 33 | M33 | 240° C./60 sec | ∘ | ∘ |
| Ex. 34 | M34 | 240° C./60 sec | ∘ | ∘ |
| Ex. 35 | M35 | 240° C./60 sec | ∘ | ∘ |
| Ex. 36 | M36 | 240° C./60 sec | ∘ | ∘ |
| Ex. 37 | M37 | 240° C./60 sec | ∘ | ∘ |
| Ex. 38 | M38 | 240° C./60 sec | ∘ | ∘ |
| Ex. 39 | M39 | 240° C./60 sec | ∘ | ∘ |
| Ex. 40 | M40 | 240° C./60 sec | ∘ | ∘ |
| Ex. 41 | M41 | 240° C./60 sec | ∘ | ∘ |
| Ex. 42 | M42 | 240° C./60 sec | ∘ | ∘ |
| Ex. 43 | M43 | 240° C./60 sec | ∘ | ∘ |
| Comp. Ex. 1 | Comp. M1 | 240° C./60 sec | ∘ | x |
| Comp. Ex. 2 | Comp. M2 | 240° C./60 sec | ∘ | x |
| Comp. Ex. 3 | Comp. M3 | 240° C./60 sec | ∘ | x |
| Comp. Ex. 4 | Comp. M4 | 240° C./60 sec | ∘ | x |
| Comp. Ex. 5 | Comp. M5 | 240° C./60 sec | ∘ | x |
| Comp. Ex. 6 | Comp. M6 | 240° C./60 sec | ∘ | x |
| Comp. Ex. 7 | Comp. M7 | 240° C./60 sec | ∘ | x |
| Comp. Ex. 8 | Comp. M8 | 240° C./60 sec | ∘ | x |
| Comp. Ex. 9 | Comp. M9 | 240° C./60 sec | ∘ | x |
| Comp. Ex. 10 | Comp. M10 | 240° C./60 sec | ∘ | x |
| Comp. Ex. 11 | Comp. M11 | 240° C./60 sec | ∘ | x |
| Comp. Ex. 12 | Comp. M12 | 240° C./60 sec | ∘ | x |
| Comp. Ex. 13 | Comp. M13 | 240° C./60 sec | ∘ | x |
| Comp. Ex. 14 | Comp. M14 | 240° C./60 sec | ∘ | x |
| Comp. Ex. 15 | Comp. M15 | 240° C./60 sec | ∘ | x |
| Comp. Ex. 16 | Comp. M16 | 240° C./60 sec | ∘ | x |
| Comp. Ex. 17 | Comp. M17 | 240° C./60 sec | ∘ | x |
| Comp. Ex. 18 | Comp. M18 | 240° C./60 sec | ∘ | x |

### [Evaluation of Gap-Filling Property]

Gap-filling property was evaluated using 200 nm thick SiO₂ substrate, SiN substrate, and TiN substrate, which had a dense pattern area consisting of 50 nm wide trenches at 100 nm pitches. Each of the resist underlayer film materials prepared in Comparative Examples 1 to 19 and Examples 1 to 44 was applied onto the pattern, and each of the coatings was baked at 240° C. for 60 seconds to form a resist underlayer film having a thickness of about 120 nm. The flattening property to the substrates was evaluated using a scanning electron microscope (S-4800) manufactured by Hitachi High-Tech Corporation, and whether the resist underlayer film-forming composition had filled the inside of the pattern was determined. The gap-filling property was rated as o when the resist underlayer film-forming composition had filled the inside of the pattern, and as x when the resist underlayer film-forming composition had failed to fill the inside of the pattern.

### [Test of Covering Performance on Non-Planar Substrate)

To test the covering performance on a non-planar substrate, each of the resist underlayer film-forming compositions prepared in Comparative Examples 1 to 19 and Examples 1 to 44 was applied to 200 nm thick SiO₂ substrate, SiN substrate, and TiN substrate, and each of the resultant coatings was baked at 240° C. for 60 seconds to form a resist underlayer film having a thickness of about 120 nm. The coating film thickness was compared between at an 800 nm trenched area (TRENCH) and at an open area (OPEN) free from patterns. The flatness of the substrates was evaluated using a scanning electron microscope (S-4800) manufactured by Hitachi High-Tech Corporation by measuring the difference in film thickness between on the trenched area (the patterned area) and on the open area (the pattern-free area) of the non-planar substrate (the step height created on the coating film between the trenched area and the open area, called the bias). Here, the flatness means how small the difference is in the film thickness (the iso-dense bias) of the coating film between on the region with the pattern (the trenched area (the patterned area)) and on the region without patterns (the open area (the pattern-free area)). Flattening property was rated as o when the bias was improved compared to Comparative Examples.

TABLE 8

| | | Film | | | |
|---|---|---|---|---|---|
| Ex./Comp. | | thick- | Sub- | Gap-filling | Flattening |
| Ex. | Composition | ness | strate | properties | properties |
| Ex. 1 | M1 | 120 nm | TiN | ∘ | ∘ |
| Ex. 2 | M2 | 120 nm | TiN | ∘ | ∘ |
| Ex. 3 | M3 | 120 nm | TiN | ∘ | ∘ |
| Ex. 4 | M4 | 120 nm | TiN | ∘ | ∘ |
| Ex. 5 | M5 | 120 nm | TiN | ∘ | ∘ |
| Ex. 6 | M6 | 120 nm | TiN | ∘ | ∘ |
| Ex. 7 | M7 | 120 nm | TiN | ∘ | ∘ |
| Ex. 8 | M8 | 120 nm | TiN | ∘ | ∘ |
| Ex. 9 | M9 | 120 nm | TiN | ∘ | ∘ |
| Ex. 10 | M10 | 120 nm | TiN | ∘ | ∘ |
| Ex. 11 | M11 | 120 nm | TiN | ∘ | ∘ |
| Ex. 12 | M12 | 120 nm | TiN | ∘ | ∘ |
| Ex. 13 | M13 | 120 nm | TiN | ∘ | ∘ |
| Ex. 14 | M14 | 120 nm | TiN | ∘ | ∘ |
| Ex. 15 | M15 | 120 nm | SiO₂ | ∘ | ∘ |
| Ex. 16 | M16 | 120 nm | SiN | ∘ | ∘ |
| Ex. 17 | M17 | 120 nm | SiO₂ | ∘ | ∘ |
| Ex. 18 | M18 | 120 nm | SiO₂ | ∘ | ∘ |
| Ex. 19 | M19 | 120 nm | SiO₂ | ∘ | ∘ |
| Ex. 20 | M20 | 120 nm | SiO₂ | ∘ | ∘ |
| Ex. 21 | M21 | 120 nm | SiO₂ | ∘ | ∘ |
| Ex. 22 | M22 | 120 nm | SiO₂ | ∘ | ∘ |
| Ex. 23 | M23 | 120 nm | SiO₂ | ∘ | ∘ |
| Ex. 24 | M24 | 120 nm | SiO₂ | ∘ | ∘ |
| Ex. 25 | M25 | 120 nm | SiO₂ | ∘ | ∘ |
| Ex. 26 | M26 | 120 nm | SiO₂ | ∘ | ∘ |
| Ex. 27 | M27 | 120 nm | SiO₂ | ∘ | ∘ |
| Ex. 28 | M28 | 120 nm | SiO₂ | ∘ | ∘ |
| Ex. 29 | M29 | 120 nm | SiO₂ | ∘ | ∘ |
| Ex. 30 | M30 | 120 nm | SiO₂ | ∘ | ∘ |
| Ex. 31 | M31 | 120 nm | SiO₂ | ∘ | ∘ |
| Ex. 32 | M32 | 120 nm | SiO₂ | ∘ | ∘ |
| Ex. 33 | M33 | 120 nm | SiO₂ | ∘ | ∘ |
| Ex. 34 | M34 | 120 nm | SiO₂ | ∘ | ∘ |
| Ex. 35 | M35 | 120 nm | SiO₂ | ∘ | ∘ |
| Ex. 36 | M36 | 120 nm | SiO₂ | ∘ | ∘ |
| Ex. 37 | M37 | 120 nm | SiO₂ | ∘ | ∘ |
| Ex. 38 | M37 | 120 nm | SiN | ∘ | ∘ |
| Ex. 39 | M38 | 120 nm | SiO₂ | ∘ | ∘ |
| Ex. 40 | M39 | 120 nm | SiO₂ | ∘ | ∘ |
| Ex. 41 | M40 | 120 nm | SiO₂ | ∘ | ∘ |
| Ex. 42 | M41 | 120 nm | SiO₂ | ∘ | ∘ |
| Ex. 43 | M42 | 120 nm | SiO₂ | ∘ | ∘ |
| Ex. 44 | M43 | 120 nm | SiO₂ | ∘ | ∘ |
| Comp. Ex. 1 | Comp. M1 | 120 nm | TiN | ∘ | x |
| Comp. Ex. 2 | Comp. M2 | 120 nm | SiO₂ | ∘ | x |
| Comp. Ex. 3 | Comp. M3 | 120 nm | SiN | ∘ | x |
| Comp. Ex. 4 | Comp. M4 | 120 nm | SiO₂ | ∘ | x |
| Comp. Ex. 5 | Comp. M5 | 120 nm | SiO₂ | ∘ | x |
| Comp. Ex. 6 | Comp. M6 | 120 nm | SiO₂ | ∘ | x |
| Comp. Ex. 7 | Comp. M7 | 120 nm | SiO₂ | ∘ | x |
| Comp. Ex. 8 | Comp. M8 | 120 nm | SiO₂ | x | x |
| Comp. Ex. 9 | Comp. M9 | 120 nm | SiO₂ | x | x |
| Comp. Ex. 10 | Comp. M10 | 120 mm | SiO₂ | ∘ | x |
| Comp. Ex. 11 | Comp. M11 | 120 nm | SiO₂ | ∘ | x |
| Comp. Ex. 12 | Comp. M12 | 120 nm | SiO₂ | ∘ | x |
| Comp. Ex. 13 | Comp. M12 | 120 nm | SiN | ∘ | x |
| Comp. Ex. 14 | Comp. M13 | 120 nm | SiO₂ | ∘ | x |
| Comp. Ex. 15 | Comp. M14 | 120 nm | SiO₂ | ∘ | x |
| Comp. Ex. 16 | Comp. M15 | 120 nm | SiO₂ | ∘ | x |
| Comp. Ex. 17 | Comp. M16 | 120 nm | SiO₂ | ∘ | x |
| Comp. Ex. 18 | Comp. M17 | 120 nm | SiO₂ | ∘ | x |
| Comp. Ex. 19 | Comp. M18 | 120 nm | SiO₂ | ∘ | x |

As seen in the tables, the acid generators synthesized from an amine component having a higher basicity than pyridine provided a similar level of curability to the conventional pyridine salt-type acid generators. In addition, the acid generators using a highly basic amine component had a higher stability as a salt and were reluctant to release sulfonic acid in a solution. Thus, the polymer including an amine component was prevented from coloration by the action of sulfonic acid and achieved a significant improvement in storage stability. Furthermore, the highly basic amine components formed a strong salt and therefore the generation of an acid component could be retarded until a higher temperature to ensure a time for the resin to flow. As a result, a flatter film could be formed on a patterned substrate, and the resin could achieve an improved gap-filling property. The above advantageous effects were achieved equally on different types of patterned substrates, such as SiN, SiO₂, and TiN.

### Synthesis of Polymers

Compounds A′, compounds B′, catalysts C′, solvents D′, and reprecipitation solvents E′ shown below were used for the synthesis of polymers with structural formulas (S′1) to (S′11) for use in resist underlayer films.

### (Compounds A‘ and B’)

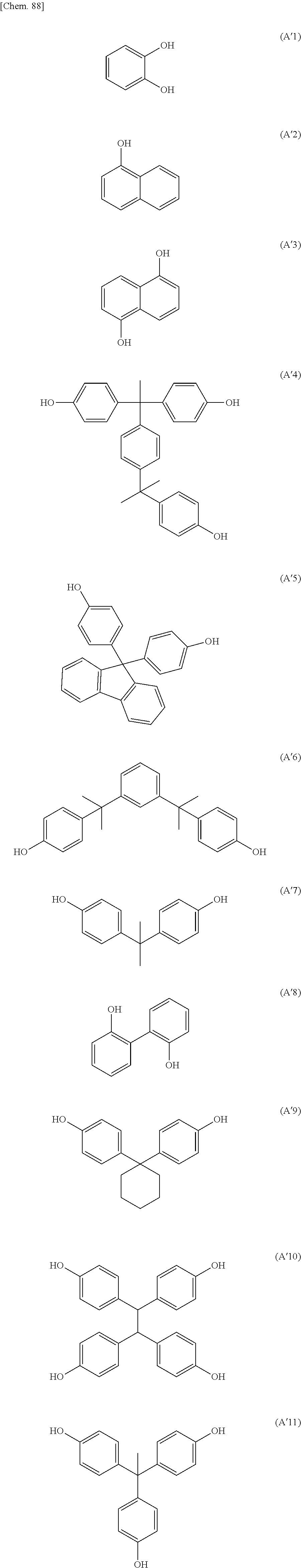

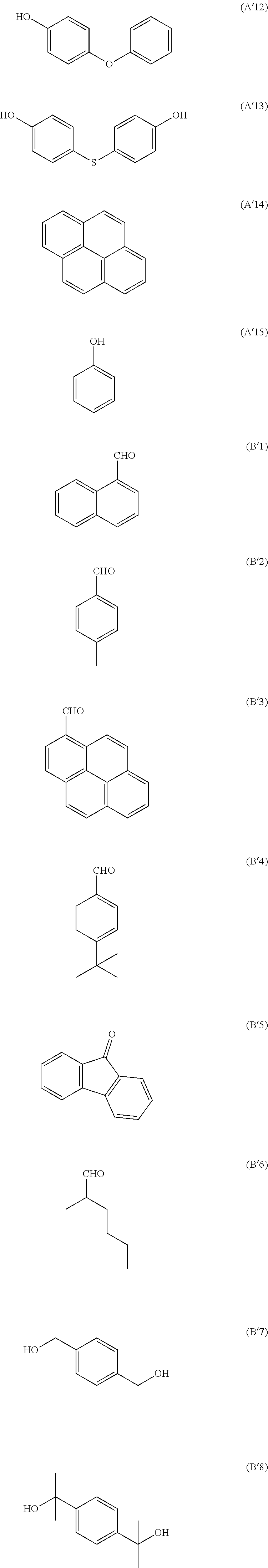

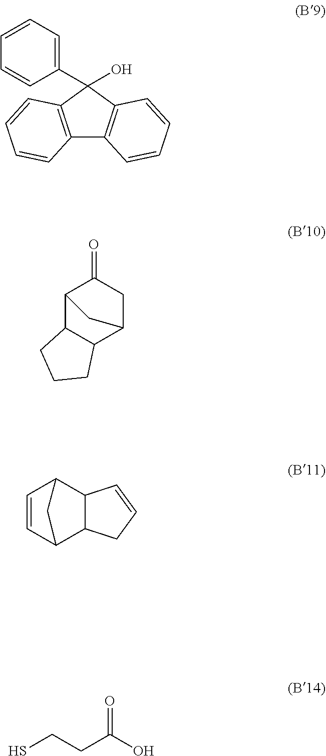 [SMILES: OC1(c2ccccc2)c2ccccc2-c2ccccc21 ; O=C1CC2CC1C1CCCC21 ; C1=CC2C3C=CC(C3)C2C1 ; O=C(O)CCS]

### (Catalysts C′)

Methanesulfonic acid: C′1
Trifluoromethanesulfonic acid: C′2

### (Solvents D′)

Propylene glycol monomethyl ether acetate: D′1
Propylene glycol monomethyl ether: D′2
   (Reprecipitation solvents E′)
Methanol/water: E′1
Methanol: E′2

### Synthesis Example 1′]

A flask was charged with 13.0 g of catechol, 18.4 g of 1-naphthaldehyde, 3.4 g of methanesulfonic acid, 24.4 g of propylene glycol monomethyl ether acetate, and 10.5 g of propylene glycol monomethyl ether. Subsequently, the mixture was allowed to react under nitrogen for about 30 hours under reflux conditions. After the reaction was discontinued, the product was reprecipitated from methanol/water mixed solvent and was dried to give a resin (S′1). The polystyrene-equivalent weight average molecular weight Mw measured by GPC was about 1,650. The resin obtained was dissolved into PGMEA, and ion exchange was performed for 4 hours using a cation exchange resin and an anion exchange resin. A target compound solution was thus obtained.

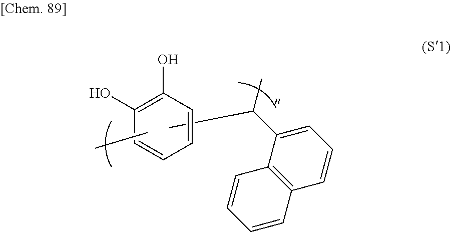

### Synthesis Examples 2′ to 11′

Polymers for use in resist underlayer films were synthesized while changing the type of the compounds A′, the compounds B′, the catalysts C′, the solvents D′, and the reprecipitation solvents E′. The experimental procedures were the same as in Synthesis Example 1′. The conditions adapted in the synthesis of polymers (S′1) to (S′11) are shown below.

TABLE 9

| | Structural | | | | | Re- |
|---|---|---|---|---|---|---|
| Syn. Ex. | formula | Compounds | Catalysts | Solvents | Temp./time | precipitation |
| 1′ | S′1 | A′1/B′1 | C′1 | D′1/D′2 | Reflux/ | E′1 |
| | | 13.0 g/18.4 g | 3.4 g | 24.4 g/ | 30 hr | |
| | | | | 10.5 g | | |
| 2′ | S′2 | A′2/B′2 | C′1 | D′1 | Reflux/ | E′2 |
| | | 10.0 g/8.3 g | 1.3 g | 19.7 g | 7 hr | |
| 3′ | S′3 | A′3/B′2 | C′1 | D′1/D′2 | Reflux/ | E′2 |
| | | 10.0 g/7.5 g | 1.2 g | 21.8 g/ | 6 hr | |
| | | | | 21.8 g | | |
| 4′ | S′4 | A′4/A′14/B′3 | C′1 | D′1/D′2 | Reflux/ | E′2 |
| | | 3.5 g/1.5 g/ | 0.8 g | 25.9 g/ | 4.75 | |
| | | 10.0 g | | 6.5 g | hr | |
| 5′ | S′5 | A′5/8′4 | C′1 | D′1 | Reflux/ | E′2 |
| | | 10.0 g/4.7 g | 0.6 g | 22.8 g | 5.5 | |
| | | | | | hr | |
| 6′ | S′6 | A′6/B′6 | C′1 | D′1 | Reflux/ | E′1 |
| | | 10.0 g/5.2 g | 0.7 g | 15.9 g | 13 hr | |
| 7′ | S′7 | A′11/A′13/ | C′1 | D′1 | Reflux/ | E′2 |
| | | B′3 | 2.1 g | 180.1 g | 18 hr | |
| | | 5.6 g/12.0 g/ | | | | |
| | | 25.5 g | | | | |
| 8′ | S′8 | A′7/A′9/B′6/B′7 | C′1 | D′1 | Reflux/ | E′1 |
| | | 6.8 g/8.0 g/ | 1.2 g | 24.0 g | 4 hr | |
| | | 4.9 g/3.1 g | | | | |
| 9′ | S′9 | A′10/A′12/ | C′1 | D′1 | Reflux/ | E′1 |
| | | B′8/B′9 | 1.0 g | 25.2 g | 4 hr | |
| | | 6.4 g/7.0 g/ | | | | |
| | | 9.4 g/1.4 g | | | | |
| 10′ | S′10 | A′15/B′11 | C′2 | — | Reflux/ | E′1 |
| | | 18.8 g/8.0 g | 0.01 g | | 2 hr | |
| 11′ | S′11 | A′5/B′10/ | C′1 | D′1 | Reflux/ | |
| | | B′14 | 2.0 g | 38.4 g | 16 hr | |
| | | 12.3 g/24.0 g/ | | | | |
| | | 2.2 g | | | | |

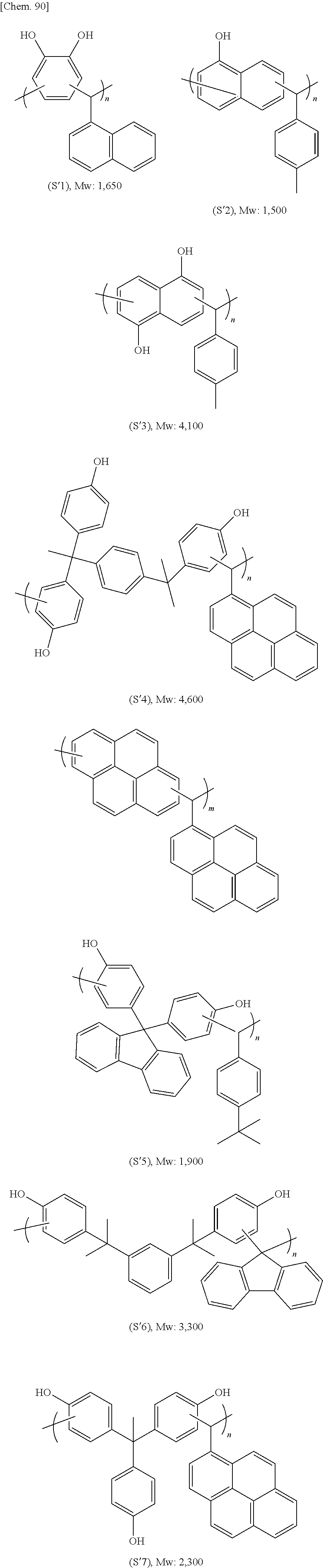

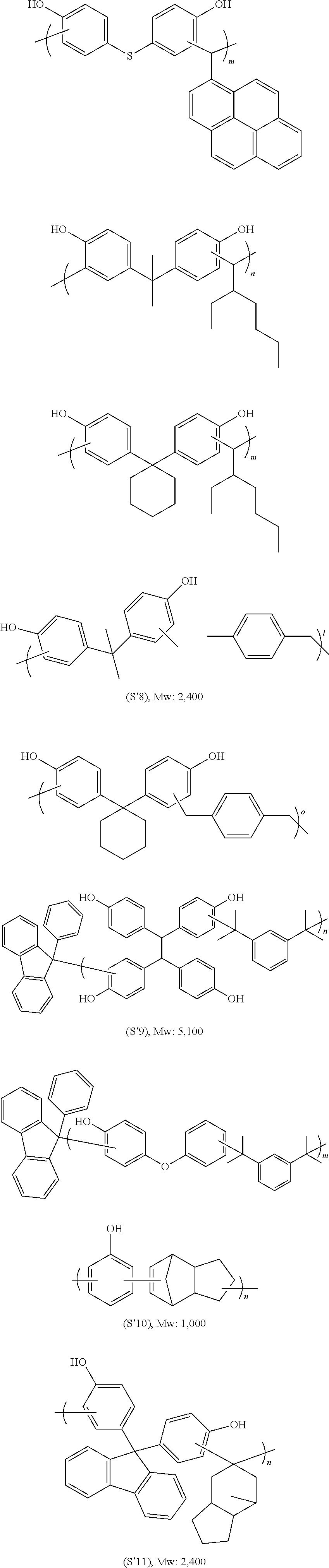

### Preparation of Resist Underlayer Film

The polymers (S′1) to (S′11), crosslinking agents (CR‘l to CR’3), the acid generators (Ad′1 to Ad′3, and S16 to S43), solvents (propylene glycol monomethyl ether acetate (PGMEA), propylene glycol monomethyl ether (PGME), cyclohexanone (CYH)), and MEGAFACE R-40 (manufactured by DIC CORPORATION, G′1) as a surfactant were mixed in proportions indicated in the tables below (the values are expressed by part by mass). The mixtures were filtered through a 0.1 m polytetrafluoroethylene microfilter. Resist underlayer film materials (M′1 to M′11 and Comparative M′1 to Comparative M′11) were thus prepared.

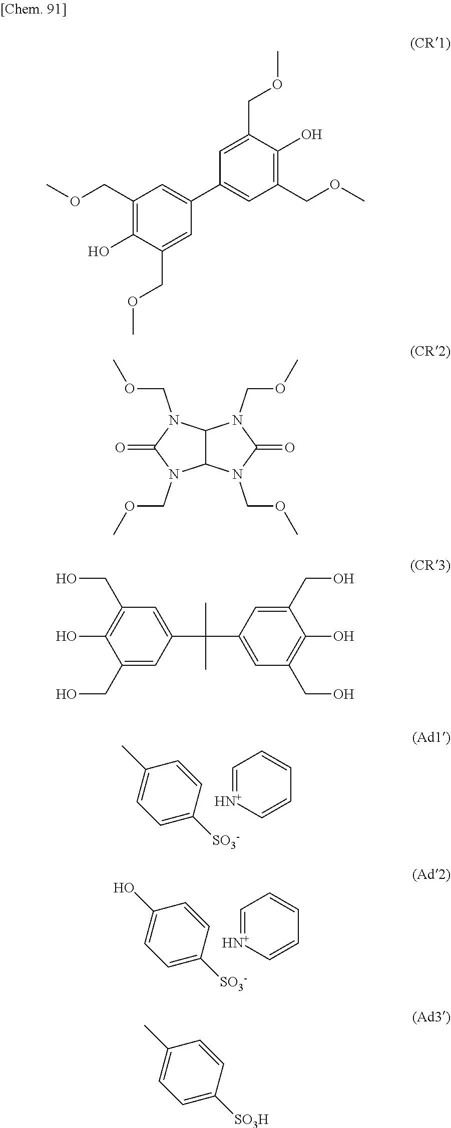

TABLE 10

| | | Crosslinking | Acid | | | | |
|---|---|---|---|---|---|---|---|
| Composition | Polymer | agent | generator | Surfactant | Solvents (100 in total) | | |
| M′1 | Syn. Ex. 1′ | CR′1 | S16 | G′1 | PGMEA | PGME | CYH |
| | 100 | 30 | 3 | 0.1 | 70 | 30 | 0 |
| M′2 | Syn. Ex. 2′ | CR′1S | G′1 | PGMEA | — | PGME | CYH |
| | | 16 | | | | | |
| | 100 | 30 | 3 | 0.1 | — | 70 | 30 |
| M′3 | Syn. Ex. 3′ | CR′3 | S27 | G′1 | PGMEA | PGME | CYH |
| | 100 | 30 | 3 | 0.1 | 30 | 70 | 0 |
| M′4 | Syn. Ex. 4′ | CR′1 | S29 | G′1 | PGMEA | PGME | CYH |
| | 100 | 30 | 3 | 0.1 | 40 | 30 | 30 |
| M′5 | Syn. Ex. 5′ | CR′1 | S32 | G′1 | PGMEA | PGME | CYH |
| | 100 | 30 | 3 | 0.1 | 70 | 30 | 0 |
| M′6 | Syn. Ex. 6′ | CR′1 | S33 | G′1 | PGMEA | PGME | CYH |
| | 100 | 30 | 3 | 0.1 | 70 | 30 | 0 |
| M′7 | Syn. Ex. 7′ | CR′2 | S34 | G′1 | PGMEA | PGME | CYH |
| | 100 | 30 | 3 | 0.1 | 40 | 30 | 30 |
| M′8 | Syn. Ex. 8′ | CR′2 | S35 | G′1 | PGMEA | PGME | CYH |
| | 100 | 30 | 3 | 0.1 | 70 | 30 | 0 |
| M′9 | Syn. Ex. 9′ | CR′1 | S37 | G′1 | PGMEA | PGME | CYH |
| | 100 | 30 | 3 | 0.1 | 70 | 30 | 0 |
| M′10 | Syn. Ex. 10′ | CR′1 | S38 | G′1 | PGMEA | PGME | CYH |
| | 100 | 30 | 3 | 0.1 | 70 | 30 | 0 |
| M′11 | Syn. Ex. 11′ | CR′1 | S40 | G1 | PGMEA | PGME | CYH |
| | 100 | 30 | 3 | 0.1 | 70 | 30 | 0 |

TABLE 11

| Comp. | Syn. Ex. 1′ | CR′1 | Ad′1 | G′1 | PGMEA | PGME | CYH |
|---|---|---|---|---|---|---|---|
| M′1 | 100 | 30 | 3 | 0.1 | 70 | 30 | 0 |
| Comp. | Syn. Ex. 2′ | CR′1 | Ad′1 | G′1 | PGMEA | PGME | CYH |
| M′2 | 100 | 30 | 3 | 0.1 | 70 | 30 | 0 |
| Comp. | Syn. Ex. 3′ | CR′3 | Ad′1 | G′1 | PGMEA | PGME | CYH |
| M′3 | 100 | 30 | 3 | 0.1 | 30 | 70 | 0 |
| Comp. | Syn. Ex. 4′ | CR′1 | Ad′2 | G′1 | PGMEA | PGME | CYH |
| M′4 | 100 | 30 | 3 | 0.1 | 40 | 30 | 30 |
| Comp. | Syn. Ex. 5′ | CR′1 | Ad′1 | G′1 | PGMEA | PGME | CYH |
| M′5 | 100 | 30 | 3 | 0.1 | 70 | 30 | 0 |
| Comp. | Syn. Ex. 6′ | CR′1 | Ad′1 | G′1 | PGMEA | PGME | CYH |
| M′6 | 100 | 30 | | 0.7 | 70 | 30 | 0 |
| Comp. | Syn. Ex. 7′ | CR′2 | Ad′1 | G′1 | PGMEA | PGME | CYH |
| M′7 | 100 | 30 | 3 | 0.1 | 40 | 30 | 30 |
| Comp. | Syn. Ex. 8′ | CR′2 | Ad′3 | G′1 | PGMEA | PGME | CYH |
| M′8 | 100 | 30 | 3 | 0.2 | 70 | 30 | 0 |
| Comp. | Syn. Ex. 9′ | CR′1 | Ad′3 | G′1 | PGMEA | PGME | CYH |
| M′9 | 100 | 30 | 3 | 0.1 | 70 | 30 | 0 |
| Comp. | Syn. Ex. 10′ | CR′1 | Ad′1 | G′1 | PGMEA | PGME | CYH |
| M′10 | 100 | 30 | 3 | 0.1 | 70 | 30 | 0 |
| Comp. | Syn. Ex. 11′ | CR′1 | Ad′1 | G′1 | PGMEA | PGME | CYH |
| M′11 | 100 | 30 | 3 | 0.1 | 70 | 30 | 0 |

[Test of Dissolution into Resist Solvent]

Each of the resist underlayer film materials prepared in Comparative Examples 1′ to 11′ and Examples 1′ to 11′ was applied onto a silicon wafer using a spin coater, and each of the resultant coatings was baked on a hot plate at 240° C. for 60 seconds to form a resist underlayer film with a film thickness of about 120 nm. The resist underlayer films formed were soaked in a general-purpose thinner, specifically, PGME/PGMEA=7/3, for 60 seconds to examine the resistance to the solvent. The evaluation was made as o when the loss in film thickness after the thinner immersion was 1% or less, and as x when the loss in film thickness was more than 1%.

TABLE 12

| Ex./Comp. Ex. | Composition | Baking temp. | Solvent resistance |
|---|---|---|---|
| Ex. 1′ | M′ 1 | 240° C./60 sec | ∘ |
| Ex. 2′ | M′ 2 | 240° C./60 sec | ∘ |
| Ex. 3′ | M′ 3 | 240° C./60 sec | ∘ |
| Ex. 4′ | M′ 4 | 240° C./60 sec | ∘ |
| Ex. 5′ | M′ 5 | 240° C./60 sec | ∘ |
| Ex. 6′ | M′ 6 | 240° C./60 sec | ∘ |
| Ex. 7′ | M′ 7 | 240° C./60 sec | ∘ |
| Ex. 8′ | M′ 8 | 240° C./60 sec | ∘ |
| Ex. 9′ | M′ 9 | 240° C./60 sec | ∘ |
| Ex. 10′ | M′ 10 | 240° C./60 sec | ∘ |
| Ex. 11′ | M′ 11 | 240° C./60 sec | ∘ |
| Comp. Ex. 1′ | Comp. M′ 1 | 240° C./60 sec | ∘ |
| Comp. Ex. 2′ | Comp. M′ 2 | 240° C./60 sec | ∘ |
| Comp. Ex. 3′ | Comp. M′ 3 | 240° C./60 sec | ∘ |
| Comp. Ex. 4′ | Comp. M′ 4 | 240° C./60 sec | ∘ |
| Comp. Ex. 5′ | Comp. M′ 5 | 240° C./60 sec | ∘ |
| Comp. Ex. 6′ | Comp. M′ 6 | 240° C./60 sec | ∘ |
| Comp. Ex. 7′ | Comp. M′ 7 | 240° C./60 sec | ∘ |
| Comp. Ex. 8′ | Comp. M′ 8 | 240° C./60 sec | ∘ |
| Comp. Ex. 9′ | Comp. M′ 9 | 240° C./60 sec | ∘ |
| Comp. Ex. 10′ | Comp. M′ 10 | 240° C./60 sec | ∘ |
| Comp. Ex. 11′ | Comp. M′ 11 | 240° C./60 sec | ∘ |

### [Evaluation of Gap-Filling Property]

Gap-filling property was evaluated using 200 nm thick SiO₂ substrate, SiN substrate, and TiN substrate, which had a dense pattern area consisting of 50 nm wide trenches at 100 nm pitches. Each of the resist underlayer film materials prepared in Comparative Examples 1′ to 11′ and Examples 1′ to 11′ was applied onto the pattern, and each of the resultant coatings was baked at 240° C. for 60 seconds to form a resist underlayer film having a thickness of about 120 nm. The flatness of the substrates was evaluated using a scanning electron microscope (5-4800) manufactured by Hitachi High-Tech Corporation, and whether the resist underlayer film-forming composition had filled the inside of the pattern was determined. The gap-filling property was rated as o when the resist underlayer film-forming composition had filled the inside of the pattern, and as x when the resist underlayer film-forming composition had failed to fill the inside of the pattern.

### [Test of Covering Performance on Non-Planar Substrates)

To test the covering performance on a non-planar substrate, each of the resist underlayer film-forming compositions prepared in Comparative Examples 1′ to 11′ and Examples 1′ to 11′ was applied to 200 nm thick SiO₂ substrate, SiN substrate, and TiN substrate, and each of the resultant coatings was baked at 240° C. for 60 seconds to form a resist underlayer film having a thickness of about 120 nm. The coating film thickness was compared between at an 800 nm trenched area (TRENCH) and at an open area (OPEN) free from patterns. The flatness of the substrates was evaluated using a scanning electron microscope (S-4800) manufactured by Hitachi High-Tech Corporation by measuring the difference in film thickness between on the trenched area (the patterned area) and on the open area (the pattern-free area) of the non-planar substrate (the step height created on the coating film between the trenched area and the open area, called the bias). Here, the flatness means how small the difference is in the film thickness (the iso-dense bias) of the coating film between on the region with the pattern (the trenched area (the patterned area)) and on the region without patterns (the open area (the pattern-free area)). Flattening property was rated as o when the bias was improved compared to Comparative Examples.

TABLE 13

| | | Film | | | |
|---|---|---|---|---|---|
| Ex./Comp. | | thick- | Sub- | Gap-filling | Flattening |
| Ex. | Composition | ness | strate | properties | properties |
| Ex. 1′ | M′ 1 | 120 nm | SiO₂ | ∘ | ∘ |
| Ex. 2′ | M′ 2 | 120 nm | SiO₂ | ∘ | ∘ |
| Ex. 3′ | M′ 3 | 120 nm | SiO₂ | ∘ | ∘ |
| Ex. 4′ | M′ 4 | 120 nm | TiN | ∘ | ∘ |
| Ex. 5′ | M′ 5 | 120 nm | SiN | ∘ | ∘ |
| Ex. 6′ | M′ 6 | 120 nm | SiO₂ | ∘ | ∘ |
| Ex. 7′ | M′ 7 | 120 nm | SiO₂ | ∘ | ∘ |
| Ex. 8′ | M′ 8 | 120 nm | SiO₂ | ∘ | ∘ |
| Ex. 9′ | M′ 9 | 120 nm | SiO₂ | ∘ | ∘ |
| Ex. 10′ | M′ 10 | 120 nm | SiO₂ | ∘ | ∘ |
| Ex. 11′ | M′ 11 | 120 nm | SiO₂ | ∘ | ∘ |
| Comp. Ex. 1′ | Comp. M′ 1 | 120 nm | SiO₂ | ∘ | x |
| Comp. Ex. 2′ | Comp. M′ 2 | 120 nm | SiO₂ | ∘ | x |
| Comp. Ex. 3′ | Comp. M′ 3 | 120 nm | SiO₂ | ∘ | x |
| Comp. Ex. 4′ | Comp. M′ 4 | 120 nm | TiN | ∘ | x |
| Comp. Ex. 5′ | Comp. M′ 5 | 120 nm | SiN | ∘ | x |
| Comp. Ex. 6′ | Comp. M′ 6 | 120 nm | SiO₂ | ∘ | x |
| Comp. Ex. 7′ | Comp. M′ 7 | 120 nm | SiO₂ | ∘ | x |
| Comp. Ex. 8′ | Comp. M′ 8 | 120 nm | SiO₂ | ∘ | x |
| Comp. Ex. 9′ | Comp. M′ 9 | 120 nm | SiO₂ | ∘ | x |
| Comp. Ex. 10′ | Comp. M′ 10 | 120 nm | SiO₂ | ∘ | x |
| Comp. Ex. 11′ | Comp. M′ 11 | 120 nm | SiO₂ | ∘ | x |

As seen in the table, the acid generators synthesized from an amine component having a higher basicity than pyridine provided a similar level of curability to the conventional pyridine salt-type acid generators. In addition, the acid generators using a highly basic amine component had a higher stability as a salt and therefore the generation of an acid component could be retarded until a higher temperature to ensure a time for the resin to flow. As a result, a flatter film could be formed on a patterned substrate. The above advantageous effects were achieved equally on different types of patterned substrates, such as SiN, SiO₂, and TiN.

### INDUSTRIAL APPLICABILITY

The underlayer film-forming composition according to the present invention involves an acid generator that includes a highly basic amine. Thus, the generation of an acid can be retarded until a high temperature to ensure a long time for the polymer to flow. As a result, a cured film can be obtained with a high flattening property and high gap-filling property on various types of substrates, such as SiO₂, TiN, and SiN. Furthermore, the composition has a high storage stability, for example, is free from coloration, and can form a film that is not dissolved by a photoresist solvent. In addition, the present invention provides a resist underlayer film obtained from the resist underlayer film-forming composition, and a resist pattern forming method and a semiconductor device manufacturing method using the resist underlayer film-forming composition.

1. A resist underlayer film-forming composition comprising:
a thermal acid generator represented by formula (I) below;
a polymer (G) which is a novolac resin comprising:
(i) a unit structure having an optionally substituted aromatic ring, and
(ii) a unit structure containing:
an optionally substituted aromatic cyclic organic group; an optionally substituted non-aromatic monocyclic organic group; or an optionally substituted, 4—to 25-membered bicyclic, tricyclic, or tetracyclic organic group containing at least one non-aromatic monocyclic ring;
the unit structure (i) and the unit structure (ii) being bonded to each other via a covalent bond between a carbon atom on the aromatic ring of the unit structure (i) and a carbon atom on a non-aromatic monocyclic ring of the unit structure (ii); and
a solvent,
[Chem. 92]
(A—SO₃)⁻(BH)⁺ (1)
[in formula (I),
A is an optionally substituted, linear, branched, or cyclic, saturated or unsaturated, aliphatic hydrocarbon group; an optionally substituted aryl group; or an optionally substituted heteroaryl group; and
B is a base having a pKa of 6.5 or more].

2. The resist underlayer film-forming composition according to claim 1, wherein
the polymer (G) includes a structure represented by formula (X) below: 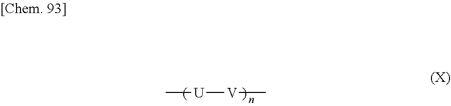
[in formula (X), n indicates a number of composite unit structures U-V,
the unit structure U is one, or two or more types of unit structures having an optionally substituted aromatic ring, and is optionally such that:
the optionally substituted aromatic ring is substituted with a substituent that contains a heteroatom;
the unit structure contains aromatic rings connected to one another by a linking group that contains a heteroatom; or
the aromatic ring is an aromatic heterocyclic ring or is an aromatic ring forming a condensed ring with one or more heterocyclic rings; and
the unit structure V comprises one, or two or more types of unit structures containing at least one structure selected from: 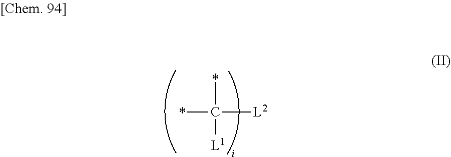
(in formula (II),
* indicates a bonding site to the unit structure U;
L¹ is:
an optionally substituted, saturated or unsaturated, linear, branched, or cyclic, aliphatic hydrocarbon group optionally containing a heteroatom;
an optionally substituted aromatic hydrocarbon group optionally containing a heteroatom;
a group comprising a combination or a condensation product of the above groups; or
a hydrogen atom;
L² is:
an optionally substituted, saturated or unsaturated, linear, branched, or cyclic, aliphatic hydrocarbon group optionally containing a heteroatom;
an optionally substituted aromatic hydrocarbon group optionally containing a heteroatom;
a group comprising a combination or a condensation product of the above groups;
a direct bond; or
a hydrogen atom;
L¹ and L² are optionally condensed to each other or are optionally bonded to each other via or without a heteroatom to form a ring;
i is an integer of 1 or more and 8 or less;
when i is 2 or more, L² is not a hydrogen atom; and
when i is 2 or more, the two to i quantity of carbon atoms are optionally connected to one another by the aliphatic hydrocarbon group or the aromatic hydrocarbon group L) 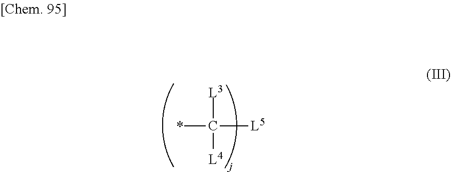
* indicates a bonding site to the unit structure U;
L³ is:
an optionally substituted, saturated or unsaturated, linear, branched, or cyclic, aliphatic hydrocarbon group optionally containing a heteroatom;
an optionally substituted aromatic hydrocarbon group optionally containing a heteroatom;
a group comprising a combination or a condensation product of the above groups;
a hydroxy group; or
a hydrogen atom;
L⁴ is:
an optionally substituted, saturated or unsaturated, linear, branched, or cyclic, aliphatic hydrocarbon group optionally containing a heteroatom;
an optionally substituted aromatic hydrocarbon group optionally containing a heteroatom;
a group comprising a combination or a condensation product of the above groups;
a hydroxy group; or
a hydrogen atom;
L⁵ is:
an optionally substituted, saturated or unsaturated, linear, branched, or cyclic, aliphatic hydrocarbon group optionally containing a heteroatom;
an optionally substituted aromatic hydrocarbon group optionally containing a heteroatom;
a group comprising a combination or a condensation product of the above groups; or
a direct bond;
j is an integer of 2 or more and 4 or less; and
L³, L⁴, and L⁵ are optionally condensed to one another or are optionally bonded to one another via or without a heteroatom to form a ring); and 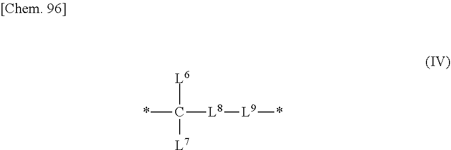
(in formula (IV),
* indicates a bonding site to the unit structure U;
L⁶ is:
an optionally substituted, saturated or unsaturated, linear, branched, or cyclic, aliphatic hydrocarbon group optionally containing a heteroatom;
an optionally substituted aromatic hydrocarbon group optionally containing a heteroatom;
a group comprising a combination or a condensation product of the above groups; or
a hydrogen atom;
L⁷ is:
an optionally substituted, saturated or unsaturated, linear, branched, or cyclic, aliphatic hydrocarbon group optionally containing a heteroatom;
an optionally substituted aromatic hydrocarbon group optionally containing a heteroatom;
a group comprising a combination or a condensation product of the above groups; or
a hydrogen atom;
L⁶, L⁷, and L⁹ are optionally condensed to one another or are optionally bonded to one another via or without a heteroatom to form a ring;
L⁸ is:
a direct bond;
an optionally substituted, saturated or unsaturated, linear or branched hydrocarbon group; or
an aromatic ring optionally containing a heteroatom; and
L⁹ is:
an aromatic ring optionally containing a heteroatom)].

3. The resist underlayer film-forming composition according to claim 1, wherein
the polymer (G) comprises:
a structural unit derived from (D) an aromatic compound having at least one hydroxy group or amino group, or a compound in which two or more optionally substituted aromatic rings are connected to one another via at least one direct bond, —O—, —S—, —C(═O)—, —SO₂—, —NR—, in which R denotes a hydrogen atom or a hydrocarbon group, or —(CR₁₁₁R₁₁₂)ₙ—, in which R₁₁₁ and R₁₁₂ each denote a hydrogen atom, an optionally substituted, C1-C10 linear or cyclic alkyl group, or an aromatic ring, n is 1 to 10, and R₁₁₁ and R₁₁₂ are optionally bonded to each other to form a ring, and (E) an optionally substituted aldehyde compound or aldehyde equivalent.

4. The resist underlayer film-forming composition according to claim 1, wherein
B in formula (I) is R R²R³N,
R¹ and R² each independently denote a hydrogen atom or an optionally substituted, linear or branched, saturated or unsaturated, aliphatic hydrocarbon group,
R¹ and R² optionally form a ring together via or without a heteroatom, or optionally form a ring together via an aromatic ring,
R³ denotes a hydrogen atom, an optionally substituted aromatic group, or an optionally substituted, linear or branched, saturated or unsaturated, aliphatic hydrocarbon group, and
when R and R² do not form a ring, R³ is a hydrogen atom or an optionally substituted aromatic group.

5. The resist underlayer film-forming composition according to claim 1, wherein
B in formula (I) is a base represented by:
R¹R²R³N [Chem. 97]
[wherein
R and R² each independently denote an optionally substituted, linear or branched, saturated or unsaturated, aliphatic hydrocarbon group, and
R³ denotes a hydrogen atom or an optionally substituted aromatic group] or
a base represented by formula (II) below: 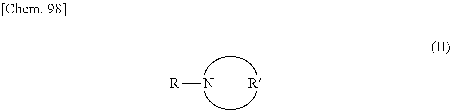
[in formula (II),
R is a hydrogen atom, a nitro group, a cyano group, an amino group, a carboxyl group, a hydroxy group, an amide group, an aldehyde group, a (meth)acryloyl group, a halogen atom, a C1-C10 alkoxy group, a C1-C10 alkyl group, a C2-C10 alkenyl group, a C2-C10 alkynyl group, a C1-C10 hydroxyalkyl group, a C6-C40 aryl group, an ether bond-containing organic group, a ketone bond-containing organic group, an ester bond-containing organic group, or a combination thereof, and
R′ is a ring through an aromatic ring or is:
(R^{a})ₙ—X—(R^{b})ₘ— [Chem. 99]
wherein R^{a} and R^{b} each independently denote an optionally substituted alkyl,
X is O, S, SO₂, CO, CONH, COO, or NH, and
n and m are each independently 2, 3, 4, 5, or 6].

6. The resist underlayer film-forming composition according to claim 5, wherein the base is such that:
R³ in the formula denotes an optionally substituted phenyl, naphthyl, anthracenyl, pyrenyl, or phenanthrenyl group, or
R in formula (II) is a hydrogen atom, a methyl group, an ethyl group, an isobutyl group, an allyl group, or a cyanomethyl group, and
R′ in formula (II) is represented by:
—(CH₂)ₙ—O—(CH₂)ₘ— [Chem. 100]

7. The resist underlayer film-forming composition according to claim 1, wherein B in formula (I) is N-methylmorpholine, N-isobutylmorpholine, N-allylmorpholine, or N,N-diethylaniline.

8. The resist underlayer film-forming composition according to claim 1, wherein A in formula (I) is a methyl group, a fluoromethyl group, a naphthyl group, a norbornanylmethyl group, a dimethylphenyl group, or a tolyl group.

9. The resist underlayer film-forming composition according to claim 3, wherein the compound (D) is selected from the following group:

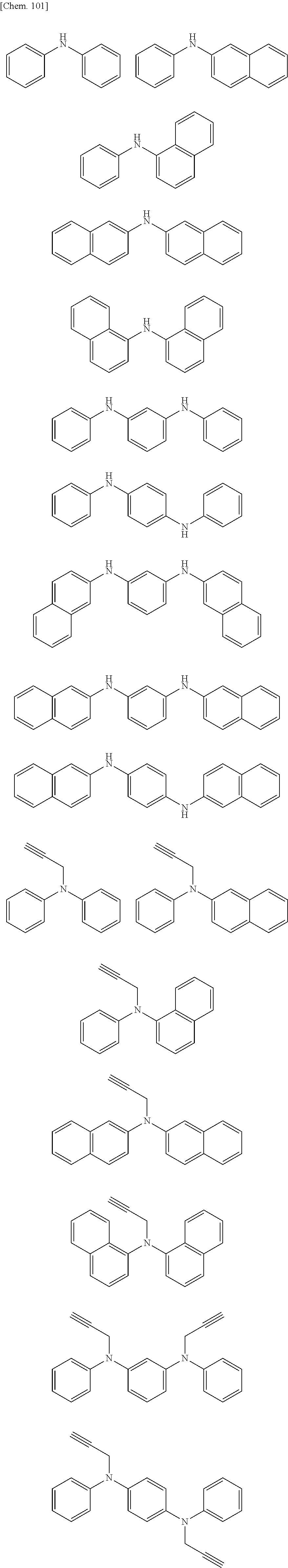

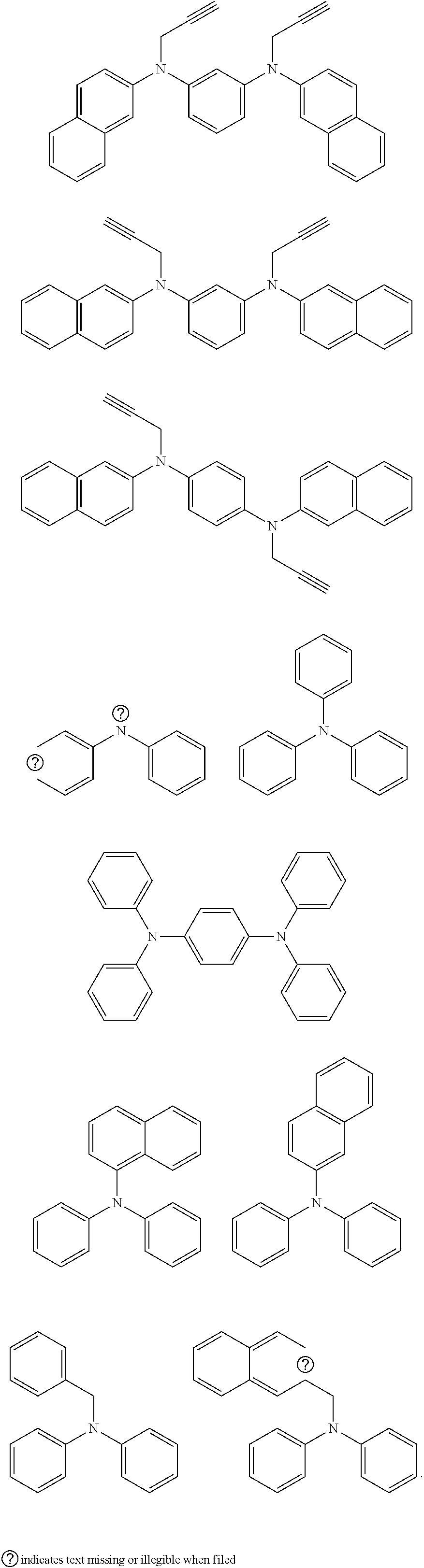

10. The resist underlayer film-forming composition according to claim 3, wherein the compound (D) is selected from the following group:

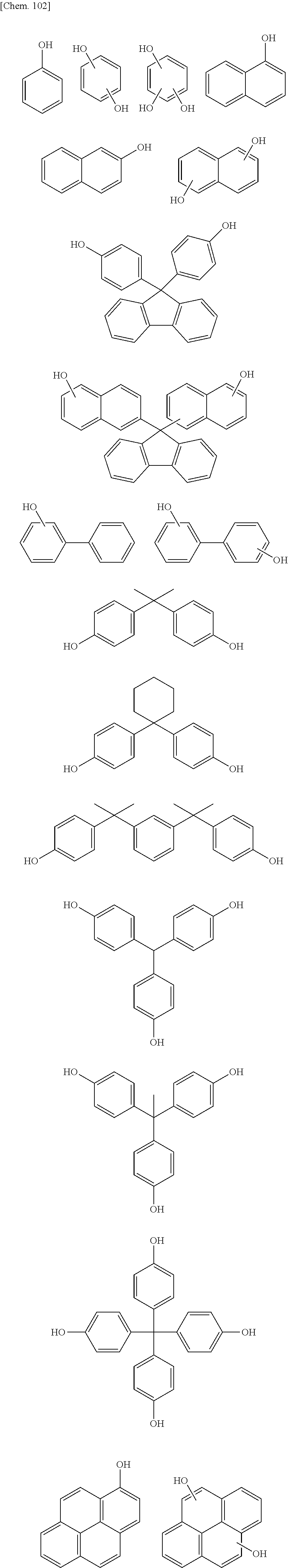

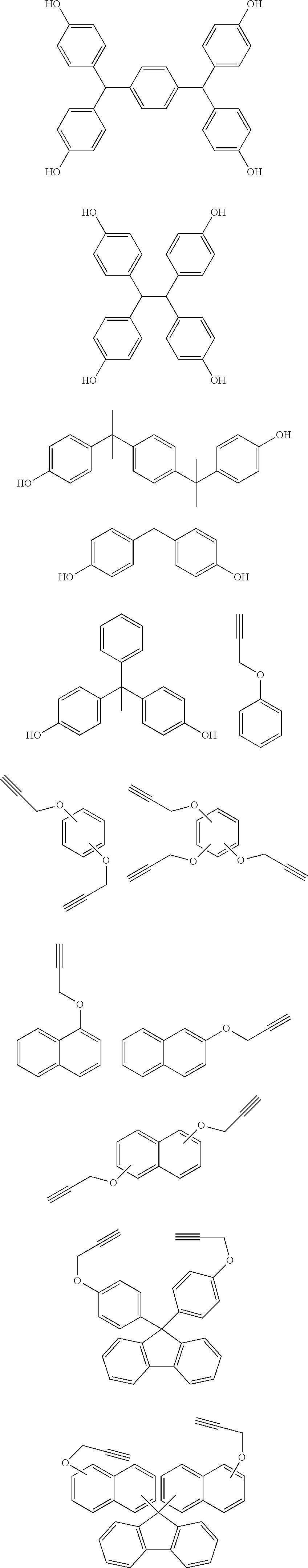

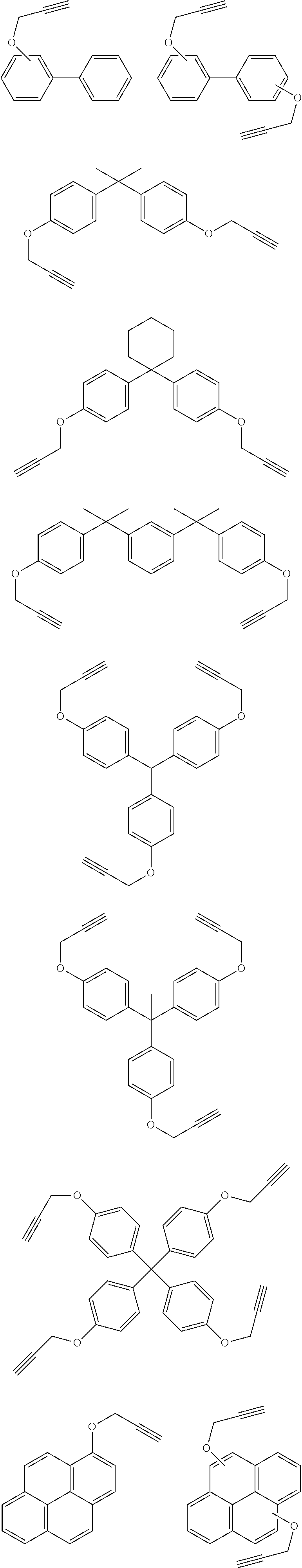

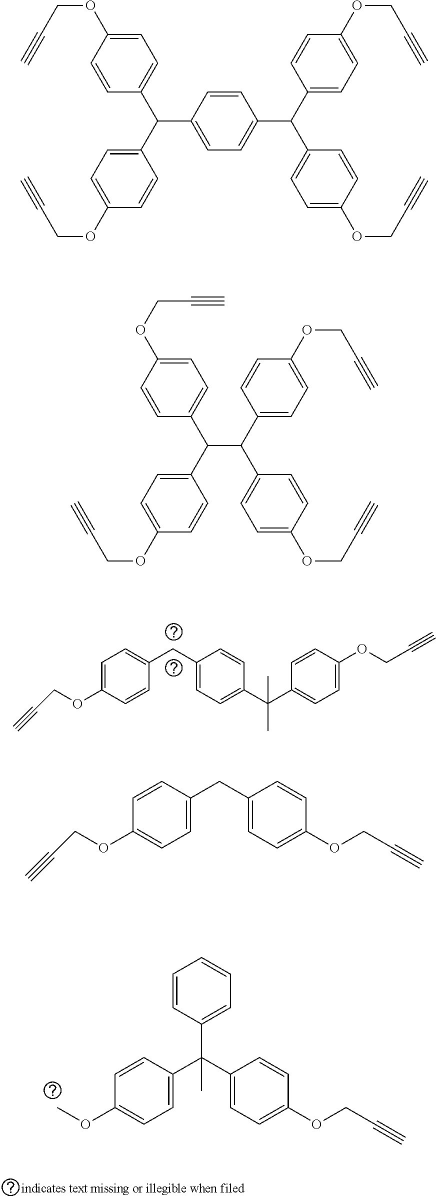

11. The resist underlayer film-forming composition according to claim 3, wherein the aldehyde compound or aldehyde equivalent (E) is selected from the following group:

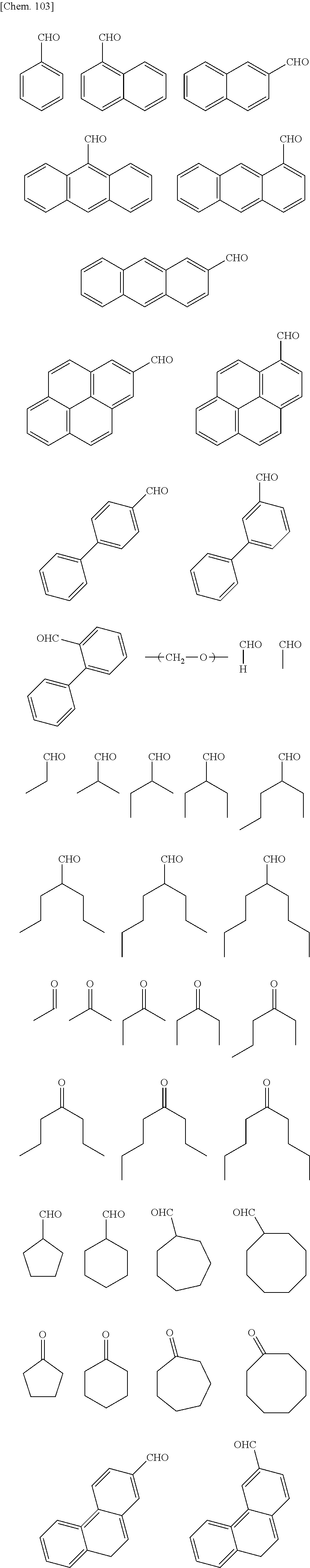

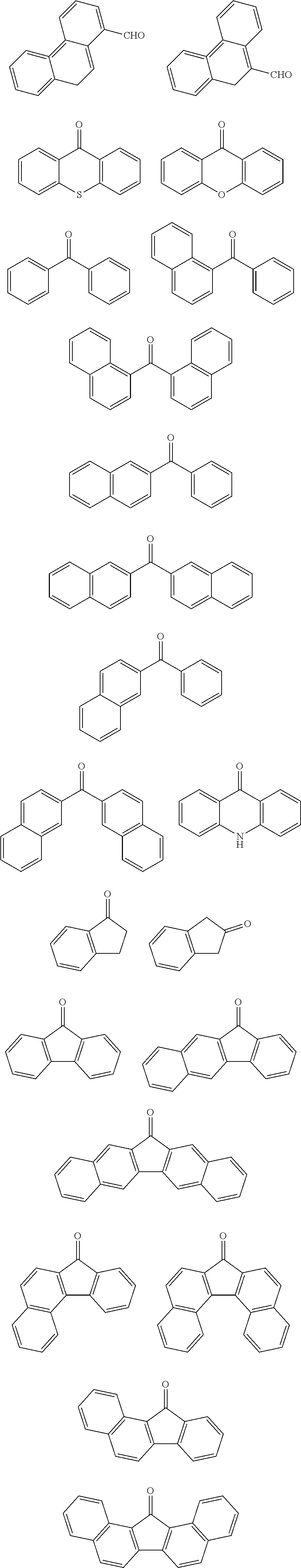

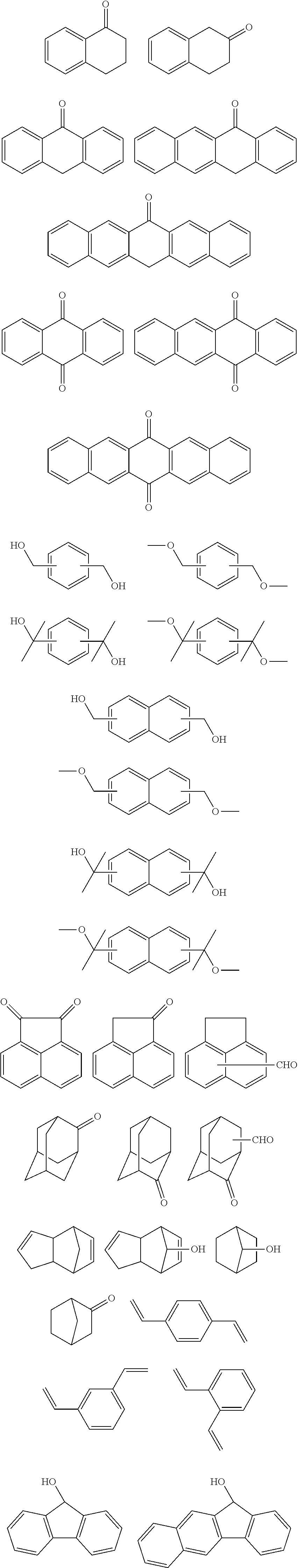

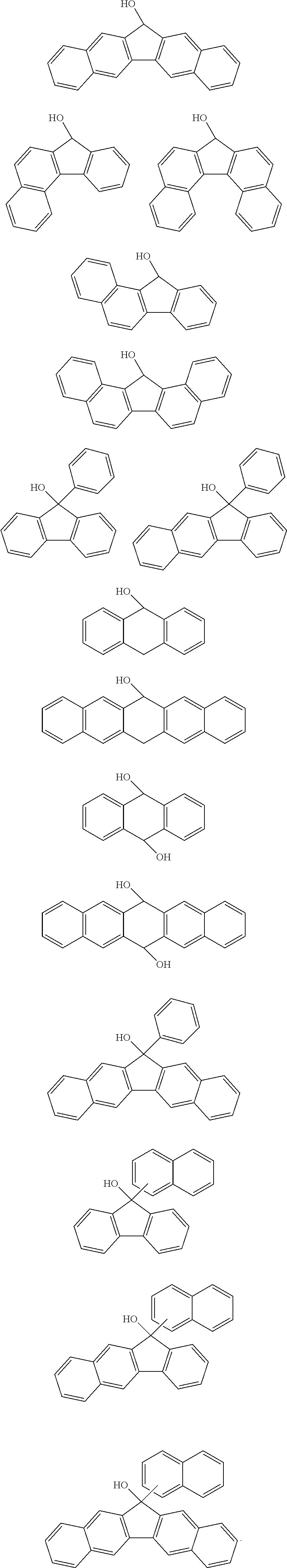

12. The resist underlayer film-forming composition according to claim 1, further comprising a crosslinking agent.

13. The resist underlayer film-forming composition according to claim 12, wherein the crosslinking agent is an aminoplast crosslinking agent or a phenoplast crosslinking agent.

14. The resist underlayer film-forming composition according to claim 13, wherein the aminoplast crosslinking agent is a highly alkylated, alkoxylated, or alkoxyalkylated melamine, benzoguanamine, glycoluril, or urea, or a polymer thereof.

15. The resist underlayer film-forming composition according to claim 13, wherein the phenoplast crosslinking agent is a highly alkylated, alkoxylated, or alkoxyalkylated aromatic, or a polymer thereof.

16. The resist underlayer film-forming composition according to claim 1, further comprising a compound having an alcoholic hydroxy group or a compound having a group capable of forming an alcoholic hydroxy group.

17. The resist underlayer film-forming composition according to claim 16, wherein the compound having an alcoholic hydroxy group or the compound having a group capable of forming an alcoholic hydroxy group is a propylene glycol solvent, a cycloaliphatic ketone solvent, an oxyisobutyric acid ester solvent, or a butylene glycol solvent.

18. The resist underlayer film-forming composition according to claim 16, wherein the compound having an alcoholic hydroxy group or the compound having a group capable of forming an alcoholic hydroxy group is propylene glycol monomethyl ether, propylene glycol monomethyl acetate, cyclohexanone, or methyl 2-hydroxy-2-methylpropionate.

19. The resist underlayer film-forming composition according to claim 1, further comprising a surfactant.

20. A resist underlayer film, which is a baked product of a coating film of the resist underlayer film-forming composition according to claim 1 on a semiconductor substrate.

21. A method for forming a resist pattern used in semiconductor manufacturing, the method comprising the step of applying the resist underlayer film-forming composition according to claim 1 onto a semiconductor substrate, and baking the resist underlayer film-forming composition to form a resist underlayer film.

22. A method for manufacturing a semiconductor device, comprising the steps of:
forming on a semiconductor substrate a resist underlayer film from the resist underlayer film-forming composition according to claim 1;
forming a resist film on the resist underlayer film;
forming a resist pattern by irradiation with a light or electron beam followed by development;
etching the resist underlayer film through the resist pattern; and
processing the semiconductor substrate through the patterned resist underlayer film.

23. A method for manufacturing a semiconductor device, comprising the steps of:
forming on a semiconductor substrate a resist underlayer film from the resist underlayer film-forming composition according to claim 1;
forming a hard mask on the resist underlayer film;
forming a resist film on the hard mask;
forming a resist pattern by irradiation with a light or electron beam followed by development;
etching the hard mask through the resist pattern;
etching the resist underlayer film through the patterned hard mask; and
processing the semiconductor substrate through the patterned resist underlayer film.

24. A method for manufacturing a semiconductor device, comprising the steps of:
forming on a semiconductor substrate a resist underlayer film from the resist underlayer film-forming composition according to claim 1;
forming a hard mask on the resist underlayer film;
forming a resist film on the hard mask;
forming a resist pattern by irradiation with a light or electron beam followed by development;
etching the hard mask through the resist pattern;
etching the resist underlayer film through the patterned hard mask;
removing the hard mask; and
processing the semiconductor substrate through the patterned resist underlayer film.

25. A method for manufacturing a semiconductor device, comprising the steps of:
forming on a semiconductor substrate a resist underlayer film from the resist underlayer film-forming composition according to claim 1;
forming a hard mask on the resist underlayer film;
forming a resist film on the hard mask;
forming a resist pattern by irradiation with a light or electron beam followed by development;
etching the hard mask through the resist pattern formed;
etching the resist underlayer film through the patterned hard mask;
removing the hard mask;
forming a deposited film or spacer on the hard mask-removed, resist underlayer film;
processing the deposited film or spacer by etching;
removing the patterned resist underlayer film to leave the patterned deposited film or spacer; and
processing the semiconductor substrate through the patterned deposited film or spacer.

26. The manufacturing method according to claim 23, wherein the hard mask is formed by applying a composition containing an inorganic substance or by depositing an inorganic substance.

27. The manufacturing method according to claim 24, wherein the hard mask is formed by applying a composition containing an inorganic substance or by depositing an inorganic substance.

28. The manufacturing method according to claim 25, wherein the hard mask is formed by applying a composition containing an inorganic substance or by depositing an inorganic substance.

29. The manufacturing method according to claim 23, wherein
the resist film is patterned by a nanoimprinting method or by using a self-assembled film.

30. The manufacturing method according to claim 24, wherein
the resist film is patterned by a nanoimprinting method or by using a self-assembled film.

31. The manufacturing method according to claim 25, wherein
the resist film is patterned by a nanoimprinting method or by using a self-assembled film.

32. The manufacturing method according to claim 23, wherein the hard mask is removed by etching or with an alkaline chemical solution.

33. The manufacturing method according to claim 24, wherein the hard mask is removed by etching or with an alkaline chemical solution.

34. The manufacturing method according to claim 25, wherein the hard mask is removed by etching or with an alkaline chemical solution.
